(19) United States
(12) Patent Application Publication
(10) Pub. No.: US 2020/0255524 A1
(43) Pub. Date: Aug. 13, 2020

(54) COMBINATION THERAPY

(71) Applicant: MacroGenics, Inc., Rockville, MD (US)

(72) Inventors: Ezio Bonvini, Potomac, MD (US); Scott Koenig, Rockville, MD (US); Leslie S. Johnson, Damestown, MD (US); Paul A. Moore, Potomac, MD (US); Ralph F. Alderson, North Potomac, MD (US); Jon Marc Wigginton, Rockville, MD (US)

(21) Appl. No.: 16/306,882

(22) PCT Filed: Jun. 6, 2017
(86) PCT No.: PCT/US2017/036075

§ 371 Date: Dec. 3, 2018

Related U.S. Application Data
(60) Provisional application No. 62/346,854, filed on Jun. 7, 2016.
(60) Provisional application No. 62/432,299, filed on Dec. 9, 2016.

(51) Int. Cl. C07K 16/28; C07K 16/30; A61P 35/00

(52) U.S. Cl. CPC C07K 16/2818; C07K 16/2878; C07K 16/2827; C07K 16/2866; C07K 16/30; C07K 16/2806; A61K 2039/507; C07K 16/2815; C07K 16/283; A61P 35/00; C07K 2317/626; C07K 2317/622; C07K 2317/31; C07K 16/2809

(57) ABSTRACT

The present invention is directed to a combination therapy for the treatment of cancer and pathogen-associated diseases, that comprises the administration of: (I) a molecule (e.g., a diabody, an scFv, an antibody, a TandAb, etc.) capable of binding PD-I or a natural ligand of PD-I, and (2) a molecule (e.g., a diabody, a BiTe, a bispecific antibody, a CAR, etc.) capable of mediating the redirected killing of a target cell (e.g., a cancer cell or a pathogeninfected cell, etc.) expressing a Disease Antigen. The invention particularly concerns the embodiment in which the molecule capable of mediating the redirected killing of the target cell is a bispecific binding molecule that comprises a first epitope-binding site capable of immuno specifically binding an epitope of a cell surface molecule of an effector cell and a second epitope-binding site that is capable of immuno specifically binding an epitope of such target cells.

### CROSS-REFERENCE TO RELATED APPLICATIONS

This application claims priority to U.S. Patent Application Ser. Nos. 62/346,854 (filed on Jun. 7, 2016; pending) and 62/432,299 (filed on Dec. 9, 2016; pending), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0142PCT_ST25.txt, created on May 31, 2017, and having a size of 225,335 bytes), which file is herein incorporated by reference in its entirety.

### FIELD OF THE INVENTION

The present invention is directed to a combination therapy for the treatment of cancer and pathogen-associated diseases, that comprises the administration of: (1) a molecule (e.g., a diabody, an scFv, an antibody, a TandAb, etc.) capable of binding PD-1 or a natural ligand of PD-1, and (2) a molecule (e.g., a diabody, a BiTe, a bispecific antibody, a CAR, etc.) capable of mediating the redirected killing of a target cell (e.g., a cancer cell or a pathogen-infected cell, etc.) expressing a Disease Antigen. The invention particularly concerns the embodiment in which the molecule capable of mediating the redirected killing of the target cell is a bispecific binding molecule that comprises a first epitope-binding site capable of immunospecifically binding an epitope of a cell surface molecule of an effector cell and a second epitope-binding site that is capable of immunospecifically binding an epitope of such target cells (i.e., a Disease Antigen such as a Cancer Antigen or a Pathogen-Associated Antigen). The present invention is also directed to pharmaceutical compositions that comprise such molecule(s).

### BACKGROUND OF THE INVENTION

### I. The Mammalian Immune System

The mammalian immune system serves as a defense against a variety of conditions, including, e.g., injury, infection and neoplasia. The efficiency with which humans and other mammals develop an immunological response to pathogens, foreign substances and cancer antigens rests on two characteristics: the exquisite specificity of the immune response for antigen recognition, and the immunological memory that allows for faster and more vigorous responses upon re-activation with the same antigen (Portoles, P. et al. (2009) “The TCR/CD3 Complex: Opening the Gate to Successful Vaccination,” Current Pharmaceutical Design 15:3290-3300; Guy, C. S. et al. (2009) “Organization of Proximal Signal Initiation at the TCR:CD3 Complex,” Immunol Rev. 232(1):7-21; Topalian, S. L. et al. (2015) “Immune Checkpoint Blockade: A Common Denominator Approach to Cancer Therapy,” Cancer Cell 27:450-461).

In healthy individuals, the immune system is in a quiescent state, inhibited by a repertoire of diverse inhibitory receptors and receptor ligands. Upon recognition of a cancer antigen, microbial pathogen, or an allergen, an array of activating receptors and receptor ligands are triggered to induce the activation of the immune system. Such activation leads to the activation of macrophages, Natural Killer (NK) cells and antigen-specific, cytotoxic, T-cells, and promotes the release of various cytokines, all of which act to counter the perceived threat to the health of the subject (Dong, C. et al. (2003) “Immune Regulation by Novel Costimulatory Molecules,” Immunolog. Res. 28(1):39-48; Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). The immune system is capable of returning to its normal quiescent state when the countervailing inhibitory immune signals outweigh the activating immune signals.

Thus, the disease state of cancer (and indeed the disease states of infectious diseases) may be considered to reflect a failure to adequately activate a subject's immune system. Such failure may reflect an inadequate presentation of activating immune signals, or it may reflect an inadequate ability to alleviate inhibitory immune signals in the subject. In some instances, researchers have determined that cancer cells can co-opt the immune system to evade being detected by the immune system (Topalian, S. L. et al. (2015) “Immune Checkpoint Blockade: A Common Denominator Approach to Cancer Therapy,” Cancer Cell 27:450-461).

The mammalian immune system is mediated by two separate but interrelated systems: the humoral immune system and the cellular immune system. Generally speaking, the humoral system is mediated by soluble molecules (antibodies or immunoglobulins) produced by B Cells. Such molecules have the ability to combine with and neutralize antigens that have been recognized as being foreign to the body. The cellular immune system involves the mobilization of certain cells, termed “T Cells,” that serve a variety of therapeutic roles. T Cells are lymphocytes that mature in the thymus and circulate between the tissues, lymphatic system and the circulatory system. In response to the presence and recognition of foreign structures (antigens), T Cells become “activated” to initiate an immune response. In many instances, these foreign antigens are expressed on host cells as a result of neoplasia or infection. Although T Cells do not themselves secrete antibodies, they are usually required for antibody secretion by the second class of lymphocytes, B Cells (which derive from bone marrow). Critically, T Cells exhibit extraordinary immunological specificity so as to be capable of discerning one antigen from another).

Two interactions are required for T Cell activation (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). In the first interaction, a cell must display the relevant target antigen bound to a cell's Class I or Class II Major Histocompatibility Complex (“MHC”) so that it can bind the T Cell Receptor (“TCR”) of a naïve T lymphocyte. Although almost all cell types can serve as antigen-presenting cells, some cells, such as macrophages, B cells, and dendritic cells, specialize in presenting foreign antigens and are “professional” “Antigen-Presenting Cells.” Immunologic detection of antigen bound to an Antigen-Presenting Cell's MEC I molecules leads to the production of cytotoxic T Cells. Immunologic detection of antigen bound to an Antigen-Presenting Cell's MHC II molecules leads to the production of cytotoxic T Cells. In the second interaction, a ligand of the Antigen-Presenting Cell must bind a co-receptor of the T Cell (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). T Cells experiencing both stimulatory signals are then capable of responding to cytokines (such as Interleukin-2 and Interleukin-12).

In the absence of both co-stimulatory signals during TCR engagement, T Cells enter a functionally unresponsive state, referred to as clonal anergy (Khawli, L. A. et al. (2008) “Cytokine, Chemokine, and Co-Stimulatory Fusion Proteins for the Immunotherapy of Solid Tumors,” Exp. Pharmacol. 181:291-328). In pathologic states, T Cells are the key players of various organ-specific autoimmune diseases, such as type I diabetes, rheumatoid arthritis, and multiple sclerosis (Dong, C. et al. (2003) “Immune Regulation by Novel Costimulatory Molecules,” Immunolog. Res. 28(1):39-48).

This immune “checkpoint” pathway is important in maintaining self-tolerance (i.e., in preventing a subject from mounting an immune system attack against his/her own cells (an “autoimmune” reaction) and in limiting collateral tissue damage during anti-microbial or anti-allergic immune responses. Where contact of a T Cell results in the generation of only one of two required signals, the T Cell does not become activated and an adaptive immune response does not occur. The “two signal” mechanism of T Cell activation thus provides a way for the immune system to avoid undesired responses, such as responses to self-antigens that would otherwise result in an immune system attack against a subject's own cells (an “autoimmune” reaction).

### II. Cell Surface Molecules of the Cellular Immune System

A. CD3, CD4 and CD8

The cells of the immune system are characterized by their expression of specialized glycoprotein cell surface molecules. Interactions between such molecules and molecules of other cells triggers, maintains or dampens the immune response. In particular, all T Cells are characterized by their expression of CD3. CD3 is a T cell co-receptor composed of four distinct chains (Wucherpfennig, K. W. et al. (2010) “Structural Biology Of The T-Cell Receptor: Insights into Receptor Assembly, Ligand Recognition, And Initiation of Signaling,” Cold Spring Harb. Perspect. Biol. 2(4):a005140; pages 1-14; Chetty, R. et al. (1994) “CD3: Structure, Function, And Role Of Immunostaining In Clinical Practice,” J. Pathol. 173(4):303-307; Guy, C. S. et al. (2009) “Organization Of Proximal Signal Initiation At The TCR: CD3 Complex,” Immunol. Rev. 232(1):7-21).

In mammals, the complex contains a CD3γ chain, a CD3δ chain, and two CD3ε chains. These chains associate with the TCR in order to generate an activation signal in T lymphocytes (Smith-Garvin, J. E. et al. (2009) “T Cell Activation,” Annu. Rev. Immunol. 27:591-619). In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3_{ε:γ} Heterodimer,” Cell 105(7):913-923; Kuhns, M. S. et al. (2006) “Deconstructing The Form And Function Of The TCR/CD3 Complex,” Immunity. 2006 February; 24(2):133-139).

The invariant CDRε signaling component of the TCR complex on T cells, has been used as a target to force the formation of an immunological synapse between T cells and cancer cells. Co-engagement of CD3 and the tumor antigen activates the T cells, triggering lysis of cancer cells expressing the tumor antigen (Baeuerle et al. (2011) “Bispecific T Cell Engager For Cancer Therapy,” In: BISPECIFIC ANTIBODIES, Kontermann, R. E. (Ed.) Springer-Verlag; 2011:273-287). This approach allows bispecific antibodies to interact globally with the T cell compartment with high specificity for cancer cells and is widely applicable to a broad array of cell-surface tumor antigens and has also been implemented to target pathogen-infected cells (see, e.g., Sloan et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog 11(11): e1005233. doi:10.1371/journal.ppat.1005233; WO 2014/159940; and WO 2016/054101).

A first subset of T Cells, known as “helper T cells,” is characterized by the expression of the CD4 (i.e., they are “CD4⁺”). CD4⁺ T Cells are the essential organizers of most mammalian immune and autoimmune responses (Dong, C. et al. (2003) “Immune Regulation by Novel Costimulatory Molecules,” Immunolog. Res. 28(1):39-48). The activation of CD4⁺ T Cells has been found to be mediated through co-stimulatory interactions between an antigen:major histocompability class II (MHC II) molecule complex that is arrayed on the surface of an Antigen-Presenting Cell (such as a B Cell, a macrophage or a dendritic cell) and a complex of two molecules, the TCR and a CD3 cell-surface receptor ligand, both of which are arrayed on the surface of a naïve CD4⁺ T Cell. Activated T helper cells are capable of proliferating into Th1 cells that are capable of mediating an inflammatory response to the target cell.

A second subset of T Cells, known as “cytotoxic T Cells,” are characterized by the expression of CD8 (i.e., they are “CD8+” as well as CD3⁺). CD8 is a T-cell co-receptor composed of two distinct chains (Leahy, D. J. (1995) “A Structural View of CD4 and CD8,” FASEB J. 9:17-25) that is expressed on Cytotoxic T-cells. The activation of CD8⁺ T Cells has been found to be mediated through co-stimulatory interactions between an antigen:major histocompability class I (MHC I) molecule complex that is arrayed on the surface of a target cell and a complex of CD8 and the T Cell Receptor, that are arrayed on surface of the CD8⁺ T Cell ((Gao, G. et al. (2000) “Molecular Interactions Of Coreceptor CD8 And MHC Class 1: The Molecular Basis For Functional Coordination With The T-Cell Receptor,” Immunol. Today 21:630-636). Unlike major histocompability class II (MHC II) molecules, which are expressed by only certain immune system cells, MHC I molecules are very widely expressed. Thus, cytotoxic T Cells are capable of binding a wide variety of cell types. Activated cytotoxic T Cells mediate cell killing through their release of the cytotoxins perforin, granzymes, and granulysin. Through the action of perforin, granzymes enter the cytoplasm of the target cell and their serine protease function triggers the caspase cascade, which is a series of cysteine proteases that eventually lead to apoptosis (programmed cell death) of targeted cells.

B. CD2

CD2 is a cell adhesion molecule found on the surface of T-cells and natural killer (NK) cells. CD2 enhances NK cell cytotoxicity, possibly as a promoter of NK cell nanotube formation (Mace, E. M. et al. (2014) “Cell Biological Steps and Checkpoints in Accessing NK Cell Cytotoxicity,” Immunol. Cell. Biol. 92(3):245-255; Comerci, C. J. et al. (2012) “CD2 Promotes Human Natural Killer Cell Membrane Nanotube Formation,” PLoS One 7(10):e47664:1-12).

C. The T Cell Receptor (“TCR”)

The T Cell Receptor (“TCR”) is natively expressed by CD4+ or CD8+ T cells, and permits such cells to recognize antigenic peptides that are bound and presented by class I or class II MHC proteins of antigen-presenting cells. Recognition of a pMHC (peptide-MHC) complex by a TCR initiates the propagation of a cellular immune response that leads to the production of cytokines and the lysis of the Antigen-Presenting Cell (see, e.g., Armstrong, K. M. et al. (2008) “Conformational Changes And Flexibility In T-Cell Receptor Recognition Of Peptide—MHC Complexes,” Biochem. J. 415(Pt 2):183-196; Willemsen, R. (2008) “Selection Of Human Antibody Fragments Directed Against Tumor T-Cell Epitopes For Adoptive T-Cell Therapy,” Cytometry A. 73(11):1093-1099; Beier, K. C. et al. (2007) “Master Switches Of T-Cell Activation And Differentiation,” Eur. Respir. J. 29:804-812; Mallone, R. et al. (2005) “Targeting T Lymphocytes For Immune Monitoring And Intervention In Autoimmune Diabetes,” Am. J. Ther. 12(6):534-550). CD3 is the receptor that binds to the TCR (Thomas, S. et al. (2010) “Molecular Immunology Lessons From Therapeutic T-Cell Receptor Gene Transfer,” Immunology 129(2):170-177; Guy, C. S. et al. (2009) “Organization Of Proximal Signal Initiation At The TCR: CD3 Complex,” Immunol. Rev. 232(1):7-21; St. Clair, E. W. (Epub 2009 Oct. 12)“Novel Targeted Therapies For Autoimmunity,” Curr. Opin. Immunol. 21(6):648-657; Baeuerle, P. A. et al. (Epub 2009 Jun. 9) “Bispecific T-Cell Engaging Antibodies For Cancer Therapy,” Cancer Res. 69(12):4941-4944; Smith-Garvin, J. E. et al. (2009) “T Cell Activation,” Annu. Rev. Immunol. 27:591-619; Renders, L. et al. (2003) “Engineered CD3 Antibodies For Immunosuppression,” Clin. Exp. Immunol. 133 (3):307-309).

The TCR and CD3 complex, along with the CD3 ζ chain zeta chain (also known as T Cell receptor T3 zeta chain or CD247) comprise the “TCR complex” (van der Merwe, P. A. etc. (epub Dec. 3, 2010) “Mechanisms For T Cell Receptor Triggering,” Nat. Rev. Immunol. 11:47-55; Wucherpfennig, K. W. et al. (2010) “Structural Biology of the T Cell Receptor: Insights into Receptor Assembly, Ligand Recognition, and Initiation of Signaling,” Cold Spring Harb. Perspect. Biol. 2:a005140). The complex is particularly significant since it contains a large number (ten) of immunoreceptor tyrosine-based activation motifs (ITAMs).

D. The Fc Receptors: CD16, CD32 and CD64

As discussed in detail below, natural IgG antibodies are composed of four polypeptide chains: two identical “light” chains and two identical “heavy” chains. The Heavy Chains contain C-terminal “CH2” and “CH3” domains, and the association of the two Heavy Chains creates an “Fc Domain” that is capable of ligating (binding) to receptors (singularly referred to as an “Fc gamma receptor” “FcγR,” and collectively as “FcγRs”) found on the surfaces of multiple types of immune system cells (e.g., B lymphocytes, follicular dendritic cells, natural killer cells, macrophages, neutrophils, eosinophils, basophils and mast cells). Such receptors have an “extracellular” portion (which is thus capable of ligating to an Fc Domain), a “transmembrane” portion (which extends through the cellular membrane), and a “cytoplasmic” portion (positioned inside the cell). Multiple types of FcγRs have been identified: CD16A (FcγRIIIA), CD16B (FcγRIIIB), CD32A (FcγRIIA), CD32B (FcγRIIB), and CD64 (FcγRI). Such binding results in the transduction of activating or inhibitory signals to the immune system.

CD16 is a generic name for the activating Fc receptors, FcγRIIIA (CD16A) and FcγRIIIB (CD16B). CD16 is expressed by neutrophils, eosinophils, natural killer (NK) cells, and tissue macrophages that bind aggregated but not monomeric human IgG (Peitz, G. A. et al. (1989) “Human Fc Gamma RIII: Cloning, Expression, And Identification Of The Chromosomal Locus Of Two Fc Receptors For IgG,” Proc. Natl. Acad. Sci. (U.S.A.) 86(3):1013-1017; Bachanova, V. et al. (2014) “NK Cells In Therapy Of Cancer,” Crit. Rev. Oncog. 19(1-2): 133-141; Miller, J. S. (2013) “Therapeutic Applications: Natural Killer Cells In The Clinic,” Hematology Am. Soc. Hematol. Educ. Program. 2013:247-253; Youinou, P. et al. (2002) “Pathogenic Effects Of Anti-Fc Gamma Receptor IIIB (CD16) On Polymorphonuclear Neutrophils In Non-Organ-Specific Autoimmune Diseases,” Autoimmun Rev. 1(1-2):13-19; Peipp, M. et al. (2002) “Bispecific Antibodies Targeting Cancer Cells,” Biochem. Soc. Trans. 30(4):507-511). These receptors bind the Fc portion of IgG antibodies, thereby triggering the release of cytokines. If such antibodies are bound to a Disease Antigen that is expressed on the surface of a cell (e.g., a cancer cell, pathogen-infected cell, etc.), then such release mediates the killing of the targeted cell. Since such killing is antibody-dependent, it is termed antibody-dependent cell-mediated cytotoxicity (ADCC).

CD32A (FcγRIIA) (Brandsma, A. M. (2015) “Fc Receptor Inside-Out Signaling And Possible Impact On Antibody Therapy,” Immunol Rev. 268(1):74-87; van Sorge, N. M. et al. (2003) “FcgammaR Polymorphisms: Implications For Function, Disease Susceptibility And Immunotherapy,” Tissue Antigens 61(3):189-202; Selvaraj, P. et al. (2004) “Functional Regulation Of Human Neutrophil Fc Gamma Receptors,” Immunol. Res. 29(1-3):219-230) and CD64 (FcγRI) (Lu, S. et al. (2015) “Structural Mechanism Of High Affinity FcγRI recognition Of Immunoglobulin G,” Immunol. Rev. 268(1):192-200; Swisher, J. F. et al. (2015) “The Many Faces Of FcγRI: Implications For Therapeutic Antibody Function,” Immunol. Rev. 268(1):160-174; Thepen, T. et al. (2009) “Fcgamma Receptor 1 (CD64), A Target Beyond Cancer,” Curr. Pharm. Des. 15(23):2712-2718; Rouard, H. et al. (1997) “Fc Receptors As Targets For Immunotherapy,” Int. Rev. Immunol. 16(1-2):147-185) are activating Fc receptors that are expressed on macrophages, neutrophils, eosinophils and dendritic cells (and for CD32A, also on platelets and Langerhan cells). In contrast, CD32B (FcγRIIB) is an inhibiting Fc receptor on B lymphocytes (macrophages, neutrophils, and eosinophils) (Stopforth, R. J. et al. (2016) “Regulation of Monoclonal Antibody Immunotherapy by FcγRIIB,” J. Clin. Immunol. [2016 Feb. 27 Epub], pp. 1-7; Bruhns, P. et al. (2009) “Specificity And Affinity Of Human Fcgamma Receptors And Their Polymorphic Variants For Human IgG Subclasses,” Blood. 113 (16):3716-3725; White, A. L. et al. (2014) “FcγRIIB As A Key Determinant Of Agonistic Antibody Efficacy,” Curr. Top. Microbiol. Immunol. 382:355-372; Selvaraj, P. et al. (2004) “Functional Regulation Of Human Neutrophil Fc Gamma Receptors,” Immunol. Res. 29(1-3): 219-230).

The ability of the different FcγRs to mediate diametrically opposing functions reflects their structural differences, and in particular whether the FcγR possesses an immunoreceptor tyrosine-based activation motif (“ITAM”) or an immunoreceptor tyrosine-based inhibitory motif (“ITIM”). The recruitment of different cytoplasmic enzymes to these structures dictates the outcome of the FcγR-mediated cellular responses. ITAM-containing FcγRs include FcγRI, FcγRIIA, FcγRIIIA, and activate the immune system when bound to Fc Domains (e.g., aggregated Fc Domains present in an immune complex). FcγRIIB is the only currently known natural ITIM-containing FcγR; it acts to dampen or inhibit the immune system when bound to aggregated Fc Domains.

E. The NKG2D Receptor

The Natural Killer Group 2D (“NKG2D”) receptor is expressed on all human (and other mammalian) Natural Killer cells (Bauer, S. et al. (1999) “Activation Of NK Cells And T Cells By NKG2D, A Receptor For Stress-Inducible MICA,” Science 285(5428):727-729; Jamieson, A. M. et al. (2002) “The Role Of The NKG2D Immunoreceptor In Immune Cell Activation And Natural Killing,” Immunity 17(1):19-29) as well as on all CD8⁺ T cells (Groh, V. et al. (2001) “Costimulation Of CD8αβ T Cells By NKG2D Via Engagement By MIC Induced On Virus-Infected Cells,” Nat. Immunol. 2(3):255-260; Jamieson, A. M. et al. (2002) “The Role Of The NKG2D Immunoreceptor In Immune Cell Activation And Natural Killing,” Immunity 17(1):19-29). NKG2D ligands are completely absent, or are present only at low levels, on the surfaces of normal cells, but they are overexpressed by infected, transformed, senescent or stressed cells. Such binding ligands, and particularly those which are not expressed on normal cells, include the histocompatibility 60 (H60) molecule, the product of the retinoic acid early inducible gene-1 (RAE-1), and the murine UL16-binding protein-like transcript 1 (MULTI) (Raulet D. H. (2003) “Roles Of The NKG2D Immunoreceptor And Its Ligands,” Nature Rev. Immunol. 3:781-790; Coudert, J. D. et al. (2005) “Altered NKG2D Function In NK Cells Induced By Chronic Exposure To Altered NKG2D Ligand-Expressing Tumor Cells,” Blood 106:1711-1717).

### III. Interacting Molecules of Immune System Cells

Interactions involving several different kinds of Antigen-Presenting Cell molecules and T Cell molecules affect the required second interaction of the immune response immune response.

A. CD80/CD86 and CD28/CTLA-4

Binding between the B7.1 (CD80) and B7.2 (CD86) ligands of Antigen-Presenting Cells and the CD28 and CTLA-4 receptors of CD4⁺ T lymphocytes is of particular importance to the required second interaction of the immune response (Sharpe, A. H. et al. (2002) “The B7-CD28 Superfamily,” Nature Rev. Immunol. 2:116-126; Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). Binding of B7.1 or of B7.2 to CD28 stimulates T-cell activation; binding of B7.1 or B7.2 to CTLA-4 inhibits such activation (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321; Greenwald, R. J. et al. (2005) “The B7 Family Revisited,” Ann. Rev. Immunol. 23:515-548). CD28 is constitutively expressed on the surface of T-cells (Gross, J., et al. (1992) “Identification And Distribution Of The Costimulatory Receptor CD28 In The Mouse,” J. Immunol. 149:380-388), whereas CTLA-4 expression is rapidly upregulated following T-cell activation (Linsley, P. et al. (1996) “Intracellular Trafficking Of CTLA4 And Focal Localization Towards Sites Of TCR Engagement,” Immunity 4:535-543). Since CTLA-4 is the higher affinity receptor (Sharpe, A. H. et al. (2002) “The B7-CD28 Superfamily,” Nature Rev. Immunol. 2:116-126; Topalian, S. L. et al. (2015) “Immune Checkpoint Blockade: A Common Denominator Approach to Cancer Therapy,” Cancer Cell 27:450-461), binding first initiates T-cell proliferation (via CD28) and then inhibits it (via nascent expression of CTLA-4), thereby dampening the effect when proliferation is no longer needed.

B. PD-1 and B7-H1/B7-DC

Programmed Death-1 (“PD-1,” also known as “CD279”) is type I membrane protein member of the extended CD28/CTLA-4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895; United States Patent Application Publications No. 2007/0202100; 2008/0311117; 2009/00110667; U.S. Pat. Nos. 6,808,710; 7,101,550; 7,488,802; 7,635,757; 7,722,868; PCT Publication No. WO 01/14557).

Although PD-1 and CTLA-4 both provide inhibitory immune signals, the signals provided by PD-1 are mounted later in the course of the disease, and can profoundly diminish the immune response by limiting the initial production (“burst”) of disease-responsive T-cells. As such PD-1 can partially convert a potentially effective T-cell response into one of tolerance (Topalian, S. L. et al. (2015) “Immune Checkpoint Blockade: A Common Denominator Approach to Cancer Therapy,” Cancer Cell 27:450-461).

The receptor-ligand interactions of the PD-1 system appear to be even more complex than those of the CD28/CTLA-4 system. PD-1 is expressed on the cell surface of activated T-cells, B-cells, and monocytes (Agata, Y. et al. (1996) “Expression Of The PD-1 Antigen On The Surface Of Stimulated Mouse T And B Lymphocytes,” Int. Immunol. 8(5):765-772; Yamazaki, T. et al. (2002) “Expression Of Programmed Death 1 Ligands By Murine T-Cells And APC,” J. Immunol. 169:5538-5545) and at low levels in natural killer (NK) T-cells (Nishimura, H. et al. (2000) “Facilitation Of Beta Selection And Modification Of Positive Selection In The Thymus Of PD-1-Deficient Mice,” J. Exp. Med. 191:891-898; Martin-Orozco, N. et al. (2007) “Inhibitory Costimulation And Anti-Tumor Immunity,” Semin. Cancer Biol. 17(4):288-298).

The extracellular region of PD-1 consists of a single immunoglobulin (Ig)V domain with 23% identity to the equivalent domain in CTLA-4 (Martin-Orozco, N. et al. (2007) “Inhibitory Costimulation And Anti-Tumor Immunity,” Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y et al. (1992) “Induced Expression Of PD-1, A Novel Member Of The Immunoglobulin Gene Superfamily, Upon Programmed Cell Death,” EMBO J. 11:3887-3895; Blank, C. et al. (2006) “Contribution Of The PD-L1/PD-1 Pathway To T-Cell Exhaustion: An Update On Implications For Chronic Infections And Tumor Evasion Cancer,” Immunol. Immunother. 56(5):739-745).

PD-1 mediates its inhibition of the immune system by binding B7-H1 and B7-DC (also known as PD-L1 and PD-L2) (Flies, D. B. et al. (2007) “The New B7 s: Playing a Pivotal Role in Tumor Immunity,” J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

B7-H1 and B7-DC are broadly expressed on the surfaces of many types of human and murine tissues, such as heart, placenta, muscle, fetal liver, spleen, lymph nodes, and thymus as well as murine liver, lung, kidney, islets cells of the pancreas and small intestine (Martin-Orozco, N. et al. (2007) “Inhibitory Costimulation And Anti-Tumor Immunity,” Semin. Cancer Biol. 17(4):288-298). In humans, B7-H1 protein expression has been found in human endothelial cells (Chen, Y. et al. (2005) “Expression of B7 H1 in Inflammatory Renal Tubular Epithelial Cells,” Nephron. Exp. Nephrol. 102:e81-e92; de Haij, S. et al. (2005) “Renal Tubular Epithelial Cells Modulate T-Cell Responses Via ICOS-L And B7-H1” Kidney Int. 68:2091-2102; Mazanet, M. M. et al. (2002) “B7-HI Is Expressed By Human Endothelial Cells And Suppresses T-Cell Cytokine Synthesis,” J. Immunol. 169:3581-3588), myocardium (Brown, J. A. et al. (2003) “Blockade Of Programmed Death-1 Ligands On Dendritic Cells Enhances T-Cell Activation And Cytokine Production,” J. Immunol. 170:1257-1266), syncyciotrophoblasts (Petroff, M. G. et al. (2002) “B7 Family Molecules: Novel Immunomodulators At The Maternal-Fetal Interface,” Placenta 23: S95-S101). The molecules are also expressed by resident macrophages of some tissues, by macrophages that have been activated with interferon (IFN)-γ or tumor necrosis factor (TNF)-α (Latchman, Y. et al. (2001) “PD-L2 Is A Second Ligand For PD-1 And Inhibits T-Cell Activation,” Nat. Immunol 2:261-268), and in tumors (Dong, H. (2003) “B7-H Pathway And Its Role In The Evasion Of Tumor Immunity,” J. Mol. Med. 81:281-287).

The interaction between B7-H1 and PD-1 has been found to provide a crucial negative costimulatory signal to T and B-cells (Martin-Orozco, N. et al. (2007) “Inhibitory Costimulation And Anti-Tumor Immunity,” Semin. Cancer Biol. 17(4):288-298) and functions as a cell death inducer (Ishida, Y. et al. (1992) “Induced Expression Of PD-1, A Novel Member Of The Immunoglobulin Gene Superfamily, Upon Programmed Cell Death,” EMBO J. 11:3887-3895; Subudhi, S. K. et al. (2005) “The Balance Of Immune Responses: Costimulation Verse Coinhibition,” J. Molec. Med. 83:193-202). More specifically, interaction between low concentrations of the PD-1 receptor and the B7-H1 ligand has been found to result in the transmission of an inhibitory signal that strongly inhibits the proliferation of antigen-specific CD8⁺ T-cells; at higher concentrations, the interactions with PD-1 do not inhibit T-cell proliferation but markedly reduce the production of multiple cytokines (Sharpe, A. H. et al. (2002) “The B7-CD28 Superfamily,” Nature Rev. Immunol. 2:116-126). T-cell proliferation and cytokine production by both resting and previously activated CD4 and CD8 T-cells, and even naïve T-cells from umbilical-cord blood, have been found to be inhibited by soluble B7-H1-Fc fusion proteins (Freeman, G. J. et al. (2000) “Engagement Of The PD-1 Immunoinhibitory Receptor By A Novel B7 Family Member Leads To Negative Regulation Of Lymphocyte Activation,” J. Exp. Med. 192:1-9; Latchman, Y. et al. (2001) “PD-L2 Is A Second Ligand For PD-1 And Inhibits T-Cell Activation,” Nature Immunol. 2:261-268; Carter, L. et al. (2002) “PD-1:PD-L Inhibitory Pathway Affects Both CD4(+) and CD8(+) T-cells And Is Overcome By IL-2,” Eur. J. Immunol. 32(3):634-643; Sharpe, A. H. et al. (2002) “The B7-CD28 Superfamily,” Nature Rev. Immunol. 2:116-126).

The role of B7-H1 and PD-1 in inhibiting T-cell activation and proliferation has suggested that these biomolecules might serve as therapeutic targets for treatments of inflammation and cancer. Thus, the use of anti-PD-1 antibodies to treat infections and tumors and to up-modulate an adaptive immune response has been proposed (see, United States Patent Application Publication Nos. 2010/0040614; 2010/0028330; 2004/0241745; 2008/0311117; 2009/0217401; U.S. Pat. Nos. 7,521,051; 7,563,869; 7,595,048; PCT Publications Nos. WO 2004/056875; WO 2008/083174). Antibodies capable of specifically binding PD-1 have been reported by Agata, T. et al. (1996) “Expression Of The PD-1 Antigen On The Surface Of Stimulated Mouse T And B Lymphocytes,” Int. Immunol. 8(5):765-772; and Berger, R. et al. (2008) “Phase I Safety And Pharmacokinetic Study Of CT-011, A Humanized Antibody Interacting With PD-1, In Patients With Advanced Hematologic Malignancies,” Clin. Cancer Res. 14(10):3044-3051 (see, also, U.S. Pat. Nos. 8,008,449 and 8,552,154; US Patent Publication Nos. 2007/0166281; 2012/0114648; 2012/0114649; 2013/0017199; 2013/0230514 and 2014/0044738; and PCT Patent Publication Nos. WO 2003/099196; WO 2004/004771; WO 2004/056875; WO 2004/072286; WO 2006/121168; WO 2007/005874; WO 2008/083174; WO 2009/014708; WO 2009/073533; WO 2012/135408, WO 2012/145549; and WO 2013/014668).

Despite such advances in identifying the molecules involved in mammalian immune responses, a need remains for improved therapies for treating cancers and infectious diseases. The present invention is directed to this and other goals.

### SUMMARY OF THE INVENTION

The present invention is directed to a combination therapy for the treatment of cancer and pathogen-associated diseases, that comprises the administration of: (1) a molecule (e.g., a diabody, an scFv, an antibody, a TandAb, etc.) capable of binding PD-1 or a natural ligand of PD-1, and (2) a molecule (e.g., a diabody, a BiTe, a bispecific antibody, a CAR, etc.) capable of mediating the redirected killing of a target cell (e.g., a cancer cell or a pathogen-infected cell, etc.) expressing a Disease Antigen. The invention particularly concerns the embodiment in which the molecule capable of mediating the redirected killing of the target cell is a bispecific binding molecule that comprises a first epitope-binding site capable of immunospecifically binding an epitope of a cell surface molecule of an effector cell and a second epitope-binding site that is capable of immunospecifically binding an epitope of such target cells (i.e., a Disease Antigen such as a Cancer Antigen or a Pathogen-Associated Antigen). The present invention is also directed to pharmaceutical compositions that comprise such molecule(s).

In detail, the invention provides a method for the treatment of cancer or a pathogen-associated disease, comprising administering to a subject in need thereof a therapeutically effective amount of:
(1) a molecule capable of binding PD-1 or a natural ligand of PD-1, and
(2) a molecule capable of mediating the redirected killing of a target cell, wherein the target cell is:
   (a) a cancer cell that expresses a Cancer Antigen; or
   (b) a pathogen-infected cell that expresses a Pathogen-Associated Antigen.

The invention particularly concerns the embodiment of such method wherein the molecule capable of binding PD-1 or a natural ligand of PD-1 is capable of inhibiting binding between PD-1 and a natural ligand of PD-1.

The invention further concerns the embodiment of such method, wherein the method comprises administration of two binding molecules that cumulatively comprise three epitope-binding domains, the two binding molecules being:
(A) a binding molecule that comprises an epitope-binding domain of an antibody that is capable of binding PD-1, or an epitope-binding domain of an antibody that is capable of binding a natural ligand of PD-1, and
(B) a binding molecule that comprises:
   (1) an epitope-binding domain of an antibody that is capable of binding a cell surface molecule of the effector cell; and
   (2) an epitope-binding domain of an antibody that that is capable of binding the Cancer Antigen or the Pathogen Antigen of the target cell;
   wherein the epitope-binding domain of the binding molecule (A) is capable of binding PD-1 or a natural ligand of PD-1, and the epitope-binding domains (1) and (2) of the binding molecule (B) are capable of mediating the redirected killing of the target cell.
      The invention further concerns the embodiment of such method, wherein the binding molecule capable of binding PD-1 or a natural ligand of PD-1 comprises a diabody, scFv, antibody or TandAb, and the binding molecule (B) comprises a bispecific diabody, a CAR, a BiTe, or bispecific antibody.

The invention further concerns the embodiment of such methods wherein the binding molecule capable of binding PD-1 or a natural ligand of PD-1 comprises an epitope-binding domain of an antibody that binds to PD-1.

The invention further concerns the embodiment of such methods wherein the binding molecule capable of binding PD-1 or a natural ligand of PD-1 comprises an epitope-binding domain of an antibody that binds to a natural ligand of PD-1

The invention further concerns the embodiment of such methods wherein the binding molecule capable of binding PD-1 or a natural ligand of PD-1 comprises a second epitope-binding domain capable of binding PD-1, wherein such epitope-binding domains:
(a) compete for binding the same epitope of PD-1; or
(b) do not compete for binding the same epitope of PD-1

The invention further concerns the embodiment of such methods wherein the PD-1-epitope-binding domains are capable of simultaneous binding to the same PD-1 molecule.

The invention further concerns the embodiment of such methods wherein the binding molecule capable of binding PD-1 or a natural ligand of PD-1 comprises a second epitope-binding domain capable of binding the natural ligand of PD-1, wherein such epitope-binding domains:
(a) compete for binding to the same epitope of such natural ligand of PD-1, or
(b) do not compete for binding to the same epitope of such natural ligand of PD-1.

The invention further concerns the embodiment of such methods wherein the PD-1 ligand-epitope-binding domains are capable of simultaneous binding the same molecule of the natural ligand of PD-1

The invention further concerns the embodiment of such methods wherein the binding molecule capable of binding PD-1 or a natural ligand of PD-1 comprises a second epitope-binding domain capable of binding an epitope of a molecule that is not PD-1 or a natural ligand of PD-1.

The invention further concerns the embodiment of such methods wherein in the second epitope-binding domain binds an epitope of CD137, LAG-3, OX40, TIGIT, TIM-3, or VISTA.

The invention further concerns the embodiment of such methods wherein the binding molecule capable of mediating the redirected killing of the target cell comprises a third epitope-binding domain capable of binding a cell surface molecule of the effector cell.

The invention further concerns the embodiment of such methods wherein the third epitope-binding-domain of the binding molecule capable of mediating the redirected killing of the target cell is capable of binding a different cell surface molecule of the effector cell, such that the binding molecule capable of mediating the redirected killing is capable of binding two different cell surface molecules of the effector cell.

The invention further concerns the embodiment of such methods wherein the binding molecule capable of mediating the redirected killing of the target cell comprises a third epitope-binding domain capable of binding to a Cancer Antigen or a Pathogen-Associated Antigen of the target cell.

The invention further concerns the embodiment of such methods wherein the third epitope-binding-domain of the binding molecule capable of mediating the redirected killing of the target cell is capable of binding a different Cancer Antigen or a different Pathogen Antigen of the target cell, such that the binding molecule capable of mediating the redirected killing is capable of binding to two different Cancer Antigens or two different Pathogen Antigens of the target cell.

The invention further concerns the embodiment of such methods wherein the cell surface molecule of the effector cell is selected from the group consisting of: CD2, CD3, CD8, CD16, TCR, and NKG2D.

The invention further concerns the embodiment of such methods wherein the Cancer Antigen is selected from the group consisting of the Cancer Antigens: 19.9, 4.2, A33, ADAM-9, AH6, ALCAM, B1, B7-H3, BAGE, beta-catenin, blood group ALe^{b}/Le^{y}, Burkitt's lymphoma antigen-38.13, C14, CA125, Carboxypeptidase M, CD5, CD19, CD20, CD22, CD23, CD25, CD27, CD28, CD33, CD36, CD40/CD154, CD45, CD56, CD46, CD52, CD56, CD79a/CD79b, CD103, CD123, CD317, CDK4, CEA, CEACAM5/CEACAM6, C017-1A, CO-43, CO-514, CTA-1, CTLA-4, Cytokeratin 8, D1.1, D156-22, DR5, E₁ series, EGFR, an Ephrin receptor, Erb, GAGE, a GD2/GD3/GM2 ganglioside, GICA 19-9, gp100, Gp37, gp75, gpA33, HER2/neu, HMFG, human papillomavirus-E6/human papillomavirus-E7, HMW-MAA, I antigen, IL13Rα2, Integrin β6, JAM-3, KID3, KID31, KS 1/4 pan-carcinoma antigen, L6,L20, LEA, LUCA-2, M1:22:25:8, M18, M39, MAGE, MART, mesothelin, MUC-1, MUM-1, Myl, N-acetylglucosaminyltransferase, neoglycoprotein, NS-10, OFA-1, OFA-2, Oncostatin M, p15, p97, PEM, PEMA, PIPA, PSA, PSMA, prostatic acid phosphate, R₂₄, ROR1, a sphingolipid, SSEA-1, SSEA-3, SSEA-4, sTn, the T cell receptor derived peptide, T₅A₇, TAG-72, TL5, TNF-receptor, TNF-γ receptor, TRA-1-85, a Transferrin Receptor, 5T4, TSTA, VEGF, a VEGF Receptor, VEP8, VEP9, VIM-D5, and Y hapten, Le^{y}.

The invention further concerns the embodiment of such methods wherein the method comprises the administration of the pharmaceutical composition, and wherein the Pathogen-Associated Antigen is selected from the group consisting of the Pathogen-Associated Antigens: Herpes Simplex Virus infected cell protein (ICP)47, Herpes Simplex Virus gD, Epstein-Barr Virus LMP-1, Epstein-Barr Virus LMP-2A, Epstein-Barr Virus LMP-2B, Human Immunodeficiency Virus gp160, Human Immunodeficiency Virus gp120, Human Immunodeficiency Virus gp41, etc.), Human Papillomavirus E6, Human Papillomavirus E7, human T-cell leukemia virus gp64, human T-cell leukemia virus gp46, and human T-cell leukemia virus gp21

The invention further provides a pharmaceutical composition that comprises:

(A) therapeutically effective amounts of:

(1) a molecule capable of binding PD-1 or a natural ligand of PD-1, and
(2) a molecule capable of mediating the redirected killing of a target cell expressing a Cancer Antigen or a Pathogen Antigen; and

(B) a pharmaceutically acceptable carrier.

The invention further concerns the embodiment of such pharmaceutical composition wherein the pharmaceutical composition comprises two binding molecules that cumulatively comprise three epitope-binding domains, the two binding molecules being: (A) a binding molecule that comprises an epitope-binding domain of an antibody that is capable of binding PD-1, or an epitope-binding domain of an antibody that is capable of binding a natural ligand of PD-1; and
(B) a binding molecule that comprises:

(1) an epitope-binding domain of an antibody that is capable of binding a cell surface molecule of the effector cell; and
(2) an epitope-binding domain of an antibody that that is capable of binding a

Cancer Antigen or a Pathogen-Associated Antigen of the target cell;
wherein the epitope-binding domain of the binding molecule (A) is capable of binding PD-1 or a natural ligand of PD-1, and the epitope-binding domains (1) and (2) of the binding molecule (B) are capable of mediating the redirected killing of the target cell.

The invention further concerns the embodiment of such pharmaceutical compositions wherein the binding molecule (A) comprises a diabody, scFv, antibody, or TandAb, and the binding molecule (B) comprises a diabody, a CAR, a BiTe, or bispecific antibody.

The invention further concerns the embodiment of such pharmaceutical compositions wherein the molecule capable of binding PD-1 or a natural ligand of PD-1 comprises an epitope-binding domain of an antibody that binds to PD-1

The invention further concerns the embodiment of such pharmaceutical compositions wherein the molecule capable of binding PD-1 or a natural ligand of PD-1 comprises an epitope-binding domain of an antibody that binds to a natural ligand of PD-1.

The invention further concerns the embodiment of such pharmaceutical compositions wherein the molecule capable of binding PD-1 or a natural ligand of PD-1 comprises a second epitope-binding domain capable of binding PD-1, wherein such PD-1-epitope-binding domains:

(a) compete for binding to the same epitope of PD-1; or

(b) do not compete for binding the same epitope of PD-1

The invention further concerns the embodiment of such pharmaceutical compositions wherein the PD-1-epitope-binding domains are capable of simultaneous binding the same PD-1 molecule.

The invention further concerns the embodiment of such pharmaceutical compositions wherein the binding molecule capable of binding PD-1 or a natural ligand of PD-1 comprises a second epitope-binding domain capable of binding the natural ligand of PD-1, wherein such epitope-binding domains:

(a) compete for binding to the same epitope of such natural ligand of PD-1; or

(b) do not compete for binding to the same epitope of such natural ligand of PD-1.

The invention further concerns the embodiment of such pharmaceutical compositions wherein the PD-1 ligand-epitope-binding domains are capable of simultaneous binding the same molecule of the natural ligand of PD-1

The invention further concerns the embodiment of such pharmaceutical compositions wherein the binding molecule capable of binding PD-1 or a natural ligand of PD-1 comprises a second epitope-binding domain capable of binding an epitope of a molecule that is not PD-1 or a natural ligand of PD-1.

The invention further concerns the embodiment of such pharmaceutical compositions wherein the second epitope-binding domain binds an epitope of CD137, LAG-3, OX40, TIGIT, TIM-3, or VISTA.

The invention further concerns the embodiment of such pharmaceutical compositions wherein the molecule capable of mediating the redirected killing of the target cell comprises a third epitope-binding domain, wherein such three epitope-binding domains are capable of simultaneous binding, and wherein the third epitope-binding site is capable of binding an epitope of a cell surface molecule of the effector cell.

The invention further concerns the embodiment of such pharmaceutical compositions wherein the third epitope-binding-domain of the binding molecule capable of mediating the redirected killing of the target cell is capable of binding a different cell surface molecule of the effector cell, such that the binding molecule capable of mediating the redirected killing is capable of binding two different cell surface molecules of the effector cell.

The invention further concerns the embodiment of such pharmaceutical compositions wherein the binding molecule capable of mediating the redirected killing of the target cell comprises a third epitope-binding domain capable of binding to a Cancer Antigen or a Pathogen-Associated Antigen of the target cell.

The invention further concerns the embodiment of such pharmaceutical compositions wherein the third epitope-binding-domain of the binding molecule capable of mediating the redirected killing of the target cell is capable of binding a different Cancer Antigen or a different Pathogen-Associated Antigen of the target cell, such that the binding molecule capable of mediating the redirected killing is capable of binding to two different Cancer Antigens or two different Pathogen-Associated Antigens of the target cell.

The invention further concerns the embodiment of such pharmaceutical compositions wherein the cell surface molecule of the effector cell is selected from the group consisting of: CD2, CD3, CD8, CD16, TCR, and NKG2D.

The invention further concerns the embodiment of such pharmaceutical compositions wherein the Cancer Antigen is selected from the group consisting of the Cancer Antigens: 19.9, 4.2, A33, ADAM-9, AH6, ALCAM, B1, B7-H3, BAGE, beta-catenin, blood group ALe^{b}/Le^{y}, Burkitt's lymphoma antigen-38.13, C14, CA125, Carboxypeptidase M, CD5, CD19, CD20, CD22, CD23, CD25, CD27, CD28, CD33, CD36, CD40/CD154, CD45, CD56, CD46, CD52, CD56, CD79a/CD79b, CD103, CD123, CD317, CDK4, CEA, CEACAM5/CEACAM6, C017-1A, CO-43, CO-514, CTA-1, CTLA-4, Cytokeratin 8, D1.1, D₁56-22, DR5, E₁ series, EGFR, an Ephrin receptor, Erb, GAGE, a GD2/GD3/GM2 ganglioside, GICA 19-9, gp100, Gp37, gp75, gpA33, HER2/neu, HMFG, human papillomavirus-E6/human papillomavirus-E7, HMW-MAA, I antigen, IL13Rα2, Integrinβ6, JAM-3, KID3, KID31, KS 1/4 pan-carcinoma antigen, L6,L20, LEA, LUCA-2, M1:22:25:8, M18, M39, MAGE, MART, mesothelin, MUC-1, MUM-1, Myl, N-acetylglucosaminyltransferase, neoglycoprotein, NS-10, OFA-1, OFA-2, Oncostatin M, p15, p97, PEM, PEMA, PIPA, PSA, PSMA, prostatic acid phosphate, R₂₄, ROR1, a sphingolipid, SSEA-1, SSEA-3, SSEA-4, sTn, the T cell receptor derived peptide, T₅A₇, TAG-72, TL5, TNF-receptor, TNF-γ receptor, TRA-1-85, a Transferrin Receptor, 5T4, TSTA, VEGF, a VEGF Receptor, VEP8, VEP9, VIM-D5, and Y hapten, Le^{y}.

The invention further concerns the embodiment of such pharmaceutical compositions wherein the Pathogen-Associated Antigen is selected from the group consisting of the Pathogen Antigens: Herpes Simplex Virus infected cell protein (ICP)47, Herpes Simplex Virus gD, Epstein-Barr Virus LMP-1, Epstein-Barr Virus LMP-2A, Epstein-Barr Virus LMP-2B, Human Immunodeficiency Virus gp160, Human Immunodeficiency Virus gp120, Human Immunodeficiency Virus gp41, etc.), Human Papillomavirus E6, Human Papillomavirus E7, human T-cell leukemia virus gp64, human T-cell leukemia virus gp46, and human T-cell leukemia virus gp21.

The invention further provides a kit comprising any of the above-described pharmaceutical compositions, wherein the binding molecules thereof are compartmentalized in one or more containers.

### BRIEF DESCRIPTION OF THE DRAWINGS

FIG. 1 provides a schematic of a representative covalently bonded diabody having two epitope-binding domains composed of two polypeptide chains, each having an E-coil or K-coil Heterodimer-Promoting Domain (alternative Heterodimer-Promoting Domains are provided below). A cysteine residue may be present in a linker and/or in the Heterodimer-Promoting Domain as shown in FIG. 3B. VL and VH Domains that recognize the same epitope are shown using the same shading or fill pattern.

FIG. 2 provides a schematic of a representative covalently bonded diabody molecule having two epitope-binding domains composed of two polypeptide chains, each having a CH2 and CH3 Domain, such that the associated chains form all or part of an Fc Domain. VL and VH Domains that recognize the same epitope are shown using the same shading or fill pattern.

FIGS. 3A-3C provide schematics showing representative covalently bonded tetravalent diabodies having four epitope-binding domains composed of two pairs of polypeptide chains (i.e., four polypeptide chains in all). One polypeptide of each pair possesses a CH2 and CH3 Domain, such that the associated chains form all or part of an Fc Domain. VL and VH Domains that recognize the same epitope are shown using the same shading or fill pattern. The two pairs of polypeptide chains may be same. In such embodiments wherein the two pairs of polypeptide chains are the same and the VL and VH Domains recognize different epitopes (as shown in FIGS. 3A-3B), the resulting molecule possesses four epitope-binding domains and is bispecific and bivalent with respect to each bound epitope. In such embodiments wherein the VL and VH Domains recognize the same epitope (e.g., the same VL Domain CDRs and the same VH Domain CDRs are used on both chains) the resulting molecule possesses four epitope-binding domains and is monospecific and tetravalent with respect to a single epitope. Alternatively, the two pairs of polypeptides may be different. In such embodiments wherein the two pairs of polypeptide chains are different and the VL and VH Domains of each pair of polypeptides recognize different epitopes (as shown by the different shading and patterns in FIG. 3C), the resulting molecule possesses four epitope-binding domains and is tetraspecific and monovalent with respect to each bound epitope. FIG. 3A shows an Fc Domain-containing diabody which contains a peptide Heterodimer-Promoting Domain comprising a cysteine residue. FIG. 3B shows an Fc Domain-containing diabody, which contains E-coil and K-coil Heterodimer-Promoting Domains comprising a cysteine residue and a linker (with an optional cysteine residue). FIG. 3C, shows an Fc Domain-Containing diabody, which contains antibody CH1 and CL domains.

FIGS. 4A-4B provide schematics of a representative covalently bonded diabody molecule having two epitope-binding domains composed of three polypeptide chains. Two of the polypeptide chains possess a CH2 and CH3 Domain, such that the associated chains form all or part of an Fc Domain. The polypeptide chains comprising the VL and VH Domain further comprise a Heterodimer-Promoting Domain. VL and VH Domains that recognize the same epitope are shown using the same shading or fill pattern.

FIG. 5 provides the schematics of a representative covalently bonded diabody molecule having four epitope-binding domains composed of five polypeptide chains. Two of the polypeptide chains possess a CH2 and CH3 Domain, such that the associated chains form an Fc Domain that comprises all or part of an Fc Domain. The polypeptide chains comprising the linked VL and VH Domains further comprise a Heterodimer-Promoting Domain. VL and VH Domains that recognize the same epitope are shown using the same shading or fill pattern.

FIGS. 6A-6F provide schematics of representative Fc Domain-containing trivalent binding molecules having three epitope-binding domains. FIGS. 6A and 6B, respectively, illustrate schematically the domains of trivalent binding molecules comprising two diabody-type binding domains and a Fab-Type Binding Domain having different domain orientations in which the diabody-type binding domains are N-terminal or C-terminal to an Fc Domain. The molecules in FIGS. 6A and 6B comprise four chains.

FIGS. 6C and 6D, respectively, illustrate schematically the domains of trivalent binding molecules comprising two diabody-type binding domains N-terminal to an Fc Domain, and a Fab-Type Binding Domain in which the Light Chain and Heavy Chain are linked via a polypeptide spacer, or an scFv-type binding domain. The trivalent binding molecules in FIGS. 6E and 6F, respectively, illustrate schematically the domains of trivalent binding molecules comprising two diabody-type binding domains C-terminal to an Fc Domain, and a Fab-Type Binding Domain in which the Light Chain and Heavy Chain are linked via a polypeptide spacer, or an scFv-type binding domain. The trivalent binding molecules in FIGS. 6C-6F comprise three chains. VL and VH Domains that recognize the same epitope are shown using the same shading or fill pattern.

FIG. 7 shows the result of providing MHCI^{-/-} mice that had received 5×10⁶ LOX-IMVI human metastatic melanoma cancer cells (ID) and 10⁶ human PBMC (IP) with the humanized anti-human PD-1 antibody, hPD-1 mAb7 (1.2) IgG4(P), the CD3 x B7-H3 bispecific diabody, DART-A, with both hPD-1 mAb7 (1.2) IgG4(P) and DART-A, or with vehicle alone (control).

FIGS. 8A-8B show the result of providing MHCI^{-/-} mice that had received 5×10⁶ Detroit562 human metastatic pharyngeal carcinoma cancer cells (ID) and 10⁶ human PBMC (IP) with the humanized anti-human PD-1 antibody, hPD-1 mAb7 (1.2) IgG4(P), the CD3 x B7-H3 bispecific diabody, DART-A, with both hPD-1 mAb7 (1.2) IgG4(P) and DART-A, or with vehicle alone (control). FIG. 8A shows the results for Vehicle Control, hPD-1 mAb7 (1.2) IgG4(P) (Q7Dx5), DART-A (Q7Dx5), and hPD-1 mAb7 (1.2) IgG4(P)+DART-A (Q7Dx5). FIG. 8B shows the results for Vehicle Control, hPD-1 mAb7 (1.2) IgG4(P) (Q7Dx5), DART-A (Q7Dx5), hPD-1 mAb7 (1.2) IgG4(P)+DART-A (Q7Dx5) and hPD-1 mAb7 (1.2) IgG4(P)+DART-A (Q14Dx3).

FIG. 9 shows the results of a study on the effect of the administration of the combination therapy of the present invention. The results show an enhancement of the immune response of recipient animals as determined by an increase in the concentration of their CD3⁺ cells.

FIGS. 10A-10B show the results of a study on the effect of the combination therapy of the present invention on T-cell signaling in a luciferase reporter assay. MDA-MB-231 tumor target cells expressing PD-1 and B7-H3 were mixed with MNFAT-luc2/PD-1 Jurkat T-cells at an effector:target cell ratio of 1:1 (FIG. 10A) or 3:1 (FIG. 10B) and cultured alone or with a fixed concentration (12.5 nM) of the PD-1 binding molecules hPD-1 mAb7 (1.2) IgG4(P), DART-1, or control antibody (hIgG), in the presence of increasing concentations of DART-A. These results show an enhancement in signaling activity in the presence of both molecules as determined by increased luminescence.

FIGS. 11A-11B show that administration of the combination therapy of the present invention reduces tumor recurrence in the presensence of anergic T-cells. NOG mice that had received 5×10⁶ A375 INFγ treated melanoma cells and 5×10⁶ activated or anergic human T-cells with vehicle alone, 0.5 mg/kg DART-2 (Q7Dx4), 0.5 mg/kg DART-B (QDx1), or both 0.5 mg/kg DART-2 (Q7Dx4) and 0.5 mg/kg DART-B (QDx1). FIG. 11A shows the results for mice that received activated T-cells and FIG. 11B shows the results for mice that received anergic T-cells.

FIGS. 12A-12H demonstrate the unexpected benefit of the combined therapy of a molecule capable of binding PD-1 and a molecule capable of mediating the redirected killing of a target cell relative to administration of either molecule alone. Tumor volume caused by A375 melanoma cells was measured as a function of time and is plotted in FIGS. 12A-12H. FIG. 12A shows the results for Groups 1, 2, 5 and 6 through day 50; FIGS. 12B-12H show the spider plots, through day 80, for the individual animals in Group 2 (FIG. 12B), Group 5 (FIG. 12C), Group 6 (FIG. 12D), Group 3 (FIG. 12E), Group 7 (FIG. 12F), Group 4 (FIG. 12G), and Group 8 (FIG. 12H).

### DETAILED DESCRIPTION OF THE INVENTION

The present invention is directed to a combination therapy for the treatment of cancer and pathogen-associated diseases, that comprises the administration of: (1) a molecule (e.g., a diabody, an scFv, an antibody, a TandAb, etc.) capable of binding PD-1 or a natural ligand of PD-1, and (2) a molecule (e.g., a diabody, a BiTe, a bispecific antibody, a CAR, etc.) capable of mediating the redirected killing of a target cell (e.g., a cancer cell or a pathogen-infected cell, etc.) expressing a Disease Antigen. The invention particularly concerns the embodiment in which the molecule capable of mediating the redirected killing of the target cell is a bispecific binding molecule that comprises a first epitope-binding site capable of immunospecifically binding an epitope of a cell surface molecule of an effector cell and a second epitope-binding site that is capable of immunospecifically binding an epitope of such target cells (i.e., a Disease Antigen such as a Cancer Antigen or a Pathogen-Associated Antigen). The present invention is also directed to pharmaceutical compositions that comprise such molecule(s).

The binding domains of the molecules of the present invention bind epitopes in an “immunospecific” manner. As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind a second target. As such, “immunospecific binding” does not necessarily require (although it can include) exclusive binding. Generally, but not necessarily, reference to binding means “immunospecific” binding. Two molecules are said to be capable of binding one another in a “physiospecific” manner, if such binding exhibits the specificity with which receptors bind their respective ligands.

As indicated above, the therapeutic molecules of the present invention particularly include bispecific binding molecules that comprises an epitope-binding site capable of immunospecifically binding an epitope of a cell surface molecule of an effector cell and also an epitope-binding site that is capable of immunospecifically binding an epitope of a target cell that expresses a Disease Antigen. As used herein, the term “Disease Antigen” denotes an antigen that is expressed on the surface of an abnormal or infected cell and that is characteristic of such abnormality of infection, or that is expressed on the surface of a foreign cell and that is characteristic of such foreign origin. As used herein, a cell that expresses a Disease Antigen on its cell surface, and that may therefore become bound by the therapeutic molecules of the present invention and thereby targeted for killing by such therapeutic molecules is a “target cell.” Of particular relevance to the present invention are Disease Antigens that are “Cancer Antigens” or “Pathogen-Associated Antigens.”

I. Antibodies and Their Binding Domains

The binding molecules of the present invention may be antibodies. “Antibodies” are immunoglobulin molecules capable of specific binding a target, such as a carbohydrate, polynucleotide, lipid, polypeptide, etc., through at least one antigen recognition site, located in the Variable Domain of the immunoglobulin molecule. As used herein, the terms “antibody” and “antibodies” refer to monoclonal antibodies, multi specific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies are capable of “immunospecifically binding” to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens.” The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂, Fv fragments, etc.), single-chain (scFv) binding molecules and mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freund's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences.

Natural antibodies (such as IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. In contrast, scFvs are single chain molecules made by linking Light and Heavy Chain Variable Domains together via a short linking peptide.

The basic structural unit of naturally occurring immunoglobulins (e.g., IgG) is thus a tetramer having two Light Chains and two Heavy Chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with Light Chains having a single Constant Domain and Heavy Chains usually having three Constant Domains and a Hinge Domain. Thus, the structure of the Light Chains of an IgG molecule is n-VL-CL-c and the structure of the IgG Heavy Chains is n-VH-CH1-H-CH2-CH3-c (where n and c represent, respectively, the N-terminus and the C-terminus of the polypeptide).

A. Characteristics of Antibody Variable Domains

The Variable Domains of an IgG molecule consist of the complementarity determining regions (“CDR”), which contain the residues in contact with epitope, and non-CDR segments, referred to as framework segments (“FR”), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind a specific epitope regardless of whether such protein is an antibody having light and Heavy Chains or is a diabody or a single-chain binding molecule (e.g., an scFv, a BiTe, etc.), or is another type of protein. Accordingly, as used herein, the term “epitope-binding fragment” denotes a fragment of a molecule capable of immunospecifically binding an epitope. An epitope-binding fragment may contain any 1, 2, 3, 4, or 5 the CDR Domains of an antibody, or may contain all 6 of the CDR Domains of an antibody and, although capable of immunospecifically binding such epitope, may exhibit an immunospecificity, affinity or selectivity towards such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the “N-terminal to C-terminal” direction.

The invention also particularly encompasses epitope-binding molecules that comprise a VL and/or VH Domain of a humanized antibody. The term “humanized antibody” refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The polynucleotide sequence of the Variable Domains of such antibodies may be used for genetic manipulation to generate such derivatives and to improve the affinity, or other characteristics of such antibodies. The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy Variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415

The epitope-binding site may comprise either a complete Variable Domain fused onto Constant Domains or only the complementarity determining regions (CDRs) of such Variable Domain grafted to appropriate framework regions. Epitope-binding domains may be wild-type or modified by one or more amino acid substitutions. This eliminates the constant region as an immunogen in human individuals, but the possibility of an immune response to the foreign Variable Domain remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the Variable Domains as well so as to reshape them as closely as possible to human form. It is known that the Variable Domains of both heavy and Light Chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the Variable Domains can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

A number of humanized antibody molecules comprising an epitope-binding site derived from a non-human immunoglobulin have been described, including chimeric antibodies having rodent or modified rodent Variable Domain and their associated complementarity determining regions (CDRs) fused to human constant domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse/Human Chimeric Monoclonal Antibody (17-1A) To A Colon Cancer Tumor Associated Antigen,” J. Immunol. 138:4534-4538, and Brown et al. (1987) “Tumor-Specific Genetically Engineered Murine/Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

B. Characteristics of Antibody Constant Regions

Throughout the present specification, the numbering of the residues in the constant region of an IgG Heavy Chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by reference. The term “EU index as in Kabat” refers to the numbering of the constant domains of human IgG1 EU antibody. Amino acids from the Variable Domains of the mature heavy and Light Chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain. 1. Constant Regions of the Heavy Chain: Fc Domains

The CH1 Domains of the two Heavy Chains of an antibody complex with the antibody's Light Chain's “CL” constant region, and are attached to the Heavy Chains CH2 Domains via an intervening Hinge Domain.

An exemplary CH1 Domain is a human IgG1 CH1 Domain. The amino acid sequence of an exemplary human IgG1 CH1 Domain is (SEQ ID NO:1):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH1 Domain. The amino acid sequence of an exemplary human IgG2 CH1 Domain is (SEQ ID NO:2):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG4 CH1 Domain. The amino acid sequence of an exemplary human IgG4 CH1 Domain is (SEQ ID NO:3):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

One exemplary Hinge Domain is a human IgG1 Hinge Domain. The amino acid sequence of an exemplary human IgG1 Hinge Domain is (SEQ ID NO:4):

EPKSCDKTHTCPPCP.

Another exemplary Hinge Domain is a human IgG2 Hinge Domain. The amino acid sequence of an exemplary human IgG2 Hinge Domain is (SEQ ID NO:5):

ERKCCVECPPCP.

Another exemplary Hinge Domain is a human IgG4 Hinge Domain. The amino acid sequence of an exemplary human IgG4 Hinge Domain is (SEQ ID NO:6):

ESKYGPPCPSCP.

mutation such as the S228P substitution. The amino acid sequence of an exemplary S228P-stabilized human IgG4 Hinge Domain is (SEQ ID NO:7):

ESKYGPPCPPCP.

The CH2 and CH3 Domains of the two Heavy Chains of an antibody interact to form an “Fc Domain,” which is a domain that is recognized by cellular Fc Receptors, including but not limited to Fc gamma Receptors (FcγRs). As used herein, the term “Fc Domain” is used to define a C-terminal region of an IgG Heavy Chain. An Fc Domain is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of an exemplary human IgG1 is (SEQ ID NO:8):

```
231 240 250 260 270 280
APELLGGPSV FLFPPKPKDT LMISRTPEVT CVVVDVSHED PEVKFNWYVD
                  290 300 310 320 330
GVEVHNAKTK PREEQYNSTY RVVSVLTVLH QDWLNGKEYK CKVSNKALPA
                  340 350 360 370 380
PIEKTISKAK GQPREPQVYT LPPSREEMTK NQVSLTCLVK GFYPSDIAVE
                  390 400 410 420 430
WESNGQPENN YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is lysine (K) or is absent.

The amino acid sequence of the CH2-CH3 Domain of an exemplary human IgG2 is (SEQ ID NO:9):

```
231 240 250 260 270 280
APPVA-GPSV FLFPPKPKDT LMISRTPEVT CVVVDVSHED PEVQFNWYVD
                  290 300 310 320 330
GVEVHNAKTK PREEQFNSTF RVVSVLTVVH QDWLNGKEYK CKVSNKGLPA
                  340 350 360 370 380
PIEKTISKTK GQPREPQVYT LPPSREEMTK NQVSLICLVK GFYPSDISVE
                  390 400 410 420 430
WESNGQPENN YKTTPPMLDS DGSFFLYSKL TVDKSRWQQG NVFSCSVMHE
                  440 447
ALHNHYTQKS LSLSPGX
```

as numbered by the EU index as set forth in Kabat, wherein X is lysine (K) or is absent.

The amino acid sequence of the CH2-CH3 Domain of an exemplary human IgG3 is (SEQ ID NO:10):

```
231 240 250 260 270 280
APELLGGPSV FLFPPKPKDT LMISRTPEVT CVVVDVSHED PEVQFKWYVD
                  290 300 310 320 330
GVEVHNAKTK PREEQYNSTF RVVSVLTVLH QDWLNGKEYK CKVSNKALPA
                  340 350 360 370 380
PIEKTISKTK GQPREPQVYT LPPSREEMTK NQVSLTCLVK GFYPSDIAVE
                  390 400 410 420 430
WESSGQPENN YNTTPPMLDS DGSFFLYSKL TVDKSRWQQG NIFSCSVMHE
                  440 447
ALHNRFTQKS LSLSPGX
```

as numbered by the EU index as set forth in Kabat, wherein X is lysine (K) or is absent.

The amino acid sequence of the CH2-CH3 Domain of an exemplary human IgG4 is (SEQ ID NO:11):

```
231 240 250 260 270 280
APEFLGGPSV FLFPPKPKDT LMISRTPEVT CVVVDVSQED PEVQFNWYVD
                  290 300 310 320 330
GVEVHNAKTK PREEQFNSTY RVVSVLTVLH QDWLNGKEYK CKVSNKGLPS
                  340 350 360 370 380
SIEKTISKAK GQPREPQVYT LPPSQEEMTK NQVSLTCLVK GFYPSDIAVE
                  390 400 410 420 430
WESNGQPENN YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is lysine (K) or is absent.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the binding molecules of the invention. Specifically encompassed by the instant invention are binding molecules lacking the C-terminal residue of the CH3 Domain. Also specifically encompassed by the instant invention are such constructs comprising the C-terminal lysine residue of the CH3 Domain.

### 2. Constant Regions of the Light Chain

As indicated above, each Light Chain of an antibody contains a Variable Domain (“VL”) and a Constant Domain (“CL”).

A preferred CL Domain is a human IgG CL Kappa Domain. The amino acid sequence of an exemplary human CL Kappa Domain is (SEQ ID NO:12):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain.

The amino acid sequence of an exemplary human CL Lambda Domain is (SEQ ID NO:13):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

### II. Chimeric Antigen Receptors

The binding molecules of the present invention that are capable of mediating the redirected killing of a target cell (i.e., a cancer cell, a pathogen-infected cell, etc.) may alternatively be monospecific single-chain molecules such Chimeric Antigen Receptors (“CARs”) incorporating a single chain variable fragment (scFv) capable of binding a Cancer Antigen or a Pathogen-Associated Antigen. As indicated above, scFvs are made by linking Light and Heavy Chain Variable Domains together via a short linking peptide. First-generation CARs typically had the intracellular domain from the CD3 ζ-chain, which is the primary transmitter of signals from endogenous TCRs. Second-generation CARs possessed additional intracellular signaling domains from various costimulatory protein receptors (e.g., CD28, 41BB, ICOS, etc.) to the cytoplasmic tail of the CAR in order to provide additional signals to the T-cell. Third-generation CARs combine multiple signaling domains, such as CD3z-CD28-41BB or CD3z-CD28-OX40, in order to further augment potency (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor Modified T Cells,” Blood 123(15): 2343-2354; Mardiros, A. et al. (2013) “T Cells Expressing CD123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD123 Antigens Efficiently Target Primary Acute Myeloid Leukemia Cells in vivo,” Leukemia doi: 10.1038/leu.2014.62.

The intracellular domain of the CARs of the present invention is preferably selected from the intracellular domain of any of: 41BB-CD3ζ, b2c-CD3ζ, CD28ζ, CD28-4-1BB-CD3ζ, CD28-CD3ζ, CD28-FcεRIγ, CD28mut-CD3ζ, CD28-OX40-CD3ζ, CD28-OX40-CD3ζ, CD3ζ, CD4-CD3ζ, CD4-FcεRIγ, CD8-CD3ζ, FcεRIγ, FcεRIγCAIX, Heregulin-CD3ζ, IL-13-CD3ζ, or Ly49H-CD3ζ (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor-Modified T Cells,” Blood 123(15): 2343-2354; Mardiros, A. et al. (2013) “T Cells Expressing CD123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD123 Antigens Efficiently Target Primary Acute Myeloid Leukemia Cells in vivo,” Leukemia doi:10.1038/leu.2014.62).

### III. Bispecific Antibodies and Multispecific Diabodies

The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two epitope-binding domains of a natural antibody, such as an IgG. Natural antibodies are capable of binding only one epitope species (i.e., they are monospecific), although they can bind multiple copies of that species (i.e., exhibiting bivalency or multivalency).

The functionality of antibodies can be enhanced by generating multispecific antibody-based molecules that can simultaneously bind two separate and distinct antigens (or different epitopes of the same antigen) and/or by generating antibody-based molecule having higher valency (i.e., more than two binding sites) for the same epitope and/or antigen.

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (WO 2005/070966, WO 2006/107786 WO 2006/107617, WO 2007/046893). PCT Publications Nos. WO 2013/174873, WO 2011/133886 and WO 2010/136172 disclose a trispecific antibody in which the CL and CH1 Domains are switched from their respective natural positions and the VL and VH Domains have been diversified (WO 2008/027236; WO 2010/108127) to allow them to bind more than one antigen. PCT Publications Nos. WO 2013/163427 and WO 2013/119903 disclose modifying the CH2 Domain to contain a fusion protein adduct comprising a binding domain. PCT Publications Nos. WO 2010/028797, WO2010028796 and WO 2010/028795 disclose recombinant antibodies whose Fc Domains have been replaced with additional VL and VH Domains, so as to form trivalent binding molecules. PCT Publications Nos. WO 2003/025018 and WO2003012069 disclose recombinant diabodies whose individual chains contain scFv Domains. PCT Publication Nos. WO 2013/006544 discloses multivalent Fab molecules that are synthesized as a single polypeptide chain and then subjected to proteolysis to yield heterodimeric structures. PCT Publications Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's Light Chain, or adding additional VL and VH Domains to the antibody's light and Heavy Chains, or adding a heterologous fusion protein or chaining multiple Fab Domains to one another).

The art has additionally noted the capability to produce diabodies that differ from such natural antibodies in being capable of binding two or more different epitope species (i.e., exhibiting bispecificity or multispecificity in addition to bivalency or multivalency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388/WO 02/02781 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng. Des. Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The design of a diabody is based on the structure of the single-chain Variable Domain fragment (scFv), in which Light and Heavy Chain Variable Domains are linked to one another using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The provision of bispecific binding molecules (e.g., non-monospecific diabodies) provides a significant advantage over antibodies, including but not limited to, a “trans” binding capability sufficient to co-ligate and/or co-localize different cells that express different epitopes and/or a “cis” binding capability sufficient to co-ligate and/or co-localize different molecules expressed by the same cell. Bispecific binding molecules (e.g., non-monospecific diabodies) thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221-1225).

The ability to produce bispecific diabodies has led to their use (in “trans”) to co-ligate two cells together, for example, by co-ligating receptors that are present on the surface of different cells (e.g., cross-linking cytotoxic T-cells to target cells, such as cancer cells or pathogen-infected cells, that express a Disease Antigen) (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305; Marvin et al. (2005) “Recombinant Approaches To IgG-Like Bispecific Antibodies,” Acta Pharmacol. Sin. 26:649-658; Sloan et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog 11(11): e1005233. doi:10.1371/journal.ppat.1005233)). Alternatively (or additionally), bispecific (or tri- or multispecific) diabodies can be used (in “cis”) to co-ligate molecules, such as receptors, etc., that are present on the surface of the same cell. Co-ligation of different cells and/or receptors is useful to modulate effector functions and/or immune cell signaling. Multispecific molecules (e.g., bispecific diabodies) comprising epitope-binding domains may be directed to a surface determinant of any immune cell such as CD2, CD3, CD8, CD16, TCR, NKG2D, etc., which are expressed on T lymphocytes, Natural Killer (NK) cells, Antigen-Presenting Cells or other mononuclear cells. In particular, epitope-binding domains directed to a cell surface receptor that is present on immune effector cells, are useful in the generation of multispecific binding molecules capable of mediating redirected cell killing.

However, the advantages of the above-described bispecific diabodies come at a salient cost. The formation of such non-monospecific diabodies requires the successful assembly of two or more distinct and different polypeptides (i.e., such formation requires that the diabodies be formed through the heterodimerization of different polypeptide chain species). This fact is in contrast to monospecific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13 (8): 583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies, termed DART® (Dual-Affinity Re-Targeting) diabodies; see, e.g., United States Patent Publication Nos. 2013-0295121; 2010-0174053 and 2009-0060910; European Patent Publication No. EP 2714079; EP 2601216; EP 2376109; EP 2158221 and PCT Publication Nos. WO 2012/162068; WO 2012/018687; WO 2010/080538; and Sloan, D. D. et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog. 11(11):e1005233. doi: 10.1371/journal.ppat.1005233; Al Hussaini, M. et al. (2015) “Targeting CD123 In AML Using A T-Cell Directed Dual-Affinity Re-Targeting (DART®) Platform,” Blood pii: blood-2014-05-575704; Chichili, G. R. et al. (2015) “A CD3xCD123 Bispecific DART For Redirecting Host T Cells To Myelogenous Leukemia: Preclinical Activity And Safety In Nonhuman Primates,” Sci. Transl. Med. 7(289):289ra82; Moore, P. A. et al. (2011) “Application Of Dual Affinity Retargeting Molecules To Achieve Optimal Redirected T-Cell Killing Of B-Cell Lymphoma,” Blood 117(17):4542-4551; Yeri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody's binding characteristics.

Many variations of such molecules have been described (see, e.g., United States Patent Publication Nos. 2015/0175697; 2014/0255407; 2014/0099318; 2013/0295121; 2010/0174053; 2009/0060910; 2007-0004909; European Patent Publication Nos. EP 2714079; EP 2601216; EP 2376109; EP 2158221; EP 1868650; and PCT Publication Nos. WO 2012/162068; WO 2012/018687; WO 2010/080538; WO 2006/113665), and are provided herein.

Alternative constructs are known in the art for applications where a bispecific or tetravalent molecule is desirable but an Fc is not required including, but not limited to, Bispecific T cell Engager molecules, also referred to as “BiTEs” (see, e.g., PCT Publication Nos: WO 1993/11161; and WO 2004/106381) and tetravalent tandem antibodies, also referred to as “TandAbs” (see, e.g. United States Patent Publications No: 2011-0206672; European Patent Publication No. EP 2371866, and; PCT Publications Nos. WO 1999/057150, WO 2003/025018, and WO 2013/013700). BiTEs are formed from a single polypeptide chain comprising tandem linked scFvs, while TandAbs are formed by the homo-dimerization of two identical polypeptide chains, each possessing a VH1, VL2, VH2, and VL2 Domain.

The present invention provides bispecific binding molecules that are capable of mediating the redirected killing of a target cell (e.g., a cancer cell or a pathogen-infected cell, etc.) expressing a Disease Antigen. Such bispecific binding molecules are capable of binding a “first epitope” and a “second epitope,” such epitopes not being identical to one another. Such bispecific molecules comprise “VL1”/“VH1” domains that are capable of binding the first epitope, and “VL2”/“VH2” domains that are capable of binding the second epitope. The notation “VL1” and “VH1” denote respectively, the Variable Light Chain Domain and Variable Heavy Chain Domain that bind the “first” epitope of such bispecific molecules. Similarly, the notation “VL2” and “VH2” denote respectively, the Light Chain Variable Domain and Heavy Chain Variable Domain that bind the “second” epitope of such bispecific molecules. It is irrelevant whether a particular epitope is designated as the first vs. the second epitope; such notation having relevance only with respect to the presence and orientation of domains of the polypeptide chains of the binding molecules of the present invention. In one embodiment, one of such epitopes is an epitope of a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell and the other epitope is an epitope of a Disease Antigen (e.g., a Cancer Antigen or a Pathogen-Associated Antigen). In certain embodiments, a bispecific molecule comprises more than two epitope-binding sites. The instant invention particular encompasses bispecific diabodies, BiTEs, antibodies, and TandAbs produced using any of the methods provided herein.

A. Diabodies Lacking Fc Domains

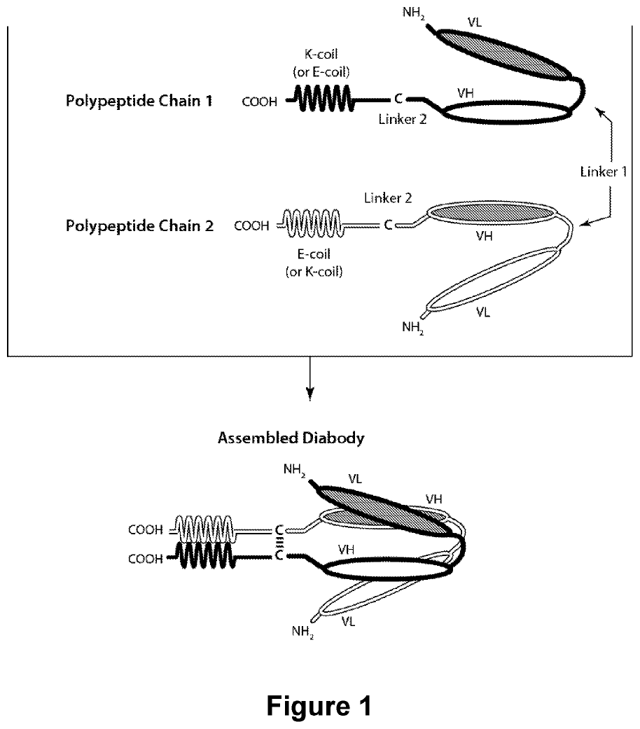

In one embodiment, the diabodies of the invention are bispecific and will comprise domains capable of binding both a first and a second epitope, but will lack an Fc Domain, and thus will be unable to bind FcγR molecules. The first polypeptide chain of such an embodiment of bispecific diabodies comprises, in the N-terminal to C-terminal direction: an N-terminus, the VL Domain of a monoclonal antibody capable of binding either the first or second epitope (i.e., either VL_{Epitope 1} or VL_{Epitope 2}), a first intervening spacer peptide (Linker 1), a VH Domain of a monoclonal antibody capable of binding the second epitope (if such first polypeptide chain contains VL_{Epitope 1}) or a VH Domain of a monoclonal antibody capable of binding the first epitope (if such first polypeptide chain contains VL_{Epitope 2}), a second intervening spacer peptide (Linker 2) optionally containing a cysteine residue, a Heterodimer-Promoting Domain and a C-terminus (FIG. 1).

The second polypeptide chain of this embodiment of bispecific diabodies comprises, in the N-terminal to C-terminal direction: an N-terminus, the VL Domain of a monoclonal antibody capable of binding the first or second epitope (i.e., VL_{Epitope 1} or VL_{Epitope 2}, and being the VL Domain not selected for inclusion in the first polypeptide chain of the diabody), an intervening spacer peptide (Linker 1), a VH Domain of a monoclonal antibody capable of binding either the first or second epitope (i.e., VH_{Epitope 1} or VH_{Epitope 2}, and being the VH Domain not selected for inclusion in the first polypeptide chain of the diabody), a second intervening spacer peptide (Linker 2) optionally containing a cysteine residue, a Heterodimer-Promoting Domain and a C-terminus (FIG. 1). The employed VL and VH Domains specific for a particular epitope are preferably obtained or derived from the same monoclonal antibody. However, such domains may be derived from different monoclonal antibodies provided that they associate to form a functional binding site capable of immunospecifically binding such epitope. Such different antibodies are referred to herein as being “corresponding” antibodies.

The VL Domain of the first polypeptide chain interacts with the VH Domain of the second polypeptide chain to form a first functional epitope-binding site that is specific for one of the epitopes (e.g., the first epitope). Likewise, the VL Domain of the second polypeptide chain interacts with the VH Domain of the first polypeptide chain in order to form a second functional epitope-binding site that is specific for the other epitope (i.e., the second epitope). Thus, the selection of the VL and VH Domains of the first and second polypeptide chains is “coordinated,” such that the two polypeptide chains of the diabody collectively comprise VL and VH Domains capable of binding both the first epitope and the second epitope (i.e., they collectively comprise VL_{Epitope 1}/VH_{Epitope 1} and VL_{Epitope 2}/VH_{Epitope 2}).

Most preferably, the length of the intervening spacer peptide (i.e., “Linker 1,” which separates such VL and VH Domains) is selected to substantially or completely prevent the VL and VH Domains of the polypeptide chain from binding one another (for example consisting of from 0, 1, 2, 3, 4, 5, 6, 7, 8 or 9 intervening linker amino acid residues). Thus the VL and VH Domains of the first polypeptide chain are substantially or completely incapable of binding one another. Likewise, the VL and VH Domains of the second polypeptide chain are substantially or completely incapable of binding one another. A preferred intervening spacer peptide (Linker 1) has the sequence (SEQ ID NO:14):

GGGSGGGG.

The length and composition of the second intervening spacer peptide (“Linker 2”) is selected based on the choice of one or more polypeptide domains that promote such dimerization (i.e., a “Heterodimer-Promoting Domain”). Typically, the second intervening spacer peptide (Linker 2) will comprise 3-20 amino acid residues. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more cysteines. A preferred cysteine-containing spacer peptide (Linker 2) has the sequence GGCGGG (SEQ ID NO:15). Alternatively, Linker 2 does not comprise a cysteine (e.g., GGG, GGGS (SEQ ID NO:16), LGGGSG (SEQ ID NO:17), GGGSGGGSGGG (SEQ ID NO:18), ASTKG (SEQ ID NO:19), LEPKSS (SEQ ID NO:20), APSSS (SEQ ID NO:21), etc.) and a cysteine-containing Heterodimer-Promoting Domain, as described below is used. Optionally, both a cysteine-containing Linker 2 and a cysteine-containing Heterodimer-Promoting Domain are used.

The Heterodimer-Promoting Domains may be GVEPKSC (SEQ ID NO:22) or VEPKSC (SEQ ID NO:23) or AEPKSC (SEQ ID NO:24) on one polypeptide chain and GFNRGEC (SEQ ID NO:25) or FNRGEC (SEQ ID NO:26) on the other polypeptide chain (US2007/0004909).

In a preferred embodiment, the Heterodimer-Promoting Domains will comprise tandemly repeated coil domains of opposing charge for example, an “E-coil” Heterodimer-Promoting Domain (SEQ ID NO:27: EVAALEK-EVAALEK-EVAALEK-EVAALEK), whose glutamate residues will form a negative charge at pH 7, or a “K-coil” Heterodimer-Promoting Domain (SEQ ID NO:28: KVAALKE-KVAALKE-KVAALKE-KVAALKE), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence EVAACEK-EVAALEK-EVAALEK-EVAALEK (SEQ ID NO:29), and a modified K-coil having the amino acid sequence KVAACKE-KVAALKE-KVAALKE-KVAALKE (SEQ ID NO:30).

As disclosed in WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of a polypeptide chain of the diabody. Albumin is the most abundant protein in plasma and has a half-life of 19 days in humans. Albumin possesses several small molecule binding sites that permit it to non-covalently bind other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120). Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of protein G of Streptococcus strain G148 (SEQ ID NO:31):

LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:31 have the ability to attenuate or eliminate MHC class II binding. Based on combinational mutation results, the following combinations of substitutions are considered to be preferred substitutions for forming such a deimmunized ABD: 66D/70S+71A; 66S/70S+71A; 66S/70S+79A; 64A/65A/71A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 32)
LAEAKVLANR ELDKYGVSDY YKNLID₆₆NAKS₇₀ A₇₁EGVKALIDE
ILAALP,

or the amino acid sequence:

(SEQ ID NO: 33)
LAEAKVLANR ELDKYGVSDY YKNA₆₄A₆₅NNAKT VEGVKALIA₇₉E
ILAALP,

or the amino acid sequence:

(SEQ ID NO: 34)
LAEAKVLANR ELDKYGVSDY YKNLIS₆₆NAKS₇₀ VEGVKALIA₇₉E
ILAALP,

are particularly preferred as such deimmunized ABD exhibit substantially wild-type binding while providing attenuated MHC class II binding. Thus, the first polypeptide chain of such a diabody having an ABD contains a third linker (Linker 3) preferably positioned C-terminally to the E-coil (or K-coil) Domain of such polypeptide chain so as to intervene between the E-coil (or K-coil) Domain and the ABD (which is preferably a deimmunized ABD). A preferred sequence for such Linker 3 is SEQ ID NO:16: GGGS.

B. Diabodies Comprising Fc Domains

One embodiment of the present invention relates to multispecific diabodies (e.g., bispecific, trispecific, tetraspecific, etc.) capable of simultaneously binding a first and to a second epitope (i.e., a different epitope of the same antigen molecule or an epitope of a molecule that is a different antigen) that comprise an Fc Domain. The Fc Domain of such molecules may be of any isotype (e.g., IgG1, IgG2, IgG3, or IgG4). The molecules may further comprise a CH1 Domain and/or a Hinge Domain. When present, the CH1 Domain and/or Hinge Domain may be of any isotype (e.g., IgG1, IgG2, IgG3, or IgG4), and is preferably of the same isotype as the desired Fc Domain.

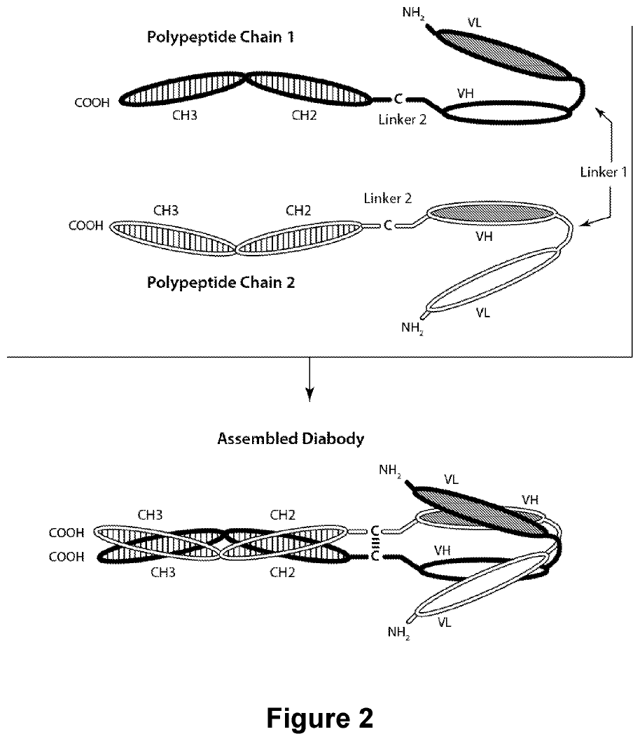

The addition of an IgG CH2-CH3 Domain to one or both of the diabody polypeptide chains, such that the complexing of the diabody chains results in the formation of an Fc Domain, increases the biological half-life and/or alters the valency of the diabody. Such diabodies comprise, two or more polypeptide chains whose sequences permit the polypeptide chains to covalently bind each other to form a covalently associated diabody that is capable of simultaneously binding a first epitope and to a second epitope. Incorporating an IgG CH2-CH3 Domains onto both of the diabody polypeptides will permit a two-chain bispecific Fc Region-containing diabody to form (FIG. 2).

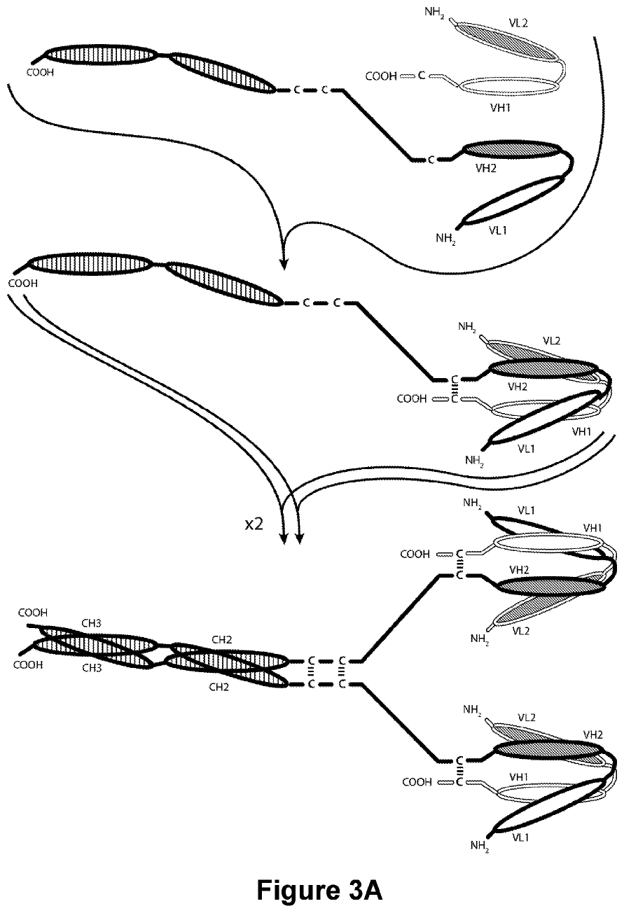

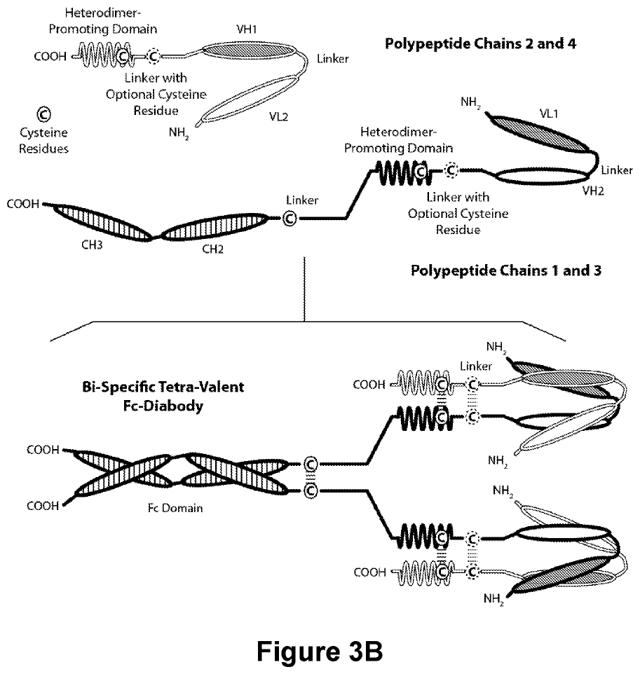

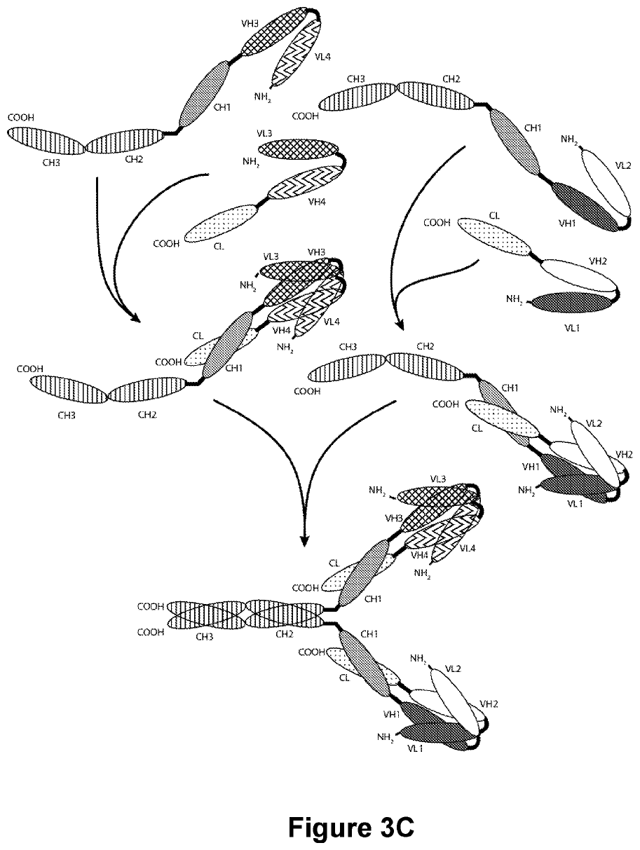

Alternatively, incorporating IgG CH2-CH3 Domains onto only one of the diabody polypeptides will permit a more complex four-chain bispecific Fc Domain-containing diabody to form (FIGS. 3A-3C). FIG. 3C shows a representative four-chain diabody possessing the Constant Light (CL) Domain and the Constant Heavy CH1 Domain, however fragments of such domains as well as other polypeptides may alternatively be employed (see, e.g., FIGS. 3A and 3B, United States Patent Publication Nos. 2013-0295121; 2010-0174053 and 2009-0060910; European Patent Publication No. EP 2714079; EP 2601216; EP 2376109; EP 2158221 and PCT Publication Nos. WO 2012/162068; WO 2012/018687; WO 2010/080538). Thus, for example, in lieu of the CH1 Domain, one may employ a peptide having the amino acid sequence GVEPKSC (SEQ ID NO:22), VEPKSC (SEQ ID NO:23), or AEPKSC (SEQ ID NO:24), derived from the Hinge Domain of a human IgG, and in lieu of the CL Domain, one may employ the C-terminal 6 amino acids of the human kappa Light Chain, GFNRGEC (SEQ ID NO:25) or FNRGEC (SEQ ID NO:26). A representative peptide containing four-chain diabody is shown in FIG. 3A. Alternatively, or in addition, one may employ a peptide comprising tandem coil domains of opposing charge such as the “E-coil” helical domains (SEQ ID NO:27: EVAALEK-EVAALEK-EVAALEK-EVAALEK or SEQ ID NO:29: EVAACEK-EVAALEK-EVAALEK-EVAALEK); and the “K-coil” domains (SEQ ID NO:28: KVAALKE-KVAALKE-KVAALKE-KVAALKE or SEQ ID NO:30: KVAACKE-KVAALKE-KVAALKE-KVAALKE).

A representative coil domain containing four-chain diabody is shown in FIG. 3B. Fc Domain-containing diabody molecules of the present invention may include additional intervening spacer peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Domain (preferably a cysteine-containing portion of an IgG Hinge Domain). Linkers that may be employed in the bispecific Fc Domain-containing diabody molecules of the present invention include: GGGS (SEQ ID NO:16), LGGGSG (SEQ ID NO:17), GGGSGGGSGGG (SEQ ID NO:18), ASTKG (SEQ ID NO:19), LEPKSS (SEQ ID NO:20), APSSS (SEQ ID NO:21), APSSSPME (SEQ ID NO:35), VEPKSADKTHTCPPCP (SEQ ID NO:36), LEPKSADKTHTCPPCP (SEQ ID NO:37), DKTHTCPPCP (SEQ ID NO:38), GGC, and GGG. LEPKSS (SEQ ID NO:20) may be used in lieu of GGG or GGC for ease of cloning. Additionally, the amino acids GGG, or LEPKSS (SEQ ID NO:20) may be immediately followed by DKTHTCPPCP (SEQ ID NO:38) to form the alternate linkers: GGGDKTHTCPPCP (SEQ ID NO:39); and LEPKSSDKTHTCPPCP (SEQ ID NO:40). Bispecific Fc Domain-containing molecules of the present invention may incorporate an IgG Hinge Domain in addition to or in place of a linker. Exemplary Hinge Domains include: EPKSCDKTHTCPPCP (SEQ ID NO:4) from IgG1, ERKCCVECPPCP (SEQ ID NO:5) from IgG2, ESKYGPPCPSCP (SEQ ID NO:6) from IgG4, and ESKYGPPCPPCP (SEQ ID NO:7) an IgG4 Hinge variant comprising a stabilizing S228P substitution (as numbered by the EU index as set forth in Kabat) to reduce strand exchange.

As provided in FIG. 3A-3C, Fc Domain-containing diabodies of the invention may comprise four chains. The first and third polypeptide chains of such a diabody contain three domains: (i) a VL1-containing Domain, (ii) a VH2-containing Domain, (iii) a Heterodimer-Promoting Domain, and (iv) a Domain containing a CH2-CH3 sequence. The second and fourth polypeptide chains contain: (i) a VL2-containing Domain, (ii) a VH1-containing Domain, and (iii) a Heterodimer-Promoting Domain, where the Heterodimer-Promoting Domains promote the dimerization of the first/third polypeptide chains with the second/fourth polypeptide chains. The VL and/or VH Domains of the third and fourth polypeptide chains, and VL and/or VH Domains of the first and second polypeptide chains may be the same or different so as to permit tetravalent binding that is either monospecific, bispecific or tetraspecific. The notation “VL3” and “VH3” denote respectively, the Light Chain Variable Domain and Variable Heavy Chain Domain that bind a “third” epitope of such diabody. Similarly, the notation “VL4” and “VH4” denote respectively, the Light Chain Variable Domain and Variable Heavy Chain Domain that bind a “fourth” epitope of such diabody. The general structure of the polypeptide chains of a representative four-chain bispecific Fc Domain-containing diabodies of invention is provided in Table 1:

TABLE 1

| | | |
|---|---|---|
| Bispecific | 2^{nd} Chain | NH₂—VL2—VH1—HPD—COOH |
| | 1^{st} Chain | NH₂—VL1—VH2—HPD—CH2—CH3—COOH |
| | 1^{st} Chain | NH₂—VL1—VH2—HPD—CH2—CH3—COOH |
| | 2^{nd} Chain | NH₂—VL2—VH1—HPD—COOH |
| Tetraspe- | 2^{nd} Chain | NH₂—VL2—VH1—HPD—COOH |
| cific | 1^{st} Chain | NH₂—VL1—VH2—HPD—CH2—CH3—COOH |
| | 3^{rd} Chain | NH₂—VL3—VH4—HPD—CH2—CH3—COOH |
| | 4^{th} Chain | NH₂—VL4—VH3—HPD—COOH |

HPD = Heterodimer-Promoting Domain

In a specific embodiment, diabodies of the present invention are bispecific, tetravalent (i.e., possess four epitope-binding domains), Fc-containing diabodies that are composed of four total polypeptide chains (FIGS. 3A-3C). The bispecific, tetravalent, Fc-containing diabodies of the invention comprise two first epitope-binding domains and two second epitope-binding domains.

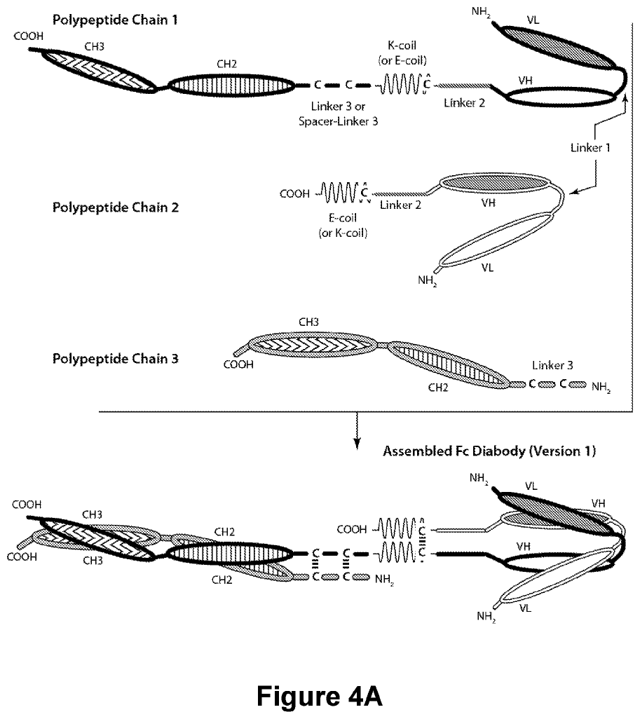

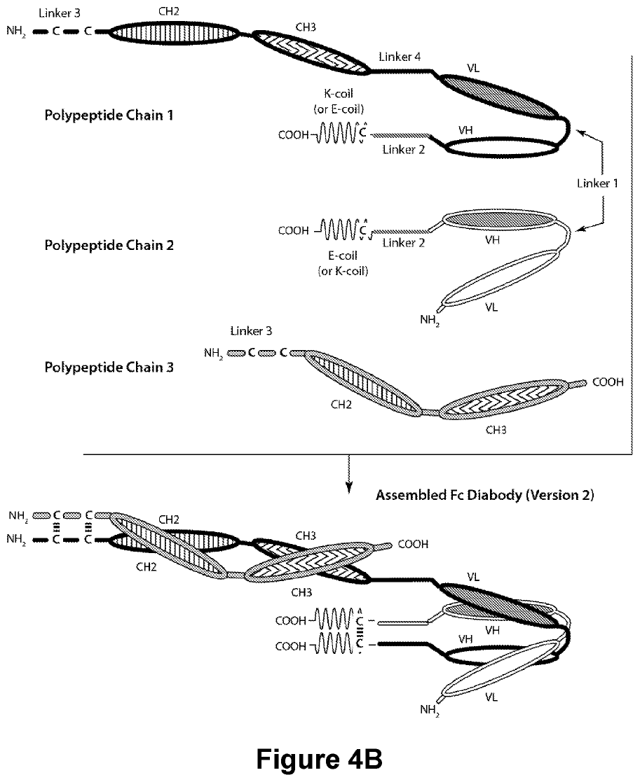

In a further embodiment, the Fc Domain-containing diabodies of the present invention may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 epitope-binding site that is capable of binding either the first or second epitope, as well as a VL2NH2 epitope-binding site that is capable of binding the other of such epitopes. The first and second polypeptides are bonded to one another through a disulfide bond involving cysteine residues in their respective Third Domains. Notably, the first and third polypeptide chains complex with one another to form an Fc Domain that is stabilized via a disulfide bond. Such bispecific diabodies have enhanced potency. FIGS. 4A and 4B illustrate the structures of such diabodies. Such Fc Region-containing diabodies may have either of two orientations (Table 2):

TABLE 2

| | | |
|---|---|---|
| First | 3^{rd} Chain | NH₂—CH2—CH3—COOH |
| Orien- | 1^{st} Chain | NH₂—VL1—VH2—HPD—CH2—CH3—COOH |
| tation | 2^{nd} Chain | NH₂—VL2—VH1—HPD—COOH |
| Second | 3^{rd} Chain | NH₂—CH2—CH3—COOH |
| Orien- | 1^{st} Chain | NH₂—CH2—CH3—VL1—VH2—HPD—COOH |
| tation | 2^{nd} Chain | NH₂—VL2—VH1—HPD—COOH |

HPD = Heterodimer-Promoting Domain

In a specific embodiment, diabodies of the present invention are bispecific, bivalent (i.e., possess two epitope-binding domains), Fc-containing diabodies that are composed of three total polypeptide chains (FIGS. 4A-4B). The bispecific, bivalent Fc-containing diabodies of the invention comprise one epitope-binding site immunospecific for either the first or second epitope, as well as a VL2/VH2 epitope-binding site that is capable of binding the other of such epitopes.

In a further embodiment, the Fc Domain-containing diabodies may comprise a total of five polypeptide chains. In a particular embodiment, two of the five polypeptide chains have the same amino acid sequence. The first polypeptide chain of such a diabody contains: (i) a VH1-containing Domain, (ii) a CH1-containing Domain, and (iii) a Domain containing a CH2-CH3 sequence. The first polypeptide chain may be the Heavy Chain of an antibody that contains a VH1 and a Heavy Chain constant region. The second and fifth polypeptide chains of such a diabody contain: (i) a VL1-containing Domain, and (ii) a CL-containing Domain. The second and/or fifth polypeptide chains of such a diabody may be Light Chains of an antibody that contains a VL1 complementary to the VH1 of the first/third polypeptide chain. The first, second and/or fifth polypeptide chains may be isolated from a naturally occurring antibody. Alternatively, they may be constructed recombinantly. The third polypeptide chain of such a diabody contains: (i) a VH1-containing Domain, (ii) a CH1-containing Domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain.

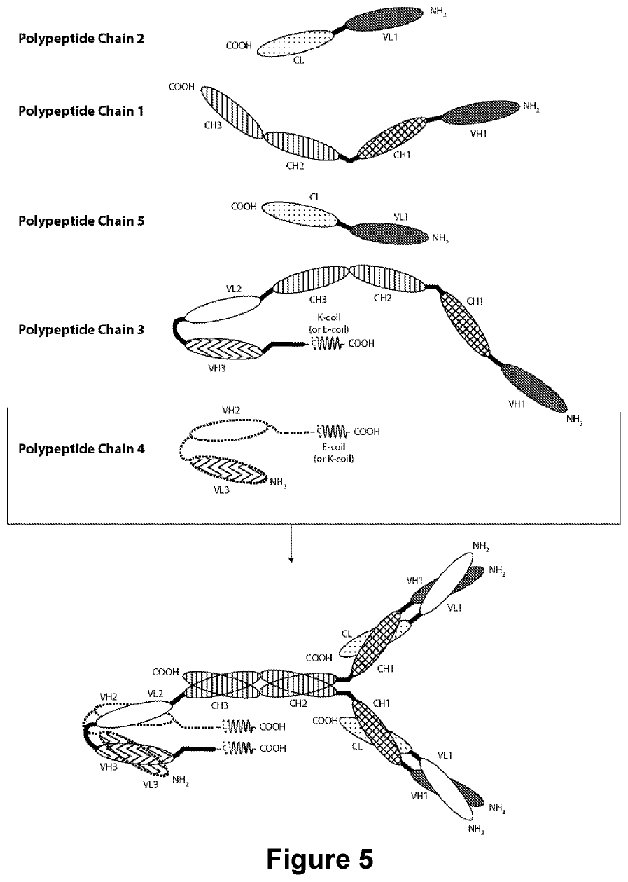

Thus, the first and second, and the third and fifth, polypeptide chains of such diabodies associate together to form two VL1/VH1 epitope-binding domains capable of binding a first epitope. The third and fourth polypeptide chains of such diabodies associate together to form a VL2/VH2 epitope-binding site that is capable of binding a second epitope, as well as a VL3/VH3 binding site that is capable of binding a third epitope. The first and third polypeptides are bonded to one another through a disulfide bond involving cysteine residues in their respective constant regions. Notably, the first and third polypeptide chains complex with one another to form an Fc Domain. Such multispecific diabodies have enhanced potency. FIG. 5 illustrates the structure of such diabodies. It will be understood that the VL1/VH1, VL2/VH2, and VL3/VH3 Domains may be the same or different so as to permit binding that is monospecific, bispecific or trispecific.

The VL and VH Domains of the polypeptide chains are selected so as to form VL/VH binding sites specific for a desired epitope. The VL/VH binding sites formed by the association of the polypeptide chains may be the same or different so as to permit tetravalent binding that is monospecific, bispecific, trispecific or tetraspecific. In particular, the VL and VH Domains maybe selected such that a multivalent diabody may comprise two binding sites for a first epitope and two binding sites for a second epitope, or three binding sites for a first epitope and one binding site for a second epitope, or two binding sites for a first epitope, one binding site for a second epitope and one binding site for a third epitope (as depicted in FIG. 5). The general structure of the polypeptide chains of representative five-chain Fc Domain-containing diabodies of invention is provided in Table 3:

TABLE 3

| | | |
|---|---|---|
| Bispecific | 2^{nd} Chain | NH₂—VL1—CL—COOH |
| (2 × 2) | 1^{st} Chain | NH₂—VH1—CH1—CH2—CH3—COOH |
| | 3^{rd} Chain | NH₂—VH1—CH1—CH2—CH3—VL2—VH2—HPD—COOH |
| | 5^{nd} Chain | NH₂—VL1—CL—COOH |
| | 4^{th} Cham | NH₂—VL2—VH2—HPD—COOH |
| Bispecific | 2^{nd} Chain | NH₂—VL1—CL—COOH |
| (3 × 1) | 1^{st} Chain | NH₂—VH1—CH1—CH2—CH3—COOH |
| | 3^{rd} Chain | NH₂—VH1—CH1—CH2—CH3—VL1—VH2—HPD—COOH |
| | 5^{nd} Chain | NH₂—VL1—CL—COOH |
| | 4^{th} Chain | NH₂—VL2—VH1—HPD—COOH |
| Trispecific | 2^{nd} Chain | NH₂—VL1—CL—COOH |
| (2 × 1 × 1) | 1^{st} Chain | NH₂—VH1—CH1—CH2—CH3—COOH |
| | 3^{rd} Chain | NH₂—VH1—CH1—CH2—CH3—VL2—VH3—HPD—COOH |
| | 5^{nd} Chain | NH₂—VL1—CL—COOH |
| | 4^{th} Chain | NH₂—VL3—VH2—HPD—COOH |

HPD = Heterodimer-Promoting Domain

In a specific embodiment, diabodies of the present invention are bispecific, tetravalent (i.e., possess four epitope-binding domains), Fc-containing diabodies that are composed of five total polypeptide chains having two epitope-binding domains immunospecific for the first epitope, and two epitope-binding domains specific for the second epitope. In another embodiment, the bispecific, tetravalent, Fc-containing diabodies of the invention comprise three epitope-binding domains immunospecific for the first epitope and one epitope-binding site specific for the second epitope. As provided above, the VL and VH Domains may be selected to permit trispecific binding. Accordingly, the invention also encompasses trispecific, tetravalent, Fc-containing diabodies. The trispecific, tetravalent, Fc-containing diabodies of the invention comprise two epitope-binding domains immunospecific for the first epitope, one epitope-binding site immunospecific for the second molecule, and one epitope-binding site immunospecific for the third epitope.

In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:8), IgG2 (SEQ ID NO:9), IgG3 (SEQ ID NO:10), and IgG4 (SEQ ID NO:11) are presented above.

Modification of the Fc Domain may lead to an altered phenotype, for example altered serum half-life, altered stability, altered susceptibility to cellular enzymes or altered effector function. It may therefore be desirable to modify an Fc Domain-containing binding molecule of the present invention with respect to effector function, for example, so as to enhance the effectiveness of such molecule in treating cancer. Reduction or elimination of Fc Domain-mediated effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Molecules of the invention possessing such conferred or altered effector function activity are useful for the treatment and/or prevention of a disease, disorder or infection in which an enhanced efficacy of effector function activity is desired.

Accordingly, in certain embodiments, the Fc Domain of the Fc Domain-containing molecules of the present invention may be an engineered variant Fc Domain. Although the Fc Domain of the bispecific Fc Domain-containing molecules of the present invention may possess the ability to bind one or more Fc receptors (e.g., FcγR(s)), more preferably such variant Fc Domain have altered binding FcγRIA (CD64), FcγRIIA (CD32A), FcγRIIB (CD32B), FcγRIIIA (CD16a) or FcγRIIIB (CD16b) (relative to the binding exhibited by a wild-type Fc Domain), e.g., will have enhanced binding an activating receptor and/or will have substantially reduced or no ability to bind inhibitory receptor(s). Thus, the Fc Domain of the Fc Domain-containing molecules of the present invention may include some or all of the CH2 Domain and/or some or all of the CH3 Domain of a complete Fc Domain, or may comprise a variant CH2 and/or a variant CH3 sequence (that may include, for example, one or more insertions and/or one or more deletions with respect to the CH2 or CH3 domains of a complete Fc Domain). Such Fc Domains may comprise non-Fc polypeptide portions, or may comprise portions of non-naturally complete Fc Domains, or may comprise non-naturally occurring orientations of CH2 and/or CH3 Domains (such as, for example, two CH2 Domains or two CH3 Domains, or in the N-terminal to C-terminal direction, a CH3 Domain linked to a CH2 Domain, etc.).

Fc Domain modifications identified as altering effector function are known in the art, including modifications that increase binding activating receptors (e.g., FcγRIIA (CD16A) and reduce binding inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Table 4 lists exemplary single, double, triple, quadruple and quintuple substitutions (numbering (according to the EU index) and substitutions are relative to the amino acid sequence of SEQ ID NO:8 as presented above) of exemplary modification that increase binding activating receptors and/or reduce binding inhibitory receptors.

TABLE 4
Variations of Preferred Activating Fc Domains†
Single-Site Variations

| | | | |
|---|---|---|---|
| F243L | R292G | D270E | R292P |
| Y300L | P396L | | |

Double-Site Variations

| | | | |
|---|---|---|---|
| F243L and R292P | F243L and Y300L | F243L and P396L | R292P and Y300L |
| D270E and P396L | R292P and V305I | P396L and Q419H | P247L and N421K |
| R292P and P396L | Y300L and P396L | R255L and P396L | R292P and P305I |
| K392T and P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D andR416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L and P396L | F243L, R292P, V305I, Y300L and P396L |
| L235P, F243L, R292P, Y300L and P396L | |

†numbering is according to the EU index as in Kabat

Exemplary variants of human IgG1 Fc Domains with reduced binding CD32B and/or increased binding CD16A contain F243L, R292P, Y300L, V305I or P296L substitutions. These amino acid substitutions may be present in a human IgG1 Fc Domain in any combination. In one embodiment, the variant human IgG1 Fc Domain contains a F243L, R292P and Y300L substitution. In another embodiment, the variant human IgG1 Fc Domain contains a F243L, R292P, Y300L, V305I and P296L substitution.

In certain embodiments, it is preferred for the Fc Domains of the Fc Domain-containing binding molecules of the present invention to exhibit decreased (or substantially no) binding FcγRIA (CD64), FcγRIIA (CD32A), FcγRIIB (CD32B), FcγRIIIA (CD16a) or FcγRIIIB (CD16b) (relative to the binding exhibited by the wild-type IgG1 Fc Domain (SEQ ID NO:8). In a specific embodiment, the Fc Domain-containing binding molecules of the present invention comprise an IgG Fc Domain that exhibits reduced ADCC effector function. In a preferred embodiment the CH2-CH3 Domains of such binding molecules include any 1, 2, 3, or 4 of the substitutions: L234A, L235A, D265A, N297Q, and N297G. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc Domain that inherently exhibits decreased (or substantially no) binding FcγRIIIA (CD16a) and/or reduced effector function (relative to the binding and effector function exhibited by the wild-type IgG1 Fc Domain (SEQ ID NO:8)) is utilized. In a specific embodiment, the Fc Domain-containing binding molecules of the present invention comprise an IgG2 Fc Domain (SEQ ID NO:9) or an IgG4 Fc Domain (SEQ ID NO:11). When an IgG4 Fc Domain is utilized, the instant invention also encompasses the introduction of a stabilizing mutation, such as the Hinge Region S228P substitution described above (see, e.g., SEQ ID NO:7). Since the N297G, N297Q, L234A, L235A and D265A substitutions abolish effector function, in circumstances in which effector function is desired, these substitutions would preferably not be employed.

A preferred IgG1 sequence for the CH2 and CH3 Domains of the Fc Domain-containing molecules of the present invention having reduced or abolished effector function will comprise the substitutions L234A/L235A (SEQ ID NO:41):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

The serum half-life of proteins comprising Fc Domains may be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered.

In some embodiments, the Fc Domain-containing binding molecules of the present invention comprise a variant Fc Domain that comprises at least one amino acid modification relative to a wild-type Fc Domain, such that the molecule has an increased half-life (relative to such molecule if comprising a wild-type Fc Domain). In some embodiments, the Fc Domain-containing binding molecules of the present invention comprise a variant IgG Fc Domain that comprises a half-life extending amino acid substitution at one or more positions selected from the group consisting of 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436. Numerous mutations capable of increasing the half-life of an Fc Domain-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and PCT Publication Nos. WO 98/23289; WO 2009/058492; and WO 2010/033279, which are herein incorporated by reference in their entireties.

In some embodiments, the Fc Domain-containing binding molecules of the present invention exhibiting enhanced half-life possess a variant Fc Domain comprising substitutions at two or more of Fc Domain residues 250, 252, 254, 256, 257, 288, 307, 308, 309, 311, 378, 428, 433, 434, 435 and 436. In particular, two or more substitutions selected from: T250Q, M252Y, S254T, T256E, K288D, T307Q, V308P, A378V, M428L, N434A, H435K, and Y436I. In a specific embodiment, such molecules may possess a variant IgG Fc Domain comprising the substitution:
(A) M252Y, S254T and T256E;
(B) M252Y and S254T;
(C) M252Y and T256E;
(D) T250Q and M428L;
(E) T307Q and N434A;
(F) A378V and N434A;
(G) N434A and Y436I;
(H) V308P and N434A; or
(I) K288D and H435K.

In a preferred embodiment, an Fc Domain-containing binding molecule of the present invention possesses a variant IgG Fc Domain comprising any 1, 2, or 3 of the substitutions: M252Y, S254T and T256E. The invention further encompasses such binding molecules that possess a variant Fc Domain comprising:
(A) one or more mutations which alter effector function and/or FcγR binding; and
(B) one or more mutations which extend serum half-life.

For certain antibodies, diabodies and trivalent binding molecules that are desired to have Fc-Domain-containing polypeptide chains of differing amino acid sequence (e.g., whose Fc Domain-containing first and third polypeptide chains are desired to not be identical), it is desirable to reduce or prevent homodimerization from occurring between the CH2-CH3 Domains of two first polypeptide chains or between the CH2-CH3 Domains of two third polypeptide chains. The CH2 and/or CH3 Domains of such polypeptide chains need not be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any pair of polypeptides comprising CH2-CH3 Domains that forms an Fc Domain to foster heterodimerization. Methods of protein engineering to favor heterodimerization over homodimerization are well-known in the art, in particular with respect to the engineering of immunoglobulin-like molecules, and are encompassed herein (see e.g., Ridgway et al. (1996) “Knobs-Into-Holes' Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

A preferred knob is created by modifying an IgG Fc Domain to contain the modification T366W. A preferred hole is created by modifying an IgG Fc Domain to contain the modification T366S, L368A and Y407V. To aid in purifying the hole-bearing third polypeptide chain homodimer from the final bispecific heterodimeric Fc Domain-containing molecule, the protein A binding site of the hole-bearing CH2 and CH3 Domains of the third polypeptide chain is preferably mutated by amino acid substitution at position 435 (H435R). Thus, the hole-bearing third polypeptide chain homodimer will not bind protein A, whereas the bispecific heterodimer will retain its ability to bind protein A via the protein A binding site on the first polypeptide chain. In an alternative embodiment, the hole-bearing third polypeptide chain may incorporate amino acid substitutions at positions 434 and 435 (N434A/N435K).

A preferred IgG amino acid sequence for the CH2 and CH3 Domains of the first polypeptide chain of an Fc Domain-containing molecule of the present invention will have the “knob-bearing” sequence (SEQ ID NO:42):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNRYTQKS LSLSPGX

wherein X is a lysine (K) or is absent.

A preferred IgG amino acid sequence for the CH2 and CH3 Domains of the second polypeptide chain of an Fc Domain-containing molecule of the present invention having two polypeptide chains (or the third polypeptide chain of an Fc Domain-containing molecule having three, four, or five polypeptide chains) will have the “hole-bearing” sequence (SEQ ID NO:43):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine (K) or is absent.

As will be noted, the CH2-CH3 Domains of SEQ ID NO:42, and SEQ ID NO:43 include a substitution at position 234 with alanine and 235 with alanine, and thus form an Fc Domain exhibit decreased (or substantially no) binding FcγRIA (CD64), FcγRIIA (CD32A), FcγRBB (CD32B), FcγRIIIA (CD16a) or FcγRIIIB (CD16b) (relative to the binding exhibited by the wild-type Fc Domain (SEQ ID NO:8). The invention also encompasses such CH2-CH3 Domains, which comprise the wild-type alanine residues, alternative and/or additional substitutions which modify effector function and/or FγR binding activity of the Fc Domain. The invention also encompasses such CH2-CH3 Domains, which further comprise one or more half-live extending amino acid substitutions. In particular, the invention encompasses such hole-bearing and such knob-bearing CH2-CH3 Domains which further comprise the M252Y/S254T/T256E.

It is preferred that the first polypeptide chain will have a “knob-bearing” CH2-CH3 sequence, such as that of SEQ ID NO:42. However, as will be recognized, a “hole-bearing”CH2-CH3 Domain (e.g., SEQ ID NO:43 could be employed in the first polypeptide chain, in which case, a “knob-bearing” CH2-CH3 Domain (e.g., SEQ ID NO:42) would be employed in the second polypeptide chain of an Fc Domain-containing molecule of the present invention having two polypeptide chains (or in the third polypeptide chain of an Fc Domain-containing molecule having three, four, or five polypeptide chains).

In other embodiments, the invention encompasses Fc Domain-containing binding molecules comprising CH2 and/or CH3 Domains that have been engineered to favor heterodimerization over homodimerization using mutations known in the art, such as those disclosed in PCT Publication No. WO 2007/110205; WO 2011/143545; WO 2012/058768; WO 2013/06867, all of which are incorporated herein by reference in their entirety.

### IV. Trivalent Binding Molecules Containing Fc Domains

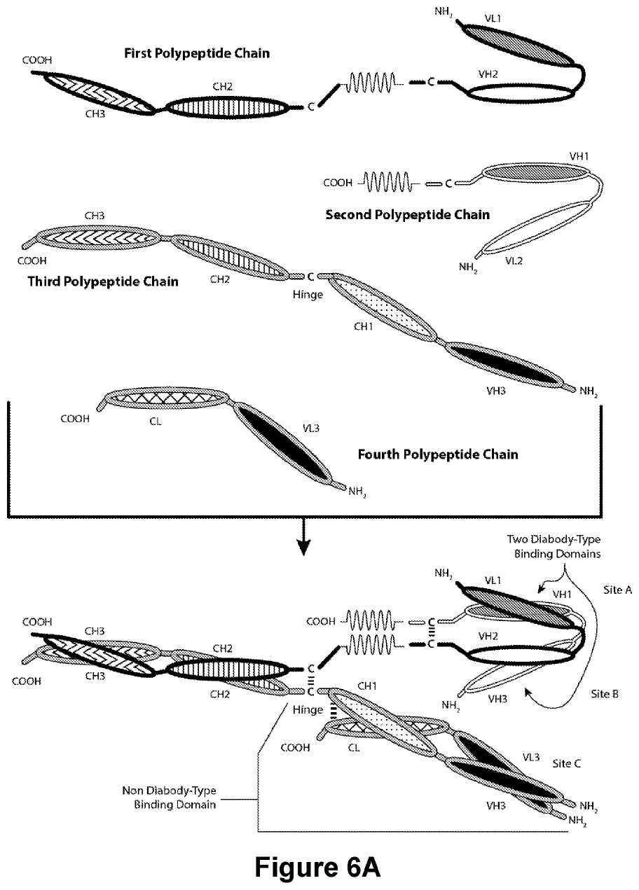

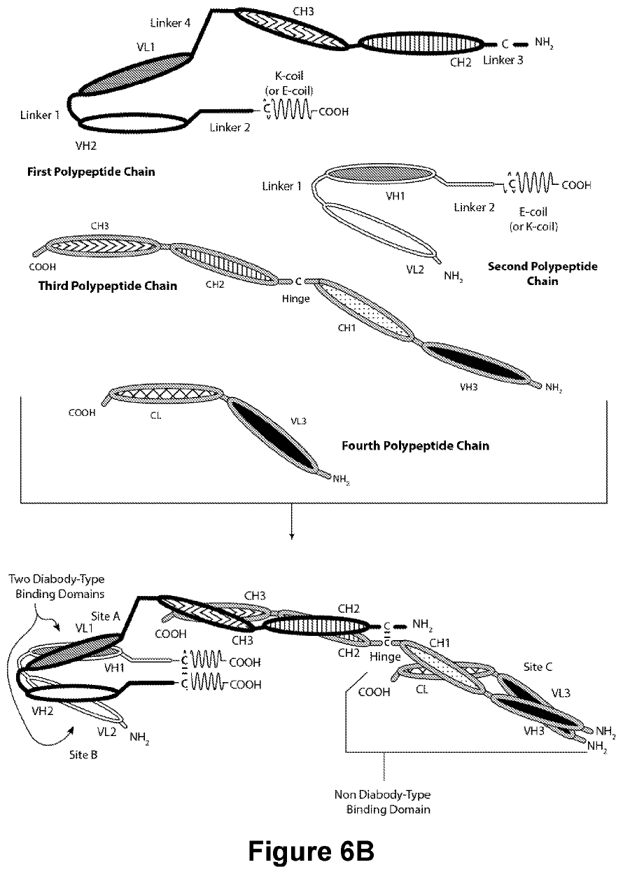

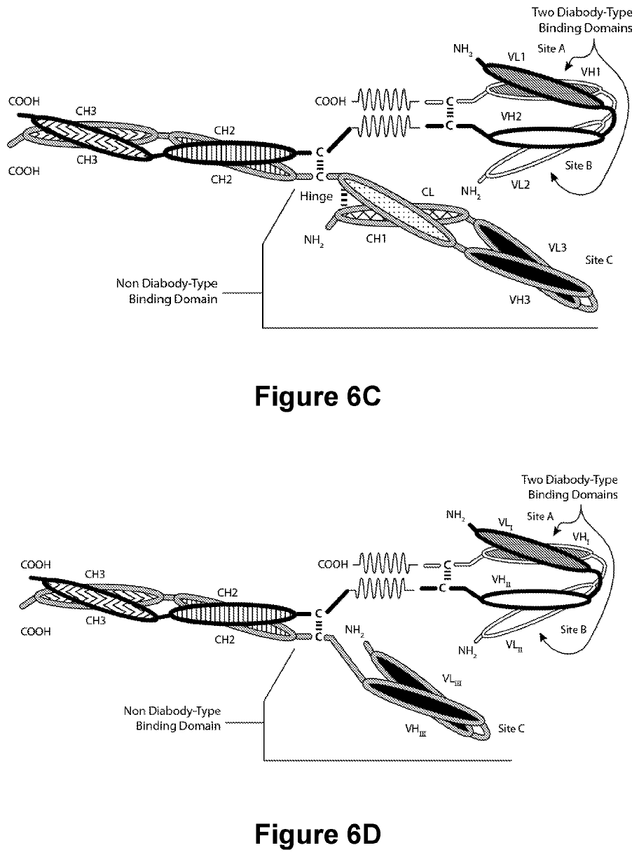

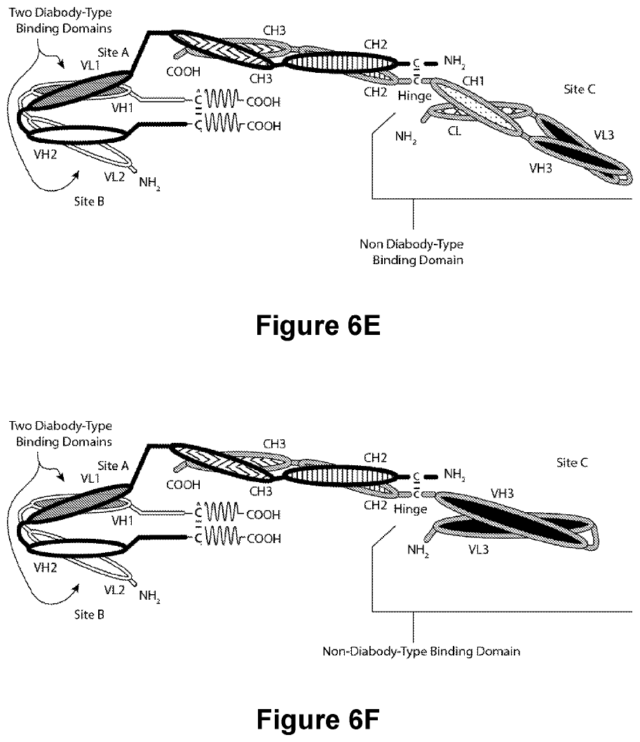

A further embodiment of the present invention relates to trivalent binding molecules comprising an Fc Domain capable of simultaneously binding a first epitope, a second epitope and a third epitope, wherein at least one of such epitopes is not identical to another. Such trivalent binding molecules comprise three epitope-binding domains, two of which are Diabody-Type Binding Domains, which provide binding Site A and binding Site B, and one of which is a Fab-Type Binding Domain, or an scFv-Type Binding Domain, which provides binding Site C (see, e.g., FIGS. 6A-6F, PCT Publication Nos. WO 2015/184207 and WO 2015/184203). Such trivalent binding molecules thus comprise “VL1”/“VH1” domains that are capable of binding the first epitope and “VL2”/“VH2” domains that are capable of binding the second epitope and “VL3” and “VH3” domains that are capable of binding the “third” epitope of such trivalent binding molecule. A “Diabody-Type Binding Domain” is the type of epitope-binding site present in a diabody, as described above. Each of a “Fab-Type Binding Domain” and an “scFv-Type Binding Domain” are epitope-binding domains that are formed by the interaction of the VL Domain of an immunoglobulin Light Chain and a complementing VH Domain of an immunoglobulin Heavy Chain. Fab-Type Binding Domains differ from Diabody-Type Binding Domains in that the two polypeptide chains that form a Fab-Type Binding Domain comprise only a single epitope-binding site, whereas the two polypeptide chains that form a Diabody-Type Binding Domain comprise at least two epitope-binding domains. Similarly, scFv-Type Binding Domains also differ from Diabody-Type Binding Domains in that they comprise only a single epitope-binding site. Thus, as used herein Fab-Type, and scFv-Type Binding Domains are distinct from Diabody-Type Binding Domains.

Typically, the trivalent binding molecules of the present invention will comprise four different polypeptide chains (see FIGS. 6A-6B), however, the molecules may comprise fewer or greater numbers of polypeptide chains, for example by fusing such polypeptide chains to one another (e.g., via a peptide bond) or by dividing such polypeptide chains to form additional polypeptide chains, or by associating fewer or additional polypeptide chains via disulfide bonds. FIGS. 6C-6F illustrate this aspect of the present invention by schematically depicting such molecules having three polypeptide chains. As provided in FIGS. 6A-6F, the trivalent binding molecules of the present invention may have alternative orientations in which the Diabody-Type Binding Domains are N-terminal (FIGS. 6A, 6C and 6D) or C-terminal (FIGS. 6B, 6E and 6F) to an Fc Domain. CH2 and CH3 Domains useful for the generation of trivalent binding molecules are provided above and include knob-bearing and hole-bearing domains.

In certain embodiments, the first polypeptide chain of such trivalent binding molecules of the present invention contains: (i) a VL1-containing Domain, (ii) a VH2-containing Domain, (iii) a Heterodimer-Promoting Domain, and (iv) a Domain containing a CH2-CH3 sequence. The VL 1 and VL2 Domains are located N-terminal or C-terminal to the CH2-CH3-containing domain as presented in Table 4 (also see, FIGS. 6A and 6B). The second polypeptide chain of such embodiments contains: (i) a VL2-containing Domain, (ii) a VH1-containing Domain, and (iii) a Heterodimer-Promoting Domain. The third polypeptide chain of such embodiments contains: (i) a VH3-containing Domain, (ii) a CH1-containing Domain and (iii) a Domain containing a CH2-CH3 sequence. The third polypeptide chain may be the Heavy Chain of an antibody that contains a VH3 and a Heavy Chain constant region, or a polypeptide that contains such domains. The fourth polypeptide of such embodiments contains: (i) a VL3-containing Domain and (ii) a CL-containing Domain. The fourth polypeptide chains may be a Light Chain of an antibody that contains a VL3 complementary to the VH3 of the third polypeptide chain, or a polypeptide that contains such domains. The third or fourth polypeptide chains may be isolated from naturally occurring antibodies. Alternatively, they may be constructed recombinantly, synthetically or by other means.

The Light Chain Variable Domain of the first and second polypeptide chains are separated from the Heavy Chain Variable Domains of such polypeptide chains by an intervening spacer peptide having a length that is too short to permit their VL1/VH2 (or their VL2/VH1) domains to associate together to form epitope-binding site capable of binding either the first or second epitope. A preferred intervening spacer peptide (Linker 1) for this purpose has the sequence (SEQ ID NO:14): GGGSGGGG. Other Domains of the trivalent binding molecules may be separated by one or more intervening spacer peptides (Linkers), optionally comprising a cysteine residue. In particular, as provided above, such Linkers will typically be incorporated between Variable Domains (i.e., VH or VL) and peptide Heterodimer-Promoting Domains (e.g., an E-coil or K-coil) and between such peptide Heterodimer-Promoting Domains (e.g., an E-coil or K-coil) and CH2-CH3 Domains. Exemplary linkers useful for the generation of trivalent binding molecules are provided above and are also provided in PCT Application Nos: PCT/US15/33081; and PCT/US15/33076. Thus, the first and second polypeptide chains of such trivalent binding molecules associate together to form a VL1/VH1 binding site capable of binding a first epitope, as well as a VL2/VH2 binding site that is capable of binding a second epitope. The third and fourth polypeptide chains of such trivalent binding molecules associate together to form a VL3/VH3 binding site that is capable of binding a third epitope.

As described above, the trivalent binding molecules of the present invention may comprise three polypeptides. Trivalent binding molecules comprising three polypeptide chains may be obtained by linking the domains of the fourth polypeptide N-terminal to the VH3-containing Domain of the third polypeptide (e.g., using an intervening spacer peptide (Linker 4)). Alternatively, a third polypeptide chain of a trivalent binding molecule of the invention containing the following domains is utilized: (i) a VL3-containing Domain, (ii) a VH3-containing Domain, and (iii) a Domain containing a CH2-CH3 sequence, wherein the VL3 and VH3 are spaced apart from one another by an intervening spacer peptide that is sufficiently long (at least 9 or more amino acid residues) so as to allow the association of these domains to form an epitope-binding site. One preferred intervening spacer peptide for this purpose has the sequence: GGGGSGGGGSGGGGS (SEQ ID NO:44).

It will be understood that the VL1/VH1, VL2/VH2, and VL3/VH3 Domains of such trivalent binding molecules may be different so as to permit binding that is monospecific, bispecific or trispecific. In particular, the VL and VH Domains may be selected such that a trivalent binding molecule comprises two binding sites for a first epitope and one binding sites for a second epitope, or one binding site for a first epitope and two binding sites for a second epitope, or one binding site for a first epitope, one binding site for a second epitope and one binding site for a third epitope.

The general structure of the polypeptide chains of representative trivalent binding molecules of invention is provided in FIGS. 6A-6F and in Table 5:

TABLE 5

| | | |
|---|---|---|
| Four | 2^{nd} Chain | NH₂—VL2—VH1—HPD—COOH |
| Chain | 1^{st} Chain | NH₂—VL1—VH2—HPD—CH2—CH3—COOH |
| 1^{st} Orien- | 3^{rd} Chain | NH₂—VH3—CH1—CH2—CH3—COOH |
| tation | 2^{nd} Chain | NH₂—VL3—CL—COOH |
| Four | 2^{nd} Chain | NH₂—VL2—VH1—HPD—COOH |
| Chain | 1^{st} Chain | NH₂—CH2—CH3—VL1—VH2—HPD—COOH |
| 2nd Orien- | 3^{rd} Chain | NH₂—VH3—CH1—CH2—CH3—COOH |
| tation | 2^{nd} Chain | NH₂—VL3—CL—COOH |
| Three | 2^{nd} Chain | NH₂—VL2—VH1—HPD—COOH |
| Chain | 1^{st} Chain | NH₂—VL1—VH2—HPD—CH2—CH3—COOH |
| 1st Orien- | 3^{rd} Chain | NH₂—VL3—VH3—HPD—CH2—CH3—COOH |
| tation | | |
| Three | 2^{nd} Chain | NH₂—VL2—VH1—HPD—COOH |
| Chain | 1^{st} Chain | NH₂—CH2—CH3—VL1—VH2—HPD—COOH |
| 2^{nd} Orien- | 3^{rd} Chain | NH₂—VL3—VH3—HPD—CH2—CH3—COOH |
| tation | | |

HPD = Heterodimer-Promoting Domain

As provided above, such trivalent binding molecules may comprise three, four, five, or more polypeptide chains.

### V. Embodiments of the Invention

As stated above, the present invention is directed to a combination therapy for the treatment of cancer that comprises the administration of:
(1) a molecule capable of binding PD-1 or a natural ligand of PD-1; and
(2) a molecule (e.g., a diabody, a BiTe, a bispecific antibody, etc.) capable of mediating the redirected killing of a target cell.
   The present invention is also directed to pharmaceutical compositions that comprise such molecule(s).

As used herein, the term “administration” relates to the provision of such molecules at a relative dosage and in temporal proximity so as to provide a recipient with both binding of PD-1 or a natural ligand of PD-1, and the redirected killing of the target cell (e.g., a cancer cell or a pathogen-infected cell).

With regard to the molecule capable of binding PD-1 or a natural ligand of PD-1, the invention particularly concerns the embodiment in which such molecule possesses the ability to immunospecifically bind an epitope of PD-1 so as to inhibit (i.e., block or interfere with) the inhibitory activity of PD-1. For example, such a molecule may bind PD-1 thereby inhibit cell signaling and/or inhibit binding between PD-1 and a natural ligand of PD-1. Alternatively, such molecule may bind a natural ligand of PD-1 (e.g., B7-H1 or B7-DC) so as to inhibit (i.e., block or interfere with) the inhibitory activity of such natural ligand. For example, such a molecule may bind a natural ligand of PD-1 to thereby inhibit cell signaling and/or binding between such ligand and PD-1. In one embodiment, such molecules will be monospecific so as to possess the ability to bind only a single epitope (e.g., an epitope of PD-1 or an epitope of a natural ligand of PD-1). Alternatively, such molecules may be multispecific, i.e., capable of binding two, or more than two, epitopes of PD-1 (e.g., 2, 3, 4, or more than 4 epitopes of PD-1), or capable of binding two, or more than two (e.g., 2, 3, 4, or more than 4) epitopes of one or more natural ligand(s) of PD-1, or be capable of binding at least one epitope of PD-1 and at least one epitope of a natural ligand of PD-1. Alternatively, such multispecific molecules are capable of binding at least one epitope of PD-1 and binding at least one epitope of a different molecule that is not PD-1, or capable of binding at least one epitope of a natural ligand of PD-1 and at least one epitope of a different molecule that is not a natural ligand of PD-1. Preferably, the epitope of the different molecule is an epitope of a molecule involved in regulating an immune check point present on the surface of an immune cell (e.g., B7-H3, B7-H4, BTLA, CD40, CD40L, CD47, CD70, CD80, CD86, CD94, CD137, CD137L, CD226, CTLA-4, Galectin-9, GITR, GITRL, HHLA2, ICOS, ICOSL, KIR, LAG-3, LIGHT, MHC class I or II, NKG2a, NKG2d, OX40, OX40L, PD1H, PVR, SIRPa, TCR, TIGIT, TIM-3 or VISTA, and particularly CD137, LAG-3, OX40, TIGIT, TIM-3, or VISTA, see for example PCT Publications Nos. WO 2015/200119 and WO 2011/159877). Thus, for example, such molecule may bind:
(1) a single epitope of PD-1;
(2) two or more epitopes of PD-1;
(3) a single epitope of a natural ligand of PD-1;
(4) two or more epitopes of the same natural ligand of PD-1;
(5) an epitope of a first natural ligand of PD-1 and an epitope of a second natural ligand of PD-1;
(6) two or more epitopes of a first natural ligand of PD-1 and one or more epitopes of a second natural ligand of PD-1;
(7) one or more epitopes of PD-1 and one or more epitopes of a natural ligand of PD-1;
(8) one or more epitopes of PD-1 and one or more epitopes of a different molecule; or
(9) one or more epitopes of natural ligand of PD-1 and one or more epitopes of a different molecule.

With regard to the molecules of the present invention that are capable of mediating the redirected killing of a target cell (e.g., a cancer cell or a pathogen-infected cell), the invention particularly concerns the embodiment in which such molecule comprises a first epitope-binding site capable of immunospecifically binding an epitope of a cell surface molecule of an effector cell and a second epitope-binding site that is capable of immunospecifically binding an epitope of a Disease Antigen that is arrayed on the surface of such target cell. In one embodiment, such molecules possess the ability to bind only a single epitope of a cell surface molecule of an effector cell and only to a single epitope of a Disease Antigen that is arrayed on the surface of the target cell. Alternatively, with respect to either or both binding specificities such molecules may be capable of binding one, two, or more than two, epitopes of cell surface molecule(s) of the effector cell, and be capable of binding one, two, or more than two epitopes of Disease Antigen(s). Thus, for example, such molecule may bind:
(1) only a single epitope of a cell surface molecule of an effector cell and a single epitope of a Disease Antigen that is arrayed on the surface of the target cell;
(2) only a single epitope of such cell surface molecule of such effector cell and two, or more than two, epitopes of such Disease Antigen;
(3) only a single epitope of such cell surface molecule of such effector cell and one, two, or more than two, epitopes of such Disease Antigen and one, two, or more than two, epitopes of a different Disease Antigen;
(4) two, or more than two epitopes of such cell surface molecule of such effector cell and a single epitope of a Disease Antigen that is arrayed on the surface of the target cell;
(5) two, or more than two epitopes of such cell surface molecule of such effector cell and two, or more than two, epitopes of such Disease Antigen;
(6) two, or more than two epitopes of such cell surface molecule of such effector cell and one, two, or more than two, epitopes of such Disease Antigen and one, two, or more than two, epitopes of such different Disease Antigen;
(7) one, two, or more than two epitopes of such cell surface molecule of such effector cell and one, two, or more than two, epitopes of a different cell surface molecule of an effector cell (which may be the same type of effector cell or may be a different type of effector cell) and a single epitope of a Disease Antigen that is arrayed on the surface of the target cell;
(8) one, two, or more than two epitopes of such cell surface molecule of such effector cell and one, two, or more than two, epitopes of a different cell surface molecule of an effector cell (which may be the same type of effector cell or may be a different type of effector cell) and two, or more than two, epitopes of such Disease Antigen; or
(9) one, two, or more than two epitopes of such cell surface molecule of such effector cell and one, two, or more than two, epitopes of a different cell surface molecule of an effector cell (which may be the same type of effector cell or may be a different type of effector cell) and one, two, or more than two, epitopes of such Disease Antigen and one, two, or more than two, epitopes of such different Disease Antigen.

As an example, the invention contemplates a binding molecule that comprises a first epitope-binding site capable of immunospecifically binding an epitope of CD3 (as the cell surface molecule of an effector cell); a second epitope-binding site that is capable of immunospecifically binding an epitope of a Disease Antigen that is arrayed on the surface of such target cell; and a third epitope-binding site capable of immunospecifically binding an epitope of CD8 (as the different cell surface molecule of an effector cell).

Table 6A illustrates possible combination binding specificities of exemplary molecules of the invention capable of binding PD-1 or a natural ligand of PD-1. Table 6B illustrates possible combination binding specificities of exemplary multispecific molecules of the invention capable of binding PD-1 or a natural ligand of PD-1 and a molecule other than PD-1 or a natural ligand of PD-1. Table 7 illustrates possible combination binding specificities of exemplary molecules of the invention capable of mediating the redirected killing of a target cell.

TABLE 6A
Number of Epitopes Recognized by Exemplary Molecule of the Invention
Capable of Binding PD-1 or a Natural Ligand of PD-1

PD-1 Ligand

| PD-1 | 1^{st} PD-1 Ligand | 2^{nd} PD-1 Ligand |
|---|---|---|
| 1 | 0 | 0 |
| 1 | 1 | |
| 1 | | 1 |
| 1 | 2 | |
| 1 | | 2 |
| 1 | >2 | |
| 1 | | >2 |
| 2 | 0 | 0 |
| 2 | 1 | |
| 2 | | 1 |
| 2 | 2 | |
| 2 | | 2 |
| 2 | >2 | |
| 2 | | >2 |
| >2 | 0 | 0 |
| >2 | 1 | |
| >2 | | 1 |
| >2 | 2 | |
| >2 | | 2 |
| >2 | >2 | |
| >2 | | >2 |
| 0 | 1 | 0 |
| 0 | 1 | 1 |
| 0 | 2 | 0 |
| 0 | 2 | 1 |
| 0 | 2 | 2 |
| 0 | >2 | 1 |
| 0 | >2 | 2 |
| 1 | 0 | 0 |
| 0 | >2 | >2 |

TABLE 6B
Number of Epitopes Recognized by Exemplary Molecule
of the Invention Capable of Binding PD-1 or a Natural
Ligand of PD-1, and a Different Molecule

| PD-1 | Different Molecule | Ligand of PD-1 | Different Molecule |
|---|---|---|---|
| 1 | 1 | 1 | 1 |
| 1 | 2 | 1 | 2 |
| 1 | >2 | 1 | >2 |
| 2 | 1 | 2 | 1 |
| 2 | 2 | 2 | 2 |
| 2 | >2 | 2 | >2 |
| >2 | 1 | >2 | 1 |
| >2 | 2 | >2 | 2 |
| >2 | >2 | >2 | >2 |

TABLE 7
Number of Epitopes Recognized by Exemplary
Molecules of the Invention Capable of Mediating
the Redirected Killing of a Target Cell

| | |
|---|---|
| Cell Surface Molecule of | |
| an Effector Cell | Disease Antigen |

| 1^{st} Surface | 2^{nd} Surface | 1^{st} Disease | 2^{nd} Disease |
|---|---|---|---|
| Molecule | Molecule | Antigen | Antigen |
| 1 | 0 | 1 | 0 |
| 1 | 0 | 1 | 1 |
| 1 | 0 | 2 | 0 |
| 1 | 0 | 2 | 1 |
| 1 | 0 | 2 | 2 |
| 1 | 0 | >2 | 0 |
| 1 | 0 | >2 | 1 |
| 1 | 0 | >2 | 2 |
| 2 | 0 | 1 | 0 |
| 2 | 0 | 1 | 1 |
| 2 | 0 | 2 | 0 |
| 2 | 0 | 2 | 1 |
| 2 | 0 | 2 | 2 |
| 2 | 0 | >2 | 0 |
| 2 | 0 | >2 | 1 |
| 2 | 0 | >2 | 2 |
| >2 | 0 | 1 | 0 |
| >2 | 0 | 1 | 1 |
| >2 | 0 | 2 | 0 |
| >2 | 0 | 2 | 1 |
| >2 | 0 | 2 | 2 |
| >2 | 0 | >2 | 0 |
| >2 | 0 | >2 | 1 |
| >2 | 0 | >2 | 2 |
| 1 | 1 | 1 | 0 |
| 1 | 1 | 1 | 1 |
| 1 | 1 | 2 | 0 |
| 1 | 1 | 2 | 1 |
| 1 | 1 | 2 | 2 |
| 1 | 1 | >2 | 0 |
| 1 | 1 | >2 | 1 |
| 1 | 1 | >2 | 2 |
| 2 | 1 | 1 | 0 |
| 2 | 1 | 1 | 1 |
| 2 | 1 | 2 | 0 |
| 2 | 1 | 2 | 1 |
| 2 | 1 | 2 | 2 |
| 2 | 1 | >2 | 0 |
| 2 | 1 | >2 | 1 |
| 2 | 1 | >2 | 2 |
| 2 | 2 | 1 | 0 |
| 2 | 2 | 1 | 1 |
| 2 | 2 | 2 | 0 |
| 2 | 2 | 2 | 1 |
| 2 | 2 | 2 | 2 |
| 2 | 2 | >2 | 0 |
| 2 | 2 | >2 | 1 |
| 2 | 2 | >2 | 2 |
| >2 | 1 | 1 | 0 |
| >2 | 1 | 1 | 1 |
| >2 | 1 | 2 | 0 |
| >2 | 1 | 2 | 1 |
| >2 | 1 | 2 | 2 |
| >2 | 1 | >2 | 0 |
| >2 | 1 | >2 | 1 |
| >2 | 1 | >2 | 2 |
| >2 | 2 | 1 | 0 |
| >2 | 2 | 1 | 1 |
| >2 | 2 | 2 | 0 |
| >2 | 2 | 2 | 1 |
| >2 | 2 | 2 | 2 |
| >2 | 2 | >2 | 0 |
| >2 | 2 | >2 | 1 |
| >2 | 2 | >2 | 2 |
| >2 | >2 | 1 | 0 |
| >2 | >2 | 1 | 1 |
| >2 | >2 | 2 | 0 |
| >2 | >2 | 2 | 1 |
| >2 | >2 | 2 | 2 |
| >2 | >2 | >2 | 0 |
| >2 | >2 | >2 | 1 |
| >2 | >2 | >2 | 2 |

No limitation is placed on the nature of epitopes or additional epitopes that may be bound by the molecules of the present invention other than that such additional binding capability does not prevent the molecule that is capable of inhibiting binding PD-1 or a natural ligand of PD-1 from such binding and does not prevent the molecule that is capable of mediating the redirected killing of a target cell from mediating such redirected killing.

A. Exemplary Molecules Capable of Binding PD-1 or a Natural Ligand of PD-1

1. Binding Molecules Immunospecific for PD-1

Antibodies that are immunospecific for PD-1 are known and may be employed or adapted to serve as a molecule (e.g., a diabody, an scFv, an antibody, a CAR, a TandAb, etc.) capable of binding PD-1 or a natural ligand of PD-1 in accordance with the present invention (see, e.g., U.S. Patent Applications No. 62/198,867; 62/239,559; 62/255,140 U.S. Pat. Nos. 8,008,449; 8,552,154; PCT Patent Publications WO 2012/135408, WO 2012/145549, and WO 2013/014668) Preferred molecules capable of binding PD-1 or a natural ligand of PD-1 will exhibit the ability to bind a continuous or discontinuous (e.g., conformational) portion (epitope) of human PD-1 (CD279) and will preferably also exhibit the ability to bind PD-1 molecules of one or more non-human species, in particular, primate species (and especially a primate species, such as cynomolgus monkey). Additional desired antibodies may be made by isolating antibody-secreting hybridomas elicited using PD-1 or a peptide fragment thereof. A representative human PD-1 polypeptide (NCBI Sequence NP_005009.2; including a 20 amino acid residue signal sequence, shown underlined) and the 268 amino acid residue mature protein) has the amino acid sequence (SEQ ID NO:45):

MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

Preferred PD-1-binding molecules that may be used to bind PD-1 are characterized by any (one or more) of the following criteria:
(1) specifically binds human PD-1 as endogenously expressed on the surface of a stimulated human T-cell;
(2) specifically binds human PD-1 with an equilibrium binding constant (K_{D}) of 40 nM or less;
(3) specifically binds human PD-1 with an equilibrium binding constant (K_{D}) of 5 nM or less;
(4) specifically binds human PD-1 with an on rate (kₐ) of 1.5×10⁴ M⁻¹ min⁻¹ or more;
(5) specifically binds human PD-1 with an on rate (kₐ) of 90.0×10⁴ M⁻¹ min⁻¹ or more;
(6) specifically binds human PD-1 with an off rate (k_{d}) of 7×10⁻⁴ min⁻¹ or less;
(7) specifically binds human PD-1 with an off rate (k_{d}) of 2×10⁻⁴ min⁻¹ or less;
(8) specifically binds non-human primate PD-1 (e.g., PD-1 of cynomolgus monkey);
(9) inhibits (i.e., blocks or interferes with) the binding/the inhibitory activity) of PD-1 ligand (PD-L1/PD-L2) to PD-1;
(10) stimulates an immune response; and/or
(11) synergizes with an anti-human LAG-3 antibody to stimulate an antigen-specific T-cell response.

The preferred anti-human PD-1-binding molecules of the present invention that may be used to bind PD-1 possess humanized VH and/or VL Domains of murine anti-human PD-1 monoclonal antibodies “PD-1 mAb 1,” “PD-1 mAb 2,” “PD-1 mAb 3,” “PD-1 mAb 4,” “PD-1 mAb 5,” “PD-1 mAb 6,” “PD-1 mAb 7,” “PD-1 mAb 8,” “PD-1 mAb 9,” “PD-1 mAb 10,” “PD-1 mAb 11,” “PD-1 mAb 12,” “PD-1 mAb 13,” “PD-1 mAb 14,” or “PD-1 mAb 15,” and more preferably possess 1, 2 or all 3 of the CDR_{H}_{^{S}} of the VH Domain and/or 1, 2 or all 3 of the CDR_{L}_{^{S}} of the VL Domain of such antibodies. The invention particularly relates to such PD-1-binding molecules comprising a PD-1 binding domain that possess:
(A) (1) the three CDR_{H}_{^{S}} of the VH Domain of PD-1 mAb 1;
(2) the three CDR_{L}_{^{S}} of the VL Domain of PD-1 mAb 1;
(3) the three CDR_{H}_{^{S}} of the VH Domain of PD-1 mAb 1 and the three CDR_{L}_{^{S}} of the VL Domain of PD-1 mAb 1;
(4) the VH Domain of hPD-1 mAb 1 VH1;
(5) the VL Domain of hPD-1 mAb 1 VL1;
(6) the VH and VL Domains of hPD-1 mAb 1;
(B) (1) the three CDR_{H}_{^{S}} of the VH Domain of PD-1 mAb 2;
(2) the three CDR_{L}_{^{S}} of the VL Domain of the PD-1 mAb 2;
(3) the three CDR_{H}_{^{S}} of the VH Domain of PD-1 mAb 2 and the three CDR_{L}_{^{S}} of the VL Domain of PD-1 mAb 2;
(4) the VH Domain of hPD-1 mAb 2 VH1;
(5) the VL Domain of hPD-1 mAb 2 VL1;
(6) the VH and VL Domains of hPD-1 mAb 2;
(C) (1) the three CDR_{H}_{^{S}} of the VH Domain of PD-1 mAb 3;
(2) the three CDR_{L}_{^{S}} of the VL Domain of PD-1 mAb 3;
(3) the three CDR_{H}_{^{S}} of the VH Domain of PD-1 mAb 3 and the three CDR_{L}_{^{S}} of the VL Domain of PD-1 mAb 3;
(D) (1) the three CDR_{H}_{^{S}} of the VH Domain of PD-1 mAb 4;
(2) the three CDR_{L}_{^{S}} of the VL Domain of PD-1 mAb 4;
(3) the three CDR_{H}_{^{S}} of the VH Domain of PD-1 mAb 4 and the three CDR_{L}_{^{S}} of the VL Domain of PD-1 mAb 4;
(E) (1) the three CDR_{H}_{^{S}} of the VH Domain of PD-1 mAb 5;
(2) the three CDR_{L}_{^{S}} of the VL Domain of PD-1 mAb 5;
(3) the three CDR_{H}_{^{S}} of the VH Domain of PD-1 mAb 5 and the three CDR_{L}_{^{S}} of the VL Domain of PD-1 mAb 5;
(F) (1) the three CDR_{H}_{^{S}} of the VH Domain of PD-1 mAb 6;
(2) the three CDR_{L}_{^{S}} of the VL Domain of PD-1 mAb 6;
(3) the three CDR_{H}_{^{S}} of the VH Domain of PD-1 mAb 6 and the three CDR_{L}_{^{S}} of the VL Domain of PD-1 mAb 6;
(G) (1) the three CDR_{H}_{^{S}} of the VH Domain of PD-1 mAb 7;
(2) the three CDR_{L}_{^{S}} of the VL Domain of PD-1 mAb 7, or hPD-1 mAb 7 VL2, or hPD-1 mAb 7 VL3;
(3) the three CDR_{H}_{^{S}} of the VH Domain of PD-1 mAb 7 and the three
CDR_{L}_{^{S}} of the VL Domain of PD-1 mAb 7, or hPD-1 mAb 7 VL2, hPD-1 mAb 7 VL3;
(4) the VH Domain of hPD-1 mAb 7 VH1, or hPD-1 mAb 7 VH2;
(5) the VL Domain of hPD-1 mAb 7 VL1, or hPD-1 mAb 7 VL2, or hPD-1 mAb 7 VL 3;
(6) the VH and VL Domains of the hPD-1 mAb 7(1.1), or hPD-1 mAb 7(1.2), or hPD-1 mAb 7(1.3), or hPD-1 mAb 7(2.1), or hPD-1 mAb 7(2.2), or hPD-1 mAb 7(2.3);
(H) (1) the three CDR_{H}_{^{S}} of the VH Domain of PD-1 mAb 8;
(2) the three CDR_{L}_{^{S}} of the VL Domain of PD-1 mAb 8;
(3) the three CDR_{H}_{^{S}} of the VH Domain of PD-1 mAb 8 and the three CDR_{L}_{^{S}} of the VL Domain of PD-1 mAb 8;
(I) (1) the three CDR_{H}_{^{S}} of the VH Domain of PD-1 mAb 9, or hPD-1 mAb 9 VH2;
(2) the three CDR_{L}_{^{S}} of the VL Domain of PD-1 mAb 9, or hPD-1 mAb 9 VL2;
(3) the three CDR_{H}_{^{S}} of the VH Domain of PD-1 mAb 9, or hPD-1 mAb 9 VH2 and the three CDR_{L}_{^{S}} of the VL Domain of PD-1 mAb 9, or hPD-1 mAb 9 VL2;
(4) the VH Domain of hPD-1 mAb 9 VH1, or hPD-1 mAb 9 VH2;
(5) the VL Domain of hPD-1 mAb 9 VL1, or hPD-1 mAb 9 VL2;
(6) the VH and VL Domains of the hPD-1 mAb 9(1.1), or hPD-1 mAb 9(1.2), or hPD-1 mAb 9(2.1), or hPD-1 mAb 9(2.2);
(J) (1) the three CDR_{H}_{^{S}} of the VH Domain of PD-1 mAb 10;
(2) the three CDR_{L}_{^{S}} of the VL Domain of PD-1 mAb 10;
(3) the three CDR_{H}_{^{S}} of the VH Domain of PD-1 mAb 10 and the three CDR_{L}_{^{S}} of the VL Domain of PD-1 mAb 10;
(K) (1) the three CDR_{H}_{^{S}} of the VH Domain of PD-1 mAb 11;
(2) the three CDR_{L}_{^{S}} of the VL Domain of PD-1 mAb 11,
(3) the three CDR_{H}_{^{S}} of the VH Domain of PD-1 mAb 11 and the three CDR_{L}_{^{S}} of the VL Domain of PD-1 mAb 11;
(L) (1) the three CDR_{H}_{^{S}} of the VH Domain of PD-1 mAb 12;
(2) the three CDR_{L}_{^{S}} of the VL Domain of the PD-1 mAb 12;
(3) the three CDR_{H}_{^{S}} of the VH Domain of the PD-1 mAb 12 and the three CDR_{L}_{^{S}} of the VL Domain of PD-1 mAb 12;
(M) (1) the three CDR_{H}_{^{S}} of the VH Domain of PD-1 mAb 13;
(2) the three CDR_{L}_{^{S}} of the VL Domain of PD-1 mAb 13;
(3) the three CDR_{H}_{^{S}} of the VH Domain of PD-1 mAb 13 and the three CDR_{L}_{^{S}} of the VL Domain of PD-1 mAb 13;
(N) (1) the three CDR_{H}_{^{S}} of the VH Domain of PD-1 mAb 14;
(2) the three CDR_{L}_{^{S}} of the VL Domain of the PD-1 mAb 14;
(3) the three CDR_{H}_{^{S}} of the VH Domain of the PD-1 mAb 14 and the three CDR_{L}_{^{S}} of the VL Domain of PD-1 mAb 14;
(O) (1) the three CDR_{H}_{^{S}} of the VH Domain of PD-1 mAb 15;
(2) the three CDR_{L}_{^{S}} of the VL Domain of PD-1 mAb 15;
(3) the three CDR_{H}_{^{S}} of the VH Domain of PD-1 mAb 15 and the three CDR_{L}_{^{S}} of the VL Domain of PD-1 mAb 15;
(4) the VH Domain of hPD-1 mAb 15 VH1;
(5) the VL Domain of hPD-1 mAb 15 VL1,
(6) the VH and VL Domains of hPD-1 mAb 15;

or

that binds, or competes for binding with, the same epitope as PD-1 mAb 1, PD-1 mAb 2, PD-1 mAb 3, PD-1 mAb 4, PD-1 mAb 5, PD-1 mAb 6, PD-1 mAb 7, PD-1 mAb 8, PD-1 mAb 9, PD-1 mAb 10, PD-1 mAb 11, PD-1 mAb 12, PD-1 mAb 13, PD-1 mAb 14, or PD-1 mAb 15.

### (a) PD-1 mAb 1

The amino acid sequence of the VH Domain of murine anti-human PD-1 mAb 1 (SEQ ID NO:46) is shown below (CDR_{H} residues are shown underlined).

DVQLQESGPG RVKPSQSLSL TCTVTGFSIT NDYAWNWIRQ
FPGNKLEWMG HITYSGSTSY NPSLKSRISI TRDTSKNHFF
LQLSSVTPED TATYYCARDY GSGYPYTLDY WGQGTSVTVS S
CDR_{H}1 of PD-1 mAb 1 (SEQ ID NO: 47):
NDYAWN
CDR_{H}2 of PD-1 mAb 1 (SEQ ID NO: 48):
HITYSGSTSYNPSLKS
CDR_{H}3 of PD-1 mAb 1 (SEQ ID NO: 49):
DYGSGYPYTLDY

The amino acid sequence of the VL Domain of murine anti-human PD-1 mAb 1 (SEQ ID NO:50) is shown below (CDR_{L} residues are shown underlined):

QIVLTQSPAL MSASPGEKVT MTCSATSIVS YVYWYQQKPG
SSPQPWIYLT SNLASGVPAR FSGSGSGTSY SLTISSMEAE
DAATYYCQQW SDNPYTFGGG TKLEIK
CDR_{L}1 of PD-1 mAb 1 (SEQ ID NO: 51):
SATSIVSYVY
CDR_{L}2 of PD-1 mAb 1 (SEQ ID NO: 52):
LTSNLAS
CDR_{L}3 of PD-1 mAb 1 (SEQ ID NO: 53):
QQWSDNPYT

The above-described murine anti-human PD-1 antibody PD-1 mAb 1 was humanized and further deimmunized when antigenic epitopes were identified in order to demonstrate the capability of humanizing an anti-human PD-1 antibody so as to decrease its antigenicity upon administration to a human recipient. The humanization yielded one humanized VH Domain, designated herein as “hPD-1 mAb 1 VH1,” and one humanized VL Domain designated herein as “hPD-1 mAb 1 VL1.” Accordingly, an antibody comprising the humanized VL Domains paired with the humanized VH Domain is referred to as “hPD-1 mAb 1.”

The amino acid sequence of the VH Domain of hPD-1 mAb 1 VH1 (SEQ ID NO:54) is shown below (CDR_{H} residues are shown underlined):

DVQLQESGPG LVKPSQTLSL TCTVSGFSIS NDYAWNWIRQ
PPGKGLEWIG HITYSGSTSY NPSLKSRLTI TRDTSKNQFV
LTMTNMDPVD TATYYCARDY GSGYPYTLDY WGQGTTVTVS S

The amino acid sequence of the VL Domain of hPD-1 mAb 1 VL1 (SEQ ID NO:55) is shown below (CDR_{H} residues are shown underlined):

EIVLTQSPAT LSVSPGEKVT ITCSATSIVS YVYWYQQKPG
QAPQPLIYLT SNLASGIPAR FSGSGSGTDF TLTISSLEAE
DAATYYCQQW SDNPYTFGGG TKVEIK

### (b) PD-1 mAb 2

The amino acid sequence of the VH Domain of murine anti-human PD-1 mAb 2 (SEQ ID NO:56) is shown below (CDR_{H} residues are shown underlined).

DVQLVESGGG LVQPGGSRKL SCAASGFVFS SFGMHWVRQA
PEKGLEWVAY ISSGSMSISY ADTVKGRFTV TRDNAKNTLF
LQMTSLRSED TAIYYCASLS DYFDYWGQGT TLTVSS
CDR_{H}1 of PD-1 mAb 2 (SEQ ID NO: 57):
SFGMH
CDR_{H}2 of PD-1 mAb 2 (SEQ ID NO: 58):
YISSGSMSISYADTVKG
CDR_{H}3 of PD-1 mAb 2 (SEQ ID NO: 59):
LSDYFDY

The amino acid sequence of the VL Domain of murine anti-human PD-1 mAb 2 (SEQ ID NO:60) is shown below (CDR_{L} residues are shown underlined):

DVVMSQTPLS LPVSLGDQAS ISCRSSQSLV HSTGNTYLHW
YLQKPGQSPK LLIYRVSNRF SGVPDRFSGS GSGTDFTLKI
SRVEAEDLGV FFCSQTTHVP WTFGGGTKLE IK
CDR_{L}1 of PD-1 mAb 2 (SEQ ID NO: 61):
RSSQSLVHSTGNTYLH
CDR_{L}2 of PD-1 mAb 2 (SEQ ID NO: 62):
RVSNRFS
CDR_{L}3 of PD-1 mAb 2 (SEQ ID NO: 63):
SQTTHVPWT

The above-described murine anti-human PD-1 antibody PD-1 mAb 2 was humanized and further deimmunized when antigenic epitopes were identified in order to demonstrate the capability of humanizing an anti-human PD-1 antibody so as to decrease its antigenicity upon administration to a human recipient. The humanization yielded one humanized VH Domain, designated herein as “hPD-1 mAb 2 VH1,” and one humanized VL Domains designated herein as “hPD-1 mAb 1 VL1.” Accordingly, any antibody comprising the humanized VL Domains paired with the humanized VH Domain is referred to as “hPD-1 mAb 2.”

The amino acid sequence of the VH Domain of hPD-1 mAb 2 VH1 (SEQ ID NO:64) is shown below (CDR_{H} residues are shown underlined):

EVQLVESGGG LVQPGGSLRL SCAASGFVFS SFGMHWVRQA
PGKGLEWVAY ISSGSMSISY ADTVKGRFTI SRDNAKNTLY
LQMNSLRTED TALYYCASLS DYFDYWGQGT TVTVSS

The amino acid sequence of the VL Domain of hPD-1 mAb 2 VL1 (SEQ ID NO:65) is shown below (CDR_{H} residues are shown underlined):

DVVMTQSPLS LPVTLGQPAS ISCRSSQSLV HSTGNTYLHW
YLQKPGQSPQ LLIYRVSNRF SGVPDRFSGS GSGTDFTLKI
SRVEAEDVGV YYCSQTTHVP WTFGQGTKLE IK

### (c) PD-1 mAb 3

The amino acid sequence of the VH Domain of murine anti-human PD-1 mAb 3 (SEQ ID NO:66) is shown below (CDR_{H} residues are shown underlined).

QVQLQQSGAE LVRPGASVTL SCKASGYTFT DYVMHWVKQT
PVHGLEWIGT IDPETGGTAY NQKFKGKAIL TADKSSNTAY
MELRSLTSED SAVYYFTREK ITTIVEGTYW YFDVWGTGTT VTVSS
CDR_{H}1 of PD-1 mAb 3 (SEQ ID NO: 67):
DYVMH
CDR_{H}2 of PD-1 mAb 3 (SEQ ID NO: 68):
TIDPETGGTAYNQKFKG
CDR_{H}3 of PD-1 mAb 3 (SEQ ID NO: 69):
EKITTIVEGTYWYFDV

The amino acid sequence of the VL Domain of murine anti-human PD-1 mAb 3 (SEQ ID NO:70) is shown below (CDR_{L} residues are shown underlined):

DVLLTQTPLS LPVSLGDQAS ISCRSSQNIV HSNGDTYLEW
YLQKPGQSPK LLIYKVSNRF SGVPDRFSGS GSGTDFTLKI
SRVEAEDLGV YYCFQGSHLP YTFGGGTKLE IK
CDR_{L}1 of PD-1 mAb 3 (SEQ ID NO: 71):
RSSQNIVHSNGDTYLE
CDR_{L}2 of PD-1 mAb 3 (SEQ ID NO: 72):
KVSNRFS
CDR_{L}3 of PD-1 mAb 3 (SEQ ID NO: 73):
FQGSHLPYT

### (d) PD-1 mAb 4

The amino acid sequence of the VH Domain of murine anti-human PD-1 mAb 4 (SEQ ID NO:74) is shown below (CDR_{H} residues are shown underlined).

DVQLVESGGG LVQPGGSRKL SCAASGFVFS SFGMHWVRQA
PEKGLEWVAY ISSGSMSISY ADTVKGRFTV TRDNAKNTLF
LQMTSLRSED TAIYYCASLT DYFDYWGQGT TLTVSS
CDR_{H}1 of PD-1 mAb 4 (SEQ ID NO: 75):
SFGMH
CDR_{H}2 of PD-1 mAb 4 (SEQ ID NO: 76):
YISSGSMSISYADTVKG
CDR_{H}3 of PD-1 mAb 4 (SEQ ID NO: 77):
LTDYFDY

The amino acid sequence of the VL Domain of murine anti-human PD-1 mAb 4 (SEQ ID NO:78) is shown below (CDR_{L} residues are shown underlined):

DVVMSQTPLS LPVSLGDQAS ISCRSSQSLV HSTGNTYFHW
YLQKPGQSPK LLIYRVSNRF SGVPDRFSGS GSGTDFILKI
SRVEAEDLGV YFCSQTTHVP WTFGGGTKLE IK
CDR_{L}1 of PD-1 mAb 4 (SEQ ID NO: 79):
RSSQSLVHSTGNTYFH
CDR_{L}2 of PD-1 mAb 4 (SEQ ID NO: 80):
RVSNRFS
CDR_{L}3 of PD-1 mAb 4 (SEQ ID NO: 81):
SQTTHVPWT

### (e) PD-1 mAb 5

The amino acid sequence of the VH Domain of murine anti-human PD-1 mAb 5 (SEQ ID NO:82) is shown below (CDR_{H} residues are shown underlined).

QVQLQQPGVE LVRPGASVKL SCKASGYSFT AYWMNWMKQR
PGQGLEWIGV IHPSDSETWL NQKFKDKATL TVDKSSSTAY
MQLISPTSED SAVYYCAREH YGSSPFAYWG QGTLVTVSA
CDR_{H}1 of PD-1 mAb 5 (SEQ ID NO: 83):
AYWMN
CDR_{H}2 of PD-1 mAb 5 (SEQ ID NO: 84):
VIHPSDSETWLNQKFED
CDR_{H}3 of PD-1 mAb 5 (SEQ ID NO: 85):
EHYGSSPFAY

The amino acid sequence of the VL Domain of murine anti-human PD-1 mAb 5 (SEQ ID NO:86) is shown below (CDR_{L} residues are shown underlined):

DIVLTQSPAS LAVSLGQRAT ISCRANESVD NYGMSFMNWF
QQKPGQPPKL LIYAASNQGS GVPARFSGSG SGTDFSLNIH
PMEEDDTAMY FCQQSKEVPY TFGGGTKLEI K
CDR_{L}1 of PD-1 mAb 5 (SEQ ID NO: 87):
RANESVDNYGMSFMN
CDR_{L}2 of PD-1 mAb 5 (SEQ ID NO: 88):
AASNQGS
CDR_{L}3 of PD-1 mAb 5 (SEQ ID NO: 89):
QQSKEVPYT

### (f) PD-1 mAb 6

The amino acid sequence of the VH Domain of murine anti-human PD-1 mAb 6 (SEQ ID NO:90) is shown below (CDR_{H} residues are shown underlined).
EVKLVESGGG LVNPGGSLKL SCAASGFTFS SYGMSWVRQT PEKRLEWVAT ISGGGSDTYY PDSVKGRFTI SRDNAKNNLY LQMSSLRSED TALYYCARQK ATTWFAYWGQ GTLVTVST
CDR_{H}1 of PD-1 mAb 6 (SEQ ID NO:91): SYGMS
CDR_{H}2 of PD-1 mAb 6 (SEQ ID NO:92): TISGGGSDTYYPDSVKG CDR_{H}3 of PD-1 mAb 6 (SEQ ID NO:93): QKATTWFAY

The amino acid sequence of the VL Domain of murine anti-human PD-1 mAb 6 (SEQ ID NO:94) is shown below (CDR_{L} residues are shown underlined):

DIVLTQSPAS LAVSLGQRAT ISCRASESVD NYGISFMNWF
QQKPGQPPKL LIYPASNQGS GVPARFSGSG SGTDFSLNIH
PMEEDDAAMY FCQQSKEVPW TFGGGTKLEI K
CDR_{L}1 of PD-1 mAb 6 (SEQ ID NO: 95):
RASESVDNYGISFMN
CDR_{L}2 of PD-1 mAb 6 (SEQ ID NO: 96):
PASNQGS
CDR_{L}3 of PD-1 mAb 6 (SEQ ID NO: 97):
QQSKEVPWT

### (g) PD-1 mAb 7

The amino acid sequence of the VH Domain of murine anti-human anti-human PD-1 mAb 7 (SEQ ID NO:98) is shown below (CDR_{H} residues are shown underlined).

QVQLQQPGAE LVRPGASVKL SCKASGYSFT SYWMNWVKQR
PGQGLEWIGV IHPSDSETWL DQKFKDKATL TVDKSSTTAY
MQLISPTSED SAVYYCAREH YGTSPFAYWG QGTLVTVSS
CDR_{H}1 of PD-1 mAb 7 (SEQ ID NO: 99)
SYWMN
CDR_{H}2 of PD-1 mAb 7 (SEQ ID NO: 100)
VIHPSDSETWLDQKFKD
CDR_{H}3 of PD-1 mAb 7 (SEQ ID NO: 101)
EHYGTSPFAY

The amino acid sequence of the VL Domain of murine anti-human PD-1 mAb 7 (SEQ ID NO:102) is shown below (CDR_{L} residues are shown underlined):

DIVLTQSPAS LAVSLGQRAT ISCRANESVD NYGMSFMNWF
QQKPGQPPKL LIHAASNQGS GVPARFSGSG FGTDFSLNIH
PMEEDDAAMY FCQQSKEVPY TFGGGTKLEI K
CDR_{L}1 of PD-1 mAb 7 (SEQ ID NO: 103):
RANESVDNYGMSFMN
CDR_{L}2 of PD-1 mAb 7 (SEQ ID NO: 104):
AASNQGS
CDR_{L}3 of PD-1 mAb 7 (SEQ ID NO: 105):
QQSKEVPYT

The above-described murine anti-human PD-1 antibody PD-1 mAb 7 was humanized and further deimmunized when antigenic epitopes were identified in order to demonstrate the capability of humanizing an anti-human PD-1 antibody so as to decrease its antigenicity upon administration to a human recipient. The humanization yielded two humanized VH Domains, designated herein as “hPD-1 mAb 7 VH1,” and “hPD-1 mAb 7 VH2,” and three humanized VL Domains designated herein as “hPD-1 mAb 7 VL1,” “hPD-1 mAb 7 VL2,” and “hPD-1 mAb 7 VL3.” Any of the humanized VL Domains may be paired with either of the humanized VH Domains. Accordingly, any antibody comprising one of the humanized VL Domains paired with the humanized VH Domain is referred to generically as “hPD-1 mAb 7,” and particular combinations of humanized VH/VL Domains are referred to by reference to the specific VH/VL Domains, for example a humanized antibody comprising hPD-1 mAb 7 VH1 and hPD-1 mAb 1 VL2 is specifically referred to as “hPD-1 mAb 7(1.2).”

The amino acid sequence of the VH Domain of hPD-1 mAb 7 VH1 (SEQ ID NO:106) is shown below (CDR_{H} residues are shown underlined):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSS

The amino acid sequence of the VH Domain of hPD-1 mAb 7 VH2 (SEQ ID NO:107) is shown below (CDR_{H} residues are shown underlined):

QVQLVQSGAE VKKPGASVKV SCKASGYSFT SYWMNWVRQA
PGQGLEWAGV IHPSDSETWL DQKFKDRVTI TVDKSTSTAY
MELSSLRSED TAVYYCAREH YGTSPFAYWG QGTLVTVSS

The amino acid sequence of the VL Domain of hPD-1 mAb 7 VL1 (SEQ ID NO:108) is shown below (CDR_{H} residues are shown underlined):

EIVLTQSPAT LSLSPGERAT LSCRANESVD NYGMSFMNWF
QQKPGQPPKL LIHAASNQGS GVPSRFSGSG SGTDFTLTIS
SLEPEDFAVY FCQQSKEVPY TFGGGTKVEI K

The amino acid sequence of the VL Domain of hPD-1 mAb 7 VL2 (SEQ ID NO:109) is shown below (CDR_{H} residues are shown underlined):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI K

The amino acid sequence of the VL Domain of hPD-1 mAb 7 VL3 (SEQ ID NO:110) is shown below (CDR_{H} residues are shown underlined):

EIVLTQSPAT LSLSPGERAT LSCRASESVD NYGMSFMNWF
QQKPGQPPKL LIHAASNRGS GVPSRFSGSG SGTDFTLTIS
SLEPEDFAVY FCQQSKEVPY TFGGGTKVEI K

The CDR_{L}1 of the VL Domain of both hPD-1 mAb 7 VL2 and hPD-1 mAb 7 VL3 comprises an asparagine to serine amino acid substitution and has the amino acid sequence: RASESVDNYGMSFMN (SEQ ID NO:111), the substituted serine is shown underlined). It is contemplated that a similar substitution may be incorporated into any of the PD-1 mAb 7 CDR_{L}1 Domains described above.

In addition, the CDR_{L}2 of the VL Domain of hPD-1 mAb 7 VL3 comprises a glutamine to arginine amino acid substitution and has the amino acid sequence: AASNRGS (SEQ ID NO:112), the substituted arginine is shown underlined). It is contemplated that a similar substitution may be incorporated into any of the PD-1 mAb 7 CDR_{L}2 Domains described above.

### (h) PD-1 mAb 8

The amino acid sequence of the VH Domain of murine anti-human PD-1 mAb 8 (SEQ ID NO:113) is shown below (CDR_{H} residues are shown underlined).

EGQLQQSGPE LVKPGASVKI SCKASGYTFT DYYMNWVKQN
HGKSLEWIGD INPKNGDTHY NQKFKGEATL TVDKSSTTAY
MELRSLTSED SAVYYCASDF DYWGQGTTLT VSS
CDR_{H}1 of PD-1 mAb 8 (SEQ ID NO: 114):
DYYMN
CDR_{H}2 of PD-1 mAb 8 (SEQ ID NO: 115):
DINPKNGDTHYNQKFKG
CDR_{H}3 of PD-1 mAb 8 (SEQ ID NO: 116):
DFDY

The amino acid sequence of the VL Domain of murine anti-human PD-1 mAb 8 (SEQ ID NO:117) is shown below (CDR_{L} residues are shown underlined):

DVVMTQTPLS LPVGLGDQAS ISCRSSQTLV YSNGNTYLNW
FLQKPGQSPK LLIYKVSNRF SGVPDRFSGS GSGTDFTLKI
SRVEAEDLGV YFCSQSTHVP FTFGSGTKLE IK
CDR_{L}1 of PD-1 mAb 8 (SEQ ID NO: 118):
RSSQTLVYSNGNTYLN
CDR_{L}2 of PD-1 mAb 8 (SEQ ID NO: 119):
KVSNRFS
CDR_{L}3 of PD-1 mAb 8 (SEQ ID NO: 120):
SQSTHVPFT

### (i) PD-1 mAb 9

The amino acid sequence of the VH Domain of murine anti-human PD-1 mAb 9 (SEQ ID NO:121) is shown below (CDR_{H} residues are shown underlined).

EVMLVESGGG LVKPGGSLKL SCAASGFTFS SYLVSWVRQT
PEKRLEWVAT ISGGGGNTYY SDSVKGRFTI SRDNAKNTLY
LQISSLRSED TALYYCARYG FDGAWFAYWG QGTLVTVSS
CDR_{H}1 of PD-1 mAb 9 (SEQ ID NO: 122):
SYLVS
CDR_{H}2 of PD-1 mAb 9 (SEQ ID NO: 123):
TISGGGGNTYYSDSVKG
CDR_{H}3 of PD-1 mAb 9 (SEQ ID NO: 124):
YGFDGAWFAY

The amino acid sequence of the VL Domain of murine anti-human PD-1 mAb 9 (SEQ ID NO:125) is shown below (CDR_{L} residues are shown underlined):

DIQMTQSPAS LSASVGDIVT ITCRASENIY SYLAWYQQKQ
EKSPQLLVYN AKTLAAGVPS RFSGSGSGTQ FSLTINSLQP
EDFGNYYCQH HYAVPWTFGG GTRLEIT
CDR_{L}1 of PD-1 mAb 9 (SEQ ID NO: 126):
RASENIYSYLA
CDR_{L}2 of PD-1 mAb 9 (SEQ ID NO: 127):
NAKTLAA
CDR_{L}3 of PD-1 mAb 9 (SEQ ID NO: 128):
QHHYAVPWT

The above-described murine anti-human PD-1 antibody PD-1 mAb 9 was humanized and further deimmunized when antigenic epitopes were identified in order to demonstrate the capability of humanizing an anti-human PD-1 antibody so as to decrease its antigenicity upon administration to a human recipient. The humanization yielded two humanized VH Domains, designated herein as “hPD-1 mAb 9 VH1,” and “hPD-1 mAb 9 VH2,” and two humanized VL Domains designated herein as “hPD-1 mAb 9 VL1,” and “hPD-1 mAb 9 VL2.” Any of the humanized VL Domains may be paired with the humanized VH Domains. Accordingly, any antibody comprising one of the humanized VL Domains paired with the humanized VH Domain is referred to generically as “hPD-1 mAb 9,” and particular combinations of humanized VH/VL Domains are referred to by reference to the specific VH/VL Domains, for example a humanized antibody comprising hPD-1 mAb 9 VH1 and hPD-1 mAb 9 VL2 is specifically referred to as “hPD-1 mAb 9(1.2).”

The amino acid sequence of the VH Domain of hPD-1 mAb 9 VH1 (SEQ ID NO:129) is shown below (CDR_{H} residues are shown underlined):

EVQLVESGGG LVRPGGSLKL SCAASGFTFS SYLVSWVRQA
PGKGLEWVAT ISGGGGNTYY SDSVKGRFTI SRDNAKNSLY
LQMNSLRAED TATYYCARYG FDGAWFAYWG QGTLVTVSS

The amino acid sequence of the VH Domain of hPD-1 mAb 9 VH2 (SEQ ID NO:130) is shown below (CDR_{H} residues are shown underlined):

EVQLVESGGG LARPGGSLKL SCAASGFTFS SYLVGWVRQA
PGKGLEWTAT ISGGGGNTYY SDSVKGRFTI SRDNAKNSLY
LQMNSARAED TATYYCARYG FDGAWFAYWG QGTLVTVSS

The CDR_{H}1 of the VH Domain of hPD-1 mAb 9 VH2 comprises a serine to glycine amino acid substitution and has the amino acid sequence: SYLVG ((SEQ ID NO:131), the substituted glycine is shown underlined). It is contemplated that a similar substitution may be incorporated into any of the PD-1 mAb 9 CDR_{H}1 Domains described above.

The amino acid sequence of the VL Domain of hPD-1 mAb 9 VL1 (SEQ ID NO:132) is shown below (CDR_{H} residues are shown underlined):

DIQMTQSPSS LSASVGDRVT ITCRASENIY SYLAWYQQKP
GKAPKLLIYN AKTLAAGVPS RFSGSGSGTD FTLTISSLQP
EDFATYYCQH HYAVPWTFGQ GTKLEIK

The amino acid sequence of the VL Domain of hPD-1 mAb 9 VL2 (SEQ ID NO:133) is shown below (CDR_{H} residues are shown underlined):

DIQMTQSPSS LSASVGDRVT ITCRASENIY NYLAWYQQKP
GKAPKLLIYD AKTLAAGVPS RFSGSGSGTD FTLTISSLQP
EDFATYYCQH HYAVPWTFGQ GTKLEIK

The CDR_{L}1 of the VL Domain of hPD-1 mAb 9 VL2 comprises a serine to asparagine amino acid substitution and has the amino acid sequence: RASENIYNYLA (SEQ ID NO:134), the substituted asparagine is shown underlined). It is contemplated that a similar substitution may be incorporated into any of the PD-1 mAb 9 CDR_{L}1 Domains described above.

The CDR_{L}2 of the VL Domain of hPD-1 mAb 9 VL2 comprises an asparagine to aspartate amino acid substitution and has the amino acid sequence: DAKTLAA ((SEQ ID NO:135), the substituted aspartate is shown underlined). It is contemplated that a similar substitution may be incorporated into any of the PD-1 mAb 7 CDR_{L}2 Domains described above.

### (j) PD-1 mAb 10

The amino acid sequence of the VH Domain of murine anti-human PD-1 mAb 10 (SEQ ID NO:136) is shown below (CDR_{H} residues are shown underlined).

EVILVESGGG LVKPGGSLKL SCAASGFTFS NYLMSWVRQT
PEKRLEWVAS ISGGGSNIYY PDSVKGRFTI SRDNAKNTLY
LQMNSLRSED TALYYCARQE LAFDYWGQGT TLTVSS
CDR_{H}1 of PD-1 mAb 10 (SEQ ID NO: 137):
NYLMS
CDR_{H}2 of PD-1 mAb 10 (SEQ ID NO: 138):
SISGGGSNIYYPDSVKG
CDR_{H}3 of PD-1 mAb 10 (SEQ ID NO: 139):
QELAFDY

The amino acid sequence of the VL Domain of murine anti-human PD-1 mAb 10 (SEQ ID NO:140) is shown below (CDR_{L} residues are shown underlined):

DIQMTQTTSS LSASLGDRVT ISCRTSQDIS NFLNWYQQKP
DGTIKLLIYY TSRLHSGVPS RFSGSGSGTD YSLTISNLEQ
EDIATYFCQQ GSTLPWTFGG GTKLEII
CDR_{L}1 of PD-1 mAb 10 (SEQ ID NO: 141):
RTSQDISNFLN
CDR_{L}2 of PD-1 mAb 10 (SEQ ID NO: 142):
YTSRLHS
CDR_{L}3 of PD-1 mAb 10 (SEQ ID NO: 143)
QQGSTLPWT

### (k) PD-1 mAb 11

The amino acid sequence of the VH Domain of murine anti-human PD-1 mAb 11 (SEQ ID NO:144) is shown below (CDR_{H} residues are shown underlined).

EVQLQQSGTV LARPGASVKM SCKTSGYTFT GYWMHWVKQR
PGQGLKWMGA IYPGNSDTHY NQKFKGKAKL TAVTSASTAY
MELSSLTNED SAIYYCTTGT YSYFDVWGTG TTVTVSS
CDR_{H}1 of PD-1 mAb 11 (SEQ ID NO: 145):
GYWMH
CDR_{H}2 of PD-1 mAb 11 (SEQ ID NO: 146):
AIYPGNSDTHYNQKFKG
CDR_{H}3 of PD-1 mAb 11 (SEQ ID NO: 147):
GTYSYFDV

The amino acid sequence of the VL Domain of murine anti-human PD-1 mAb 11 (SEQ ID NO:148) is shown below (CDR_{L} residues are shown underlined):

DILLTQSPAI LSVSPGERVS FSCRASQSIG TSIHWYQHRT
NGSPRLLIKY ASESISGIPS RFSGSGSGTD FTLSINSVES
EDIADYYCQQ SNSWLTFGAG TKLELK
CDR_{L}1 of PD-1 mAb 11 (SEQ ID NO: 149):
RASQSIGTSIH
CDR_{L}2 of PD-1 mAb 11 (SEQ ID NO: 150):
YASESIS
CDR_{L}3 of PD-1 mAb 11 (SEQ ID NO: 151):
QQSNSWLT

### (l) PD-1 mAb 12

The amino acid sequence of the VH Domain of murine anti-human PD-1 mAb 12 (SEQ ID NO:152) is shown below (CDR_{H} residues are shown underlined).

QGHLQQSGAE LVRPGASVTL SCKASGFTFT DYEMHWVKQT
PVHGLEWIGT IDPETGGTAY NQKFKGKAIL TVDKSSTTTY
MELRSLTSED SAVFYCSRER ITTVVEGAYW YFDVWGTGTT
VTVSS
CDR_{H}1 of PD-1 mAb 12 (SEQ ID NO: 153):
DYEMH
CDR_{H}2 of PD-1 mAb 12 (SEQ ID NO: 154):
TIDPETGGTAYNQKFKG
CDR_{H}3 of PD-1 mAb 12 (SEQ ID NO: 155):
ERITTVVEGAYWYFDV

The amino acid sequence of the VL Domain of murine anti-human PD-1 mAb 12 (SEQ ID NO:156) is shown below (CDR_{L} residues are shown underlined):

DVLMTQTPLS LPVSLGDQAS ISCRSSQNIV HSNGNTYLEW
YLQKPGQSPK LLICKVSTRF SGVPDRFSGS GSGTDFTLKI
SRVEAEDLGV YYCFQGSHVP YTFGGGTKLE IK
CDR_{L}1 of PD-1 mAb 12 (SEQ ID NO: 157):
RSSQNIVHSNGNTYLE
CDR_{L}2 of PD-1 mAb 12 (SEQ ID NO: 158):
KVSTRFS
CDR_{L}3 of PD-1 mAb 12 (SEQ ID NO: 159):
FQGSHVPYT

### (m) PD-1 mAb 13

The amino acid sequence of the VH Domain of murine anti-human PD-1 mAb 13 (SEQ ID NO:160) is shown below (CDR_{H} residues are shown underlined).

EVMLVESGGG LVKPGGSLKL SCAASGFTFS SHTMSWVRQT
PEKRLEWVAT ISGGGSNIYY PDSVKGRFTI SRDNAKNTLY
LQMSSLRSED TALYYCARQA YYGNYWYFDV WGTGTTVTVS S
CDR_{H}1 of PD-1 mAb 13 (SEQ ID NO: 161):
SHTMS
CDR_{H}2 of PD-1 mAb 13 (SEQ ID NO: 162):
TISGGGSNIYYPDSVKG
CDR_{H}3 of PD-1 mAb 13 (SEQ ID NO: 163):
QAYYGNYWYFDV

The amino acid sequence of the VL Domain of murine anti-human PD-1 mAb 13 (SEQ ID NO:164) is shown below (CDR_{L} residues are shown underlined):

DIQMTQSPAT QSASLGESVT ITCLASQTIG TWLAWYQQKP
GKSPQLLIYA ATSLADGVPS RFSGSGSGTK FSFKISSLQA
EDFVSYYCQQ LDSIPWTFGG GTKLEIK
CDR_{L}1 of PD-1 mAb 13 (SEQ ID NO: 165):
LASQTIGTWLA
CDR_{L}2 of PD-1 mAb 13 (SEQ ID NO: 166):
AATSLAD
CDR_{L}3 of PD-1 mAb 13 (SEQ ID NO: 167):
QQLDSIPWT

### (n) PD-1 mAb 14

The amino acid sequence of the VH Domain of murine anti-human PD-1 mAb 14 (SEQ ID NO:168) is shown below (CDR_{H} residues are shown underlined).

QVQLQQPGAE LVKPGASVKM SCKASGYNFI SYWITWVKQR
PGQGLQWIGN IYPGTDGTTY NEKFKSKATL TVDTSSSTAY
MHLSRLTSED SAVYYCATGL HWYFDVWGTG TTVTVSS
CDR_{H}1 of PD-1 mAb 14 (SEQ ID NO: 169):
SYWIT
CDR_{H}2 of PD-1 mAb 14 (SEQ ID NO: 170):
NIYPGTDGTTYNEKFKS
CDR_{H}3 of PD-1 mAb 14 (SEQ ID NO: 171):
GLHWYFDV

The amino acid sequence of the VL Domain of murine anti-human PD-1 mAb 14 (SEQ ID NO:172) is shown below (CDR_{L} residues are shown underlined):

DIVMTQSQKF MSTSVGDRVS VTCKASQSVG TNVAWYQQKP
GQSPKALIYS ASSRFSGVPD RFTGSGSGTD FTLTISNVQS
EDLAEYFCQQ YNSYPYTFGG GTKLEIK
CDR_{L}1 of PD-1 mAb 14 (SEQ ID NO: 173):
KASQSVGTNVA
CDR_{L}2 of PD-1 mAb 14 (SEQ ID NO: 174):
SASSRFS
CDR_{L}3 of PD-1 mAb 14 (SEQ ID NO: 175):
QQYNSYPYT

### (o) PD-1 mAb 15

The amino acid sequence of the VH Domain of murine anti-human PD-1 mAb 15 (SEQ ID NO:176) is shown below (CDR_{H} residues are shown underlined).

EVMLVESGGG LVKPGGSLKL SCAASGFIFS SYLISWVRQT
PEKRLEWVAA ISGGGADTYY ADSVKGRFTI SRDNAKNTLY
LQMSSLRSED TALYYCTRRG TYAMDYWGQG TSVTVSS
CDR_{H}1 of PD-1 mAb 15 (SEQ ID NO: 177):
SYLIS
CDR_{H}2 of PD-1 mAb 15 (SEQ ID NO: 178):
AISGGGADTYYADSVKG
CDR_{H}3 of PD-1 mAb 15 (SEQ ID NO: 179):
RGTYAMDY

The amino acid sequence of the VL Domain of murine anti-human PD-1 mAb 15 (SEQ ID NO:180) is shown below (CDR_{L} residues are shown underlined):

DIQMTQSPAS QSASLGESVT ITCLASQTIG TWLAWYQQKP
GKSPQLLIYA ATSLADGVPS RFSGSGSGTK FSFKISSLQA
EDFVNYYCQQ LYSIPWTFGG GTKLEIK
CDR_{L}1 of PD-1 mAb 15 (SEQ ID NO: 181):
LASQTIGTWLA
CDR_{L}2 of PD-1 mAb 15 (SEQ ID NO: 182):
AATSLAD
CDR_{L}3 of PD-1 mAb 15 (SEQ ID NO: 183):
QQLYSIPWT

The above-described murine anti-human PD-1 antibody PD-1 mAb 15 was humanized and further deimmunized when antigenic epitopes were identified in order to demonstrate the capability of humanizing an anti-human PD-1 antibody so as to decrease its antigenicity upon administration to a human recipient. The humanization yielded one humanized VH Domain, designated herein as “hPD-1 mAb 15 VH1,” and one humanized VL Domain designated herein as “hPD-1 mAb 15 VL1.” An antibody comprising the humanized VL Domain paired with the humanized VH Domain is referred to as “hPD-1 mAb 15.”

The amino acid sequence of the VH Domain of hPD-1 mAb 15 VH1 (SEQ ID NO:184) is shown below (CDR_{H} residues are shown underlined):

EVQLVESGGG LVRPGGSLRL SCAASGFTFS SYLISWVRQA
PGKGLEWVAA ISGGGADTYY ADSVKGRFTI SRDNAKNSLY
LQMNSLRAED TATYYCARRG TYAMDYWGQG TLVTVSS

The amino acid sequence of the VL Domain of hPD-1 mAb 15 VL1 (SEQ ID NO:185) is shown below (CDR_{H} residues are shown underlined):

DIQMTQSPSS LSASVGDRVT ITCLASQTIG TWLAWYQQKP
GKAPKLLIYA ATSLADGVPS RFSGSGSGTD FTFTISSLQP
EDFATYYCQQ LYSIPWTFGQ GTKLEIK

### (p) Additional Anti-PD-1 Antibodies

Alternative anti-PD-1 antibodies useful in the generation of molecules capable of binding PD-1 or a natural ligand of PD-1 possess the VL and/or VH Domains of the anti-human PD-1 monoclonal antibody nivolumab (CAS Reg. No.:946414-94-4, also known as 5C4, BMS-936558, ONO-4538, MDX-1106, and marketed as OPDIVO® by Bristol-Myers Squibb); pembrolizumab (formerly known as lambrolizumab), CAS Reg. No.:1374853-91-4, also known as MK-3475, SCH-900475, and marketed as KEYTRUDA® by Merck); EH12.2H7 (Dana Farber); pidilizumab (CAS Reg. No.: 1036730-42-3 also known as CT-011, CureTech,), or any of the anti-PD-1 antibodies in Table 8; and more preferably possess 1, 2 or all 3 of the CDR_{L}_{^{S}} of the VL Region and/or 1, 2 or all 3 of the CDR_{H}_{^{S}} of the VH Domain of such anti-PD-1 monoclonal antibodies. The amino acid sequences of the complete Heavy and Light Chains of nivolumab (WHO Drug Information, 2013, Recommended INN: List 69, 27(1):68-69), pembrolizumab (WHO Drug Information, 2014, Recommended INN: List 75, 28(3):407) and pidilizumab (WHO Drug Information, 2013, Recommended INN: List 70, 27(3):303-304) are known in the art. Additional anti-PD-1 antibodies possessing unique binding characteristics useful in the methods and compositions of the instant inventions have recently been identified (see, United States Patent Application Nos. 62/198,867; 62/239,559; 62/255,140).

TABLE 8
Additional Anti-PD-1 Antibodies

| PD-1 Antibodies | Reference/Source |
|---|---|
| PD1-17; PD1-28; PD1-33; PD1-35; and PD1-F2 | U.S. Pat. Nos. 7,488,802; |
| | 7,521,051 and 8,088,905; PCT |
| | Patent Publication WO |
| | 2004/056875 |
| 17D8; 2D3; 4H1; 5C4; 4A11; 7D3; and 5F4 | U.S. Pat. Nos. 8,008,449; |
| | 8,779,105 and 9,084,776; PCT |
| | Patent Publication WO |
| | 2006/121168 |
| hPD-1.08A; hPD-1.09A; 109A; K09A; 409A; | U.S. Pat. Nos. 8,354,509; |
| h409A11; h409A16; h409A17; Codon optimized | 8,900,587 and 5,952,136; PCT |
| 109A; and Codon optimized 409A | Patent Publication WO |
| | 2008/156712 |
| 1E3; 1E8; and 1H3 | US Patent Publication |
| | 2014/0044738; PCT Patent |
| | Publication WO 2012/145493 |
| 9A2; 10B11; 6E9; APE1922; APE1923; | PCT Patent Publication WO |
| APE1924; APE1950; APE1963; and APE2058 | 2014/179664 |
| GA1; GA2; GB1; GB6; GH1; A2; C7; H7; SH- | US Patent Publication |
| A4; SH-A9; RG1H10; RG1H11; RG2H7; | 2014/0356363; PCT Patent |
| RG2H10; RG3E12; RG4A6; RG5D9; RG1H10- | Publication WO 2014/194302 |
| H2A-22-1S; RG1H10-H2A-27-2S; RG1H10-3C; | |
| RG1H10-16C; RG1H10-17C; RG1H10-19C; | |
| RG1H10-21C; and RG1H10-23C2 | |
| H1M7789N; H1M7799N; H1M7800N; | US Patent Publication |
| H2M7780N; H2M7788N; H2M7790N; | 2015/0203579; PCT Patent |
| H2M7791N; H2M7794N; H2M7795N; | Publication WO 2015/112800 |
| H2M7796N; H2M7798N; H4H9019P; | |
| H4xH9034P2; H4xH9035P2; H4xH9037P2; | |
| H4xH9045P2; H4xH9048P2; H4H9057P2; | |
| H4H9068P2; H4xH9119P2; H4xH9120P2; | |
| H4Xh9128p2; H4Xh9135p2; H4Xh9145p2; | |
| H4Xh8992p; H4Xh8999p; and H4Xh9008p; | |
| PD-1 mAb 1; PD-1 mAb 2; hPD-1 mAb 2; PD-1 | US patent application Nos. |
| mAb 3; PD-1 mAb 4; PD-1 mAb 5; PD-1 mAb 6; | 62/198,867 and 62/239,559 |
| PD-1 mAb 7; hPD-1 mAb 7; PD-1 mAb 8; PD-1 | |
| mAb 9; hPD-1 mAb 9; PD-1 mAb 10; PD-1 mAb | |
| 11; PD-1 mAb 12; PD-1 mAb 13; PD-1 mAb 14; | |
| PD-1 mAb 15; and hPD-1 mAb 15 | |

### (q) Exemplary IgG4 PD-1 Antibodies

In certain embodiments anti-PD-1 antibodies useful in the methods and compositions of the instant inventions comprise the VL and VH Domains of any of the antibodies provided above (e.g., PD-1 mAb 1, PD-1 mAb 2, PD-1 mAb 3, PD-1 mAb 4, PD-1 mAb 5, PD-1 mAb 6, PD-1 mAb 7, PD-1 mAb 8, etc., or any of the anti-PD-1 antibodies in Table 6), a kappa CL Domain (SEQ ID NO:12), and an IgG4 Fc Domain, optionally lacking the C-terminal lysine residue. Such antibodies will preferably comprise an IgG4 CH1 Domain (SEQ ID NO:3) and a Hinge Domain, and more preferably comprise a stabilized IgG4 Hinge comprising an S228P substitution (wherein the numbering is according to the EU index as in Kabat, SEQ ID NO:7), and IgG4 CH2-CH3 Domains (SEQ ID NO:7).

An exemplary anti-PD-1 antibody designated “hPD-1 mAb 7 (1.2) IgG4 (P)” is a humanized anti-human PD-1 antibody. As indicated above, hPD-1 mAb 7(1.2) comprises the VH Domain of hPD-1 mAb 7 VH1 and the VL Domain of antibody hPD-1 mAb 7 VL2.

The amino acid sequence of the complete Heavy Chain of hPD-1 mAb7 (1.2) IgG4 (P) is SEQ ID NO:186 (CDR_{H} residues and the S228P residue are shown underlined):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVIVSSA
STKGPSVFPL APCSRSTSES TAALGCLVKD YFPEPVTVSW
NSGALTSGVH TFPAVLQSSG LYSLSSVVTV PSSSLGTKTY
TCNVDHKPSN TKVDKRVESK YGPPCPPCPA PEFLGGPSVF
LFPPKPKDTL MISRTPEVTC VVVDVSQEDP EVQFNWYVDG
VEVHNAKTKP REEQFNSTYR VVSVLTVLHQ DWLNGKEYKC
KVSNKGLPSS IEKTISKAKG QPREPQVYTL PPSQEEMTKN
QVSLTCLVKG FYPSDIAVEW ESNGQPENNY KTTPPVLDSD
GSFFLYSRLT VDKSRWQEGN VFSCSVMHEA LHNHYTQKSL SLSLG

In SEQ ID NO:186, residues 1-119 correspond to the VH Domain of hPD-1 mAb 7 VH1 (SEQ ID NO:106), amino acid residues 120-217 correspond to the human IgG4 CH1 Domain is (SEQ ID NO:3), amino acid residues 218-229 correspond to the human IgG4 Hinge Domain comprising the S228P substitution (SEQ ID NO:7), amino acid residues 230-245 correspond to the human IgG4 CH2-CH3 Domains (SEQ ID NO:11, wherein X is absent).

The amino acid sequence of the complete Light Chain of antibody hPD-1 mAb7 (1.2) IgG4 (P) possesses a kappa constant region and is (SEQ ID NO:187):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In SEQ ID NO:187, amino acid residues 1-111 correspond to the VL Domain of hPD-1 mAb 7 VL2 (SEQ ID NO:109), and amino acid residues 112-218 correspond to the Light Chain kappa constant region (SEQ ID NO:12)

Other exemplary anti-PD-1 antibodies having IgG4 constant regions are nivolumab, which is a human antibody, and pembrolizumab, which is a humanized antibody. Each comprise a kappa CL Domain, an IgG4 CHI Domain, a stabilized IgG4 Hinge, and an IgG4 CH2-CH3 Domain as described above.

### (r) Exemplary Bispecific Molecules Capable of Binding PD-1 and LAG-3

As provided herein, the molecule capable of binding PD-1 or a natural ligand of PD-1 may a bispecific molecule. In certain embodiments, bispecific molecules will preferably comprise the VL and VH Domains of any of the anti-PD-1 antibodies provided above (e.g., PD-1 mAb 1, PD-1 mAb 2, PD-1 mAb 3, PD-1 mAb 4, PD-1 mAb 5, PD-1 mAb 6, PD-1 mAb 7, PD-1 mAb 8, etc., or any of the anti-PD-1 antibodies in Table 6), and the VL and VH Domains of an antibody that binds an epitope of CD137, LAG-3, OX40, TIGIT, TIM-3, or VISTA. Such bispecific molecules may be diabodies, BITEs®, bispecific antibodies, or trivalent binding molecules.

An exemplary bispecific molecule capable of binding PD-1 and LAG-3 designated “DART-1” is a diabody comprising four polypeptide chains. DART-1 is a bispecific, four chain, Fc Region-containing diabody having two binding sites specific for PD-1, two binding sites specific for LAG-3, a variant IgG4 Fc Region engineered for extended half-life, and cysteine-containing E/K-coil Heterodimer-Promoting Domains (see, e.g., FIG. 3B). The first and third polypeptide chains of DART-1 comprise, in the N-terminal to C-terminal direction: an N-terminus, a VL Domain of a monoclonal antibody capable of binding to LAG-3 (underlined in SEQ ID NO:274); an intervening linker peptide (Linker 1: GGGSGGGG (SEQ ID NO:14)), a VH Domain of hPD-1 mAb 7 VH1 (SEQ ID NO:106); a cysteine-containing intervening linker peptide (Linker 2: GGCGGG (SEQ ID NO:15)); a cysteine-containing Heterodimer-Promoting (E-coil) Domain (EVAACEK-EVAALEK-EVAALEK-EVAALEK (SEQ ID NO:29)); a stabilized IgG4 Hinge region (SEQ ID NO:7); a variant IgG4 CH2-CH3 Domain (SEQ ID NO:11) further comprising amino acid substitutions M252Y/S254T/T256E and lacking the C-terminal residue); and a C-terminus. The amino acid sequence of the first and third polypeptide chains of DART-1 is (SEQ ID NO:274):

DIQMTQSPSS LSASVGDRVT ITCRASQDVS SVVAWYQQKP
GKAPKLLIYS ASYRYTGVPS RFSGSGSGTD FTLTISSLQP
EDFATYYCQQ HYSTPWTFGG GTKLEIKGGG SGGGGQVQLV
QSGAEVKKPG ASVKVSCKAS GYSFTSYWMN WVRQAPGQGL
EWIGVIHPSD SETWLDQKFK DRVTITVDKS TSTAYMELSS
LRSEDTAVYY CAREHYGTSP FAYWGQGTLV TVSSGGCGGG
EVAACEKEVA ALEKEVAALE KEVAALEKES KYGPPCPPCP
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLG

The second and fourth polypeptide chains of DART-1 comprise, in the N-terminal to C-terminal direction: an N-terminus, a VL Domain of hPD-1 mAb 7 VL2 (SEQ ID NO:109); an intervening linker peptide (Linker 1: GGGSGGGG (SEQ ID NO:14)); a VH Domain of a monoclonal antibody capable of binding LAG-3 (underlined in SEQ ID NO:275); a cysteine-containing intervening linker peptide (Linker 2: GGCGGG (SEQ ID NO:15)); a cysteine-containing Heterodimer-Promoting (K-coil) Domain (KVAACKE-KVAALKE-KVAALKE-KVAALKE (SEQ ID NO:30); and a C-terminus. The amino acid sequence of the second and fourth polypeptide chains of DART-1 is (SEQ ID NO:275):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYTFTD YNMDWVRQAP
GQGLEWMGDI NPDNGVTIYN QKFEGRVTMT TDTSTSTAYM
ELRSLRSDDT AVYYCAREAD YFYFDYWGQG TTLTVSSGGC
GGGKVAACKE KVAALKEKVA ALKEKVAALK E

Another exemplary bispecific molecule capable of binding PD-1 and LAG-3 designated “DART-2” has the same structure as DART-1 but incorporates alternative LAG-3 VL and VH Domains.

### 2. Binding Molecules Immunospecific For Natural Ligands of PD-1

As discussed above, natural ligands of PD-1, for example, B7-H1 (PD-L1) and B7-DC (PD-L2), have been described (Ohigashi et al. (2005) “Clinical Significance Of Programmed Death-1 Ligand-1 And Programmed Death-1 Ligand-2 Expression In Human Esophageal Cancer,” Clin. Cancer Res. 11:2947-2953; Dong, H. et al. (1999) “B7-H1, A Third Member Of The B7 Family, Co-Stimulates Cell Proliferation And Interleukin-10 Secretion,” Nat. Med. 5:1365-1369; Freeman, G. J. et al. (2000) “Engagement Of The PD-1 Immunoinhibitory Receptor By A Novel B7 Family Member Leads To Negative Regulation Of Lymphocyte Activation,” J. Exp. Med. 192:1027-1034; Tseng, S. Y. et al. (2001) “B7-DC, A New Dendritic Cell Molecule With Potent Costimulatory Properties For T Cells,” J. Exp. Med 193:839-846; Latchman, Y. et al. (2001) “PD-L2 Is A Second Ligand For PD-1 And Inhibits T Cell Activation,” Nat. Immunol. 2:261-268; Iwai et al. (2002) “Involvement Of PD-L1 On Tumor Cells In The Escape From Host Immune System And Tumor Immunotherapy By PD-L1 Blockade,” Proc. Natl. Acad. Sci. (U.S.A.) 99:12293-12297).

A representative human B7-H1 (PD-L1) polypeptide (NCBI Sequence NP_001254635.1, including a predicted 18 amino acid signal sequence) has the amino acid sequence (SEQ ID NO:188):

MRIFAVFIFM TYWHLLNAPY NKINQRILVV DPVTSEHELT
CQAEGYPKAE VIWTSSDHQV LSGKTTTTNS KREEKLFNVT
STLRINTTTN EIFYCTFRRL DPEENHTAEL VIPELPLAHP
PNERTHLVIL GAILLCLGVA LTFIFRLRKG RMMDVKKCGI
QDTNSKKQSD THLEET

A representative human B7-DC (PD-L2) polypeptide (NCBI Sequence NP_079515.2; including a predicted 18 amino acid signal sequence) has the amino acid sequence (SEQ ID NO:189):

MIFLLLMLSL ELQLHQIAAL FTVTVPKELY IIEHGSNVTL
ECNFDTGSHV NLGAITASLQ KVENDTSPHR ERATLLEEQL
PLGKASFHIP QVQVRDEGQY QCIIIYGVAW DYKYLTLKVK
ASYRKINTHI LKVPETDEVE LTCQATGYPL AEVSWPNVSV
PANTSHSRTP EGLYQVTSVL RLKPPPGRNF SCVFWNTHVR
ELTLASIDLQ SQMEPRTHPT WLLHIFIPFC IIAFIFIATV
IALRKQLCQK LYSSKDTTKR PVTTTKREVN SAI

Although B7-H1 and B7-DC share 34% identity of amino acid sequence, their expression has been suggested to be differentially regulated (Youngnak, P. et al. (2003) “Differential Binding Properties Of B7-H1 And B7-DC To Programmed Death-1,” Biochem. Biophys. Res. Commun. 307:672-677; Loke, P. et al. (2003) “PD-L1 And PD-L2 Are Differentially Regulated By Th1 And Th2 Cells,” Proc. Natl. Acad. Sci. (U.S.A.) 100:5336-5341). PD-L1 has been suggested to play a role in tumor immunity by increasing apoptosis of antigen-specific T-cell clones (Dong et al. (2002) “Tumor Associated B7-H1 Promotes T-Cell Apoptosis: A Potential Mechanism Of Immune Evasion,” Nat Med 8:793-800). It has also been suggested that B7-H1 might be involved in intestinal mucosal inflammation and inhibition of B7-H1 suppresses wasting disease associated with colitis (Kanai et al. (2003) “Blockade Of B7-HI Suppresses The Development Of Chronic Intestinal Inflammation,” J. Immunol. 171:4156-4163). B7-H1 expression has been reported in human carcinoma of lung, ovary, and colon and in melanomas (Dong et al. (2002) “Tumor-Associated B7-H1 Promotes T-Cell Apoptosis: A Potential Mechanism Of Immune Evasion,” Nat Med 8:793-800). On the other hand, the function of B7-DC in tumors remains largely unknown (Liu, X. et al. (2003) “B7-DC/PD-L2 Promotes Tumor Immunity By A PD-1-Independent Mechanism,” J. Exp. Med. 197:1721-1730; Radhakrishnan, S. et al. (2004) “Immunotherapeutic Potential Of B7-DC (PD-L2) Cross-Linking Antibody In Conferring Antitumor Immunity,” Cancer Res 64:4965-4972. B7-DC expression on the cancer cells has been shown to promote CD8 T-cell-mediated rejection at both the induction and effector phase of antitumor immunity (Liu, X. et al. (2003) “B7-DC/PD-L2 Promotes Tumor Immunity By A PD-1-Independent Mechanism,” J. Exp. Med. 197:1721-1730).

Anti-B7-H1 antibodies may be obtained using proteins having the above-provided B7-H1 amino acid sequence as an immunogen. Alternatively, anti-B7-H1 antibodies useful in the generation of molecules capable of binding a natural ligand of PD-1 may possess the VL and/or VH Domains of the anti-human B7-H1 antibody atezolizumab (CAS Reg No. 1380723-44-3, also known as MPDL3280A), durvalumab (CAS Reg No. 1428935-60-7, also known as MEDI-4736), avelumab, MDX1105 (CAS Reg No. 1537032-82-8, also known as BMS-936559), 5H1); (also see, U.S. Pat. Nos. 9,273,135, 9,062,112, 8,981,063, 8,779,108, 8,609,089 and 8,460,927; McDermott, D. F. et al. (2016) “Atezolizumab, an Anti Programmed Death-Ligand I Antibody, in Metastatic Renal Cell Carcinoma: Long-Term Safety, Clinical Activity, and Immune Correlates From a Phase Ia Study,” J. Clin. Oncol. 34(8):833-842; Antonia, S. et al. (2016) “Safety And Antitumour Activity Of Durvalumab Plus Tremelimumab In Non-Small Cell Lung Cancer: A Multicentre, Phase 1b Study,” Lancet Oncol. 17(3):299-308; Boyerinas, B. et al. (2015) “Antibody-Dependent Cellular Cytotoxicity Activity of a Novel Anti-PD-L1 Antibody Avelumab (MSB0010718C) on Human Tumor Cells,” Cancer Immunol Res. 3(10):1148-1157; Katy, K. et al. (2014) “PD-1 And PD-L1 Antibodies For Melanoma,” Hum. Vaccin. Immunother. 10(11):3111-3116; Voena, C. et al. (2016) “Advances In Cancer Immunology And Cancer Immunotherapy,” Discov. Med. 21(114):125-133) and/or of a commercially available antibody (e.g., rabbit anti-human PDL-1 monoclonal, 1:25, clone SP142; Ventana, Tucson, Ariz.).

Exemplary anti-human B7-H1 antibodies that may be used in accordance with the present invention include atezolizumab, durvalumab and avelumab. The amino acid sequences of the complete heavy and Light Chains of atezolizumab (WHO Drug Information, 2015, Recommended INN: List 74, 29(3):387), durvalumab (WHO Drug Information, 2015, Recommended INN: List 74, 29(3):393-394) and avelumab (WHO Drug Information, 2016, Recommended INN: List 74, 30(1):100-101) are known in the art.

Anti-B7-DC antibodies may likewise be obtained using proteins having the above-provided B7-DC amino acid sequence as an immunogen. Alternatively, previously described anti-B7-DC antibodies (e.g., 2C9, MIH18, etc.) or commercially available anti-B7-DC antibodies (e.g., MIH18, Affymetrix eBioscience) may be employed in accordance with the present invention (see, U.S. Patent Publication No. 2015/0299322; Ritprajak, P. et al. (2012) “Antibodies Against B7-DC With Differential Binding Properties Exert Opposite Effects,” Hybridoma (Larchmt). 31(1):40-47; Tsushima, F. et al. (2003) “Preferential Contribution Of B7-H1 To Programmed Death-1 Mediated Regulation Of Hapten-Specific Allergic Inflammatory Responses,” Eur. J. Immunol. 33 (10): 2773-2782).

An exemplary anti-human anti-B7-DC antibody that may be used in accordance with the present invention is the commercially available anti-B7-DC antibody MIH18 (eBioscience, Inc.)

### B. Molecules Capable Of Mediating The Redirected Killing Of A Target Cell

The molecules of the present invention have the ability to mediate the redirected killing of a target cell (e.g., a cancer cell or a pathogen-infected cell) will preferably have two binding affinities. First, such molecules will have the ability to immunospecifically bind an epitope of a cell surface molecule of an effector cell. Second, such molecules will have the ability to immunospecifically bind an epitope of a Disease Antigen (e.g., a Cancer Antigen or a Pathogen-Associated Antigen) that is arrayed on the surface of the target cell. The combined presence of both such binding affinities serves to localize the effector cell to the site of the target cell (i.e., to “redirect” the effector cell) so that it may mediate the killing of the target cell. As discussed above, such molecules may be bispecific, or may be capable of binding more than two epitopes.

### 1. Exemplary Cell Surface Molecules Of An Effector Cell

As used herein, the term “effector cell” denotes a cell that directly or indirectly mediates the killing of target cells (e.g., foreign cells, infected cells or cancer cells). Examples of effector cells include helper T Cells, cytotoxic T Cells, Natural Killer (NK) cells, plasma cells (antibody-secreting B cells), macrophages and granulocytes. Preferred cell surface molecules of such cells include CD2, CD3, CD8, CD16, TCR, and the NKG2D receptor. Accordingly, molecules capable of immunospecifically binding an epitope of such molecules, or to other effector cell surface molecules may be used in accordance with the principles of the present invention. Exemplary antibodies, whose VH and VL Domains may be used to construct molecules capable of mediating the redirected killing of a target cell are provided below.

### (a) CD2 Binding Capabilities

In one embodiment, the molecules of the present invention that are capable of mediating the redirected killing of a target cell will bind an effector cell by immunospecifically binding an epitope of CD2 present on the surface of such effector cell. Molecules that specifically bind CD2 include the anti-CD2 antibody “CD2 mAb Lo-CD2a.”

The amino acid sequence of the VH Domain of CD2 mAb Lo-CD2a (ATCC Accession No: 11423); SEQ ID NO:190) is shown below (CDR_{H} residues are shown underlined):

EVQLQQSGPE LQRPGASVKL SCKASGYIFT EYYMYWVKQR
PKQGLELVGR IDPEDGSIDY VEKFKKKATL TADTSSNTAY
MQLSSLTSED TATYFCARGK FNYRFAYWGQ GTLVTVSS

The amino acid sequence of the VL Domain of CD2 mAb Lo-CD2a (ATCC Accession No: 11423; SEQ ID NO:191) is shown below (CDR_{L} residues are shown underlined):

DVVLIQTPPT LLATIGQSVS ISCRSSQSLL HSSGNTYLNW
LLQRTGQSPQ PLIYLVSKLE SGVPNRFSGS GSGTDFTLKI
SGVEAEDLGV YYCMQFTHYP YTFGAGTKLE LK

### (b) CD3 Binding Capabilities

In one embodiment, the molecules of the present invention that are capable of mediating the redirected killing of a target cell will bind an effector cell by immunospecifically binding an epitope of CD3 present on the surface of such effector cell. Molecules that specifically binds CD3 include the anti-CD3 antibodies “CD3 mAb 1” and “OKT3.” The anti-CD3 antibody CD3 mAb 1 is capable of binding non-human primates (e.g., cynomolgus monkey).

The amino acid sequence of the VH Domain of CD3 mAb 1 (SEQ ID NO:192) is shown below (CDR_{H} residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL VTVSS

The amino acid sequence of the VL Domain of CD3 mAb 1 (SEQ ID NO:193) is shown below (CDR_{L} residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLIVLG

A preferred variant of such antibody is termed “CD3 mAb 1 (D65G),” and comprises a CD3 mAb 1 VH Domain having a D65G substitution (Kabat position 65, corresponding to residue 68 of SEQ ID NO:192) and the VL Domain of CD3 mAb 1 (SEQ ID NO:193). The amino acid sequence of the VH Domain of CD3 mAb 1 (D65G) (SEQ ID NO:194) is shown below (CDR_{H} residues are shown underlined, the substituted position (D65G) is shown in double underline):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL VTVSS

Alternatively, an affinity variant of CD3 mAb 1 may be employed. Variants include a low affinity variant designated “CD3 mAb 1 Low” and a variant having a faster off rate designated “CD3 mAb 1 Fast.” The amino acid sequences of the VH Domains of each of CD3 mAb 1 Low and CD3 mAb1 Fast are provided below.

The amino acid sequence of the VH Domain of anti-human CD3 mAb 1 Low (SEQ ID NO:195) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVT WFAYWGQGTL VTVSS

The amino acid sequence of the VH Domain of anti-human CD3 mAb 1 Fast (SEQ ID NO:196) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HKNFGNSYVT WFAYWGQGTL VTVSS

The VL Domain of CD3 mAb 1 (SEQ ID NO:193) is common to CD3 mAb 1 Low and CD3 mAbl Fast and is provided above.

Another anti-CD3 antibody that may be utilized is antibody Muromonab-CD3 “OKT3” (Xu et al. (2000) “In Vitro Characterization Of Five Humanized OKT3 Effector Function Variant Antibodies,” Cell. Immunol. 200:16-26); Norman, D. J. (1995) “Mechanisms Of Action And Overview Of OKT3,” Ther. Drug Monit. 17(6):615-620; Canafax, D. M. et al. (1987) “Monoclonal Antilymphocyte Antibody (OKT3) Treatment Of Acute Renal Allograft Rejection,” Pharmacotherapy 7(4):121-124; Swinnen, L. J. et al. (1993) “OKT3 Monoclonal Antibodies Induce Interleukin-6 And Interleukin-10: A Possible Cause Of Lymphoproliferative Disorders Associated With Transplantation,” Curr. Opin. Nephrol. Hypertens. 2(4):670-678).

The amino acid sequence of the VH Domain of OKT3 (SEQ ID NO:197) is shown below (CDR_{H} residues are shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSS

The amino acid sequence of the VL Domain of OKT3 (SEQ ID NO:198) is shown below (CDR_{L} residues are shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

Additional anti-CD3 antibodies that may be utilized include, but are not limited to, those described in PCT Publication Nos. WO 2008/119566; and WO 2005/118635.

### (c) CD8 Binding Capabilities

In one embodiment, the molecules of the present invention that are capable of mediating the redirected killing of a target cell will bind an effector cell by immunospecifically binding an epitope of CD8 present on the surface of such effector cell. Antibodies that specifically bind CD8 include the anti-CD8 antibodies “OKT8” and “TRX2.”

### (i) OKT8

The amino acid sequence of the VH Domain of OKT8 (SEQ ID NO:199) is shown below (CDR_{H} residues are shown underlined):

QVQLLESGPE LLKPGASVKM SCKASGYTFT DYNMHWVKQS
HGKSLEWIGY IYPYTGGTGY NQKFKNKATL TVDSSSSTAY
MELRSLTSED SAVYYCARNF RYTYWYFDVW GQGTTVTVSS

The amino acid sequence of the VL Domain of OKT8 (SEQ ID NO:200) is shown below (CDR_{L} residues are shown underlined):

DIVMTQSPAS LAVSLGQRAT ISCRASESVD SYDNSLMHWY
QQKPGQPPKV LIYLASNLES GVPARFSGSG SRTDFTLTID
PVEADDAATY YCQQNNEDPY TFGGGTKLEI KR

### (ii) TRX2

The amino acid sequence of the VH Domain of TRX2 (SEQ ID NO:201) is shown below (CDR_{H} residues are shown underlined):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCANPH YDGYYHFFDS WGQGTLVIVS
S

The amino acid sequence of the VL Domain of TRX2 (SEQ ID NO:202) is shown below (CDR_{L} residues are shown underlined):

DIQMIQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIK

### (d) CD16 Binding Capabilities

In one embodiment, the molecules of the present invention that are capable of mediating the redirected killing of a target cell will bind an effector cell by immunospecifically binding an epitope of CD16 present on the surface of such effector cell. Molecules that specifically bind CD16 include the anti-CD16 antibodies “3G8” and “A9.” Humanized A9 antibodies are described in PCT Publication WO 03/101485.

### (i) 3G8

The amino acid sequence of the VH Domain of 3G8 (SEQ ID NO:203) is shown below (CDR_{H} residues are shown underlined):

QVTLKESGPG ILQPSQTLSL TCSFSGFSLR TSGMGVGWIR
QPSGKGLEWL AHIWWDDDKR YNPALKSRLT ISKDTSSNQV
FLKIASVDTA DTATYYCAQI NPAWFAYWGQ GTLVTVSA

The amino acid sequence of the VL Domain of 3G8 (SEQ ID NO:204) is shown below (CDR_{L} residues are shown underlined):

DTVLTQSPAS LAVSLGQRAT ISCKASQSVD FDGDSFMNWY
QQKPGQPPKL LIYTTSNLES GIPARFSASG SGTDFTLNIH
PVEEEDTATY YCQQSNEDPY TFGGGTKLEI K

### (ii) A9

The amino acid sequence of the VH Domain of A9 (SEQ ID NO:205) is shown below (CDR_{H} residues are shown underlined):

QVQLQQSGAE LVRPGISVKI SCKASGYTFT NYWLGWVKQR
PGHGLEWIGD IYPGGGYTNY NEKFKGKATV TADTSSRTAY
VQVRSLTSED SAVYFCARSA SWYFDVWGAR TTVTVSS

The amino acid sequence of the VL Domain of A9 (SEQ ID NO:206) is shown below (CDR_{L} residues are shown underlined):

DIQAVVTQES ALTTSPGETV TLTCRSNTGT VTTSNYANWV
QEKPDHLFTG LIGHTNNRAP GVPARFSGSL IGDKAALTIT
GAQTEDEAIY FCALWYNNHW VFGGGTKLTVL

Additional anti-CD19 antibodies that may be utilized include but are not limited to those described in PCT Publication Nos. WO 03/101485; and WO 2006/125668.

### (e) TCR Binding Capabilities

In one embodiment, the molecules of the present invention that are capable of mediating the redirected killing of a target cell will bind an effector cell by immunospecifically binding an epitope of TCR present on the surface of such effector cell.

Molecules that specifically bind the T Cell Receptor include the anti-TCR antibody “BMA 031” (EP 0403156; Kurrle, R. et al. (1989) “BMA 031—A TCR-Specific Monoclonal Antibody For Clinical Application,” Transplant Proc. 21(1 Pt 1):1017-1019; Nashan, B. et al. (1987) “Fine Specificity Of A Panel Of Antibodies Against The TCR/CD3 Complex,” Transplant Proc. 19(5):4270-4272; Shearman, C. W. et al. (1991) “Construction, Expression, And Biologic Activity Of Murine/Human Chimeric Antibodies With Specificity For The Human α/β T Cell,” J. Immunol. 146(3):928-935; Shearman, C. W. et al. (1991) “Construction, Expression And Characterization of Humanized Antibodies Directed Against The Human α/β T Cell Receptor,” J. Immunol. 147(12):4366-4373).

The amino acid sequence of a VH Domain of BMA 031 (SEQ ID NO:207) is shown below (CDR_{H} residues are shown underlined):

QVQLVQSGAE VKKPGASVKV SCKASGYKFT SYVMHWVRQA
PGQGLEWIGY INPYNDVTKY NEKFKGRVTI TADKSTSTAY
LQMNSLRSED TAVHYCARGS YYDYDGFVYW GQGTLVTVSS

The amino acid sequence of the VL Domain of BMA 031 (SEQ ID NO:208) is shown below (CDR_{L} residues are shown underlined):

EIVLTQSPAT LSLSPGERAT LSCSATSSVS YMHWYQQKPG
KAPKRWIYDT SKLASGVPSR FSGSGSGTEF TLTISSLQPE
DFATYYCQQW SSNPLTFGQG TKLEIK

### (f) NKG2D Binding Capabilities

In one embodiment, the molecules of the present invention that are capable of mediating the redirected killing of a target cell will bind an effector cell by immunospecifically binding an epitope of the NKG2D receptor present on the surface of such effector cell. Molecules that specifically bind the NKG2D receptor include the anti-NKG2D antibodies “KYK-1.0” and “KYK-2.0” (Kwong, K Y et al. (2008) “Generation, Affinity Maturation, And Characterization Of A Human Anti-Human NKG2D Monoclonal Antibody With Dual Antagonistic And Agonistic Activity,” J. Mol. Biol. 384:1143-1156; and PCT/US09/54911).

### (i) KYK-1.0

The amino acid sequence of the VH Domain of KYK-1.0 (SEQ ID NO:209) is shown below (CDR_{H} residues are shown underlined):

EVQLVESGGG VVQPGGSLRL SCAASGFTFS SYGMHWVRQA
PGKGLEWVAF IRYDGSNKYY ADSVKGRFTI SRDNSKNTKY
LQMNSLRAED TAVYYCAKDR FGYYLDYWGQ GTLVTVSS

The amino acid sequence of the VL Domain of KYK-1.0 (SEQ ID NO:210) is shown below (CDR_{L} residues are shown underlined):

QPVLTQPSSV SVAPGETARI PCGGDDIETK SVHWYQQKPG
QAPVLVIYDD DDRPSGIPER FFGSNSGNTA TLSISRVEAG
DEADYYCQVW DDNNDEWVFG GGTQLTVL

### (ii) KYK-2.0

The amino acid sequence of a VH Domain of KYK-2.0 (SEQ ID NO:211) is shown below (CDR_{H} residues are shown underlined):

QVQLVESGGG LVKPGGSLRL SCAASGFTFS SYGMHWVRQA
PGKGLEWVAF IRYDGSNKYY ADSVKGRFTT SRDNSKNTLY
LQMNSLRAED TAVYYCAKDR GLGDGTYFDY WGQGTTVTVS
S

The amino acid sequence of a VL Domain of KYK-2.0 (SEQ ID NO:212) is shown below (CDR_{L} residues are shown underlined):

QSALTQPASV SGSPGQSITI SCSGSSSNIG NNAVNWYQQL
PGKAPKLLIY YDDLLPSGVS DRFSGSKSGT SAFLAISGLQ
SEDEADYYCA AWDDSLNGPV FGGGTKLTVL

### C. Exemplary Cancer Antigens Arrayed on the Surface of Cancer Cells

As used herein, the term “Cancer Antigen” denotes an antigen that is characteristically expressed on the surface of a cancer cell, and that may thus be treated with an Antibody-Based Molecule or an Immunomodulatory Molecule. Examples of Cancer Antigens include, but are not limited to: 19.9 as found in colon cancer, gastric cancer mucins; 4.2; A33 (a colorectal carcinoma antigen; Almqvist, Y. (2006) “In vitro and in vivo Characterization of 177Lu-huA33: A Radioimmunoconjugate Against Colorectal Cancer,” Nucl. Med. Biol. 33(8):991-998); ADAM-9 (United States Patent Publication No. 2006/0172350; PCT Publication No. WO 06/084075); AH6 as found in gastric cancer; ALCAM (PCT Publication No. WO 03/093443); APO-1 (malignant human lymphocyte antigen) (Trauth, B. C. et al. (1989) “Monoclonal Antibody Mediated Tumor Regression By Induction Of Apoptosis,” Science 245:301-304); B1 (Egloff, A. M. et al. (2006) “Cyclin B1 And Other Cyclins As Tumor Antigens In Immunosurveillance And Immunotherapy Of Cancer,” Cancer Res. 66(1):6-9); B7-H3 (Collins, M. et al. (2005) “The B7 Family Of Immune-Regulatory Ligands,” Genome Biol. 6:223.1-223.7). Chapoval, A. et al. (2001) “B7-H3: A Costimulatory Molecule For T Cell Activation and IFN-γ Production,” Nature Immunol. 2:269-274; Sun, M. et al. (2002) “Characterization of Mouse and Human B7-H3 Genes,” J. Immunol. 168:6294-6297); BAGE (Bodey, B. (2002) “Cancer-Testis Antigens: Promising Targets For Antigen Directed Antineoplastic Immunotherapy,” Expert Opin. Biol. Ther. 2(6):577-584); beta-catenin (Prange W. et al. (2003) “Beta-Catenin Accumulation In The Progression Of Human Hepatocarcinogenesis Correlates With Loss Of E-Cadherin And Accumulation Of P53, But Not With Expression Of Conventional WNT-1 Target Genes,” J. Pathol. 201(2):250-259); blood group ALe^{b}/Le^{y} as found in colonic adenocarcinoma; Burkitt's lymphoma antigen-38.13; C14 as found in colonic adenocarcinoma; CA125 (ovarian carcinoma antigen) (Bast, R. C. Jr. et al. (2005) “New Tumor Markers: CA125 And Beyond,” Int. J. Gynecol. Cancer 15(Suppl 3):274-281; Yu et al. (1991) “Coexpression Of Different Antigenic Markers On Moieties That Bear CA 125 Determinants,” Cancer Res. 51(2):468-475); Carboxypeptidase M (United States Patent Publication No. 2006/0166291); CD5 (Cahn, G. A. et al. (2006) “Genomics Of Chronic Lymphocytic Leukemia MicroRNAs As New Players With Clinical Significance,” Semin. Oncol. 33(2):167-173; CD19 (Ghetie et al. (1994) “Anti-CD19 Inhibits The Growth Of Human B-Cell Tumor Lines In Vitro And Of Daudi Cells In SCID Mice By Inducing Cell Cycle Arrest,” Blood 83:1329-1336; Troussard, X. et al. 1998 Hematol Cell Ther. 40(4):139-48); CD20 (Reff et al. (1994) “Depletion Of B Cells In Vivo By A Chimeric Mouse Human Monoclonal Antibody To CD20,” Blood 83:435-445; Thomas, D. A. et al. 2006 Hematol Oncol Clin North Am. 20(5):1125-36); CD22 (Kreitman, R. J. (2006) “Immunotoxins For Targeted Cancer Therapy,” AAPS J. 8(3):E532-51); CD23 (Rosati, S. et al. (2005) “Chronic Lymphocytic Leukaemia: A Review Of The Immuno-Architecture,” Curr. Top. Microbiol. Immunol. 294:91-107); CD25 (Troussard, X. et al. (1998) “Hairy Cell Leukemia. What Is New Forty Years After The First Description?” Hematol. Cell. Ther. 40(4):139-148); CD27 (Bataille, R. (2006) “The Phenotype Of Normal, Reactive And Malignant Plasma Cells. Identification Of “Many And Multiple Myelomas” And Of New Targets For Myeloma Therapy,” Haematologica 91(9):1234-1240); CD28 (Bataille, R. (2006) “The Phenotype Of Normal, Reactive And Malignant Plasma Cells. Identification Of “Many And Multiple Myelomas” And Of New Targets For Myeloma Therapy,” Haematologica 91(9):1234-1240); CD33 (Sgouros et al. (1993) “Modeling And Dosimetry Of Monoclonal Antibody M195 (Anti-CD33) In Acute Myelogenous Leukemia,” J. Nucl. Med. 34:422-430); CD36 (Ge, Y. (2005) “CD36: A Multiligand Molecule,” Lab Hematol. 11(1):31-7); CD40/CD154 (Messmer, D. et al. (2005) “CD154 Gene Therapy For Human B-Cell Malignancies,” Ann. N. Y. Acad. Sci. 1062:51-60); CD45 (Jurcic, J. G. (2005) “Immunotherapy For Acute Myeloid Leukemia,” Curr. Oncol. Rep. 7(5):339-346); CD56 (Bataille, R. (2006) “The Phenotype Of Normal, Reactive And Malignant Plasma Cells. Identification Of “Many And Multiple Myelomas” And Of New Targets For Myeloma Therapy,” Haematologica 91(9):1234-1240); CD46 (U.S. Pat. No. 7,148,038; PCT Publication No. WO 03/032814); CD52 (Eketorp, S. S. et al. (2014) “Alemtuzumab (Anti-CD52 Monoclonal Antibody) As Single-Agent Therapy In Patients With Relapsed/Refractory Chronic Lymphocytic Leukaemia (CLL)-A Single Region Experience On Consecutive Patients,” Ann Hematol. 93 (10): 1725-1733; Suresh, T. et al. (2014) “New Antibody Approaches To Lymphoma Therapy,” J. Hematol. Oncol. 7:58; Hoelzer, D. (2013) “Targeted Therapy With Monoclonal Antibodies In Acute Lymphoblastic Leukemia,” Curr. Opin. Oncol. 25(6):701-706); CD56 (Bataille, R. (2006) “The Phenotype Of Normal, Reactive And Malignant Plasma Cells. Identification Of “Many And Multiple Myelomas” And Of New Targets For Myeloma Therapy,” Haematologica 91(9):1234-1240); CD79a/CD79b (Troussard, X. et al. (1998) “Hairy Cell Leukemia. What Is New Forty Years After The First Description?” Hematol. Cell. Ther. 40(4):139-148; Chu, P. G. et al. (2001) “CD79: A Review,” Appl. Immunohistochem. Mol. Morphol. 9(2):97-106); CD103 (Troussard, X. et al. (1998) “Hairy Cell Leukemia. What Is New Forty Years After The First Description?” Hematol. Cell. Ther. 40(4):139-148); CD317 (Kawai, S. et al. (2008) “Interferon-A Enhances CD317 Expression And The Antitumor Activity Of Anti-CD317 Monoclonal Antibody In Renal Cell Carcinoma Xenograft Models,” Cancer Science 99(12):2461-2466; Wang, W. et al. (2009) HM1.24 (CD317) Is A Novel Target Against Lung Cancer For Immunotherapy Using Anti-HM1.24 Antibody,” Cancer Immunology, Immunotherapy 58(6):967-976; Wang, W. et al. (2009) “Chimeric And Humanized Anti-HM1.24 Antibodies Mediate Antibody-Dependent Cellular Cytotoxicity Against Lung Cancer Cells. Lung Cancer,” 63(1):23-31; Sayeed, A. et al. (2013) “Aberrant Regulation Of The BST2 (Tetherin) Promoter Enhances Cell Proliferation And Apoptosis Evasion In High Grade Breast Cancer Cells,” PLoS ONE 8(6)e67191, pp. 1-10); CDK4 (Lee, Y. M. et al. (2006) “Targeting Cyclins And Cyclin-Dependent Kinases In Cancer: Lessons From Mice, Hopes For Therapeutic Applications In Human,” Cell Cycle 5(18):2110-2114); CEA (carcinoembryonic antigen; Foon et al. (1995) “Immune Response To The Carcinoembryonic Antigen In Patients Treated With An Anti-Idiotype Antibody Vaccine,” J. Clin. Invest. 96(1):334-42); Mathelin, C. (2006) “Circulating Proteinic Biomarkers And Breast Cancer,” Gynecol. Obstet. Fertil. 34(7-8):638-646; Tellez-Avila, F. I. et al. (2005) “The Carcinoembryonic Antigen: Apropos Of An Old Friend,” Rev. Invest. Clin. 57(6):814-819); CEACAM5/CEACAM6 (Zheng, C. et al. (2011) “A Novel Anti-CEACAM5 Monoclonal Antibody, CC4, Suppresses Colorectal Tumor Growth and Enhances NK Cells-Mediated Tumor Immunity,” PLoS One 6(6):e21146, pp. 1-11); CO17-1A (Ragnhammar et al. (1993) “Effect Of Monoclonal Antibody 17-1A And GM-CSF In Patients With Advanced Colorectal Carcinoma—Long-Lasting, Complete Remissions Can Be Induced,” Int. J. Cancer 53:751-758); CO-43 (blood group Le^{b}); CO-514 (blood group Lea) as found in adenocarcinoma; CTA-1; CTLA-4 (Peggs, K. S. et al. (2006) “Principles And Use Of Anti-CTLA4 Antibody In Human Cancer Immunotherapy,” Curr. Opin. Immunol. 18(2):206-13); Cytokeratin 8 (PCT Publication No. WO 03/024191); D1.1; D₁56-22; DR5 (Abdulghani, J. et al. (2010) “TRAIL Receptor Signaling And Therapeutics,” Expert Opin. Ther. Targets 14(10): 1091-1108; Andera, L. (2009) “Signaling Activated By The Death Receptors Of The TNFR Family,” Biomed. Pap. Med. Fac. Univ. Palacky Olomouc Czech. Repub. 153(3):173-180; Carlo-Stella, C. et al. (2007) “Targeting TRAIL Agonistic Receptors for Cancer Therapy,” Clin, Cancer 13(8):2313-2317; Chaudhari, B. R. et al. (2006) “Following the TRAIL to Apoptosis,” Immunologic Res. 35(3):249-262); E₁ series (blood group B) as found in pancreatic cancer; EGFR (Epidermal Growth Factor Receptor; Adenis, A. et al. (2003) “Inhibitors Of Epidermal Growth Factor Receptor And Colorectal Cancer,” Bull. Cancer. 90 Spec No:S228-S232); Ephrin receptors (and in particular EphA2 (U.S. Pat. No. 7,569,672; PCT Publication No. WO 06/084226); Erb (ErbB1; ErbB3; ErbB4; Zhou, H. et al. (2002) “Lung Tumorigenesis Associated With Erb-B-2 And Erb-B-3 Overexpression In Human Erb-B-3 Transgenic Mice Is Enhanced By Methylnitrosourea,” Oncogene 21(57):8732-8740; Rimon, E. et al. (2004) “Gonadotropin Induced Gene Regulation In Human Granulosa Cells Obtained From IVF Patients: Modulation Of Genes Coding For Growth Factors And Their Receptors And Genes Involved In Cancer And Other Diseases,” Int. J. Oncol. 24(5):1325-1338); GAGE (GAGE-1; GAGE-2; Akcakanat, A. et al. (2006) “Heterogeneous Expression Of GAGE, NY-ESO-1, MAGE-A and SSX Proteins In Esophageal Cancer: Implications For Immunotherapy,” Int. J. Cancer. 118(1):123-128); GD2/GD3/GM2 (Livingston, P. O. et al. (2005) “Selection Of GM2, Fucosyl GM1, Globo H And Polysialic Acid As Targets On Small Cell Lung Cancers For Antibody Mediated Immunotherapy,” Cancer Immunol. Immunother. 54(10):1018-1025); ganglioside GD2 (GD2; Saleh et al. (1993) “Generation Of A Human Anti-Idiotypic Antibody That Mimics The GD2 Antigen,” J. Immunol., 151, 3390-3398); ganglioside GD3 (G_{D3}; Shitara et al. (1993) “A Mouse/Human Chimeric Anti-(Ganglioside GD3) Antibody With Enhanced Antitumor Activities,” Cancer Immunol. Immunother. 36:373-380); ganglioside GM2 (G_{M2}; Livingston et al. (1994) “Improved Survival In Stage III Melanoma Patients With GM2 Antibodies: A Randomized Trial Of Adjuvant Vaccination With GM2 Ganglioside,” J. Clin. Oncol. 12:1036-1044); ganglioside GM3 (GM3; Hoon et al. (1993) “Molecular Cloning Of A Human Monoclonal Antibody Reactive To Ganglioside GM3 Antigen On Human Cancers,” Cancer Res. 53:5244-5250); GICA 19-9 (Herlyn et al. (1982) “Monoclonal Antibody Detection Of A Circulating Tumor Associated Antigen. I. Presence Of Antigen In Sera Of Patients With Colorectal, Gastric, And Pancreatic Carcinoma,” J. Clin. Immunol. 2:135-140); gp100 (Lotem, M. et al. (2006) “Presentation Of Tumor Antigens By Dendritic Cells Genetically Modified With Viral And Nonviral Vectors,” J. Immunother. 29(6):616-27); Gp37 (human leukemia T cell antigen; Bhattacharya-Chatterjee et al. (1988) “Idiotype Vaccines Against Human T Cell Leukemia. II. Generation And Characterization Of A Monoclonal Idiotype Cascade (Ab1, Ab2, and Ab3),” J. Immunol. 141:1398-1403); gp75 (melanoma antigen; Vijayasardahl et al. (1990) “The Melanoma Antigen Gp75 Is The Human Homologue Of The Mouse B (Brown) Locus Gene Product,” J. Exp. Med. 171(4):1375-1380); gpA33 (Heath, J. K. et al. (1997) “The Human A33 Antigen Is A Transmembrane Glycoprotein And A Novel Member Of The Immunoglobulin Superfamily,” Proc. Natl. Acad. Sci. (U.S.A.) 94(2):469-474; Ritter, G. et al. (1997) “Characterization Of Posttranslational Modifications Of Human A33 Antigen, A Novel Palmitoylated Surface Glycoprotein Of Human Gastrointestinal Epithelium,” Biochem. Biophys. Res. Commun. 236(3):682-686; Wong, N. A. et al. (2006) “EpCAM and gpA33 Are Markers Of Barrett's Metaplasia,” J. Clin. Pathol. 59(3):260-263); HER2 antigen (HER2/neu, p185^{HER2}; Pal, S. K. et al. (2006) “Targeting HER2 Epitopes,” Semin. Oncol. 33(4):386-391); HMFG (human milk fat globule antigen; WO1995015171); human papillomavirus-E6/human papillomavirus-E7 (DiMaio, D. et al. (2006) “Human Papillomaviruses And Cervical Cancer,” Adv. Virus Res. 66:125-59; HMW-MAA (high molecular weight melanoma antigen; Natali et al. (1987) “Immunohistochemical Detection Of Antigen In Human Primary And Metastatic Melanomas By The Monoclonal Antibody 140.240 And Its Possible Prognostic Significance,” Cancer 59:55-63; Mittelman et al. (1990) “Active Specific Immunotherapy In Patients With Melanoma. A Clinical Trial With Mouse Antiidiotypic Monoclonal Antibodies Elicited With Syngeneic Anti-High-Molecular-Weight-Melanoma-Associated Antigen Monoclonal Antibodies,” J. Clin. Invest. 86:2136-2144); I antigen (differentiation antigen; Feizi (1985) “Demonstration By Monoclonal Antibodies That Carbohydrate Structures Of Glycoproteins And Glycolipids Are Onco-Developmental Antigens,” Nature 314:53-57); IL13Rα2 (PCT Publication No. WO 2008/146911; Brown, C. E. et al. (2013) “Glioma IL13Rα2 Is Associated With Mesenchymal Signature Gene Expression And Poor Patient Prognosis,” PLoS One. 18; 8(10):e77769; Barderas, R. et al. (2012) “High Expression Of IL-13 Receptor A2 In Colorectal Cancer Is Associated With Invasion, Liver Metastasis, And Poor Prognosis,” Cancer Res. 72(11):2780-2790; Kasaian, M. T. et al. (2011) “IL-13 Antibodies Influence IL-13 Clearance In Humans By Modulating Scavenger Activity Of IL-13Rα2,” J. Immunol. 187(1):561-569; Bozinov, O. et al. (2010) “Decreasing Expression Of The Interleukin-13 Receptor IL-13Ralpha2 In Treated Recurrent Malignant Gliomas,” Neurol. Med. Chir. (Tokyo) 50(8):617-621; Fujisawa, T. et al. (2009) “A novel role of interleukin-13 receptor alpha2 in pancreatic cancer invasion and metastasis,” Cancer Res. 69(22):8678-8685); Integrin 06 (PCT Publication No. WO 03/087340); JAM-3 (PCT Publication No. WO 06/084078); KID3 (PCT Publication No. WO 05/028498); K1D31 (PCT Publication No. WO 06/076584); KS 1/4 pan-carcinoma antigen (Perez et al. (1989) “Isolation And Characterization Of A cDNA Encoding The Ks1/4 Epithelial Carcinoma Marker,” J. Immunol. 142:3662-3667; Willer et al. (1991) “Bi-specific-Monoclonal-Antibody-Directed Lysis Of Ovarian Carcinoma Cells By Activated Human T Lymphocytes,” Cancer Immunol. Immunother. 33(4):210-216; Ragupathi, G. 2005 Cancer Treat Res. 123:157-80); L6 and L20 (human lung carcinoma antigens; Hellström et al. (1986) “Monoclonal Mouse Antibodies Raised Against Human Lung Carcinoma,” Cancer Res. 46:3917-3923); LEA; LUCA-2 (United States Patent Publication No. 2006/0172349; PCT Publication No. WO 06/083852); M1:22:25:8; M18; M39; MAGE (MAGE-1; MAGE-3; (Bodey, B. (2002) “Cancer-Testis Antigens: Promising Targets For Antigen Directed Antineoplastic Immunotherapy,” Expert Opin. Biol. Ther. 2(6):577-584); MART (Kounalakis, N. et al. (2005) “Tumor Cell And Circulating Markers In Melanoma: Diagnosis, Prognosis, And Management,” Curr. Oncol. Rep. 7(5):377-382; mesothelin (Chang, K. et al. (1996) “Molecular Cloning Of Mesothelin, A Differentiation Antigen Present On Mesothelium, Mesotheliomas, And Ovarian Cancers,” Proc. Natl. Acad. Sci. (U.S.A.) 93:136-140); MUC-1 (Mathelin, C. (2006) “Circulating Proteinic Biomarkers And Breast Cancer,” Gynecol. Obstet. Fertil. 34(7-8):638-646); MUM-1 (Castelli, C. et al. (2000) “T-Cell Recognition Of Melanoma-Associated Antigens,” J. Cell. Physiol. 182(3):323-331); Myl; N-acetylglucosaminyltransferase (Dennis, J. W. (1999) “Glycoprotein Glycosylation And Cancer Progression,” Biochim. Biophys. Acta. 6; 1473(1):21-34); neoglycoprotein; NS-10 as found in adenocarcinomas; OFA-1; OFA-2; Oncostatin M (Oncostatin Receptor Beta; U.S. Pat. No. 7,572,896; PCT Publication No. WO 06/084092); p15 (Gil, J. et al. (2006) “Regulation Of The INK4b-ARF-INK4a Tumour Suppressor Locus: All For One Or One For All,” Nat. Rev. Mol. Cell Biol. 7(9):667-677); p97 (melanoma-associated antigen; Estin et al. (1989) “Transfected Mouse Melanoma Lines That Express Various Levels Of Human Melanoma-Associated Antigen p97,” J. Natl. Cancer Instit. 81(6):445-454); PEM (polymorphic epithelial mucin; Hilkens et al. (1992) “Cell Membrane-Associated Mucins And Their Adhesion-Modulating Property,” Trends in Biochem. Sci. 17:359-363); PEMA (polymorphic epithelial mucin antigen); PIPA (U.S. Pat. No. 7,405,061; PCT Publication No. WO 04/043239); PSA (prostate-specific antigen; Henttu et al. (1989) “cDNA Coding For The Entire Human Prostate Specific Antigen Shows High Homologies To The Human Tissue Kallikrein Genes,” Biochem. Biophys. Res. Comm. 10(2):903-910; Israeli et al. (1993) “Molecular Cloning Of A Complementary DNA Encoding A Prostate-Specific Membrane Antigen,” Cancer Res. 53:227-230; Cracco, C. M. et al. (2005) “Immune Response In Prostate Cancer,” Minerva Urol. Nefrol. 57(4):301-311); PSMA (prostate-specific membrane antigen; Ragupathi, G. (2005) “Antibody Inducing Polyvalent Cancer Vaccines,” Cancer Treat. Res. 123:157-180); prostatic acid phosphate (Tailor et al. (1990) “Nucleotide Sequence Of Human Prostatic Acid Phosphatase Determined From A Full-Length cDNA Clone,” Nucl. Acids Res. 18(16):4928); R24 as found in melanoma; ROM (U.S. Pat. No. 5,843,749); sphingolipids; SSEA-1; SSEA-3; SSEA-4; sTn (Holmberg, L. A. (2001) “Theratope Vaccine (STn-KLH),” Expert Opin. Biol. Ther. 1(5):881-91); T cell receptor derived peptide from a cutaneous T cell lymphoma (see Edelson (1998) “Cutaneous T-Cell Lymphoma: A Model For Selective Immunotherapy,” Cancer J. Sci. Am. 4:62-71); T₅A₇ found in myeloid cells; TAG-72 (Yokota et al. (1992) “Rapid Tumor Penetration Of A Single-Chain Fv And Comparison With Other Immunoglobulin Forms,” Cancer Res. 52:3402-3408); TL5 (blood group A); TNF-receptor (TNF-α receptor, TNF-β receptor; TNF-γ receptor (van Horssen, R. et al. (2006) “TNF-Alpha In Cancer Treatment: Molecular Insights, Antitumor Effects, And Clinical Utility,” Oncologist 11(4):397-408; Gardnerova, M. et al. (2000) “The Use Of TNF Family Ligands And Receptors And Agents Which Modify Their Interaction As Therapeutic Agents,” Curr. Drug Targets 1(4):327-364); TRA-1-85 (blood group H); Transferrin Receptor (U.S. Pat. No. 7,572,895; PCT Publication No. WO 05/121179); 5T4 (TPBG, trophoblast glycoprotein; Boghaert, E. R. et al. (2008) “The Oncofetal Protein, 5T4, Is A Suitable Target For Antibody-Guided Anti-Cancer Chemotherapy With Calicheamicin,” Int. J. Oncol. 32(1):221-234; Eisen, T. et al. (2014) “Naptumomab Estafenatox: Targeted Immunotherapy with a Novel Immunotoxin,” Curr. Oncol. Rep. 16:370, pp. 1-6); TSTA (tumor-specific transplantation antigen) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellström et al. (1985) “Monoclonal Antibodies To Cell Surface Antigens Shared By Chemically Induced Mouse Bladder Carcinomas,” Cancer. Res. 45:2210-2188); VEGF (Pietrantonio, F. et al. (2015) “Bevacizumab Based Neoadjuvant Chemotherapy For Colorectal Cancer Liver Metastases: Pitfalls And Helpful Tricks In A Review For Clinicians,” Crit. Rev. Oncol. Hematol. 95(3):272-281; Grabowski, J. P. (2015) “Current Management Of Ovarian Cancer,” Minerva Med. 106(3):151-156; Field, K. M. (2015) “Bevacizumab And Glioblastoma: Scientific Review, Newly Reported Updates, And Ongoing Controversies,” Cancer 121(7):997-1007; Suh, D. H. et al. (2015) “Major Clinical Research Advances In Gynecologic Cancer In 2014,” J. Gynecol. Oncol. 26(2):156-167; Liu, K. J. et al. (2015) “Bevacizumab In Combination With Anticancer Drugs For Previously Treated Advanced Non-Small Cell Lung Cancer,” Tumour Biol. 36(3):1323-1327; Di Bartolomeo, M. et al. (2015) “Bevacizumab Treatment In The Elderly Patient With Metastatic Colorectal Cancer,” Clin. Interv. Aging 10:127-133); VEGF Receptor (O'Dwyer. P. J. (2006) “The Present And Future Of Angiogenesis-Directed Treatments Of Colorectal Cancer,” Oncologist 11(9):992-998); VEP8; VEP9; VIM-D5; and Y hapten, Le^{y} as found in embryonal carcinoma cells. Additional Cancer Antigens, and molecules (e.g., antibodies) that bind them are disclosed in Table 10. 5T4, B7-H3, CEACAM5/CEACAM6, CD123, DR5, EGFR, an Ephrin receptor, gpA33,HER2/neu, IL13Rα2, ROR1, and VEGF are particularly preferred “Cancer Antigens” of the present invention.

TABLE 10
Antibody and Antibody-Based Molecules

| Antibody | | |
|---|---|---|
| Name | Cancer Antigens | Therapeutic Target Application |
| 3F8 | Gd2 | Neuroblastoma |
| 8H9 | B7-H3 | Neuroblastoma, Sarcoma, |
| | | Metastatic Brain Cancers |
| Abagovomab | CA-125 | Ovarian Cancer |
| Adecatumumab | Epcam | Prostate and Breast Cancer |
| Afutuzumab | CD20 | Lymphoma |
| Alacizumab | VEGFR2 | Cancer |
| Altumomab | CEA | Colorectal Cancer |
| Amatuximab | Mesothelin | Cancer |
| Anatumomab | TAG-72 | Non-Small Cell Lung Carcinoma |
| Mafenatox | | |
| Anifrolumab | Interferon A/B | Systemic Lupus Erythematosus |
| | Receptor | |
| Anrukinzumab | IL-13 | Cancer |
| Apolizumab | HLA-DR | Hematological Cancers |
| Arcitumomab | CEA | Gastrointestinal Cancer |
| Atinumab | RTN4 | Cancer |
| Bectumomab | CD22 | Non-Hodgkin's Lymphoma |
| | | (Detection) |
| Belimumab | BAFF | Non-Hodgkin Lymphoma |
| Bevacizumab | VEGF-A | Metastatic Cancer, Retinopathy |
| | | of Prematurity |
| Bivatuzumab | CD44 V6 | Squamous Cell Carcinoma |
| Blinatumomab | CD19 | Cancer |
| Brentuximab | CD30 (TNFRSF8) | Hematologic Cancers |
| Cantuzumab | MUC1 | Cancers |
| Cantuzumab | Mucin Canag | Colorectal Cancer |
| Mertansine | | |
| Caplacizumab | VWF | Cancers |
| Capromab | Prostatic Carcinoma | Prostate Cancer (Detection) |
| | Cells | |
| Carlumab | MCP-1 | Oncology/Immune Indications |
| Catumaxomab | Epcam, CD3 | Ovarian Cancer, Malignant |
| | | Ascites, Gastric Cancer |
| Cc49 | Tag-72 | Tumor Detection |
| Cetuximab | EGFR | Metastatic Colorectal Cancer |
| | | and Head and Neck Cancer |
| Ch.14.18 | Undetermined | Neuroblastoma |
| Citatuzumab | Epcam | Ovarian Cancer and other Solid |
| | | Tumors |
| Cixutumumab | IGF-1 Receptor | Solid Tumors |
| Clivatuzumab | MUC1 | Pancreatic Cancer |
| Conatumumab | TRAIL-R2 | Cancer |
| Dacetuzumab | CD40 | Hematologic Cancers |
| Dalotuzumab | Insulin-Like Growth | Cancer |
| | Factor I Receptor | |
| Daratumumab | CD38 | Cancer |
| Demcizumab | DLL4 | Cancer |
| Detumomab | B-Lymphoma Cell | Lymphoma |
| Drozitumab | DR5 | Cancer |
| Duligotumab | HER3 | Cancer |
| Dusigitumab | ILGF2 | Cancer |
| Ecromeximab | GD3 Ganglioside | Malignant Melanoma |
| Eculizumab | C5 | Paroxysmal Nocturnal |
| | | Hemoglobinuria |
| Edrecolomab | Epcam | Colorectal Carcinoma |
| Elotuzumab | SLAMF7 | Multiple Myeloma |
| Elsilimomab | IL-6 | Cancer |
| Enavatuzumab | TWEAK Receptor | Cancer |
| Enlimomab | ICAM-1 (CD54) | Cancer |
| Enokizumab | IL9 | Asthma |
| Enoticumab | DLL4 | Cancer |
| Ensituximab | 5AC | Cancer |
| Epitumomab | Episialin | Cancer |
| Cituxetan | | |
| Epratuzumab | CD22 | Cancer, SLE |
| Ertumaxomab | HER2/Neu, CD3 | Breast Cancer |
| Etaracizumab | Integrin Aᵥβ₃ | Melanoma, Prostate Cancer, |
| | | Ovarian Cancer |
| Faralimomab | Interferon Receptor | Cancer |
| Farletuzumab | Folate Receptor 1 | Ovarian Cancer |
| Fasinumab | HNGF | Cancer |
| Fbta05 | CD20 | Chronic Lymphocytic |
| | | Leukaemia |
| Ficlatuzumab | HGF | Cancer |
| Figitumumab | IGF-1 Receptor | Adrenocortical Carcinoma, |
| | | Non-Small Cell Lung Carcinoma |
| Flanvotumab | TYRP1 | Melanoma |
| | (Glycoprotein 75) | |
| Fontolizumab | IFN-γ | Crohn's Disease |
| Fresolimumab | TGF-B | Idiopathic Pulmonary Fibrosis, |
| | | Focal Segmental |
| | | Glomerulosclerosis, Cancer |
| Futuximab | EGFR | Cancer |
| Galiximab | CD80 | B Cell Lymphoma |
| Ganitumab | IGF-I | Cancer |
| Gemtuzumab | CD33 | Acute Myelogenous Leukemia |
| Ozogamicin | | |
| Gevokizumab | IL-1β | Diabetes |
| Girentuximab | Carbonic Anhydrase | Clear Cell Renal Cell Carcinoma |
| | 9 (CA-IX) | |
| Glembatumumab | GPNMB | Melanoma, Breast Cancer |
| Vedotin | | |
| Golimumab | TNF-A | Rheumatoid Arthritis, Psoriatic |
| | | Arthritis, Ankylosing |
| | | Spondylitis |
| Ibritumomab | CD20 | Non-Hodgkin's Lymphoma |
| Tiuxetan | | |
| Icrucumab | VEGFR-1 | Cancer |
| Igovomab | CA-125 | Ovarian Cancer (Diagnosis) |
| Imab362 | Cldn18.2 | Gastrointestinal |
| | | Adenocarcinomas and Pancreatic |
| | | Tumor |
| Imgatuzumab | EGFR | Cancer |
| Inclacumab | Selectin P | Cancer |
| Indatuximab | SDC1 | Cancer |
| Ravtansine | | |
| Inotuzumab | CD22 | Cancer |
| Ozogamicin | | |
| Intetumumab | CD51 | Solid Tumors (Prostate Cancer, |
| | | Melanoma) |
| Ipilimumab | CD152 | Melanoma |
| Iratumumab | CD30 (TNFRSF8) | Hodgkin's Lymphoma |
| Itolizumab | CD6 | Cancer |
| Labetuzumab | CEA | Colorectal Cancer |
| Lambrolizumab | PDCD1 | Antineoplastic Agent |
| Lampalizumab | CFD | Cancer |
| Lexatumumab | TRAIL-R2 | Cancer |
| Libivirumab | Hepatitis B Surface | Hepatitis B |
| | Antigen | |
| Ligelizumab | IGHE | Cancer |
| Lintuzumab | CD33 | Cancer |
| Lirilumab | KIR2D | Cancer |
| Lorvotuzumab | CD56 | Cancer |
| Lucatumumab | CD40 | Multiple Myeloma, |
| | | Non-Hodgkin's Lymphoma, |
| | | Hodgkin's Lymphoma |
| Lumiliximab | CD23 | Chronic Lymphocytic |
| | | Leukemia |
| Mapatumumab | TRAIL-R1 | Cancer |
| Margetuximab | Ch4d5 | Cancer |
| Matuzumab | EGFR | Colorectal, Lung and Stomach |
| | | Cancer |
| Milatuzumab | CD74 | Multiple Myeloma and Other |
| | | Hematological Malignancies |
| Minretumomab | TAG-72 | Cancer |
| Mitumomab | GD3 Ganglioside | Small Cell Lung Carcinoma |
| Mogamulizumab | CCR4 | Cancer |
| Morolimumab | Rhesus Factor | Cancer |
| Moxetumomab | CD22 | Cancer |
| Pasudotox | | |
| Nacolomab | C242 Antigen | Colorectal Cancer |
| Tafenatox | | |
| Namilumab | CSF2 | Cancer |
| Naptumomab | 5T4 | Non-Small Cell Lung |
| Estafenatox | | Carcinoma, Renal Cell |
| | | Carcinoma |
| Narnatumab | RON | Cancer |
| Nebacumab | Endotoxin | Sepsis |
| Necitumumab | EGFR | Non-Small Cell Lung |
| | | Carcinoma |
| Nerelimomab | TNF-A | Cancer |
| Nesvacumab | Angiopoietin 2 | Cancer |
| Nimotuzumab | EGFR | Squamous Cell Carcinoma, |
| | | Head and Neck Cancer, |
| | | Nasopharyngeal Cancer, Glioma |
| Nivolumab | PD-1 | Cancer |
| Nofetumomab | Undetermined | Cancer |
| Merpentan | | |
| Ocaratuzumab | CD20 | Cancer |
| Ofatumumab | CD20 | Chronic Lymphocytic |
| | | Leukemia |
| Olaratumab | PDGF-R A | Cancer |
| Olokizumab | IL6 | Cancer |
| Onartuzumab | Human Scatter | Cancer |
| | Factor Receptor | |
| | Kinase | |
| Ontuxizumab | TEM1 | Cancer |
| Oportuzumab | Epcam | Cancer |
| Monatox | | |
| Oregovomab | CA-125 | Ovarian Cancer |
| Orticumab | Oxldl | Cancer |
| Otlertuzumab | CD37 | Cancer |
| Panitumumab | EGFR | Colorectal Cancer |
| Pankomab | Tumor Specific | Ovarian Cancer |
| | Glycosylation of | |
| | MUC1 | |
| Parsatuzumab | EGFL7 | Cancer |
| Patritumab | HER3 | Cancer |
| Pembrolizumab | PD-1 | Cancer |
| Pemtumomab | MUC1 | Cancer |
| Perakizumab | IL17A | Arthritis |
| Pertuzumab | HER2/Neu | Cancer |
| Pidilizumab | PD-1 | Cancer and Infectious Diseases |
| Pinatuzumab | CD22 | Cancer |
| Vedotin | | |
| Pintumomab | Adenocarcinoma | Adenocarcinoma |
| | Antigen | |
| Placulumab | Human TNF | Cancer |
| Polatuzumab | CD79B | Cancer |
| Vedotin | | |
| Pritoxaximab | E. Coli Shiga Toxin | Cancer |
| | Type-1 | |
| Pritumumab | Vimentin | Brain Cancer |
| Quilizumab | IGHE | Cancer |
| Racotumomab | N- | Cancer |
| | Glycolylneuraminic | |
| | Acid | |
| Radretumab | Fibronectin Extra | Cancer |
| | Domain-B | |
| Ramucirumab | VEGFR2 | Solid Tumors |
| Rilotumumab | HGF | Solid Tumors |
| Rituximab | CD20 | Lymphomas, Leukemias, Some |
| | | Autoimmune Disorders |
| Robatumumab | IGF-1 Receptor | Cancer |
| Roledumab | RHD | Cancer |
| Samalizumab | CD200 | Cancer |
| Satumomab | TAG-72 | Cancer |
| Pendetide | | |
| Seribantumab | ERBB3 | Cancer |
| Setoxaximab | E. Coli Shiga Toxin | Cancer |
| | Type-1 | |
| Sgn-CD19a | CD19 | Acute Lymphoblastic |
| | | Leukemia and B Cell |
| | | Non-Hodgkin Lymphoma |
| Sgn-CD33a | CD33 | Acute Myeloid Leukemia |
| Sibrotuzumab | FAP | Cancer |
| Siltuximab | IL-6 | Cancer |
| Solitomab | Epcam | Cancer |
| Sontuzumab | Episialin | Cancer |
| Tabalumab | BAFF | B Cell Cancers |
| Tacatuzumab | Alpha-Fetoprotein | Cancer |
| Tetraxetan | | |
| Taplitumomab | CD19 | Cancer |
| Paptox | | |
| Telimomab | Undetermined | Cancer |
| Tenatumomab | Tenascin C | Cancer |
| Teneliximab | CD40 | Cancer |
| Teprotumumab | CD221 | Hematologic Tumors |
| Ticilimumab | CTLA-4 | Cancer |
| Tigatuzumab | TRAIL-R2 | Cancer |
| Tnx-650 | Il-13 | Hodgkin's Lymphoma |
| Tositumomab | CD20 | Follicular Lymphoma |
| Tovetumab | CD140a | Cancer |
| Trastuzumab | HER2/Neu | Breast Cancer |
| Trbs07 | Gd2 | Melanoma |
| Tremelimumab | CTLA-4 | Cancer |
| Tucotuzumab | Epcam | Cancer |
| Celmoleukin | | |
| Ublituximab | MS4A1 | Cancer |
| Urelumab | 4-1BB | Cancer |
| Vantictumab | Frizzled Receptor | Cancer |
| Vapaliximab | AOC3 (VAP-1) | Cancer |
| Vatelizumab | ITGA2 | Cancer |
| Veltuzumab | CD20 | Non-Hodgkin's Lymphoma |
| Vesencumab | NRP1 | Cancer |
| Volociximab | Integrin A5β1 | Solid Tumors |
| Vorsetuzumab | CD70 | Cancer |
| Votumumab | Tumor Antigen | Colorectal Tumors |
| | CTAA16.88 | |
| Zalutumumab | EGFR | Squamous Cell Carcinoma of |
| | | The Head And Neck |
| Zatuximab | HER1 | Cancer |
| Ziralimumab | CD147 | Cancer |

### D. Exemplary Antibodies Capable Of Binding A Cancer Antigen

Exemplary antibodies, whose VH and VL Domains may be used to construct molecules capable of binding a Cancer Antigen arrayed on the surface of a cancer cell and mediating the redirected killing of such cancer cells are listed in Table 10 above, additional antibodies that may be used to construct molecules capable of binding a Cancer Antigen arrayed on the surface of a cancer cell and mediating the redirected killing of such cancer cells are provided below.

### 1. Antibodies that Bind B7-H3

B7-H3 is a Cancer Antigen that is over-expressed on a wide variety of solid tumor types and is a member of the B7 family of molecules that are involved in immune regulation (see, U.S. Pat. No. 8,802,091; US 2014/0328750; US 2013/0149236; Loo, D. et al. (2012) “Development Of An Fc-Enhanced Anti-B7-H3 Monoclonal Antibody With Potent Antitumor Activity,” Clin. Cancer Res. 18(14):3834-3845). In particular, several independent studies have shown that human malignant cancer cells (e.g., cancer cells of neuroblastomas and gastric, ovarian and non-small cell lung cancers) exhibit a marked increase in expression of B7-H3 protein and that this increased expression was associated with increased disease severity (Zang, X. et al. (2007) “The B7 Family And Cancer Therapy: Costimulation And Coinhibition,” Clin. Cancer Res. 13:5271-5279), suggesting that B7-H3 is exploited by tumors as an immune evasion pathway (Hofmeyer, K. et al. (2008) “The Contrasting Role Of B7-H3,” Proc. Natl. Acad. Sci. (U.S.A.) 105(30):10277-10278).

B7-H3 has also been found to co-stimulate CD4+ and CD8+ T-cell proliferation. B7-H3 also stimulates IFN-γ production and CD8+ lytic activity (Chapoval, A. et al. (2001) “B7-H3: A Costimulatory Molecule For T Cell Activation and IFN-γ Production,” Nature Immunol. 2:269-274; Sharpe, A. H. et al. (2002) “The B7-CD28 Superfamily,” Nature Rev. Immunol. 2:116-126). However, the protein also possibly acts through NFAT (nuclear factor for activated T cells), NF-κB (nuclear factor kappa B), and AP-1 (Activator Protein-1) factors to inhibit T-cell activation (Yi. K. H. et al. (2009) “Fine Tuning The Immune Response Through B7-H3 And B7-H4,” Immunol. Rev. 229:145-151). B7-H3 is also believed to inhibit Th1, Th2, or Th17 in vivo (Prasad, D. V. et al. (2004) “Murine B7-H3 Is A Negative Regulator Of T Cells,” J. Immunol. 173:2500-2506; Fukushima, A. et al. (2007) “B7-H3 Regulates The Development Of Experimental Allergic Conjunctivitis In Mice,” Immunol. Lett. 113:52-57; Yi. K. H. et al. (2009) “Fine Tuning The Immune Response Through B7-H3 And B7-H4,” Immunol. Rev. 229:145-151).

Preferred B7-H3-binding molecules possess the VL and/or VH Domains, of the anti-human B7-H3 monoclonal antibody “B7-H3 mAb 1,” “B7-H3 mAb 2,” or “B7-H3 mAb 3,” or any of the anti-B7-H3 antibodies provided herein; and more preferably possess 1, 2 or all 3 of the CDR_{L}_{^{S}} of the VL Region and/or 1, 2 or all 3 of the CDR_{H}_{^{S}} of the VH Domain of such anti-B7-H3 monoclonal antibodies. Particularly preferred, are B7-H3-binding molecules which possess a humanized VH and/or VL Domain including but not limited to “Enoblituzumab” (also known as MGA271; CAS Reg No. 1353485-38-7). Enoblituzumab is an Fc-optimized monoclonal antibody that binds to HER2/neu and mediates enhanced ADCC activity. The amino acid sequences of the complete Heavy and Light Chains of Enoblituzumab are known in the art (see., e.g., WHO Drug Information, 2017, Recommended INN: List 77, 31(1):49).

The present invention specifically includes and encompasses B7-H3 x CD3 bispecific binding molecules that are capable of binding to B7-H3 and to CD3, and particularly such bispecific binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of any of anti-B7-H3 monoclonal antibodies B7-H3 mAb 1, B7-H3 mAb 2, or B7-H3 mAb 3, or of any of the B7-H3 x CD3 bispecific binding molecules provided herein, or of any of the B7-H3 x CD3 bispecific binding molecules provided in WO 2017/030926.

### (a) B7-H3 mAb 1

The amino acid sequence of the VH Domain of B7-H3 mAb 1 (SEQ ID NO:213) is shown below (CDR_{H} residues are shown underlined).

QVQLQQSGAE LARPGASVKL SCKASGYTFT SYWMQWVKQR
PGQGLEWIGT IYPGDGDTRY TQKFKGKATL TADKSSSTAY
MQLSSLASED SAVYYCARRG IPRLWYFDVW GAGTTVTVSS

The amino acid sequence of the VL Domain of B7-H3 mAb 1 (SEQ ID NO:214) is shown below (CDR_{L} residues are shown underlined).

DIQMTQTTSS LSASLGDRVT ISCRASQDIS NYLNWYQQKP
DGTVKLLIYY TSRLHSGVPS RFSGSGSGTD YSLTIDNLEQ
EDIATYFCQQ GNTLPPTFGG GTKLEIK

Two exemplary humanized VH Domains of B7-H3 mAb 1 designated herein as “hB7-H3 mAb 1 VH1,” and “hB7-H3 mAb 1 VH2,” and two exemplary humanized VL Domains of B7-H3 mAb 1 designated herein as “hB7-H3 mAb 1 VL1,” and “hB7-H3 mAb 1 VL2,” are provided below. It will be noted that hB7-H3 mAb 1 VL2 includes amino acid substitutions in CDR_{L}1 and CDR_{L}2, and that hB7-H3 mAb 1 VH2 includes amino acid substitutions in CDR_{H}2. Any of the humanized VL Domains may be paired with any of the humanized VH Domains to generate a B7-H3 binding domain. Accordingly, any antibody comprising one of the humanized VL Domains paired with the humanized VH Domain is referred to generically as “hB7-H3 mAb 1,” and particular combinations of humanized VH/VL Domains are referred to by reference to the specific VH/VL Domains, for example a humanized antibody comprising hB7-H3 mAb 1 VH1 and hB7-H3 mAb 1 VL2 is specifically referred to as “hB7-H3 mAb 1 (1.2).”

The amino acid sequence of the VH Domain of hB7-H3 mAb 1 VH1 is (SEQ ID NO:215) (CDR_{H} residues are shown underlined):

QVQLVQSGAE VKKPGASVKV SCKASGYTFT SYWMQWVRQA
PGQGLEWMGT IYPGDGDTRY TQKFKGRVTI TADKSTSTAY
MELSSLRSED TAVYYCARRG IPRLWYFDVW GQGTTVTVSS

The amino acid sequence of the VH Domain of hB7-H3 mAb 1 VH2 is (SEQ ID NO:216) (CDR_{H} residues are shown underlined):

QVQLVQSGAE VKKPGASVKV SCKASGYTFT SYWMQWVRQA
PGQGLEWMGT IYPGGGDTRY TQKFQGRVTI TADKSTSTAY
MELSSLRSED TAVYYCARRG IPRLWYFDVW GQGTTVTVSS

The amino acid sequence of the VL Domain of hB7-H3 mAb 1 VL1 (SEQ ID NO:217) is shown below (CDR_{L} residues are shown underlined).

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIK

The amino acid sequence of the VL Domain of hB7-H3 mAb 1 VL2 (SEQ ID NO:218) is shown below (CDR_{L} residues are shown underlined).

DIQMTQSPSS LSASVGDRVT ITCRASQSIS SYLNWYQQKP
GKAPKLLIYY TSRLQSGVPS RFSGSGSGTD FTLTISSLQP
EDIATYYCQQ GNTLPPTFGG GTKLEIK

### (b) B7-H3 mAb 2

The amino acid sequence of the VH Domain of B7-H3 mAb 2 (SEQ ID NO:219) is shown below (CDR_{H} residues are shown underlined).

DVQLVESGGG LVQPGGSRKL SCAASGFTFS SFGMHWVRQA
PEKGLEWVAY ISSDSSAIYY ADTVKGRFTI SRDNPKNTLF
LQMTSLRSED TAMYYCGRGR ENIYYGSRLD YWGQGTTLTV SS

The amino acid sequence of the VL Domain of B7-H3 mAb 2 (SEQ ID NO:220) is shown below (CDR_{L} residues are shown underlined).

DIAMTQSQKF MSTSVGDRVS VTCKASQNVD TNVAWYQQKP
GQSPKALIYS ASYRYSGVPD RFTGSGSGTD FTLTINNVQS
EDLAEYFCQQ YNNYPFTFGS GTKLEIK

Four exemplary humanized VH Domains of B7-H3 mAb 2, designated herein as “hB7-H3 mAb 2 VH1,” “hB7-H3 mAb 2 VH2,” “hB7-H3 mAb 2 VH3,” and “hB7-H3 mAb 2 VH4,” and six exemplary humanized VL Domains of B7-H3 mAb 2, designated herein as “hB7-H3 mAb 2 VL1,” “hB7-H3 mAb 2 VL2,” “hB7-H3 mAb 2 VL3,” “hB7-H3 mAb 2 VL4,” “hB7-H3 mAb 2 VL5,” and “hB7-H3 mAb 2 VL6,” and are provided below. Any of the humanized VL Domains may be paired with any of the humanized VH Domains to generate a B7-H3 binding domain. Accordingly, any antibody comprising one of the humanized VL Domains paired with the humanized VH Domain is referred to generically as “hB7-H3 mAb 2,” and particular combinations of humanized VH/VL Domains are referred to by reference to the specific VH/VL Domains, for example a humanized antibody comprising hB7-H3 mAb 2 VH1 and hB7-H3 mAb 2 VL2 is specifically referred to as “hB7-H3 mAb 2 (1.2).”

The amino acid sequence of the VH Domain of hB7-H3 mAb 2 VH1 (SEQ ID NO:221) is shown below (CDR_{H} residues are shown underlined).

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCARGR ENIYYGSRLD YWGQGTTVTV SS

The amino acid sequence of the VH Domain of hB7-H3 mAb 2 VH2 (SEQ ID NO:222) is shown below (CDR_{H} residues are shown underlined).

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCGRGR ENIYYGSRLD YWGQGTTVTV SS

The amino acid sequence of the VH Domain of hB7-H3 mAb 2 VH3 (SEQ ID NO:223) is shown below (CDR_{H} residues are shown underlined).

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAMYYCGRGR ENIYYGSRLD YWGQGTTVTV SS

The amino acid sequence of the VH Domain of hB7-H3 mAb 2 VH4 (SEQ ID NO:224) is shown below (CDR_{H} residues are shown underlined).

EVQLVESGGG LVQPGGSLRL SCAASGFTFS SFGMHWVRQA
PGKGLEWVAY ISSDSSAIYY ADTVKGRFTI SRDNAKNSLY
LQMNSLRSED TAVYYCARGR ENIYYGSRLD YWGQGTTVTV SS

The amino acid sequence of the VL Domain of hB7-H3 mAb 2 VL1 (SEQ ID NO:225) is shown below (CDR_{L} residues are shown underlined).

DIQLTQSPSF LSASVGDRVT ITCKASQNVD TNVAWYQQKP
GKAPKLLIYS ASYRYSGVPS RFSGSGSGTD FTLTISSLQP
EDFATYYCQQ YNNYPFTFGQ GTKLEIK

The amino acid sequence of the VL Domain of hB7-H3 mAb 2 VL2 (SEQ ID NO:226) is shown below (CDR_{L} residues are shown underlined).

DIQLTQSPSF LSASVGDRVT ITCKASQNVD TNVAWYQQKP
GKAPKALIYS ASYRYSGVPS RFSGSGSGTD FTLTISSLQP
EDFATYYCQQ YNNYPFTFGQ GTKLEIK

The amino acid sequence of the VL Domain of hB7-H3 mAb 2 VL3 (SEQ ID NO:227) is shown below (CDR_{L} residues are shown underlined).

DIQLTQSPSF LSASVGDRVS VTCKASQNVD TNVAWYQQKP
GKAPKLLIYS ASYRYSGVPS RFSGSGSGTD FTLTISSLQP
EDFATYYCQQ YNNYPFTFGQ GTKLEIK

The amino acid sequence of the VL Domain of hB7-H3 mAb 2 VL4 (SEQ ID NO:228) is shown below (CDR_{L} residues are shown underlined).

DIQLTQSPSF LSASVGDRVT ITCKASQNVD TNVAWYQQKP
GQAPKLLIYS ASYRYSGVPS RFSGSGSGTD FTLTISSLQP
EDFATYYCQQ YNNYPFTFGQ GTKLEIK

The amino acid sequence of the VL Domain of hB7-H3 mAb 2 VL5 (SEQ ID NO:229) is shown below (CDR_{L} residues are shown underlined).

DIQLTQSPSF LSASVGDRVT ITCKASQNVD TNVAWYQQKP
GQAPKALIYS ASYRYSGVPS RFSGSGSGTD FTLTISSLQP
EDFATYYCQQ YNNYPFTFGQ GTKLEIK

The amino acid sequence of the VL Domain of hB7-H3 mAb 2 VL6 (SEQ ID NO:230) is shown below (CDR_{L} residues are shown underlined).

DIQLTQSPSF LSASVGDRVT ITCKASQNVD TNVAWYQQKP
GKAPKLLIYS ASYRYSGVPS RFSGSGSGTD FTLTISSLQP
EDFAEYYCQQ YNNYPFTFGQ GTKLEIK

### (c) B7-H3 mAb 3

The amino acid sequence of the VH Domain of B7-H3 mAb 3 (SEQ ID NO:231) is shown below (CDR_{H} residues are shown underlined).

EVQQVESGGD LVKPGGSLKL SCAASGFTFS SYGMSWVRQT
PDKRLEWVAT INSGGSNTYY PDSLKGRFTI SRDNAKNTLY
LQMRSLKSED TAMYYCARHD GGAMDYWGQG TSVTVSS

The amino acid sequence of the VL Domain of B7-H3 mAb 3 (SEQ ID NO:232) is shown below (CDR_{L} residues are shown underlined).

DIQMTQSPAS LSVSVGETVT ITCRASESIY SYLAWYQQKQ
GKSPQLLVYN TKTLPEGVPS RFSGSGSGTQ FSLKINSLQP
EDFGRYYCQH HYGTPPWTFG GGTNLEIK

### (d) Other Anti-B7-H3 Binding Molecules

In addition to the above-identified preferred anti-B7-H3 Binding Molecules, the invention contemplates the use of any of the following anti-B7-H3 Binding Molecules: LUCA1; BLAB; PA20; or SKN2 (see, U.S. Pat. Nos. 7,527,969; 8,779,098 and PCT Patent Publication WO 2004/001381); M30; cM30; M30-H1-L1; M30-H1-L2; M30-H1-L3; M30-H1-L4; M30-H1-L5; M30-H1-L6; M30-H1-L7; M30-H4-L1; M30-H4-L2; M30-H4-L3; and M30-H4-L4 (see, US Patent Publication 2013/0078234 and PCT Patent Publication WO 2012/147713); and 8H9 (see U.S. Pat. Nos. 7,666,424; 7,737,258; 7,740,845; 8,148,154; 8,414,892; 8,501,471; 9,062,110; US Patent Publication 2010/0143245 and PCT Patent Publication WO 2008/116219).

### 2. Antibodies That Bind CEACAM5 and CEACAM6

Carcinoembryonic Antigen-Related Cell Adhesion Molecules 5 (CEACAM5) and 6 (CEACAM6) have been found to be associated with various types of cancers including medullary thyroid cancer, colorectal cancer, pancreatic cancer, hepatocellular carcinoma, gastric cancer, lung cancer, head and neck cancers, urinary bladder cancer, prostate cancer, uterine cancer, endometrial cancer, breast cancer, hematopoietic cancer, leukemia and ovarian cancer (PCT Pubmication No. WO 2011/034660), and particularly colorectal, gastrointestinal, pancreatic, non-small cell lung cancer (NSCL), breast, thyroid, stomach, ovarian and uterine carcinomas (Zheng, C. et al. (2011) “A Novel Anti-CEACAM5 Monoclonal Antibody, CC4, Suppresses Colorectal Tumor Growth and Enhances NK Cells-Mediated Tumor Immunity,” PLoS One 6(6):e21146, pp. 1-11).

CEACAM5 has been found to be overexpressed in 90% of gastrointestinal, colorectal and pancreatic cancers, 70% of non-small cell lung cancer cells and 50% of breast cancers (Thompson, J. A. et al. (1991) “Carcinoembryonic Antigen Gene Family: Molecular Biology And Clinical Perspectives,” J. Clin. Lab. Anal. 5:344-366). Overexpressed carcinoembryonic antigen-related cellular adhesion molecule 6 (CEACAM6) plays important roles in the invasion and metastasis of a variety of human cancers, including medullary thyroid cancer, colorectal cancer, pancreatic cancer, hepatocellular carcinoma, gastric cancer, lung cancer, head and neck cancers, urinary bladder cancer, prostate cancer, uterine cancer, endometrial cancer, breast cancer, hematopoietic cancer, leukemia and ovarian cancer (PCT Pubmication No. WO 2011/034660; Deng, X. et al. (2014) “Expression Profiling Of CEACAM6 Associated With The Tumorigenesis And Progression In Gastric Adenocarcinoma,” Genet. Mol. Res. 13(3):7686-7697; Cameron, S. et al. (2012) “Focal Overexpression Of CEACAM6 Contributes To Enhanced Tumourigenesis In Head And Neck Cancer Via Suppression Of Apoptosis,” Mol. Cancer 11:74, pp. 1-11; Chapin, C. et al. (2012) “Distribution And Surfactant Association Of Carcinoembryonic Cell Adhesion Molecule 6 In Human Lung,” Amer. J. Physiol. Lung Cell. Mol. Physiol. 302(2):L216-L25; Riley, C. J. et al. (2009) “Design And Activity Of A Murine And Humanized Anti-CEACAM6 Single-Chain Variable Fragment In The Treatment Of Pancreatic Cancer,” Cancer Res. 69(5):1933-1940; Lewis-Wambi, J. S. et al. (2008) “Overexpression Of CEACAM6 Promotes Migration And Invasion Of Oestrogen-Deprived Breast Cancer Cells,” Eur. J. Cancer 44(12):1770-1779; Blumenthal, R. D. et al. (2007) “Expression Patterns Of CEACAM5 And CEACAM6 In Primary And Metastatic Cancers,” BMC Cancer. 7:2, pp. 1-15). Antibodies that immunospecifically bind CEACAM5 and CEACAM6 are commercially available (Santa Cruz Biotechnology, Inc., Novus Biologicals LLC; Abnova Corporation).

### (a) Antibody 16C3

The amino acid sequence of the VH Domain of the humanized anti-CEACAM5/ANTI-CEACAM6 antibody 16C3 (EP 2585476) (SEQ ID NO:233) is shown below (CDR_{H} residues are shown underlined):

QVQLQQSGPE VVRPGVSVKI SCKGSGYTFT DYAMHWVKQS
HAKSLEWIGL ISTYSGDTKY NQNFKGKATM TVDKSASTAY
MELSSLRSED TAVYYCARGD YSGSRYWFAY WGQGTLVTVS S

The amino acid sequence of the VL Domain of the humanized anti-CEACAM5/ANTI-CEACAM6 antibody 16C3 (EP 2585476) (SEQ ID NO:234) is shown below (CDR_{L} residues are shown underlined):

DIQMTQSPSS LSASVGDRVT ITCGASENIY GALNWYQRKP
GKSPKLLIWG ASNLADGMPS RFSGSGSGRQ YTLTISSLQP
EDVATYYCQN VLSSPYTFGG GTKLEIK

### (b) Antibody hMN15

The amino acid sequence of the VH Domain of the humanized anti-CEACAM5/CEACAM6 antibody hMN15 (WO 2011/034660) (SEQ ID NO:235) is shown below (CDR_{H} residues are shown underlined):

QVQLVESGGG VVQPGRSLRL SCSSSGFALT DYYMSWVRQA
PGKGLEWLGF IANKANGHTT DYSPSVKGRF TISRDNSKNT
LFLQMDSLRP EDTGVYFCAR DMGIRWNFDV WGQGTPVTVS S

The amino acid sequence of the VL Domain of the humanized anti-CEACAM5/CEACAM6 antibody hMN15 (WO 2011/034660) (SEQ ID NO:236) is shown below (CDR_{L} residues are shown underlined):

DIQLTQSPSS LSASVGDRVT MTCSASSRVS YIHWYQQKPG
KAPKRWIYGT STLASGVPAR FSGSGSGTDF TFTISSLQPE
DIATYYCQQW SYNPPTFGQG TKVEIKR

The present invention specifically includes and encompasses CEACAM5/CEACAM6 binding molecules (e.g., CEACAM5/CEACAM6 x CD3 bispecific binding molecules) that are capable of binding to CEACAM5 and/or CEACAM6, and particularly such binding molecules that comprise the VL and/or VH Domain, and/or 1, 2 or all 3 of the CDR_{L}5 of the VL Region and/or 1, 2 or all 3 of the CDR_{H}5 of the VH Domain of the anti-CEACAM5/CEACAM6 monoclonal antibodies 16C3 or hMN15.

### 3. Antibodies That Bind EGFR

Epidermal Growth Factor Receptor (EGFR) is a Cancer Antigen of certain metastatic colorectal cancer, metastatic non-small cell lung cancer and head and neck cancer. Exemplary antibodies that bind human EGRF are “Cetuximab” and “Panitumumab.” Cetuximab is a recombinant human-mouse chimeric epidermal growth factor receptor (EGFR) IgG1 monoclonal antibody (Govindan R. (2004) “Cetuximab In Advanced Non-Small Cell Lung Cancer,” Clin. Cancer Res. 10(12 Pt 2):4241s-4244s; Bou-Assaly, W. et al. (2010) “Cetuximab (Erbitux),” Am. J. Neuroradiol. 31(4):626-627). Panitumumab (Vectibix®, Amgen) is a fully humanized epidermal growth factor receptor (EGFR) IgG2 monoclonal antibody (Foon, K. A. et al. (2004) “Preclinical And Clinical Evaluations Of ABX-EGF, A Fully Human Anti-Epidermal Growth Factor Receptor Antibody,” Int. J. Radiat. Oncol. Biol. Phys. 58(3):984-990; Yazdi, M. H. et al. (2015) “A Comprehensive Review of Clinical Trials on EGFR Inhibitors Such as Cetuximab and Panitumumab as Monotherapy and in Combination for Treatment of Metastatic Colorectal Cancer,” Avicenna J. Med. Biotechnol. 7(4):134-144).

### (a) Cetuximab

The amino acid sequence of the VH Domain of the chimeric anti-EGFR antibody Cetuximab (SEQ ID NO:237) is shown below (CDR_{H} residues are shown underlined):

QVQLKQSGPG LVQPSQSLSI TCTVSGFSLT NYGVHWVRQS
PGKGLEWLGV IWSGGNTDYN TPFTSRLSIN KDNSKSQVFF
KMNSLQSNDT AIYYCARALT YYDYEFAYWG QGTLVTVSA

The amino acid sequence of the VL Domain of the chimeric anti-EGFR antibody Cetuximab (SEQ ID NO:238) is shown below (CDR_{L} residues are shown underlined):

DILLTQSPVI LSVSPGERVS FSCRASQSIG TNIHWYQQRT
NGSPRLLIKY ASESISGIPS RFSGSGSGTD FTLSINSVES
EDIADYYCQQ NNNWPTTFGA GTKLELKR

### (b) Panitumumab

The amino acid sequence of the VH Domain of Panitumumab (SEQ ID NO:239) is shown below (CDR_{H} residues are shown underlined):

QVQLQESGPG LVKPSETLSL TCTVSGGSVS SGDYYWTWIR
QSPGKGLEWI GHIYYSGNTN YNPSLKSRLT ISIDTSKTQF
SLKLSSVTAA DTAIYYCVRD RVTGAFDIWG QGTMVTVSS

The amino acid sequence of the VL Domain of Panitumumab (SEQ ID NO:240) is shown below (CDR_{L} residues are shown underlined):

DIQMTQSPSS LSASVGDRVT ITCQASQDIS NYLNWYQQKP
GKAPKLLIYD ASNLETGVPS RFSGSGSGTD FTFTISSLQP
EDIATYFCQH FDHLPLAFGG GTKVEIKR

The present application specifically includes and encompasses EGFR binding molecules (e.g., EGFR x CD3 bispecific binding molecules) that are capable of binding to EGFR, and particularly such binding molecules that comprise the VL and/or VH Domain, and/or 1, 2 or all 3 of the CDR_{L}_{^{S}} of the VL Region and/or 1, 2 or all 3 of the CDR_{H}5 of the VH Domain of the anti-EGFR monoclonal antibodies Cetuximab or Panitumumab.

### 4. Antibodies That Bind EphA2

The receptor tyrosine kinase, Ephrin type-A receptor 2 (EphA2) is normally expressed at sites of cell-to-cell contact in adult epithelial tissues, however, recent studies have shown that it is also overexpressed in various types of epithelial carcinomas, with the greatest level of EphA2 expression observed in metastatic lesions. High expression levels of EphA2 have been found in a wide range of cancers and in numerous cancer cell lines, including prostate cancer, breast cancer, non-small cell lung cancer and melanoma (Xu, J. et al. (2014) “High EphA2 Protein Expression In Renal Cell Carcinoma Is Associated With A Poor Disease Outcome,” Oncol. Lett. August 2014; 8(2): 687-692; Miao, B. et al. (2014) “EphA2 is a Mediator of Vemurafenib Resistance and a Novel Therapeutic Target in Melanoma,” Cancer Discov. pii: CD-14-0295). EphA2 does not appear to be merely a marker for cancer, but rather appears to be persistently overexpressed and functionally changed in numerous human cancers (Chen, P. et al. (2014) “EphA2 Enhances The Proliferation And Invasion Ability Of LnCap Prostate Cancer Cells,” Oncol. Lett. 8(1):41-46). Exemplary antibodies that bind human EphA2 are “EphA2 mAb 1,” “EphA2 mAb 2” and “EphA2 mAb 3.”

### (a) EphA2 mAb 1

The amino acid sequence of the VH Domain of EphA2 mAb 1 (SEQ ID NO:241) is shown below (CDR_{H} residues are shown underlined):

QVQLKESGPG LVAPSQSLSI TCTVSGFSLS RYSVHWVRQP
PGKGLEWLGM IWGGGSTDYN SALKSRLSIS KDNSKSQVFL
KMNSLQTDDT AMYYCARKHG NYYTMDYWGQ GTSVTVSS

The amino acid sequence of the VL Domain of EphA2 mAb 1 (SEQ ID NO:242) is shown below (CDR_{L} residues are shown underlined):

DIQMIQTTSS LSASLGDRIT ISCRASQDIS NYLNWYQQKP
DGIVKLLIYY TSRLHSGVPS RFSGSGSGTD YSLTISNLEQ
EDIATYFCQQ GYTLYTFGGG TKLEIK

### (b) EphA2 mAb 2

The amino acid sequence of the VH Domain of EphA2 mAb 2 (SEQ ID NO:243) is shown below (CDR_{H} residues are shown underlined):

QIQLVQSGPE LKKPGETVKI SCKASGFTFT NYGMNWVKQA
PGKGLKWMGW INTYIGEPTY ADDFKGRFVF SLETSASTAY
LQINNLKNED MATYFCAREL GPYYFDYWGQ GTTLTVSS

The amino acid sequence of the VL Domain of EphA2 mAb 2 (SEQ ID NO:244) is shown below (CDR_{L} residues are shown underlined):

DVVMTQTPLS LPVSLGDQAS ISCRSSQSLV HSSGNTYLHW
YLQKPGQSPK LLIYKVSNRF SGVPDRFSGS GSGTDFILKI
SRVEAEDLGV YFCSQSTHVP TFGSGTKLEI K

### (c) EphA2 mAb 3

The amino acid sequence of the VH Domain of EphA2 mAb 3 (SEQ ID NO:245) is shown below (CDR_{H} residues are shown underlined):

EVQLVESGGG SVKPGGSLKL SCAASGFTFT DHYMYWVRQT
PEKRLEWVAT ISDGGSFTSY PDSVKGRFTI SRDIAKNNLY
LQMSSLKSED TAMYYCTRDE SDRPFPYWGQ GTLVTVSS

The amino acid sequence of the VL Domain of EphA2 mAb 3 (SEQ ID NO:246) is shown below (CDR_{L} residues are shown underlined):

DIVLTQSHRS MSTSVGDRVN ITCKASQDVT TAVAWYQQKP
GQSPKLLIFW ASTRHAGVPD RFTGSGSGTD FTLTISSVQA
GDLALYYCQQ HYSTPYTFGG GTKLEIK

The present application specifically includes and encompasses EphA2 binding molecules (e.g., EphA2 x CD3 bispecific binding molecules) that are capable of binding to EphA2, and particularly such binding molecules that comprise the VL and/or VH Domain, and/or 1, 2 or all 3 of the CDR_{L}_{^{S}} of the VL Region and/or 1, 2 or all 3 of the CDR_{H}5 of the VH Domain of anti-EphA2 monoclonal antibodies EphA2 mAb 1, EphA2 mAb 2 and EphA2 mAb 3.

### 5. Antibodies That Bind gpA33

The 43 kD transmembrane glycoprotein A33 (gpA33) is expressed in >95% of all colorectal carcinomas (Heath, J. K. et al. (1997) “The Human A33 Antigen Is A Transmembrane Glycoprotein And A Novel Member Of The Immunoglobulin Superfamily,” Proc. Natl. Acad. Sci. (U.S.A.) 94(2):469-474; Ritter, G. et al. (1997) “Characterization Of Posttranslational Modifications Of Human A33 Antigen, A Novel Palmitoylated Surface Glycoprotein Of Human Gastrointestinal Epithelium,” Biochem. Biophys. Res. Commun. 236(3):682-686; Wong, N. A. et al. (2006) “EpCAM and gpA33 Are Markers Of Barrett's Metaplasia,” J. Clin. Pathol. 59(3):260-263). An exemplary antibody that binds to human gpA33 is “gpA33 mAb 1.”

The amino acid sequence of the VH Domain of gpA33 mAb 1 (SEQ ID NO:247) is shown below (CDR_{H} residues are shown underlined):

QVQLVQSGAE VKKPGASVKV SCKASGYTFT GSWMNWVRQA
PGQGLEWICR IYPGDGETNY NGKFKDRVTI TADKSTSTAY
MELSSLRSED TAVYYCARIY GNNVYFDVWG QGTTVTVSS

The amino acid sequence of the VL Domain of gpA33 mAb 1 (SEQ ID NO:248) is shown below (CDR_{L} residues are shown underlined):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIK

The present application specifically includes and encompasses gpA33 binding molecules (e.g., gpA33x CD3 bispecific binding molecules) that are capable of binding to gpA33, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of anti-gpA33 monoclonal antibodies gpA33 mAb 1, or of any of the anti-gpA33 monoclonal antibodies provided in WO 2015/026894. The present invention additionally includes and encompasses the exemplary gpA33 x CD3 bispecific binding molecules provided in WO 2015/026894.

### 6. Antibodies That Bind HER2/neu

HER2/neu is a 185 kDa receptor protein that was originally identified as the product of the transforming gene from neuroblastomas of chemically treated rats. HER2/neu has been extensively investigated because of its role in several human carcinomas and in mammalian development (Hynes et al. (1994) Biochim. Biophys. Acta 1198:165-184; Dougall et al. (1994) Oncogene 9:2109-2123; Lee et al. (1995) Nature 378:394-398). Exemplary antibodies that bind human HER2/neu include “Margetuximab,” “Trastuzumab” and “Pertuzumab.” Margetuximab (also known as MGAH22; CAS Reg No. 1350624-75-7) is an Fc-optimized monoclonal antibody that binds to HER2/neu and mediates enhanced ADCC activity. Trastuzumab (also known as rhuMAB4D5, and marketed as HERCEPTIN®; CAS Reg No 180288-69-1; see, U.S. Pat. No. 5,821,337) is the humanized version of antibody 4D5, having IgG1/kappa constant regions. Pertuzumab (also known as rhuMAB2C4, and marketed as PERJETA™; CAS Reg No 380610-27-5; see for example, WO2001/000245) is a humanized version of antibody 2C4 having IgG1/kappa constant regions.

The present application specifically includes and encompasses Her2/Neu binding molecule (e.g., Her2/Neu x CD3 bispecific binding molecules) that are capable of binding to Her2/Neu, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-Her2/Neu monoclonal antibodies Margetuximab, Trastuzumab or Pertuzumab.

### (a) Margetuximab

The amino acid sequence of the VH Domain of Margetuximab is (SEQ ID NO:249) (CDR_{H} residues are shown underlined):

QVQLQQSGPE LVKPGASLKL SCTASGFNIK DTYIHWVKQR
PEQGLEWIGR IYPTNGYTRY DPKFQDKATI IADTSSNTAY
LQVSRLTSED TAVYYCSRWG GDGFYANDYW GQGASVTVSS

The amino acid sequence of the VL Domain of Margetuximab is (SEQ ID NO:250) (CDR_{L} residues are shown underlined):

DIVMTQSHKF MSTSVGDRVS ITCKASQDVN TAVAWYQQKP
GHSPKLLIYS ASFRYTGVPD RFTGSRSGTD FTFTISSVQA
EDLAVYYCQQ HYTTPPTFGG GTKVEIK

The amino acid sequences of the complete Heavy and Light Chains of Margetuximab are known in the art (see., e.g., WHO Drug Information, 2014, Recommended INN: List 71, 28(1):93-94).

### (b) Trastuzumab

The amino acid sequence of the VH Domain of Trastuzumab is (SEQ ID NO:251) (CDR_{H} residues are shown underlined):

EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS

The amino acid sequence of the VL Domain of Trastuzumab is (SEQ ID NO:252) (CDR_{L} residues are shown underlined):

DIQMTQSPSS LSASVGDRVT ITCRASQDVN TAVAWYQQKP
GKAPKLLIYS ASFLYSGVPS RFSGSRSGTD FTLTISSLQP
EDFATYYCQQ HYTTPPTFGQ GTKVEIK

### (c) Pertuzumab

The amino acid sequence of the VH Domain of Pertuzumab is (SEQ ID NO:253) (CDR_{H} residues are shown underlined):

EVQLVESGGG LVQPGGSLRL SCAASGFTFT DYTMDWVRQA
PGKGLEWVAD VNPNSGGSIY NQRFKGRFTL SVDRSKNTLY
LQMNSLRAED TAVYYCARNL GPSFYFDYWG QGTLVTVSS

The amino acid sequence of the VL Domain of Pertuzumab is (SEQ ID NO:254)

(CDR_{L} residues are shown underlined):

DIQMIQSPSS LSASVGDRVT ITCKASQDVS IGVAWYQQKP
GKAPKLLIYS ASYRYTGVPS RFSGSGSGTD FTLTISSLQP
EDFATYYCQQ YYIYPYTFGQ STKVEIK

### (d) Other Anti-HER2/neu Antibodies

In addition to the above-identified preferred anti-HER2/neu Binding Molecules, the invention contemplates Her2/Neu binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of any of the following anti-Her-2 Binding Molecules: 1.44.1; 1.140; 1.43; 1.14.1; 1.100.1; 1.96; 1.18.1; 1.20; 1.39; 1.24; and 1.71.3 (U.S. Pat. Nos. 8,350,011; 8,858,942; and PCT Patent Publication WO 2008/019290); F5 and C1 (U.S. Pat. Nos. 7,892,554; 8,173,424; 8,974,792; and PCT Patent Publication WO 99/55367); and also the anti-Her-2 Binding Molecules of US Patent Publication US2013017114 and PCT Patent Publications WO2011/147986 and WO 2012/143524). The present invention additionally includes and encompasses the exemplary Her2/Neu x CD3 bispecific binding molecules provided in WO 2012/143524.

### 7. Antibodies that Bind VEGF

VEGF-A is a chemical signal that stimulates angiogenesis in a variety of diseases, especially in certain metastatic cancers such as metastatic colon cancer, and in certain lung cancers, renal cancers, ovarian cancers, and glioblastoma multiforme of the brain. An exemplary antibody that binds to human VEGF-A is “Bevacizumab” (Avastin®). Bevacizumab is a recombinant humanized IgG1 monoclonal antibody (Midgley, R. et al. (2005) “Bevacizumab—Current Status And Future Directions,” Ann. Oncol. 16(7):999-1004; Hall, R. D. et al. (2015) “Angiogenesis Inhibition As A Therapeutic Strategy In Non-Small Cell Lung Cancer (NSCLC),” Transl. Lung Cancer Res. 4(5):515-523; Narita, Y. (2015) “Bevacizumab For Glioblastoma,” Ther. Clin. Risk Manag. 11:1759-1765).

The amino acid sequence of the VH Domain of Bevacizumab (SEQ ID NO:255) is shown below (CDR_{H} residues are shown underlined):

EVQLVESGGG LVQPGGSLRL SCAASGYTFT NYGMNWVRQA
PGKGLEWVGW INTYTGEPTY AADFKRRFTF SLDTSKSTAY
LQMNSLRAED TAVYYCAKYP HYYGSSHWYF DVWGQGTLVT VSS

The amino acid sequence of the VL Domain of Bevacizumab (SEQ ID NO:256) is shown below (CDR_{L} residues are shown underlined):

DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKR

The present application specifically includes and encompasses VEGF binding molecules (e.g., VEGF x CD3 bispecific binding molecules) that are capable of binding to VEGF, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-VEGF monoclonal antibody Bevacizumab.

### 8. Antibodies that Bind 5T4

The oncofetal protein, 5T4, is a tumor-associated protein displayed on the cell membrane of many carcinomas, including kidney, colon, prostate, lung, carcinoma and in acute lymphoblastic leukemia (see, Boghaert, E. R. et al. (2008) “The Oncofetal Protein, 5T4, Is A Suitable Target For Antibody-Guided Anti-Cancer Chemotherapy With Calicheamicin,” Int. J. Oncol. 32(1):221-234; Eisen, T. et al. (2014) “Naptumomab Estafenatox: Targeted Immunotherapy with a Novel Immunotoxin,” Curr. Oncol. Rep. 16:370, pp. 1-6). Exemplary antibodies that bind to human 5T4 include “5T4 mAb 1” and “5T4 mAb 2.”

### (a) 5T4 mAb 1

The amino acid sequence of the VH Domain of 5T4 mAb 1 (SEQ ID NO:257) is shown below (CDR residues are shown underlined):

QVQLVQSGAE VKKPGASVKV SCKASGYTFT SFWMHWVRQA
PGQGLEWMGR IDPNRGGTEY NEKAKSRVTM TADKSTSTAY
MELSSLRSED TAVYYCAGGN PYYPMDYWGQ GTTVTVSS

The amino acid sequence of the VL Domain of an exemplary 5T4 mAb 1 (SEQ ID NO:258) is shown below (CDR residues are shown underlined):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIK

### (b) 5T4 mAb 2

The amino acid sequence of the VH Domain of 5T4 mAb 2 (SEQ ID NO:259) is shown below (CDR residues are shown underlined):

QVQLQQPGAE LVKPGASVKM SCKASGYTFT SYWITWVKQR
PGQGLEWIGD IYPGSGRANY NEKFKSKATL TVDTSSSTAY
MQLSSLTSED SAVYNCARYG PLFTTVVDPN SYAMDYWGQG
TSVTVSS

The amino acid sequence of the VL Domain of 5T4 mAb 2 (SEQ ID NO:260) is shown below (CDR residues are shown underlined):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IK

The present application specifically includes and encompasses 5T4 binding molecules (e.g., 5T4 x CD3 bispecific binding molecules) that are capable of binding to 5T4 that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-5T4 monoclonal antibodies 5T4 mAb 1 or 5T4 mAb 2, or of any of the anti-5T4 antibodies provided in WO 2013/041687 or WO 2015/184203. The present invention additional includes and encompasses the exemplary 5T4 x CD3 bispecific binding molecules provided in WO 2015/184203.

The present application additionally specifically includes and encompasses 5T4 x CD3 x CD8 trispecific binding molecules that are capable of binding to 5T4, to CD3 and to CD8, and particularly such trispecific binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-5T4 monoclonal antibodies 5T4 mAb 1 or 5T4 mAb 2 or of any of the anti-5T4 monoclonal antibodies provided in WO 2015/184203, and/or the VL and/or VH Domain, and/or 1, 2 or all 3 of the CDR_{L}_{^{S}} of the VL Region and/or 1, 2 or all 3 of the CDR_{H}_{^{S}} of the VH Domain of any of the anti-CD8 monoclonal antibodies provided in WO 2015/184203. The present invention additional includes and encompasses the exemplary 5T4 x CD3 x CD8 trispecific molecules provided in WO 2015/184203.

### 9. Antibodies that Bind IL13Rα2

Interleukin-13 Receptor a2 (IL13Rα2) is overexpressed in a variety of cancers, including glioblastoma, colorectal cancer, cervical cancer, pancreatic cencer, multiple melanoma, osteosarcoma, leukemia, lymphoma, prostate cancer and lung cancer (PCT Pubmication No. WO 2008/146911; Brown, C. E. et al. (2013) “Glioma IL13Rα2 Is Associated With Mesenchymal Signature Gene Expression And Poor Patient Prognosis,” PLoS One. 18; 8(10):e77769; Barderas, R. et al. (2012) “High Expression Of IL-13 Receptor A2 In Colorectal Cancer Is Associated With Invasion, Liver Metastasis, And Poor Prognosis,” Cancer Res. 72(11):2780-2790; Kasaian, M. T. et al. (2011) “IL-13 Antibodies Influence IL-13 Clearance In Humans By Modulating Scavenger Activity Of IL-13Rα2,” J. Immunol. 187(1):561-569; Bozinov, O. et al. (2010) “Decreasing Expression Of The Interleukin-13 Receptor IL-13Ralpha2 In Treated Recurrent Malignant Gliomas,” Neurol. Med. Chir. (Tokyo) 50(8):617-621; Fujisawa, T. et al. (2009) “A Novel Role Of Interleukin-13 Receptor Alpha2 In Pancreatic Cancer Invasion And Metastasis,” Cancer Res. 69(22):8678-8685). Antibodies that immunospecifically bind to IL13Rα2 are commercially available and have been described in the art (Abnova Corporation, Biorbyt, LifeSpan BioSciences, United States Biologicals; see also PCT Publication No. WO 2008/146911). Exemplary antibodies that bind to human IL13Rα2 include “hu08” (see, e.g., PCT Publication No. WO 2014/072888).

The amino acid sequence of the VH Domain of hu08 (SEQ ID NO:261) is shown below (CDR residues are shown underlined):

EVQLVESGGG LVQPGGSLRL SCAASGFTFS RNGMSWVRQA
PGKGLEWVAT VSSGGSYIYY ADSVKGRFTI SRDNAKNSLY
LQMNSLRAED TAVYYCARQG TTALATRFFD VWGQGTLVTV
SS

The amino acid sequence of the VL Domain of hu08 (SEQ ID NO:262) is shown below (CDR residues are shown underlined):

DIQMTQSPSS LSASVGDRVT ITCKASQDVG TAVAWYQQKP
GKAPKLLIYS ASYRSTGVPS RFSGSGSGTD FTLTISSLQP
EDFATYYCQH HYSAPWTFGG GTKVEIK

The present application specifically includes and encompasses IL13Rα2 binding molecules (e.g., IL13Rα2 x CD3 bispecific binding molecules) that are capable of binding to IL13Rα2, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-IL13Rα2 monoclonal antibody hu08.

### 10. Antibodies that Bind CD123

CD123 (interleukin 3 receptor alpha, IL-3Ra) is a 40 kDa molecule and is part of the interleukin 3 receptor complex (Stomski, F. C. et al. (1996) “Human Interleukin-3 (IL-3) Induces Disulfide-Linked IL-3 Receptor Alpha-And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 has been reported to be overexpressed on malignant cells in a wide range of hematologic malignancies including acute myeloid leukemia (AML) and myelodysplastic syndrome (MDS) (Muñoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401).

An exemplary antibody that binds to human CD123, and that may be employed in the present invention, is “CD123 mAb 1” (see, e.g., PCT Patent Publication WO 2015/026892).

The amino acid sequence of the VH Domain of CD123 mAb 1 (SEQ ID NO:263) is shown below (CDR_{H} residues are shown underlined):

EVQLVQSGAE LKKPGASVKV SCKASGYTFT DYYMKWVRQA
PGQGLEWIGD IIPSNGATFY NQKFKGRVTI TVDKSTSTAY
MELSSLRSED TAVYYCARSH LLRASWFAYW GQGTLVTVSS

The amino acid sequence of the VL Domain of CD123 mAb 1 (SEQ ID NO:264) is shown below (CDR_{L} residues are shown underlined):

DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIK

The present application specifically includes and encompasses CD123 binding molecules (e.g., CD123 x CD3 bispecific binding molecules) that are capable of binding to CD123, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-CD123 monoclonal antibody CD123 mAb 1, and also any of the anti-CD123 antibodies disclosed in US 2017/081424 and WO 2016/036937 The present invention additionally includes and encompasses exemplary CD123 x CD3 bispecific binding molecules, including: flotetuzumab (aka MGD007; CAS Registry No. 1664355-28-5), JNJ-63709178 (Johnson & Johnson, also see, WO 2016/036937) and XmAb14045 (Xencor, also see, US 2017/081424).

### 11. Antibodies that Bind CD19

CD19 (B lymphocyte surface antigen B4, Genbank accession number M28170) is a component of the B cell-receptor (BCR) complex, and is a positive regulator of B cell signaling that modulates the threshold for B cell activation and humoral immunity. CD19 is one of the most ubiquitously expressed antigens in the B cell lineage and is expressed on >95% of B cell malignancies, including acute lymphoblastic leukemia (ALL), chronic lymphocytic leukemia (CLL), and non-Hodgkin's Lymphoma (NHL). Notably, CD19 expression is maintained on B cell lymphomas that become resistant to anti-CD20 therapy (Davis et al. (1999) “Therapy of B-cell Lymphoma With Anti-CD20 Antibodies Can Result In The Loss Of CD20 Antigen Expression.” Clin Cancer Res, 5:611-615, 1999). CD19 has also been suggested as a target to treat autoimmune diseases (Tedder (2009) “CD19: A Promising B Cell Target For Rheumatoid Arthritis,” Nat. Rev. Rheumatol. 5:572-577).

An exemplary antibody that binds to human CD19, and that may be employed in the present invention, is the anti-CD19 antibody disclosed in WO 2016/048938 (referred to herein as “CD19 mAb 1”).

The amino acid sequence of the VH Domain of CD19 mAb 1 (SEQ ID NO:265) is shown below (CDR_{H} residues are shown underlined):

QVTLRESGPA LVKPTQTLTL TCTFSGFSLS TSGMGVGWIR
QPPGKALEWL AHIWWDDDKR YNPALKSRLT ISKDTSKNQV
FLTMTNMDPV DTATYYCARM ELWSYYFDYW GQGTTVTVSS

The amino acid sequence of the VL Domain of CD19 mAb 1 (SEQ ID NO:266) is shown below (CDR_{L} residues are shown underlined):

ENVLTQSPAT LSVTPGEKAT ITCRASQSVS YMHWYQQKPG
QAPRLLIYDA SNRASGVPSR FSGSGSGTDH TLTISSLEAE
DAATYYCFQG SVYPFTFGQG TKLEIK

The present application specifically includes and encompasses CD19 binding molecules (e.g., CD19 x CD3 bispecific binding molecules) that are capable of binding to CD19, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-CD19 monoclonal antibody CD19 mAb 1, or any of the anti-CD19 antibodies disclosed in U.S. Pat. No. 7,112,324. The present invention specifically includes and encompasses exemplary CD19 x CD3 bispecific binding molecules that may be employed in the present invention, including: blinatumomab (BLINCYTO®; amino acid sequence found in WHO Drug Information, 2009, Recommended INN: List 62, 23(3):240-241) and duvortuxizumab (aka MGD011; amino acid sequence found in WHO Drug Information, 2016, Proposed INN: List 116, 30(4):627-629).

### E. Exemplary Pathogen-Associated Antigens

As used herein, the term “Pathogen Antigen” denotes an antigen that is characteristically expressed on the surface of a pathogen-infected cell, and that may thus be treated with an Antibody-Based Molecule or an Immunomodulatory Molecule. Examples of Pathogen Antigens include, but are not limited to antigens expressed on the surface of a cell infected with: a Herpes Simplex Virus (e.g., infected cell protein (ICP)47, gD, etc.), a varicella-zoster virus, a Kaposi's sarcoma-associated herpesvirus, an Epstein-Barr Virus (e.g., LMP-1, LMP-2A, LMP-2B, etc.), a Cytomegalovirus (e.g., UL11, etc.), Human Immunodeficiency Virus (e.g., env proteins gp160, gp120, gp41, etc.), a Human Papillomavirus (e.g., E6, E7, etc.), a human T-cell leukemia virus (e.g., env proteins gp64, gp46, gp21, etc.), Hepatitis A Virus, Hepatitis B Virus, Hepatitis C Virus, Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (fumigatus, niger, etc.), Blastomyces dermatitidis, Candida (albicans, krusei, glabrata, tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia Zambia, trichinella, Dientamoeba Fragilis, Trypanosoma brucei, Trypanosoma cruzi, and Leishmania donovani). Such antibodies are available commercially from a wide number of sources, or can be obtained by immunizing mice or other animals (including for the production of monoclonal antibodies) with such antigens.

### F. Exemplary Antibodies Capable of Binding a Pathogen-Associated Antigen

Exemplary antibodies, whose VH and VL Domains may be used to construct molecules capable of binding a Pathogen Antigen arrayed on the surface of a pathogen-infected cell are antibodies are provided below, additional antibodies are known in the art.

The env protein of HIV is an exemplary Pathogen-Associated Antigen, and antibodies that bind the env protein of HIV are exemplary of antibodies capable of binding a Pathogen-Associated Antigen.

The initial step in HIV-1 infection occurs with the binding of cell surface CD4 to trimeric HIV-1 envelope glycoproteins (env), a heterodimer of a transmembrane glycoprotein (gp41) and a surface glycoprotein (gp120). The gp120 and gp41 glycoproteins are initially synthesized as a single gp160 polypeptide that is subsequently cleaved to generate the non-covalently associated gp120/gp41 complex. The ectodomain of env is a heterodimer with mass of approximately 140 kDa, composed of the entire gp120 component, and approximately 20 kDa of gp41 (Harris, A. et al. (2011) “Trimeric HIV-1 Glycoprotein Gp140 Immunogens And Native HIV-1 Envelope Glycoproteins Display The Same Closed And Open Quaternary Molecular Architectures,” Proc. Natl. Acad. Sci. (U.S.A.) 108(28):11440-11445). Antibodies that that immunospecifically bind to env proteins are commercially available and have been described in the art (see, e.g., GenBank Accession No. AFQ31503; Buchacher, A. et al. (1994) “Generation Of Human Monoclonal Antibodies Against HIV-1 Proteins; Electrofusion And Epstein-Barr Virus Transformation For Peripheral Blood Lymphocyte Immortalization,” AIDS Res. Hum. Retroviruses 10(4):359-369; Shen, R. (2010) “GP41-Specific Antibody Blocks Cell-Free HIV Type 1 Transcytosis Through Human Rectal Mucosa And Model Colonic Epithelium,” J. Immunol. 184(7):3648-3655; WO 2012/162068; and WO 2016/054101). Exemplary antibodies that bind to HIV env include “7B2” (GenBank Accession No. AFQ31503) and “A32” (PCT Publication No. WO 2014/159940).

The amino acid sequence of the VH Domain of 7B2 (SEQ ID NO:267) is shown below (CDR residues are shown underlined):

QVQLVQSGGG VFKPGGSLRL SCEASGFTFT EYYMTWVRQA
PGKGLEWLAY ISKNGEYSKY SPSSNGRFTI SRDNAKNSVF
LQLDRLSADD TAVYYCARAD GLTYFSELLQ YIFDLWGQGA
RVTVSS

The amino acid sequence of the VL Domain of 7B2 (SEQ ID NO:268) is shown below (CDR residues are shown underlined):

DIVMTQSPDS LAVSPGERAT IHCKSSQTLL YSSNNRHSIA
WYQQRPGQPP KLLLYWASMR LSGVPDRFSG SGSGTDFTLT
INNLQAEDVA IYYCHQYSSH PPTFGHGTRV EIK

The amino acid sequence of the VH Domain of A32 (SEQ ID NO:269) is shown below (CDR residues are shown underlined):

QVQLQESGPG LVKPSQTLSL SCTVSGGSSS SGAHYWSWIR
QYPGKGLEWI GYIHYSGNTY YNPSLKSRIT ISQHTSENQF
SLKLNSVTVA DTAVYYCARG TRLRTLRNAF DIWGQGTLVT
VSS

The amino acid sequence of the VL Domain of A32 (SEQ ID NO:270) is shown below (CDR residues are shown underlined):

QSALTQPPSA SGSPGQSVTI SCTGTSSDVG GYNYVSWYQH
HPGKAPKLII SEVNNRPSGV PDRFSGSKSG NTASLTVSGL
QAEDEAEYYC SSYTDIHNFV FGGGTKLTVL

The present application specifically includes and encompasses HIV binding molecules (e.g., HIV x CD3 bispecific binding molecules) that are capable of binding to HIV, and particularly such binding molecules that comprise the VL and/or VH Domain, and/or 1, 2 or all 3 of the CDR_{L}_{^{S}} of the VL Region and/or 1, 2 or all 3 of the CDR_{H}_{^{S}} of the VH Domain of the anti-HIV monoclonal antibodies 7B2, A32, and also any of the anti-HIV antibodies disclosed in WO 2016/054101, WO 2017/011413, WO 2017/011414. The present invention specifically includes and encompasses the exemplary HIV x CD3 bispecific binding molecules provided in WO 2014/159940, WO 2015/184203, WO 2017/011413, and WO 2017/011414.

The present application additionally specifically includes and encompasses HIV x CD3 x CD8 tri specific binding molecules that are capable of binding to HIV, to CD3 and to CD8, and particularly such trispecific binding molecules that comprise the VL and/or VH Domain, and/or 1, 2 or all 3 of the CDR_{L}_{^{S}} of the VL Region and/or 1, 2 or all 3 of the CDR_{L}_{^{S}} of the VH Domain of the anti-HIV monoclonal antibodies 7B2 or A32 or of any of the anti-HIV monoclonal antibodies provided in WO 2015/184203, WO 2016/054101, WO 2017/011413, WO 2017/011414, and/or the VL and/or VH Domain, and/or 1, 2 or all 3 of the CDR_{L}_{^{S}} of the VL Region and/or 1, 2 or all 3 of the CDR_{H}_{^{S}} of the VH Domain of any of the anti-CD8 monoclonal antibodies provided in WO 2015/184203. The present invention specifically includes and encompasses the exemplary HIV x CD3 x CD8 trispecific binding molecules provided in WO 2015/184203, WO 2017/011413, and WO 2017/011414.

### G. Exemplary Binding Molecules of the Present Invention

As discussed below, the present invention is illustrated using a combination therapy of two administered molecules: a molecule capable of binding PD-1 (e.g., hPD-1 mAb7 (1.2) IgG4 (P), DART-1 or DART-2, described above), and a molecule capable of mediating the redirected killing of a tumor cell (e.g., “DART-A,” or “DART-B,” described below).

DART-A is a bispecific diabody capable of binding the CD3 cell surface molecule of an effector cell and the B7-H3 Cancer Antigen. It is an Fc Region-containing diabody composed of three polypeptide chains having one binding site for B7-H3, one binding site for B7-H3, Knob and Hole bearing IgG1 Fc Regions, and E/K-coil Heterodimer-Promoting Domains (see, e.g., FIG. 4A).

The first polypeptide chain of DART-A comprises, in the N-terminal to C-terminal direction, an N-terminus, a VL Domain of a monoclonal antibody capable of binding B7-H3 (hB7-H3 mAb 2 VL2) (SEQ ID NO:226), an intervening linker peptide (Linker 1; GGGSGGGG (SEQ ID NO:14)), a VH Domain of a monoclonal antibody capable of binding CD3 (CD3 mAb 1 VH) (SEQ ID NO:192), an intervening linker peptide (Linker 2; GGCGGG (SEQ ID NO:15)), a Heterodimer-Promoting (E-coil) Domain (EVAALEK-EVAALEK-EVAALEK-EVAALEK (SEQ ID NO:27)), an intervening linker peptide (Spacer-Linker 3; GGGDKTHTCPPCP (SEQ ID NO:39)), a “knob-bearing” Fc Domain (SEQ ID NO:42), and a C-terminus. Thus, the first polypeptide chain of DART-A is composed of: SEQ ID NO:226-SEQ ID NO:14-SEQ ID NO:192-SEQ ID NO:15-SEQ ID NO:27-SEQ ID NO:39-SEQ ID NO:42. The amino acid sequence of the first polypeptide chain of DART-A is (SEQ ID NO:271):

DIQLTQSPSF LSASVGDRVT ITCKASQNVD TNVAWYQQKP
GKAPKALIYS ASYRYSGVPS RFSGSGSGTD FTLTISSLQP
EDFATYYCQQ YNNYPFTFGQ GTKLEIKGGG SGGGGEVQLV
ESGGGLVQPG GSLRLSCAAS GFTFSTYAMN WVRQAPGKGL
EWVGRIRSKY NNYATYYADS VKDRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

The second polypeptide chain of DART-A comprises, in the N-terminal to C-terminal direction, an N-terminus, a VL Domain of a monoclonal antibody capable of binding CD3 (CD3 mAb 1 VL) (SEQ ID NO:193), an intervening linker peptide (Linker 1; GGGSGGGG (SEQ ID NO:14)), a VH Domain of a monoclonal antibody capable of binding B7-H3 (hB7-H3 mAb 2 VH2) (SEQ ID NO:222), an intervening linker peptide (Linker 2; GGCGGG (SEQ ID NO:15)), a Heterodimer-Promoting (K-coil) Domain (KVAALKE-KVAALKE-KVAALKE-KVAALKE (SEQ ID NO:28)), and a C-terminus. Thus, the second polypeptide of DART-A is composed of: SEQ ID NO:193-SEQ ID NO:14-SEQ ID NO:222-SEQ ID NO:15-SEQ ID NO:28. The amino acid sequence of the second polypeptide chain of DART-A is (SEQ ID NO:272):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVESGGGLV QPGGSLRLSC AASGFTFSSF GMHWVRQAPG
KGLEWVAYIS SDSSAIYYAD TVKGRFTISR DNAKNSLYLQ
MNSLRDEDTA VYYCGRGREN IYYGSRLDYW GQGTTVTVSS
GGCGGGKVAA LKEKVAALKE KVAALKEKVA ALKE

The third polypeptide chain of DART-A comprises, in the N-terminal to C-terminal direction, an N-terminus, a peptide (Linker 3; DKTHTCPPCP (SEQ ID NO:38)), a “hole-bearing” Fc Domain (SEQ ID NO:43), and a C-terminus. Thus, the third polypeptide of DART-A is composed of: SEQ ID NO:38-SEQ ID NO:43. The amino acid sequence of the third polypeptide of DART-A is (SEQ ID NO:273):

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

Another exemplary molecule capable of mediating the redirected killing of a tumor cell is DART-B. DART-B is a bispecific diabody capable of binding the CD3 cell surface molecule of an effector cell and the IL13Rα2 Cancer Antigen. DART-B is composed of three polypeptide chain and has the same general structure as DART-A.

Additional, exemplary molecules capable of mediating the redirected killing of a tumor cell which may be used in the methods of the present invention include bispecific molecules capable of binding: CD19 and CD3 (see, e.g., U.S. Pat. No. 7,235,641 and WO 2016/048938); CD123 and CD3 (see, e.g., Kuo, S. R. et al., (2012) “Engineering a CD123xCD3 bispecific scFv immunofusion for the treatment of leukemia and elimination of leukemia stem cells,” Protein Eng Des Sel. 25:561-9; PCT Publication WO 2015/026892); gpA33 and CD3 (e.g., WO 2015/026894); CEA and CD3 (e.g., WO 2013/012414); B7-113 and CD3 (e.g., WO 2017/030926); HER2 and CD3 (e.g., WO 2012/143524); 5T4 and CD3 (e.g., WO 2015/184203 and WO 2013/041687), and trispecific molecules (see, e.g., WO 2015/184203; and WO 2015/184207).

### VI. Methods of Production

The molecules of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

Antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol. Methods 231:147-157). Suitable methods for making derivatives of antibodies, e.g., humanized, single-chain, etc. are known in the art, and have been described above. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

Vectors containing polynucleotides of interest (e.g., polynucleotides encoding the polypeptide chains of the binding molecules of the present invention) can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cell capable of overexpressing heterologous DNAs can be used for the purpose of expressing a polypeptide or protein of interest. Non-limiting examples of suitable mammalian host cells include but are not limited to COS, HeLa, and CHO cells.

The invention includes polypeptides comprising an amino acid sequence of a binding molecule of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

The invention includes variants of the disclosed binding molecules, including functionally equivalent polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues that can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention encompasses fusion proteins comprising one or more of the VH and/or VL Domains of an antibody that binds to PD-1 (or a natural ligand of PD-1) or of an antibody that binds to a cell surface molecule of an effector cell or of an antibody that binds to a Disease Antigen (e.g., a Cancer Antigen or a Pathogen-Associated Antigen). In one embodiment, a fusion polypeptide is provided that comprises a Light Chain, a Heavy Chain or both a Light and Heavy Chain. In another embodiment, the fusion polypeptide contains a heterologous immunoglobulin constant region. In another embodiment, the fusion polypeptide contains a VH and a VL Domain of an antibody produced from a publicly-deposited hybridoma. For purposes of this invention, an antibody fusion protein contains one or more polypeptide domains that specifically bind PD-1 (or a natural ligand of PD-1) or to a cell surface molecule of an effector cell, and which contains another amino acid sequence to which it is not attached in the native molecule, for example, a heterologous sequence or a homologous sequence from another region.

The present invention particularly encompasses such binding molecules (e.g., antibodies, diabodies, trivalent binding molecules, etc.) conjugated to a diagnostic or therapeutic moiety. For diagnostic purposes, the binding molecules of the invention may be coupled to a detectable substance. Such binding molecules are useful for monitoring and/or prognosing the development or progression of a disease as part of a clinical testing procedure, such as determining the efficacy of a particular therapy. Examples of detectable substances include various enzymes (e.g., horseradish peroxidase, beta-galactosidase, etc.), prosthetic groups (e.g., avidin/biotin), fluorescent materials (e.g., umbelliferone, fluorescein, or phycoerythrin), luminescent materials (e.g., luminol), bioluminescent materials (e.g., luciferase or aequorin), radioactive materials (e.g., carbon-14, manganese-54, strontium-85 or zinc-65), positron emitting metals, and nonradioactive paramagnetic metal ions. The detectable substance may be coupled or conjugated either directly to thebinding molecule or indirectly, through an intermediate (e.g., a linker) using techniques known in the art.

For therapeutic purposes, the binding molecules of the invention may be conjugated to a therapeutic moiety such as a cytotoxin, (e.g., a cytostatic or cytocidal agent), a therapeutic agent or a radioactive metal ion, e.g., alpha-emitters. A cytotoxin or cytotoxic agent includes any agent that is detrimental to cells such as, for example, Pseudomonas exotoxin, Diptheria toxin, a botulinum toxin A through F, ricin abrin, saporin, and cytotoxic fragments of such agents. A therapeutic agent includes any agent having a therapeutic effect to prophylactically or therapeutically treat a disorder. Such therapeutic agents may be may be chemical therapeutic agents, protein or polypeptide therapeutic agents, and include therapeutic agents that possess a desired biological activity and/or modify a given biological response. Examples of therapeutic agents include alkylating agents, angiogenesis inhibitors, anti-mitotic agents, hormone therapy agents, and antibodies useful for the treatment of cell proliferative disorders. The therapeutic moiety may be coupled or conjugated either directly to the binding molecule or indirectly, through an intermediate (e.g., a linker) using techniques known in the art.

### VII. Uses of the Binding Molecules of the Present Invention

As discussed above molecules capable of binding PD-1 or a natural ligand of PD-1 and molecules capable of mediating the redirected cell killing of a target cell (i.e., a cancer cell, or a pathogen-infected cell) may be used for therapeutic purposes, for example in subjects with cancer or an infection. Thus, binding molecules of the present invention have the ability to treat any disease or condition associated with or characterized by the expression of a Disease Antigen, particularly a Cancer Antigen or a Pathogen-Associated Antigen, on the surface of such target cell. Thus, without limitation, the binding molecules of the present invention may be employed in the treatment of cancer, particularly a cancer characterized by the expression of a Cancer Antigen. The binding molecules of the present invention may be employed in the treatment of infection, particularly an infection characterized by the expression of a Pathogen-Associated Antigen.

In particular, the present invention encompasses such methods wherein the molecule capable of binding PD-1 or a natural ligand of PD-1 comprises an epitope-binding domain of an antibody that is capable of binding PD-1 or an epitope-binding domain of an antibody that is capable of binding a natural ligand of PD-1 and wherein the molecule capable of mediating redirected killing comprises an eptiope-binding domain capable of binding a cell surface molecule (e.g., CD2, CD3, CD8, CD16, TCR, NKG2D, etc.) of an effector cell (e.g., a helper T Cell, a cytotoxic T Cell, a Natural Killer (NK) cell, a plasma cell (an antibody-secreting B cell), a macrophage and a granulocyte) and also comprises an epitope-binding domain capable of binding a Disease Antigen (in particular a Cancer Antigen or a Pathogen-Associated Antigen) on the surface of a target cell so as to mediate the redirected killing of the target cell (for example, by mediating redirected cell killing (e.g., redirected T-cell cytotoxicity)).

In a specific embodiment, the molecule capable of binding PD-1 or a natural ligand of PD-1 is an antibody and the molecule capable of mediating redirected cell killing is a diabody. In another specific embodiment, the molecule capable of binding PD-1 or a natural ligand of PD-1 is an antibody and the molecule capable of mediating redirected cell killing is a trivalent binding molecule.

In a specific embodiment, the molecule capable of binding PD-1 or a natural ligand of PD-1 is a diabody and the molecule capable of mediating redirected cell killing is a diabody. In another specific embodiment, the molecule capable of binding PD-1 or a natural ligand of PD-1 is an diabody and the molecule capable of mediating redirected cell killing is a trivalent binding molecule.

In one embodiment, the molecule capable of binding PD-1 or a natural ligand of PD-1, and the molecule capable of mediating redirected cell killing are administered concurrently. As used herein, such “concurrent” administration is intended to denote:
(A) the administration of a single pharmaceutical composition that contains both a molecule capable of binding PD-1 or a natural ligand of PD-1, and a molecule capable of mediating redirected cell killing; or
(B) the separate administration of two or more pharmaceutical compositions, one composition of which contains the molecule capable of binding PD-1 or a natural ligand of PD-1, and another composition of which contains a molecule capable of mediating redirected cell killing, wherein the compositions are administered within a 48 hour period.

In a second embodiment, the molecules are administered “sequentially” (e.g., a molecule capable of binding PD-1 or a natural ligand of PD-1 is administered and, at a later time, a molecule capable of mediating redirected cell killing is administered, or vice versa). In such sequential administration, the second administered composition is administered at least 48 hours, or more after the administration of the first administered composition.

“Providing a therapy” or “treating” refers to any administration of a composition that is associated with any indicia of beneficial or desired result, including, without limitation, any clinical result such as decreasing symptoms resulting from the disease, attenuating a symptom of infection (e.g., viral load, fever, pain, sepsis, etc.) a shrinking of the size of a tumor (in the cancer context, for example, a tumor of breast, gastric or prostate cancer), a retardation of cancer cell growth, a delaying of the onset, development or progression of metastasis, a decreasing of a symptom resulting from the disease, an increasing of the quality of life of the recipient subject, a decreasing of the dose of other medications being provided to treat a subject's disease, an enhancing of the effect of another medication such as via targeting and/or internalization, a delaying of the progression of the disease, and/or a prolonging of the survival of recipient subject.

Subjects for treatment include animals, most preferably mammalian species such as non-primate (e.g., bovine, equine, feline, canine, rodent, etc.) or a primate (e.g., monkey such as, a cynomolgus monkey, human, etc.). In a preferred embodiment, the subject is a human.

Exemplary disorders that may be treated by various embodiments of the present invention include, but are not limited to, proliferative disorders, cell proliferative disorders, and cancer (especially a cancer expressing a Cancer Antigen bound by a molecule capable of mediating redirected cell killing), pathogen-associated diseases (especially a chronic viral infection associated with expression of a Pathogen-Associated Antigen bound by a molecule capable of mediating redirected cell killing). In various embodiments, the invention encompasses methods and compositions for treatment, prevention or management of a disease or disorder in a subject, comprising administering to the subject a therapeutically effective amount a molecule capable of binding PD-1 or a natural ligand of PD-1 and a molecule capable of mediating the redirected killing of a target cell (e.g., a tumor cell, a pathogen-infected cell or a foreign cell). The combination of such molecules is particularly useful for the prevention, inhibition, reduction of growth, or regression of primary tumors, and metastasis of tumors, and for reducing pathogen load, or eliminating pathogen-infected cells. Although not intending to be bound by a particular mechanism of action, such molecules may mediate effector function against target cells, promote the activation of the immune system against target cells, cross-link cell-surface antigens and/or receptors on target cells and enhance apoptosis or negative growth regulatory signaling, or a combination thereof, resulting in clearance and/or reduction in the number of target cells.

The cancers that may be treated by molecules of the present invention, and by the methods of the present invention, include, but are not limited to: an adrenal gland cancer, including but not limited to, a pheochromocytom or an adrenocortical carcinoma; an AIDS-associated cancer; an alveolar soft part sarcoma; an astrocytic tumor; a basal cancer; a bladder cancer, including but not limited to, a transitional cell carcinoma, a squamous cell cancer, an adenocarcinoma, or a carcinosarcoma; a bone and connective tissue sarcoma, such as but not limited to, a bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, or a synovial sarcoma; a brain cancer, including, but not limited to, a glioma, astrocytoma, brain stem glioma, ependymoma, oligodendroglioma, nonglial tumor, acoustic neurinoma, craniopharyngioma, medulloblastoma, meningioma, pineocytoma, pineoblastoma, or a primary brain lymphoma; a brain and spinal cord cancer; a breast cancer, including, but not limited to, an adenocarcinoma, lobular (small cell) carcinoma, intraductal carcinoma, medullary breast cancer, mucinous breast cancer, tubular breast cancer, papillary breast cancer, Paget's disease, or an inflammatory breast cancer; a carotid body tumor; a cervical cancer, including but not limited to, a squamous cell carcinoma, or a adenocarcinoma; a cholangiocarcinoma, including but not limited to, a papillary, nodular, or diffuse cholangiocarcinoma; a chondrosarcoma; a chordoma; a chromophobe renal cell carcinoma; a clear cell carcinoma; a colon cancer; a colorectal cancer; a cutaneous benign fibrous histiocytoma; a desmoplastic small round cell tumor; an ependymoma; an eye cancer, including, but not limited to, an ocular melanoma such as iris melanoma, choroidal melanoma, and cilliary body melanoma, and retinoblastoma; an esophageal cancer, including but not limited to, a squamous cancer, adenocarcinoma, adenoid cyctic carcinoma, mucoepidermoid carcinoma, adenosquamous carcinoma, sarcoma, melanoma, plasmacytoma, verrucous carcinoma, and an oat cell (small cell) carcinoma; a Ewing's tumor; an extraskeletal myxoid chondrosarcoma; a fibrogenesis imperfecta ossium; a fibrous dysplasia of the bone; a gallbladder or bile duct cancer, including but not limited to, an adenocarcinoma; a gastric cancer; a gestational trophoblastic disease; a germ cell tumor; a head and neck cancer; a hepatocellular carcinoma; Heavy Chain disease; an islet cell tumor; a Kaposi's sarcoma; a leukemia, including, but not limited to, an acute leukemia; acute lymphocytic leukemia; an acute myelocytic leukemia, such as, but not limited to, a myeloblastic, promyelocytic, myelomonocytic, monocytic, or erythroleukemia leukemia or a myelodysplastic syndrome; a chronic leukemia, such as but not limited to, a chronic myelocytic (granulocytic) leukemia, a chronic lymphocytic leukemia, a hairy cell leukemia; a lipoma/benign lipomatous tumor; a liposarcoma/malignant lipomatous tumor; a liver cancer, including but not limited to, a hepatocellular carcinoma, or a hepatoblastoma; a lymphoma, such as but not limited to, Hodgkin's disease; non-Hodgkin's disease; a lung cancer, including but not limited to, a non-small cell lung cancer, squamous cell carcinoma (epidermoid carcinoma), adenocarcinoma, large-cell carcinoma or a small-cell lung cancer; a medulloblastoma; a melanoma; a meningioma; a benign monoclonal gammopathy; a monoclonal gammopathy of undetermined significance; a multiple endocrine neoplasia; a multiple myeloma, such as but not limited to, a smoldering multiple myeloma, nonsecretory myeloma, osteosclerotic myeloma, plasma cell leukemia, solitary plasmacytoma and extramedullary plasmacytoma; a myelodysplastic syndrome; a neuroblastoma; a neuroendocrine tumor; an oral cancer, including but not limited to, a squamous cell carcinoma; an ovarian cancer; including, but not limited to, an ovarian epithelial carcinoma, borderline tumor, germ cell tumor, and stromal tumor; a pancreatic cancer, including but not limited to, an insulinoma, gastrinoma, glucagonoma, vipoma, somatostatin-secreting tumor, or a carcinoid or islet cell tumor; a parathyroid tumor; a pediatric cancer; a penal cancer; a peripheral nerve sheath tumor; a phaeochromocytoma; a pharynx cancer, including but not limited to, a squamous cell cancer, or a verrucous cancer; a pituitary cancer, including but not limited to, Cushing's disease, a prolactin-secreting tumor, acromegaly, or a diabetes insipius tumor; a prostate cancer, including but not limited to, an adenocarcinoma, leiomyosarcoma, or rhabdomyosarcoma; polycythemia vera; a posterious uveal melanoma; a rare hematologic disorder; a renal cancer, including but not limited to, an adenocarcinoma, hypernephroma, fibrosarcoma, a renal metastatic cancer, or a transitional cell cancer (renal pelvis and/or uterer); a rhabdoid tumor; a rhabdomysarcoma; a salivary gland cancer, including but not limited to, an adenocarcinoma, mucoepidermoid carcinoma, or an adenoidcystic carcinoma; a sarcoma; a skin cancer, including but not limited to, a basal cell carcinoma, a squamous cell carcinoma and melanoma, a superficial spreading melanoma, a nodular melanoma, a lentigo malignant melanoma, or an acral lentiginous melanoma; a soft-tissue sarcoma; a squamous cell cancer; a stomach cancer, including but not limited to, an adenocarcinoma, a fungating (polypoid), ulcerating, superficial spreading, diffusely spreading, or malignant lymphoma, liposarcoma, fibrosarcoma, and carcinosarcoma; a synovial sarcoma; a testicular cancer, including but not limited to, a germinal tumor, seminoma, anaplastic, classic (typical), spermatocytic, nonseminoma, embryonal carcinoma, teratoma carcinoma, or a choriocarcinoma (yolk-sac tumor); a thymic carcinoma; a thymoma; a thyroid cancer, such as but not limited to, papillary or follicular thyroid cancer, metastatic thyroid cancer, medullary thyroid cancer or anaplastic thyroid cancer; a uterine cancer, including but not limited to, an endometrial carcinoma or a uterine sarcoma; a vaginal cancer, including but not limited to, a squamous cell carcinoma, adenocarcinoma, or melanoma; a vulvar cancer, including but not limited to, a squamous cell carcinoma, melanoma, adenocarcinoma, basal cell carcinoma, sarcoma, or Paget's disease; a Waldenström's macroglobulinemia, or Wilms' tumor. In addition, cancers include myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc.).

In particular, the binding molecules of the present invention may be used in the treatment of adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.

Pathogen-associated diseases that may be treated by the LAG-3-binding molecules of the present invention include chronic viral, bacterial, fungal and parasitic infections. Chronic infections that may be treated by the LAG-3-binding molecules of the present invention include Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (fumigatus, niger, etc.), Blastomyces dermatitidis, Candida (albicans, krusei, glabrata, tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, and Leishmania donovani).

### VIII. Pharmaceutical Compositions

The present invention encompasses compositions comprising a molecule capable of binding PD-1 or a natural ligand of PD-1, a molecule capable of mediating the redirected killing of a tumor cell, or a combination of such molecules. The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of a molecule capable of binding PD-1 or a natural ligand of PD-1, a molecule capable of mediating the redirected killing of a target cell (e.g., a cancer cell, a pathogen-infected cell, etc.), or a combination of such agents and a pharmaceutically acceptable carrier. Preferably, compositions of the invention comprise a prophylactically or therapeutically effective amount of the binding molecules of the present invention and a pharmaceutically acceptable carrier. In a preferred aspect, such compositions are substantially purified (i.e., substantially free from substances that limit its effect or produce undesired side effects).

Where more than one therapeutic agent is to be administered the agents may be formulated together in the same formulation or may be formulated into separate compositions. Accordingly, in some embodiments, the molecule capable of binding PD-1 or a natural ligand of PD-1 and the molecule capable of mediating the redirected killing of a target cell (e.g., a cancer cell, a pathogen-infected cell, etc.) are formulated together in the same pharmaceutical composition. In alternative embodiments, the molecules are formulated in separate pharmaceutical compositions.

Various formulations of a molecule capable of binding PD-1 or a natural ligand of PD-1, a molecule capable of mediating the redirected killing of a target cell (e.g., a cancer cell, a pathogen-infected cell, etc.), or a combination of such molecules, may be used for administration. In addition to the pharmacologically active agent(s), the compositions of the present invention may contain suitable pharmaceutically acceptable carriers comprising excipients and auxiliaries that are well-known in the art and are relatively inert substances that facilitate administration of a pharmacologically effective substance or which facilitate processing of the active compounds into preparations that can be used pharmaceutically for delivery to the site of action. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with a binding molecule of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the binding molecules of the present invention. The kit can further comprise one or more other prophylactic and/or therapeutic agents useful for the treatment of cancer, in one or more containers.

### IX. Methods of Administration

The compositions of the present invention may be provided for the treatment, prophylaxis, and amelioration of one or more symptoms associated with a disease, disorder or infection by administering to a subject an effective amount of a pharmaceutical composition comprising molecule capable of binding PD-1 or a natural ligand of PD-1 of the invention, and a pharmaceutical composition comprising a molecule capable of mediating the redirected killing of a tumor cell of the invention; or a pharmaceutical composition comprising a combination of such molecules of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Methods of administering a molecule or composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, the binding molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

The invention also provides that preparations of the binding molecules of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the binding molecules of the present invention are supplied as a dry sterile lyophilized powder in a hermetically sealed container.

The lyophilized preparations of the binding molecules of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such binding molecules, when provided in liquid form, are supplied in a hermetically sealed container.

The amount of such preparations of the invention that will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

As used herein, an “effective amount” of a pharmaceutical composition is an amount sufficient to effect beneficial or desired results including, without limitation, clinical results such as decreasing symptoms resulting from the disease, attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals. When applied to an individual active ingredient, administered alone, the term refers to that ingredient alone. When applied to a combination, the term refers to combined amounts of the active ingredients that result in the therapeutic effect, whether administered in combination, serially, or simultaneously.

An effective amount can be administered in one or more administrations. For purposes of this invention, an effective amount of drug, compound, or pharmaceutical composition is an amount sufficient: to kill and/or reduce the proliferation of cancer cells, and/or to eliminate, reduce and/or delay the development of metastasis from a primary site of cancer; or to reduce the proliferation of (or the effect of) an infectious pathogen and to reduce and/or delay the development of thepathogen-mediated disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art.

For the binding molecules encompassed by the invention, the dosage administered to a patient is preferably determined based upon the body weight (kg) of the recipient subject. For the binding molecules encompassed by the invention, the dosage administered to a patient is typically from about 0.01 μg/kg to about 30 mg/kg or more of the subject's body weight.

The dosage and frequency of administration of a binding molecule of the present invention may be reduced or altered by enhancing uptake and tissue penetration of the molecule by modifications such as, for example, lipidation.

The dosage of a binding molecule of the invention administered to a patient may be calculated for use as a single agent therapy. Alternatively, the molecule may be used in combination with other therapeutic compositions and the dosage administered to a patient are lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND C^{ANCER}, Lopez-Berestein and Fidler (eds.), Liss, New York, pp. 353-365 (1989); Lopez-Berestein, ibid., pp. 3 17-327).

Where the composition of the invention is a nucleic acid encoding a binding molecule of the present invention, the nucleic acid can be administered in vivo to promote expression of its encoded binding molecule by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of a binding molecule of the present invention can include a single treatment or, preferably, can include a series of treatments. In a preferred example, a subject is treated with a pharmaceutical composition of the invention for between about 1 to 10 weeks, preferably between 2 to 8 weeks, more preferably between about 3 to 7 weeks, and even more preferably for about 4, 5, or 6 weeks. The pharmaceutical compositions of the invention can be administered once a day with such administration occurring once a week, twice a week, once every two weeks, once a month, once every six weeks, once every two months, twice a year or once per year, etc. Alternatively, the pharmaceutical compositions of the invention can be administered twice a day with such administration occurring once a week, twice a week, once every two weeks, once a month, once every six weeks, once every two months, twice a year or once per year, etc. Alternatively, the pharmaceutical compositions of the invention can be administered three times a day with such administration occurring once a week, twice a week, once every two weeks, once a month, once every six weeks, once every two months, twice a year or once per year, etc. It will also be appreciated that the effective dosage of the molecules used for treatment may increase or decrease over the course of a particular treatment.

### EXAMPLES

Having now generally described the invention, the same will be more readily understood through reference to the following examples, which are provided by way of illustration and are not intended to be limiting of the present invention unless specified.

### Example 1

### Combination Treatment Study: LOX-IMVI Tumor Model

To illustrate the principles of the present invention, a combination treatment study was undertaken using a reconstituted tumor model in which LOX-IMVI human metastatic melanoma cancer cells were subcutaneously injected into MHCI^{-/-} mice reconstituted with human PBMCs. Mice were then administered vehicle or a treatment of:
(1) The humanized anti-human PD-1 antibody: hPD-1 mAb7 (1.2) IgG4(P), such antibody being a molecule capable of binding PD-1; and/or
(2) The CD3 x B7-H3 bispecific diabody DART-A, such diabody being a molecule capable of binding a cell surface molecule of an effector cell (i.e., CD3) and to a Cancer Antigen (i.e., B7-H3), and thereby being able to mediate the redirected killing of cancer cells expressing B7-H3.
   The amino acid sequences of such administered molecules are described above. Table 11 shows the parameters of the study. Each group consisted of 6 female mice. For all groups, mice received 5×10⁶ LOX-IMVI cancer cells (ID; administered at Study Day 33) and 10⁶ human PBMC (IP; administered at Study Day 0). Treatment (administered molecule(s) or vehicle commencing at Study Day 42) was provided weekly for three doses (Q7Dx3); doses were administered by intravenous injection.

TABLE 11

Administered Dose (mg/kg)

| Group | hPD-1 mAb7 (1.2) IgG4(P) | DART-A |
|---|---|---|
| 1 | 0 | 0 |
| 2 | 0 | 0.5 |
| 3 | 1.0 | 0 |
| 4 | 1.0 | 0.5 |

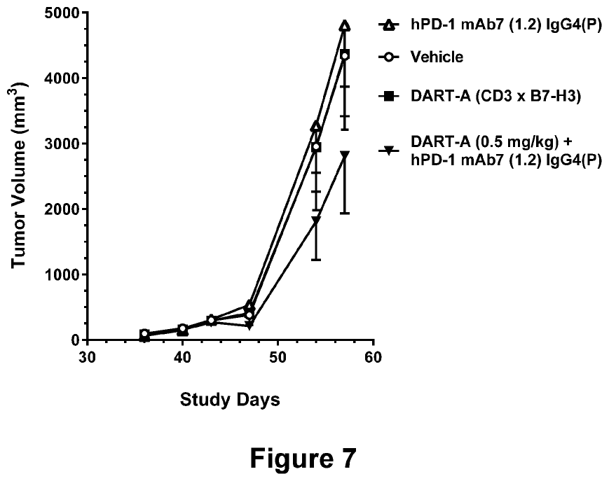

Tumor volume was measured as a function of time. FIG. 7 shows the results of this study, and demonstrates the unexpected benefit of the combined therapy relative to administration of only hPD-1 mAb7 (1.2) IgG4(P) or of only DART-A.

### Example 2

### Combination Treatment Study: Detroit562 Tumor Model

To further illustrate the principles of the present invention, a combination treatment study was undertaken using a reconstituted tumor model in which Detroit562 human metastatic pharyngeal carcinoma cancer cells were subcutaneously injected into MHCI^{-/-} mice reconstituted with human PBMCs. Mice were then administered vehicle control, 1 mg/kg hPD-1 mAb7 (1.2) IgG4(P), 0.5 mg/kg DART-A, or both 1 mg/kg hPD-1 mAb7 (1.2) IgG4(P) and 0.5 mg/kg DART-A. Table 12 shows the parameters of the study. Each group consisted of 8 male mice. For all groups, mice received 5×10⁶ Detroit562 cancer cells (ID) and 10⁶ human PBMC (IP; administered at Study Day 0). Treatment (administered molecule(s) or vehicle commencing at Study Day 7) was provided weekly for four weeks (Q7Dx4) or every 14 days for 2 doses (Q14Dx2); doses were administered by intravenous injection.

TABLE 12

| | |
|---|---|
| Dosage | Administered Dose (mg/kg) |

| Group | Regimen | hPD-1 mAb7 (1.2) IgG4(P) | DART-A |
|---|---|---|---|
| 1 | Q7Dx4 | 0 | 0 |
| 2 | Q7Dx4 | 1.0 | 0 |
| 3 | Q7Dx4 | 0 | 0.5 |
| 4 | Q14Dx2 | 0 | 0.5 |
| 5 | Q7Dx4 | 1.0 | 0.5 |
| 6 | Q14Dx2 | 1.0 | 0.5 |

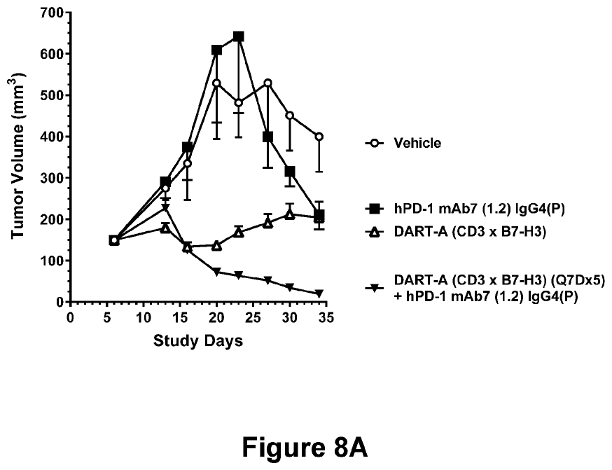

Tumor volume was measured as a function of time. FIGS. 8A-8B show the results of this study, which again demonstrates the unexpected benefit of the combined therapy relative to administration of only hPD-1 mAb7 (1.2) IgG4(P) or of only DART-A. FIG. 8A shows the results for Groups 1-3 and 5; FIG. 8B shows the results for Groups 1-4 and 6.

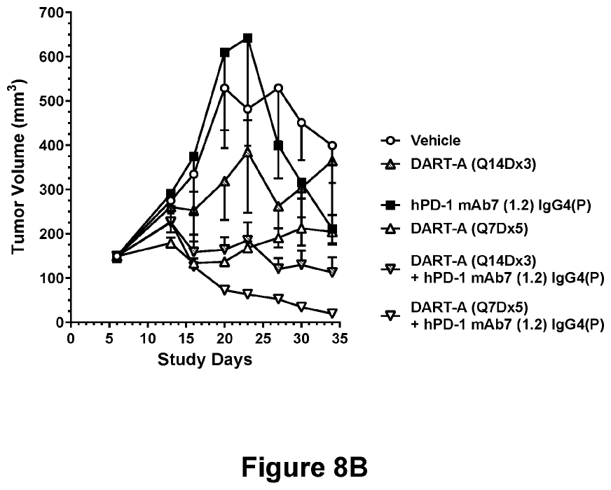

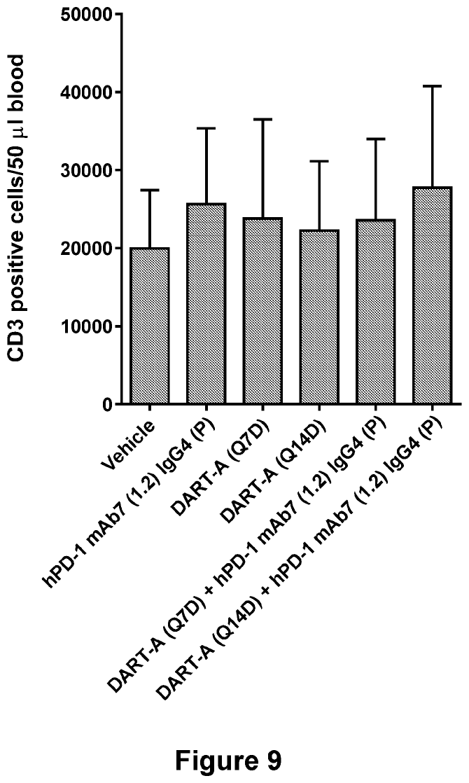

The concentration of CD3⁺ cells in the mice was determined at the conclusion of the study. It was surprisingly found that the concentration of such cells had increased in mice that had received the combination therapy (FIG. 9), thus indicating that the therapy of the present invention had enhanced the animals' immune responses.

### Example 3

### Signaling Model

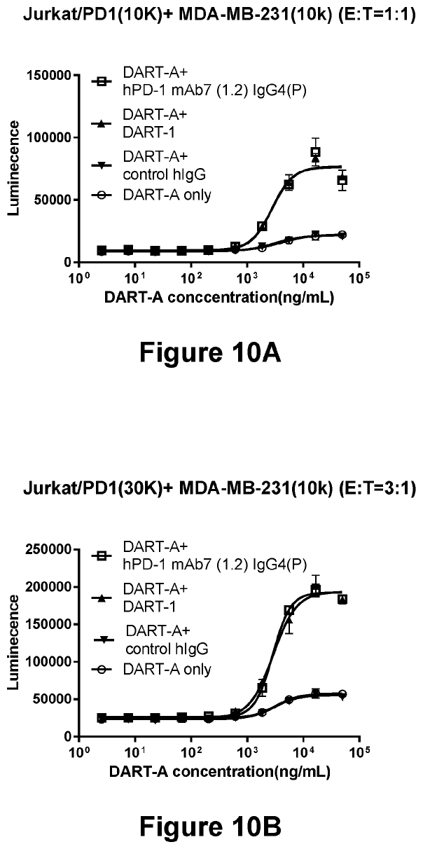

To further illustrate the principles of the present invention, cooperative T-cell signaling was examined in a T-cell/tumor cell co-culture system using a Jurkat-luc-NFAT/tumor cell luciferase reporter assay. Briefly, MDA-MB-231 tumor target cells expressing PD-1 and B7-H3 were mixed with MNFAT-luc2/PD-1 Jurkat T-cells at an effector:target cell ratio of 1:1 (FIG. 10A) or 3:1 (FIG. 10B) and cultured alone or with a fixed concentration (12.5 nM) of the PD-1 binding molecules hPD-1 mAb7 (1.2) IgG4(P), DART-1, a control antibody, in the presence of increasing concentrations of DART-A. Luminescence was measured as an indicator of cell activation and signaling. FIGS. 10A-10B show the results of this study which demonstrate that the combination of a molecule capable of binding PD-1 (e.g., hPD-1 mAb7 (1.2) IgG4(P), DART-1) and a molecule capable of mediating the redirected killing of a target cell (e.g., DART-A) enhances effector cell signaling activity.

### Example 4

### Combination Treatment Study: Comparing Normal to Anergic T-Cell in a A375 Tumor Model

To further illustrate the principles of the present invention, a combination treatment study was undertaken using a reconstituted tumor model in which A375 human melanoma cells were subcutaneously injected into NOG mice reconstituted with activated or anergic human T-cells. Mice were then administered vehicle or a treatment of:
(1) The PD-1 x LAG-3 bispecific diabody: DART-2 such diabody being a molecule capable of binding PD-1; and/or
(2) The CD3 x IL13Rα2 bispecific diabody DART-B, such diabody being a molecule capable of binding a cell surface molecule of an effector cell (i.e., CD3) and to a Cancer Antigen (i.e., IL13Rα2), and thereby being able to mediate the redirected killing of cancer cells expressing IL13Rα2.

Activated T-cells were prepared by two rounds of culturing purified human T-cells with CD3/CD28 activation beads in the presence of IL-2. Anergic T-cell were prepared by one round of culturing purified human T-cells with CD3/CD28 activation beads in the presence of IL-2 followed by one round of culturing with CD3/CD28 activation beads without IL-2. Groups of mice (n=8 female) received subcutaneous inoculation of 5×10⁶ A375 melanoma cells (pretreated with 0.1 μg/mL IFNγ for 24 hours) and 5×10⁶ human T-cells (activated or anergic) at Study Day 0. and were then administered vehicle control, 0.5 mg/kg DART-2, 0.5 mg/kg DART-B, or both 0.5 mg/kg DART-2 and 0.5 mg/kg DART-B. Treatment (administered molecule(s) or vehicle) was provided weekly for four doses (Q7Dx4) or as a single treatment on Study Day 0 (QD (SD)); doses were administered by intravenous injection. Table 13 shows the parameters of the study.

TABLE 13

| | | | Dose | |
|---|---|---|---|---|
| Group | T-cells | Treatment | (mg/kg) | Route/Schedule |
| 1 | Activated | Vehicle | 0 | IV/QD (SD 0) |
| 2 | Activated | DART-B | 0.01 | IV/QD (SD 0) |
| 3 | Activated | DART-B | 0.01 | IV/QD (SD 0) |
| | | DART-2 | 0.5 | IV/Q7Dx4 |
| 4 | Activated | DART-2 | 0.5 | IV/Q7Dx4 |
| 5 | Anergic | Vehicle | 0 | IV/QD (SD 0) |
| 6 | Anergic | DART-B | 0.01 | IV/QD (SD 0) |
| 7 | Anergic | DART-B | 0.01 | IV/QD (SD 0) |
| | | DART-2 | 0.5 | IV/Q7Dx4 |
| 8 | Anergic | DART-2 | 0.5 | IV/Q7Dx4 |

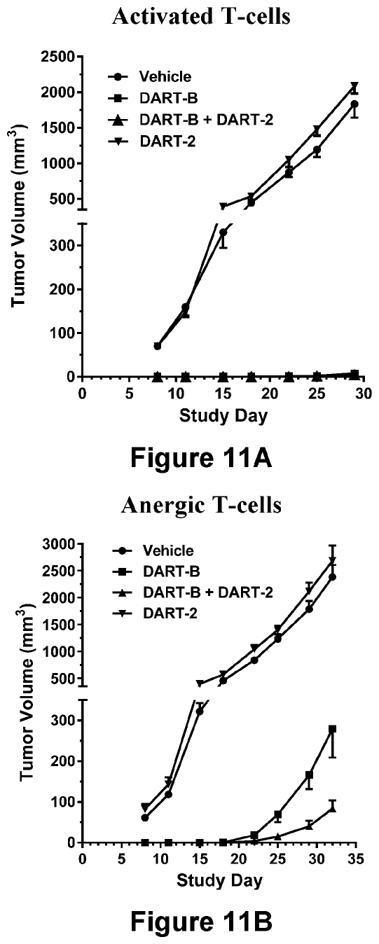

Tumor volume was measured as a function of time. FIGS. 11A-11B show the results of this study, demonstrate that the combined therapy of a molecule capable of binding PD-1 (e.g., hPD-1 mAb7 (1.2) IgG4(P), DART-1, DART-2) and a molecule capable of mediating the redirected killing of a target cell (e.g., DART-A, DART-B) reduces tumor recurrence in the presence of anergic T-cells. These results again demonstrate the unexpected benefit of the combined therapy of a molecule capable of binding PD-1 and a molecule capable of mediating the redirected killing of a target cell relative to administration of either molecule alone. FIG. 11A shows the results for Groups 1-4 inoculated with normal active T-cells; FIG. 11B shows the results for Groups 5-8 inoculated with allergic T-cells.

### Example 5

### Combination Treatment Study: A375 Tumor Model

To further illustrate the principles of the present invention, a combination treatment study was undertaken using a co-mix tumor model in which A375 melanoma cells were subcutaneously injected into NOG mice reconstituted with human T-cells. Mice were then administered Mice were then administered vehicle or a treatment of:
(1) The PD-1 x LAG-3 bispecific diabody: DART-2 such diabody being a molecule capable of binding PD-1; and/or
(2) The CD3 x IL13Rα2 bispecific diabody DART-B, such diabody being a molecule capable of binding a cell surface molecule of an effector cell (i.e., CD3) and to a Cancer Antigen (i.e., IL13Rα2), and thereby being able to mediate the redirected killing of cancer cells expressing IL13Rα2.

Table 14 shows the parameters of the study. Each group consisted of 8 female mice. For all groups, mice received 1.25×10⁶ A375 melanoma cells (pretreated for 24 hours with 100 ng/ml IFNγ) co-mixed with 1.25×10⁶ human T-cells (pretreated with 120 μg/ml DART-2 for 20 min) (SC; administered at Study Day 0). Mice in groups 5-8 were pretreated with DART-2 (500 μg/kg) 24 hours prior to cell injections (Study Day −1) and received addition doses of DART-2 (500 μg/kg) every 7 days starting on Study Day 7, for a total of 10 doses. Mice in groups 2-4 and 6-8 received a single dose of DART-B (1, 5 or 10 μg/kg) on Study Day 0. Group 1, received vehicle alone. All doses were administered by intravenous injection.

TABLE 14

| | | | Dose | |
|---|---|---|---|---|
| Group | N/sex | Treatment | (μg/kg) | Route/Schedule |
| 1 | 8/F | Vehicle | 0 | IV/QDx1 |
| 2 | 8/F | DART-B | 1 | IV/QDx1 |
| 3 | 8/F | DART-B | 5 | IV/QDx1 |
| 4 | 8/F | DART-B | 10 | IV/QDx1 |
| 5 | 8/F | DART-2 | 500 | IV/Q7Dx7 |
| 6 | 8/F | DART-B | 1 | IV/QDx1 |
| | | DART-2 | 500 | IV/Q7Dx7 |
| 7 | 8/F | DART-B | 5 | IV/QDx1 |
| | | DART-2 | 500 | IV/Q7Dx7 |
| 8 | 8/F | DART-B | 10 | IV/QDx1 |
| | | DART-2 | 500 | IV/Q7Dx7 |

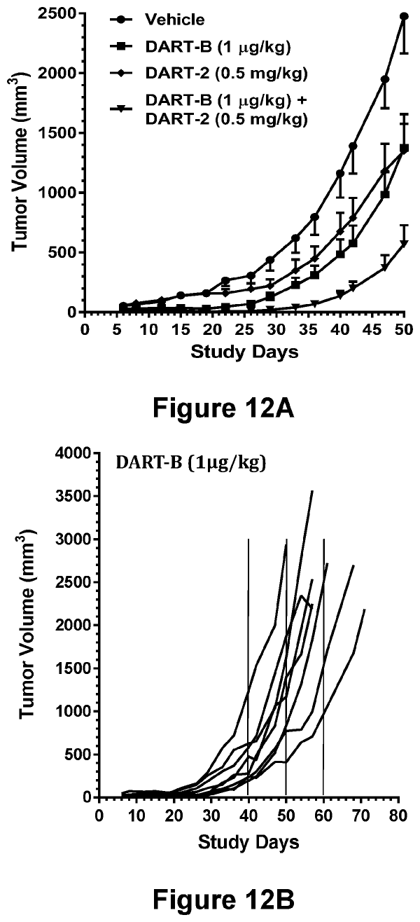

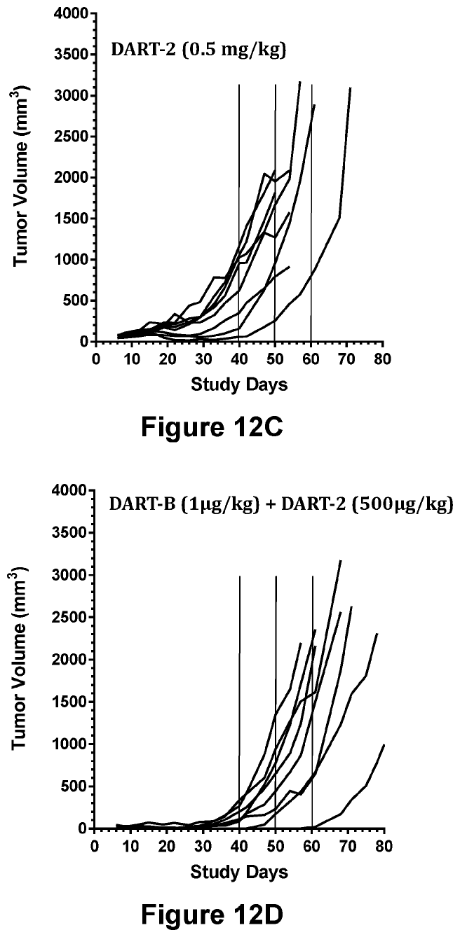

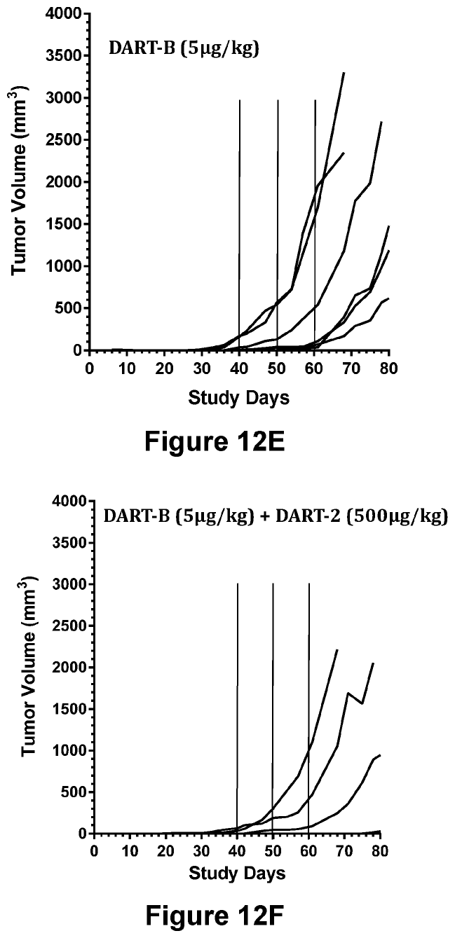

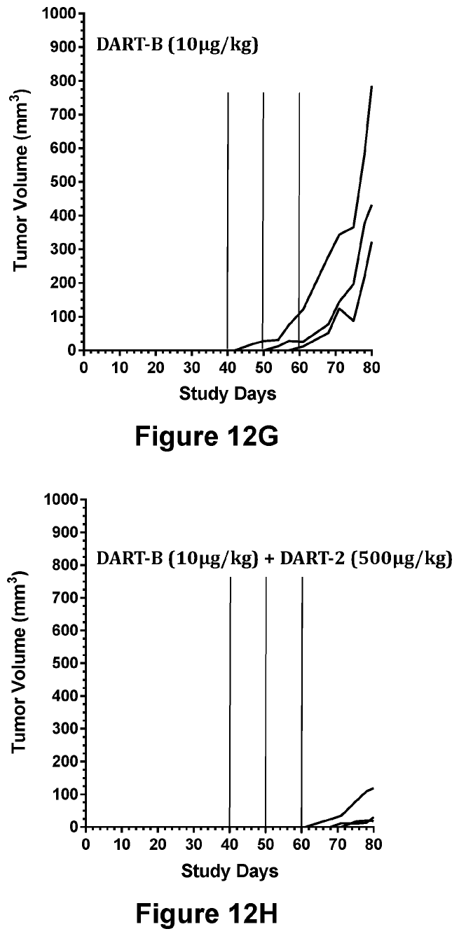

Tumor volume was measured as a function of time and is plotted in FIGS. 12A-12H. FIG. 12A shows the results for Groups 1, 2, 5 and 6 through day 50; FIGS. 12B-12H show the spider plots, through day 80, for the individual animals in Group 2 (FIG. 12B), Group 5 (FIG. 12C), Group 6 (FIG. 12D), Group 3 (FIG. 12E), Group 7 (FIG. 12F), Group 4 (FIG. 12G), and Group 8 (FIG. 12H). The results of this study demonstrate the unexpected benefit of the combined therapy of a molecule capable of binding PD-1 and a molecule capable of mediating the redirected killing of a target cell relative to administration of either molecule alone.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-38. (canceled)

39. A method for treating a cancer or a pathogen-associated disease in a subject, comprising: administering to a subject an amount of a first binding molecule and an amount of a second binding molecule effective to treat a cancer or a pathogen-associated disease, wherein:
(1) the first binding molecule is capable of immunospecifically binding to a PD-1 or a natural ligand of PD-1, and
(2) the second binding molecule is capable of mediating the redirected killing of a target cell, wherein the target cell is:
(a) a cancer cell that expresses a Cancer Antigen; or
(b) a pathogen-infected cell that expresses a Pathogen-Associated Antigen.

40. The method of claim 39, wherein:
(A) the first binding molecule is capable of inhibiting binding between PD-1 and a natural ligand of PD-1; and
(B) the second binding molecule comprises:
(1) an epitope-binding domain of an antibody capable of immunospecifically binding to a cell surface molecule of an effector cell; and
(2) an epitope-binding domain of an antibody capable of immunospecifically binding to the Cancer Antigen or an epitope-binding domain of an antibody capable of immunospecifically binding to the Pathogen-Associated Antigen.

41. The method of claim 39, wherein:
the first binding molecule comprises a diabody, scFv, antibody or TandAb;
the second binding molecule comprises a bispecific diabody, a CAR, a BiTe, or bispecific antibody; and
the diabody optionally consists of two polypeptide chains, three polypeptide chains, four polypeptide chains or five polypeptide chains.

42. The method of claim 41, wherein:
the first binding molecule, and/or the second binding molecule, comprises a CH2-CH3 Domain and optionally comprises one or more of a Hinge Domain, CL Domain or CH1 Domain;
the CH2-CH3 Domain, Hinge Domain, CL Domain or CH1 Domain optionally are from an IgG1, IgG2, IgG3 or IgG4 antibody;
the CH2-CH3 Domain optionally comprises one or more amino acid substitutions selected from: L234A, L235A, D265A, N297Q, and N297G;
the CH2-CH3 Domain optionally comprises two or more amino acid substitutions selected from: T250Q, M252Y, S254T, T256E, K288D, T307Q, V308P, A378V, M428L, N434A, H435K, and Y436I;
the CH2-CH3 Domain optionally comprises a T366W amino acid substitution or T366S, L368A and Y407V amino acid substitutions; and/or
the Hinge Domain optionally comprises a S228P amino acid substitution.

43. The method of claim 39, wherein the first binding molecule comprises a first epitope-binding domain of an antibody capable of immunospecifically binding to PD-1.

44. The method of claim 43, wherein the first epitope-binding domain comprises:
the six CDRs of SEQ ID NO:106 and 109, 106 and 108, 106 and 110, 107 and 108, 107 and 109, or 107 and 110; or
the VH Domain of SEQ ID NO:106 and the VL Domain of SEQ ID NO:109, the VH Domain of SEQ ID NO:106 and the VL Domain of SEQ ID NO:108, the VH Domain of SEQ ID NO:106 and the VL Domain of SEQ ID NO:110, the VH Domain of SEQ ID NO:107 and the VL Domain of SEQ ID NO:108, the VH Domain of SEQ ID NO:107 and the VL Domain of SEQ ID NO:109, or the VH Domain of SEQ ID NO:107 and the VL Domain of SEQ ID NO:110.

45. The method of claim 44, wherein the epitope-binding domain of the antibody capable of immunospecifically binding to PD-1 comprises the six CDRs of SEQ ID NO:106 and 109; or the VH Domain of SEQ ID NO:106 and the VL Domain of SEQ ID NO:109.

46. The method of claim 45, wherein the first binding molecule is an antibody comprising a Heavy Chain of SEQ ID NO:186 and a Light Chain of SEQ ID NO:187.

47. The method of claim 39, wherein the first binding molecule comprises a second epitope-binding domain of an antibody capable of immunospecifically binding to an epitope of a molecule that is not PD-1 or a natural ligand of PD-1.

48. The method of claim 47, wherein the second epitope-binding domain is of an antibody capable of immunospecifically binding to an epitope of CD137, LAG-3, OX40, TIGIT, TIM-3, or VISTA.

49. The method of claim 48, wherein the second epitope-binding domain is of an antibody capable of immunospecifically binding to an epitope of LAG-3 and comprises:
the six CDRs in the VL Domain of an antibody capable of immunospecifically binding to LAG-3 in the polypeptide of SEQ ID NO:274 and in the VH Domain of an antibody capable of immunospecifically binding to LAG-3 in the polypeptide of SEQ ID NO:275; or
the VL Domain of an antibody capable of immunospecifically binding to LAG-3 in the polypeptide of SEQ ID NO:274 and the VH Domain of an antibody capable of immunospecifically binding to LAG-3 in the polypeptide of SEQ ID NO:275.

50. The method of claim 49, wherein the first binding molecule is a bispecific diabody comprising the polypeptide of SEQ ID NO:274 and the polypeptide of SEQ ID NO:275.

51. The method of claim 39, wherein the second binding molecule comprises:
an epitope-binding domain of an antibody capable of immunospecifically binding to an epitope of the Pathogen-Associated Antigen, and
the Pathogen-Associated Antigen is selected from the group consisting of: Herpes Simplex Virus infected cell protein (ICP)47, Herpes Simplex Virus gD, Epstein-Barr Virus LMP-1, Epstein-Barr Virus LMP-2A, Epstein-Barr Virus LMP-2B, Human Immunodeficiency Virus envelope glycoprotein, Human Immunodeficiency Virus gp160, Human Immunodeficiency Virus gp120, Human Immunodeficiency Virus gp41, Human Papillomavirus E6, Human Papillomavirus E7, human T-cell leukemia virus gp64, human T-cell leukemia virus gp46, and human T-cell leukemia virus gp21.

52. The method of claim 51, wherein the Pathogen-Associated Antigen is Human Immunodeficiency Virus gp120 or Human Immunodeficiency Virus gp41 and the epitope-binding domain capable of immunospecifically binding to an epitope of the Pathogen-Associated Antigen comprises:
the six CDRs of SEQ ID NO:267 and 268, or 269 and 270; or
the VH Domain of SEQ ID NO:267 and the VL Domain of SEQ ID NO:268, or the VH Domain of SEQ ID NO:269 and the VL Domain of SEQ ID NO:270.

53. The method of claim 39, wherein the second binding molecule comprises:
an epitope-binding domain of an antibody capable of immunospecifically binding to the epitope of a Cancer Antigen, and
the Cancer Antigen is selected from the group consisting of: 19.9, 4.2, A33, ADAM-9, AH6, ALCAM, B1, B7-H3, BAGE, beta-catenin, blood group ALe^{b}/Le^{y}, Burkitt's lymphoma antigen-38.13, C14, CA125, Carboxypeptidase M, CD5, CD19, CD20, CD22, CD23, CD25, CD27, CD28, CD33, CD36, CD40/CD154, CD45, CD56, CD46, CD52, CD56, CD79a/CD79b, CD103, CD123, CD317, CDK4, CEA, CEACAM5/CEACAM6, C017-1A, CO-43, CO-514, CTA-1, CTLA-4, Cytokeratin 8, D1.1, D₁56-22, DR5, E₁ series, EGFR, an Ephrin receptor, Erb, GAGE, a GD2/GD3/GM2 ganglioside, GICA 19-9, gp100, Gp37, gp75, gpA33, HER2/neu, HMFG, human papillomavirus-E6/human papillomavirus-E7, HMW-MAA, I antigen, IL13Rα2, Integrin β6, JAM-3, KID3, KID31, KS 1/4 pan-carcinoma antigen, L6,L20, LEA, LUCA-2, M1:22:25:8, M18, M39, MAGE, MART, mesothelin, MUC-1, MUM-1, Myl, N-acetylglucosaminyltransferase, neoglycoprotein, NS-10, OFA-1, OFA-2, Oncostatin M, p15, p97, PEM, PEMA, PIPA, PSA, PSMA, prostatic acid phosphate, R24, ROR1, a sphingolipid, SSEA-1, SSEA-3, SSEA-4, sTn, the T cell receptor derived peptide, T₅A₇, TAG-72, TL5, TNF-receptor, TNF-γ receptor, TRA-1-85, a Transferrin Receptor, 5T4, TSTA, VEGF, a VEGF Receptor, VEP8, VEP9, VIM-D5, and Y hapten, Le^{y}.

54. The method of claim 53, wherein:
the Cancer Antigen is selected from the group consisting of: B7-H3, CD123, gpA33, CD19, 5T4 and IL13Rα2, and
the epitope-binding domain capable of immunospecifically binding to the epitope of a Cancer Antigen comprises:
the six CDRs of SEQ ID NO:213 and 214, 215 and 217, 215 and 218, 216 and 217, 216 and 218, 219 and 220, 221 and 225, 221 and 226, 221 and 227, 221 and 228, 221 and 229, 221 and 230, 222 and 225, 222 and 226, 222 and 227, 222 and 228, 222 and 229, 222 and 230, 223 and 225, 223 and 226, 223 and 227, 223 and 228, 223 and 229, 223 and 230, 224 and 225, 224 and 226, 224 and 227, 224 and 228, 224 and 229, 224 and 230, 231 and 232, 247 and 248, 263 and 264, 265 and 266, 257 and 258, 259 and 260, or 261 and 262; or
the VH Domain of SEQ ID NO:213 and the VL Domain of SEQ ID NO:214, the VH Domain of SEQ ID NO:215 and the VL Domain of SEQ ID NO:217, the VH Domain of SEQ ID NO:215 and the VL Domain of SEQ ID NO:218, the VH Domain of SEQ ID NO:216 and the VL Domain of SEQ ID NO:217, the VH Domain of SEQ ID NO:216 and the VL Domain of SEQ ID NO:218, the VH Domain of SEQ ID NO:219 and the VL Domain of SEQ ID NO:220, the VH Domain of SEQ ID NO:221 and the VL Domain of SEQ ID NO:225, the VH Domain of SEQ ID NO:221 and the VL Domain of SEQ ID NO:226, the VH Domain of SEQ ID NO:221 and the VL Domain of SEQ ID NO:227, the VH Domain of SEQ ID NO:221 and the VL Domain of SEQ ID NO:228, the VH Domain of SEQ ID NO:221 and the VL Domain of SEQ ID NO:229, the VH Domain of SEQ ID NO:221 and the VL Domain of SEQ ID NO:230, the VH Domain of SEQ ID NO:222 and the VL Domain of SEQ ID NO:225, the VH Domain of SEQ ID NO:222 and the VL Domain of SEQ ID NO:226, the VH Domain of SEQ ID NO:222 and the VL Domain of SEQ ID NO:227, the VH Domain of SEQ ID NO:222 and the VL Domain of SEQ ID NO:228, the VH Domain of SEQ ID NO:222 and the VL Domain of SEQ ID NO:229, the VH Domain of SEQ ID NO:222 and the VL Domain of SEQ ID NO:230, the VH Domain of SEQ ID NO:223 and the VL Domain of SEQ ID NO:225, the VH Domain of SEQ ID NO:223 and the VL Domain of SEQ ID NO:226, the VH Domain of SEQ ID NO:223 and the VL Domain of SEQ ID NO:227, the VH Domain of SEQ ID NO:223 and the VL Domain of SEQ ID NO:228, the VH Domain of SEQ ID NO:223 and the VL Domain of SEQ ID NO:229, the VH Domain of SEQ ID NO:223 and the VL Domain of SEQ ID NO:230, the VH Domain of SEQ ID NO:224 and the VL Domain of SEQ ID NO:225, the VH Domain of SEQ ID NO:224 and the VL Domain of SEQ ID NO:226, the VH Domain of SEQ ID NO:224 and the VL Domain of SEQ ID NO:227, the VH Domain of SEQ ID NO:224 and the VL Domain of SEQ ID NO:228, the VH Domain of SEQ ID NO:224 and the VL Domain of SEQ ID NO:229, the VH Domain of SEQ ID NO:224 and the VL Domain of SEQ ID NO:230, the VH Domain of SEQ ID NO:231 and the VL Domain of SEQ ID NO:232, the VH Domain of SEQ ID NO:247 and the VL Domain of SEQ ID NO:248, the VH Domain of SEQ ID NO:263 and the VL Domain of SEQ ID NO:264, the VH Domain of SEQ ID NO:265 and the VL Domain of SEQ ID NO:266, the VH Domain of SEQ ID NO:257 and the VL Domain of SEQ ID NO:258, the VH Domain of SEQ ID NO:259 and the VL Domain of SEQ ID NO:260, or the VH Domain of SEQ ID NO:261 and the VL Domain of SEQ ID NO:262.

55. The method of claim 39, wherein:
the second binding molecule comprises an epitope-binding domain of an antibody capable of immunospecifically binding to the epitope of a cell surface molecule of an effector cell; and
the cell surface molecule of the effector cell is selected from the group consisting of: CD2, CD3, CD8, CD16, TCR, and NKG2D.

56. The method of claim 55, wherein the cell surface molecule is CD3 and the epitope-binding domain comprises:
the six CDRs of SEQ ID NO:192 and 193, or 194 and 193; or
the VH Domain of SEQ ID NO:192 and the VL Domain of SEQ ID NO:193, or
the VH Domain of SEQ ID NO:194 and the VL Domain of SEQ ID NO:193.

57. The method of claim 39, wherein the second binding molecule is a bispecific diabody.

58. The method of claim 57, wherein the bispecific diabody comprises a first polypeptide chain comprising the polypeptide of SEQ ID NO:271, a second polypeptide chain comprising the polypeptide of SEQ ID NO:272, and a third polypeptide chain comprising the polypeptide of SEQ ID NO:273.

59. The method of claim 39, wherein:
(A) the first binding molecule is an antibody or a bispecific diabody comprising a first epitope-binding domain of an antibody capable of immunospecifically binding to PD-1, and the first epitope-binding domain comprises the six CDRs of SEQ ID NO:106 and 109, or the VH Domain of SEQ ID NO:106 and the VL Domain of SEQ ID NO:109; and
(B) the second binding molecule is a bispecific diabody comprising:
(1)(a) an epitope-binding domain of an antibody capable of immunospecifically binding to a B7-H3 Cancer Antigen and comprising the six CDRs of SEQ ID NO:222 and 226, or the VH Domain of SEQ ID NO:222 and the VL Domain of SEQ ID NO:226; and
(b) an epitope-binding domain of an antibody capable of immunospecifically binding to a CD3 and comprising the six CDRs of SEQ ID NO:192 and 193, or 194 and 193, or the VH Domain of SEQ ID NO:192 and the VL Domain of SEQ ID NO:193, or the VH Domain of SEQ ID NO:194 and the VL Domain of SEQ ID NO:193; or
(2)(a) an epitope-binding domain of an antibody capable of immunospecifically binding to a CD123 Cancer Antigen and comprising the six CDRs of SEQ ID NO:263 and 264, or the VH Domain of SEQ ID NO:263 and the VL Domain of SEQ ID NO:264; and
(b) an epitope-binding domain of an antibody capable of immunospecifically binding to a CD3 and comprising the six CDRs of SEQ ID NO:192 and 193, or 194 and 193, or the VH Domain of SEQ ID NO:192 and the VL Domain of SEQ ID NO:193, or the VH Domain of SEQ ID NO:194 and the VL Domain of SEQ ID NO:193; or
(3)(a) an epitope-binding domain of an antibody capable of immunospecifically binding to a gpA33 Cancer Antigen and comprising the six CDRs of SEQ ID NO:247 and 248, or the VH Domain of SEQ ID NO:247 and the VL Domain of SEQ ID NO:248; and
(b) an epitope-binding domain of an antibody capable of immunospecifically binding to a CD3 and comprising the six CDRs of SEQ ID NO:192 and 193, or 194 and 193, or the VH Domain of SEQ ID NO:192 and the VL Domain of SEQ ID NO:193, or the VH Domain of SEQ ID NO:194 and the VL Domain of SEQ ID NO:193; or
(4)(a) an epitope-binding domain of an antibody capable of immunospecifically binding to a Human Immunodeficiency Virus Antigen and comprising the six CDRs of SEQ ID NO:267 and 268, or the VH Domain of SEQ ID NO:267 and the VL Domain of SEQ ID NO:268; and
(b) an epitope-binding domain of an antibody capable of immunospecifically binding to a CD3 and comprising the six CDRs of SEQ ID NO:192 and 193, or 194 and 193, or the VH Domain of SEQ ID NO:192 and the VL Domain of SEQ ID NO:193, or the VH Domain of SEQ ID NO:194 and the VL Domain of SEQ ID NO:193; or
(5)(a) an epitope-binding domain of an antibody capable of immunospecifically binding to a Human Immunodeficiency Virus Antigen and comprising the six CDRs of SEQ ID NO:269 and 270, or the VH Domain of SEQ ID NO:269 and the VL Domain of SEQ ID NO:270; and
(b) an epitope-binding domain of an antibody capable of immunospecifically binding to a CD3 and comprising the six CDRs of SEQ ID NO:192 and 193, or 194 and 193, or the VH Domain of SEQ ID NO:192 and the VL Domain of SEQ ID NO:193, or the VH Domain of SEQ ID NO:194 and the VL Domain of SEQ ID NO:193.

60. The method of claim 59, wherein the first binding molecule is:
(1) an antibody comprising a Heavy Chain of SEQ ID NO:186 and a Light Chain of SEQ ID NO:187; or
(2) a bispecific diabody comprising a second epitope-binding domain that comprises:
(a) the six CDRs in the VL Domain of an antibody capable of immunospecifically binding to LAG-3 in the polypeptide of SEQ ID NO:274 and in the VH Domain of an antibody capable of immunospecifically binding to LAG-3 in the polypeptide of SEQ ID NO:275, or
(b) the VL Domain of an antibody capable of immunospecifically binding to LAG-3 in the polypeptide of SEQ ID NO:274 and the VH Domain of an antibody capable of immunospecifically binding to LAG-3 in the polypeptide of SEQ ID NO:275; or
(3) a bispecific diabody comprising the polypeptide of SEQ ID NO:274 and the polypeptide of SEQ ID NO:275.

61. The method of claim 59, wherein the second binding molecule comprises a first polypeptide chain comprising the polypeptide of SEQ ID NO:271, a second polypeptide chain comprising the polypeptide of SEQ ID NO:272, and a third polypeptide chain comprising the polypeptide of SEQ ID NO:273.

62. A pharmaceutical composition, comprising:
a first molecule capable of immunospecifically binding to a PD-1 or a natural ligand of PD-1;
a second binding molecule capable of mediating the redirected killing of a target cell,
wherein the target cell is:
(a) a cancer cell that expresses a Cancer Antigen; or
(b) a pathogen-infected cell that expresses a Pathogen-Associated Antigen; and
a pharmaceutically acceptable carrier.

63. A kit comprising the pharmaceutical composition of claim 62, wherein the binding molecules are compartmentalized in one or more containers.


SEQUENCE LISTING (275 sequences)

SEQ ID NO: 1 - protein, 98 aa, Homo sapiens
  MISC_FEATURE (1)..(98): Exemplary Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 2 - protein, 98 aa, Homo sapiens
  MISC_FEATURE (1)..(98): Exemplary Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 3 - protein, 98 aa, Homo sapiens
  MISC_FEATURE (1)..(98): Exemplary Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 4 - protein, 15 aa, Homo sapiens
  MISC_FEATURE (1)..(15): Exemplary Human IgG1 Hinge Domain
EPKSCDKTHTCPPCP

SEQ ID NO: 5 - protein, 12 aa, Homo sapiens
  MISC_FEATURE (1)..(12): Exemplary Human IgG2 Hinge Domain
ERKCCVECPPCP

SEQ ID NO: 6 - protein, 12 aa, Homo sapiens
  MISC_FEATURE (1)..(12): Exemplary Human IgG4 Hinge Domain
ESKYGPPCPSCP

SEQ ID NO: 7 - protein, 12 aa, Artificial Sequence
  Exemplary S228P-Stabilized Human IgG4 Hinge Domain
ESKYGPPCPPCP

SEQ ID NO: 8 - protein, 217 aa, Homo sapiens
  MISC_FEATURE (1)..(217): CH2-CH3 Domain of Exemplary Human IgG1
  MISC_FEATURE (217)..(217): XAA is Lysine (K) or Absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 9 - protein, 216 aa, Homo sapiens
  MISC_FEATURE (1)..(216): CH2-CH3 Domain of Exemplary Human IgG2
  MISC_FEATURE (216)..(216): XAA is Lysine (K) or Absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 10 - protein, 217 aa, Homo sapiens
  MISC_FEATURE (1)..(217): CH2-CH3 Domain of Exemplary Human IgG3
  MISC_FEATURE (217)..(217): XAA is Lysine (K) or Absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 11 - protein, 217 aa, Homo sapiens
  MISC_FEATURE (1)..(217): CH2-CH3 Domain of Exemplary Human IgG4
  MISC_FEATURE (217)..(217): XAA is Lysine (K) or Absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 12 - protein, 107 aa, Homo sapiens [sequence omitted]

SEQ ID NO: 13 - protein, 104 aa, Homo sapiens
  MISC_FEATURE (1)..(104): Exemplary Human CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 14 - protein, 8 aa, Artificial Sequence
  Preferred Intervening Spacer Peptide (Linker 1)
GGGSGGGG

SEQ ID NO: 15 - protein, 6 aa, Artificial Sequence
  Preferred Cysteine-Containing Spacer Peptide (Linker 2)
GGCGGG

SEQ ID NO: 16 - protein, 4 aa, Artificial Sequence
  Alternative Spacer Peptide (Linker 2)
GGGS

SEQ ID NO: 17 - protein, 6 aa, Artificial Sequence
  Alternative Spacer Peptide (Linker 2)
LGGGSG

SEQ ID NO: 18 - protein, 11 aa, Artificial Sequence
  Alternative Spacer Peptide (Linker 2)
GGGSGGGSGGG

SEQ ID NO: 19 - protein, 5 aa, Artificial Sequence
  Alternative Spacer Peptide (Linker 2)
ASTKG

SEQ ID NO: 20 - protein, 6 aa, Artificial Sequence
  Alternative Spacer Peptide (Linker 2)
LEPKSS

SEQ ID NO: 21 - protein, 5 aa, Artificial Sequence
  Alternative Spacer Peptide (Linker 2)
APSSS

SEQ ID NO: 22 - protein, 7 aa, Artificial Sequence
  Heterodimer-Promoting Domain
GVEPKSC

SEQ ID NO: 23 - protein, 6 aa, Artificial Sequence
  Heterodimer-Promoting Domain
VEPKSC

SEQ ID NO: 24 - protein, 6 aa, Artificial Sequence
  Heterodimer-Promoting Domain
AEPKSC

SEQ ID NO: 25 - protein, 7 aa, Artificial Sequence
  Heterodimer-Promoting Domain
GFNRGEC

SEQ ID NO: 26 - protein, 6 aa, Artificial Sequence
  Heterodimer-Promoting Domain
FNRGEC

SEQ ID NO: 27 - protein, 28 aa, Artificial Sequence
  “E-coil” Heterodimer-Promoting Domain
EVAALEKEVAALEKEVAALEKEVAALEK

SEQ ID NO: 28 - protein, 28 aa, Artificial Sequence
  “K-coil” Heterodimer-Promoting Domain
KVAALKEKVAALKEKVAALKEKVAALKE

SEQ ID NO: 29 - protein, 28 aa, Artificial Sequence
  Cysteine-Containing “E-Coil” Heterodimer- Promoting Domain
EVAACEKEVAALEKEVAALEKEVAALEK

SEQ ID NO: 30 - protein, 28 aa, Artificial Sequence
  Cysteine-Containing “K-Coil” Heterodimer- Promoting Domain
KVAACKEKVAALKEKVAALKEKVAALKE

SEQ ID NO: 31 - protein, 46 aa, Streptococcus dysgalactiae
  MISC_FEATURE (1)..(46): Albumin-Binding Domain 3 (ABD3) of Protein G of Streptococcus strain G148
LAEAKVLANRELDKYGVSDYYKNLIDNAKSAEGVKALIDEILAALP

SEQ ID NO: 32 - protein, 46 aa, Artificial Sequence; identical to SEQ ID NO: 31
  Deimmunized Variant Albumin-Binding Domain 3 (ABD3) of Protein G of Streptococcus strain G148

SEQ ID NO: 33 - protein, 46 aa, Artificial Sequence
  Deimmunized Variant Albumin-Binding Domain 3 (ABD3) of Protein G of Streptococcus strain G148
LAEAKVLANRELDKYGVSDYYKNAANNAKTVEGVKALIAEILAALP

SEQ ID NO: 34 - protein, 46 aa, Artificial Sequence
  Deimmunized Variant Albumin-Binding Domain 3 (ABD3) of Protein G of Streptococcus strain G148
LAEAKVLANRELDKYGVSDYYKNLISNAKSVEGVKALIAEILAALP

SEQ ID NO: 35 - protein, 8 aa, Artificial Sequence
  Intervening Spacer Peptide (Linker)
APSSSPME

SEQ ID NO: 36 - protein, 16 aa, Artificial Sequence
  Intervening Spacer Peptide (Linker)
VEPKSADKTHTCPPCP

SEQ ID NO: 37 - protein, 16 aa, Artificial Sequence
  Intervening Spacer Peptide (Linker)
LEPKSADKTHTCPPCP

SEQ ID NO: 38 - protein, 10 aa, Artificial Sequence
  Intervening Spacer Peptide (Linker)
DKTHTCPPCP

SEQ ID NO: 39 - protein, 13 aa, Artificial Sequence
  Alternative Linker
GGGDKTHTCPPCP

SEQ ID NO: 40 - protein, 16 aa, Artificial Sequence
  Alternative Linker
LEPKSSDKTHTCPPCP

SEQ ID NO: 41 - protein, 217 aa, Artificial Sequence
  CH2-CH3 Domain of Exemplary Human IgG1 Having L234A/L235A Substitutions
  MISC_FEATURE (217)..(217): XAA is Lysine (K) or Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 42 - protein, 217 aa, Artificial Sequence
  “Knob-Bearing” IgG1 CH2-CH3 Domain
  MISC_FEATURE (217)..(217): XAA is Lysine (K) or Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 43 - protein, 217 aa, Artificial Sequence
  “Hole-Bearing” IgG1 CH2-CH3 Domain
  MISC_FEATURE (217)..(217): XAA is Lysine (K) or Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 44 - protein, 15 aa, Artificial Sequence
  Preferred Intervening Spacer Peptide
GGGGSGGGGSGGGGS

SEQ ID NO: 45 - protein, 288 aa, Homo sapiens
  MISC_FEATURE (1)..(288): Human PD-1 Protein (NCBI Sequence NP_005009.2), Including Signal Sequence
  MISC_FEATURE (1)..(20): Signal Sequence of Human PD-1 Protein (NCBI Sequence NP_005009.2)
  MISC_FEATURE (21)..(288): Human PD-1 Protein (NCBI Sequence NP_005009.2); Mature Protein
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 46 - protein, 121 aa, Mus musculus
  MISC_FEATURE (1)..(121): VH Domain of Murine Anti-Human PD-1 mAb 1
DVQLQESGPGRVKPSQSLSLTCTVTGFSITNDYAWNWIRQFPGNKLEWMGHITYSGSTSYNPSLKSRISITRDTSKNHFFLQLSSVTPEDTATYYCARDYGSGYPYTLDYWGQGTSVTVSS

SEQ ID NO: 47 - protein, 6 aa, Mus musculus
  MISC_FEATURE (1)..(6): CDRH1 of PD-1 mAb 1
NDYAWN

SEQ ID NO: 48 - protein, 16 aa, Mus musculus
  MISC_FEATURE (1)..(16): CDRH2 of PD-1 mAb 1
HITYSGSTSYNPSLKS

SEQ ID NO: 49 - protein, 12 aa, Mus musculus
  MISC_FEATURE (1)..(12): CDRH3 of PD-1 mAb 1
DYGSGYPYTLDY

SEQ ID NO: 50 - protein, 106 aa, Mus musculus
  MISC_FEATURE (1)..(106): VL Domain of Murine Anti-Human PD-1 mAb 1
QIVLTQSPALMSASPGEKVTMTCSATSIVSYVYWYQQKPGSSPQPWIYLTSNLASGVPARFSGSGSGTSYSLTISSMEAEDAATYYCQQWSDNPYTFGGGTKLEIK

SEQ ID NO: 51 - protein, 10 aa, Mus musculus
  MISC_FEATURE (1)..(10): CDRL1 of PD-1 mAb 1
SATSIVSYVY

SEQ ID NO: 52 - protein, 7 aa, Mus musculus
  MISC_FEATURE (1)..(7): CDRL2 of PD-1 mAb 1
LTSNLAS

SEQ ID NO: 53 - protein, 9 aa, Mus musculus
  MISC_FEATURE (1)..(9): CDRL3 of PD-1 mAb 1
QQWSDNPYT

SEQ ID NO: 54 - protein, 121 aa, Artificial Sequence
  VH Domain of Humanized Anti-Human hPD-1 mAb 1 VH1
DVQLQESGPGLVKPSQTLSLTCTVSGFSISNDYAWNWIRQPPGKGLEWIGHITYSGSTSYNPSLKSRLTITRDTSKNQFVLTMTNMDPVDTATYYCARDYGSGYPYTLDYWGQGTTVTVSS

SEQ ID NO: 55 - protein, 106 aa, Artificial Sequence
  VL Domain of Humanized Anti-Human hPD-1 mAb 1 VL1
EIVLTQSPATLSVSPGEKVTITCSATSIVSYVYWYQQKPGQAPQPLIYLTSNLASGIPARFSGSGSGTDFTLTISSLEAEDAATYYCQQWSDNPYTFGGGTKVEIK

SEQ ID NO: 56 - protein, 116 aa, Mus musculus
  MISC_FEATURE (1)..(116): VH Domain of Murine Anti-Human PD-1 mAb 2
DVQLVESGGGLVQPGGSRKLSCAASGFVFSSFGMHWVRQAPEKGLEWVAYISSGSMSISYADTVKGRFTVTRDNAKNTLFLQMTSLRSEDTAIYYCASLSDYFDYWGQGTTLTVSS

SEQ ID NO: 57 - protein, 5 aa, Mus musculus
  MISC_FEATURE (1)..(5): CDRH1 of PD-1 mAb 2
SFGMH

SEQ ID NO: 58 - protein, 17 aa, Mus musculus
  MISC_FEATURE (1)..(17): CDRH2 of PD-1 mAb 2
YISSGSMSISYADTVKG

SEQ ID NO: 59 - protein, 7 aa, Mus musculus
  MISC_FEATURE (1)..(7): CDRH3 of PD-1 mAb 2
LSDYFDY

SEQ ID NO: 60 - protein, 112 aa, Mus musculus
  MISC_FEATURE (1)..(112): VL Domain of Murine Anti-Human PD-1 mAb 2
DVVMSQTPLSLPVSLGDQASISCRSSQSLVHSTGNTYLHWYLQKPGQSPKLLIYRVSNRFSGVPDRFSGSGSGTDFTLKISRVEAEDLGVFFCSQTTHVPWTFGGGTKLEIK

SEQ ID NO: 61 - protein, 16 aa, Mus musculus
  MISC_FEATURE (1)..(16): CDRL1 of PD-1 mAb 2
RSSQSLVHSTGNTYLH

SEQ ID NO: 62 - protein, 7 aa, Mus musculus
  MISC_FEATURE (1)..(7): CDRL2 of PD-1 mAb 2
RVSNRFS

SEQ ID NO: 63 - protein, 9 aa, Mus musculus
  MISC_FEATURE (1)..(9): CDRL3 of PD-1 mAb 2
SQTTHVPWT

SEQ ID NO: 64 - protein, 116 aa, Artificial Sequence
  VH Domain of Humanized Anti-Human hPD-1 mAb 2 VH1
EVQLVESGGGLVQPGGSLRLSCAASGFVFSSFGMHWVRQAPGKGLEWVAYISSGSMSISYADTVKGRFTISRDNAKNTLYLQMNSLRTEDTALYYCASLSDYFDYWGQGTTVTVSS

SEQ ID NO: 65 - protein, 112 aa, Artificial Sequence
  VL Domain of Humanized Anti-Human hPD-1 mAb 2 VH1
DVVMTQSPLSLPVTLGQPASISCRSSQSLVHSTGNTYLHWYLQKPGQSPQLLIYRVSNRFSGVPDRFSGSGSGTDFTLKISRVEAEDVGVYYCSQTTHVPWTFGQGTKLEIK

SEQ ID NO: 66 - protein, 125 aa, Mus musculus
  MISC_FEATURE (1)..(125): VH Domain of Murine Anti-Human PD-1 mAb 3
QVQLQQSGAELVRPGASVTLSCKASGYTFTDYVMHWVKQTPVHGLEWIGTIDPETGGTAYNQKFKGKAILTADKSSNTAYMELRSLTSEDSAVYYFTREKITTIVEGTYWYFDVWGTGTTVTVSS

SEQ ID NO: 67 - protein, 5 aa, Mus musculus
  MISC_FEATURE (1)..(5): CDRH1 of PD-1 mAb 3
DYVMH

SEQ ID NO: 68 - protein, 17 aa, Mus musculus
  MISC_FEATURE (1)..(17): CDRH2 of PD-1 mAb 3
TIDPETGGTAYNQKFKG

SEQ ID NO: 69 - protein, 16 aa, Mus musculus
  MISC_FEATURE (1)..(16): CDRH3 of PD-1 mAb 3
EKITTIVEGTYWYFDV

SEQ ID NO: 70 - protein, 112 aa, Mus musculus
  MISC_FEATURE (1)..(112): VL Domain of Murine Anti-Human PD-1 mAb 3
DVLLTQTPLSLPVSLGDQASISCRSSQNIVHSNGDTYLEWYLQKPGQSPKLLIYKVSNRFSGVPDRFSGSGSGTDFTLKISRVEAEDLGVYYCFQGSHLPYTFGGGTKLEIK

SEQ ID NO: 71 - protein, 16 aa, Mus musculus
  MISC_FEATURE (1)..(16): CDRL1 of PD-1 mAb 3
RSSQNIVHSNGDTYLE

SEQ ID NO: 72 - protein, 7 aa, Mus musculus
  MISC_FEATURE: CDRL2 of PD-1 mAb 3
KVSNRFS

SEQ ID NO: 73 - protein, 9 aa, Mus musculus
  MISC_FEATURE (1)..(9): CDRL3 of PD-1 mAb 3
FQGSHLPYT

SEQ ID NO: 74 - protein, 116 aa, Mus musculus
  MISC_FEATURE (1)..(116): VH Domain of Murine Anti-Human PD-1 mAb 4
DVQLVESGGGLVQPGGSRKLSCAASGFVFSSFGMHWVRQAPEKGLEWVAYISSGSMSISYADTVKGRFTVTRDNAKNTLFLQMTSLRSEDTAIYYCASLTDYFDYWGQGTTLTVSS

SEQ ID NO: 75 - protein, 5 aa, Mus musculus
  MISC_FEATURE (1)..(5): CDRH1 of PD-1 mAb 4
SFGMH

SEQ ID NO: 76 - protein, 17 aa, Mus musculus
  MISC_FEATURE (1)..(17): CDRH2 of PD-1 mAb 4
YISSGSMSISYADTVKG

SEQ ID NO: 77 - protein, 7 aa, Mus musculus
  MISC_FEATURE (1)..(7): CDRH3 of PD-1 mAb 4
LTDYFDY

SEQ ID NO: 78 - protein, 112 aa, Mus musculus
  MISC_FEATURE (1)..(112): VL Domain of Murine Anti-Human PD-1 mAb 4
DVVMSQTPLSLPVSLGDQASISCRSSQSLVHSTGNTYFHWYLQKPGQSPKLLIYRVSNRFSGVPDRFSGSGSGTDFTLKISRVEAEDLGVYFCSQTTHVPWTFGGGTKLEIK

SEQ ID NO: 79 - protein, 16 aa, Mus musculus
  MISC_FEATURE (1)..(16): CDRL1 of PD-1 mAb 4
RSSQSLVHSTGNTYFH

SEQ ID NO: 80 - protein, 7 aa, Mus musculus
  MISC_FEATURE (1)..(7): CDRL2 of PD-1 mAb 4
RVSNRFS

SEQ ID NO: 81 - protein, 9 aa, Mus musculus
  MISC_FEATURE (1)..(9): CDRL3 of PD-1 mAb 4
SQTTHVPWT

SEQ ID NO: 82 - protein, 119 aa, Mus musculus
  MISC_FEATURE (1)..(119): VH Domain of Murine Anti-Human PD-1 mAb 5
QVQLQQPGVELVRPGASVKLSCKASGYSFTAYWMNWMKQRPGQGLEWIGVIHPSDSETWLNQKFKDKATLTVDKSSSTAYMQLISPTSEDSAVYYCAREHYGSSPFAYWGQGTLVTVSA

SEQ ID NO: 83 - protein, 5 aa, Mus musculus
  MISC_FEATURE (1)..(5): CDRH1 of PD-1 mAb 5
AYWMN

SEQ ID NO: 84 - protein, 17 aa, Mus musculus
  MISC_FEATURE (1)..(17): CDRH2 of PD-1 mAb 5
VIHPSDSETWLNQKFKD

SEQ ID NO: 85 - protein, 10 aa, Mus musculus
  MISC_FEATURE (1)..(10): CDRH3 of PD-1 mAb 5
EHYGSSPFAY

SEQ ID NO: 86 - protein, 111 aa, Mus musculus
  MISC_FEATURE (1)..(111): VL Domain of Murine Anti-Human PD-1 mAb 5
DIVLTQSPASLAVSLGQRATISCRANESVDNYGMSFMNWFQQKPGQPPKLLIYAASNQGSGVPARFSGSGSGTDFSLNIHPMEEDDTAMYFCQQSKEVPYTFGGGTKLEIK

SEQ ID NO: 87 - protein, 15 aa, Mus musculus
  MISC_FEATURE (1)..(15): CDRL1 of PD-1 mAb 5
RANESVDNYGMSFMN

SEQ ID NO: 88 - protein, 7 aa, Mus musculus
  MISC_FEATURE (1)..(7): CDRL2 of PD-1 mAb 5
AASNQGS

SEQ ID NO: 89 - protein, 9 aa, Mus musculus
  MISC_FEATURE (1)..(9): CDRL3 of PD-1 mAb 5
QQSKEVPYT

SEQ ID NO: 90 - protein, 118 aa, Mus musculus
  MISC_FEATURE (1)..(118): VH Domain of Murine Anti-Human PD-1 mAb 6
EVKLVESGGGLVNPGGSLKLSCAASGFTFSSYGMSWVRQTPEKRLEWVATISGGGSDTYYPDSVKGRFTISRDNAKNNLYLQMSSLRSEDTALYYCARQKATTWFAYWGQGTLVTVST

SEQ ID NO: 91 - protein, 5 aa, Mus musculus
  MISC_FEATURE (1)..(5): CDRH1 of PD-1 mAb 6
SYGMS

SEQ ID NO: 92 - protein, 17 aa, Mus musculus
  MISC_FEATURE (1)..(17): CDRH2 of PD-1 mAb 6
TISGGGSDTYYPDSVKG

SEQ ID NO: 93 - protein, 9 aa, Mus musculus
  MISC_FEATURE (1)..(9): CDRH3 of PD-1 mAb 6
QKATTWFAY

SEQ ID NO: 94 - protein, 111 aa, Mus musculus
  MISC_FEATURE (1)..(111): VL Domain of Murine Anti-Human PD-1 mAb 6
DIVLTQSPASLAVSLGQRATISCRASESVDNYGISFMNWFQQKPGQPPKLLIYPASNQGSGVPARFSGSGSGTDFSLNIHPMEEDDAAMYFCQQSKEVPWTFGGGTKLEIK

SEQ ID NO: 95 - protein, 15 aa, Mus musculus
  MISC_FEATURE (1)..(15): CDRL1 of PD-1 mAb 6
RASESVDNYGISFMN

SEQ ID NO: 96 - protein, 7 aa, Mus musculus
  MISC_FEATURE (1)..(7): CDRL2 of PD-1 mAb 6
PASNQGS

SEQ ID NO: 97 - protein, 9 aa, Mus musculus
  MISC_FEATURE (1)..(9): CDRL3 of PD-1 mAb 6
QQSKEVPWT

SEQ ID NO: 98 - protein, 119 aa, Mus musculus
  MISC_FEATURE (1)..(119): VH Domain of Murine Anti-Human PD-1 mAb 7
QVQLQQPGAELVRPGASVKLSCKASGYSFTSYWMNWVKQRPGQGLEWIGVIHPSDSETWLDQKFKDKATLTVDKSSTTAYMQLISPTSEDSAVYYCAREHYGTSPFAYWGQGTLVTVSS

SEQ ID NO: 99 - protein, 5 aa, Mus musculus
  MISC_FEATURE (1)..(5): CDRH1 of PD-1 mAb 7
SYWMN

SEQ ID NO: 100 - protein, 17 aa, Mus musculus
  MISC_FEATURE (1)..(17): CDRH2 of PD-1 mAb 7
VIHPSDSETWLDQKFKD

SEQ ID NO: 101 - protein, 10 aa, Mus musculus
  MISC_FEATURE (1)..(10): CDRH3 of PD-1 mAb 7
EHYGTSPFAY

SEQ ID NO: 102 - protein, 111 aa, Mus musculus
  MISC_FEATURE (1)..(111): VL Domain of Murine Anti-Human PD-1 mAb 7
DIVLTQSPASLAVSLGQRATISCRANESVDNYGMSFMNWFQQKPGQPPKLLIHAASNQGSGVPARFSGSGFGTDFSLNIHPMEEDDAAMYFCQQSKEVPYTFGGGTKLEIK

SEQ ID NO: 103 - protein, 15 aa, Mus musculus
  MISC_FEATURE (1)..(15): CDRL1 of PD-1 mAb 7
RANESVDNYGMSFMN

SEQ ID NO: 104 - protein, 7 aa, Mus musculus
  MISC_FEATURE (1)..(7): CDRL2 of PD-1 mAb 7
AASNQGS

SEQ ID NO: 105 - protein, 9 aa, Mus musculus
  MISC_FEATURE (1)..(9): CDRL3 of PD-1 mAb 7
QQSKEVPYT

SEQ ID NO: 106 - protein, 119 aa, Artificial Sequence
  VH Domain of Humanized Anti-Human hPD-1 mAb 7 VH1
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 107 - protein, 119 aa, Artificial Sequence
  VH Domain of Humanized Anti-Human hPD-1 mAb 7 VH2
QVQLVQSGAEVKKPGASVKVSCKASGYSFTSYWMNWVRQAPGQGLEWAGVIHPSDSETWLDQKFKDRVTITVDKSTSTAYMELSSLRSEDTAVYYCAREHYGTSPFAYWGQGTLVTVSS

SEQ ID NO: 108 - protein, 111 aa, Artificial Sequence
  VL Domain of Humanized Anti-Human hPD-1 mAb 7 VL1
EIVLTQSPATLSLSPGERATLSCRANESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 109 - protein, 111 aa, Artificial Sequence
  VL Domain of Humanized Anti-Human hPD-1 mAb 7 VL2
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 110 - protein, 111 aa, Artificial Sequence
  VL Domain of Humanized Anti-Human hPD-1 mAb 7 VL3
EIVLTQSPATLSLSPGERATLSCRASESVDNYGMSFMNWFQQKPGQPPKLLIHAASNRGSGVPSRFSGSGSGTDFTLTISSLEPEDFAVYFCQQSKEVPYTFGGGTKVEIK

SEQ ID NO: 111 - protein, 15 aa, Artificial Sequence
  CDRL1 of the VL Domain of hPD-1 mAb 7 VL2 and hPD-1 mAb 7 VL3
RASESVDNYGMSFMN

SEQ ID NO: 112 - protein, 7 aa, Artificial Sequence
  CDRL2 of hPD-1 mAb 7 VL3
AASNRGS

SEQ ID NO: 113 - protein, 113 aa, Mus musculus
  MISC_FEATURE (1)..(113): VH Domain of Murine Anti-Human PD-1 mAb 8
EGQLQQSGPELVKPGASVKISCKASGYTFTDYYMNWVKQNHGKSLEWIGDINPKNGDTHYNQKFKGEATLTVDKSSTTAYMELRSLTSEDSAVYYCASDFDYWGQGTTLTVSS

SEQ ID NO: 114 - protein, 5 aa, Mus musculus
  MISC_FEATURE (1)..(5): CDRH1 of PD-1 mAb 8
DYYMN

SEQ ID NO: 115 - protein, 17 aa, Mus musculus
  MISC_FEATURE (1)..(17): CDRH2 of PD-1 mAb 8
DINPKNGDTHYNQKFKG

SEQ ID NO: 116 - protein, 4 aa, Mus musculus
  MISC_FEATURE (1)..(4): CDRH3 of PD-1 mAb 8
DFDY

SEQ ID NO: 117 - protein, 112 aa, Mus musculus
  MISC_FEATURE (1)..(112): VL Domain of Murine Anti-Human PD-1 mAb 8
DVVMTQTPLSLPVGLGDQASISCRSSQTLVYSNGNTYLNWFLQKPGQSPKLLIYKVSNRFSGVPDRFSGSGSGTDFTLKISRVEAEDLGVYFCSQSTHVPFTFGSGTKLEIK

SEQ ID NO: 118 - protein, 16 aa, Mus musculus
  MISC_FEATURE (1)..(16): CDRL1 of PD-1 mAb 8
RSSQTLVYSNGNTYLN

SEQ ID NO: 119 - protein, 7 aa, Mus musculus
  MISC_FEATURE (1)..(7): CDRL2 of PD-1 mAb 8
KVSNRFS

SEQ ID NO: 120 - protein, 9 aa, Mus musculus
  MISC_FEATURE (1)..(9): CDRL3 of PD-1 mAb 8
SQSTHVPFT

SEQ ID NO: 121 - protein, 119 aa, Mus musculus
  MISC_FEATURE (1)..(119): VH Domain of Murine Anti-Human PD-1 mAb 9
EVMLVESGGGLVKPGGSLKLSCAASGFTFSSYLVSWVRQTPEKRLEWVATISGGGGNTYYSDSVKGRFTISRDNAKNTLYLQISSLRSEDTALYYCARYGFDGAWFAYWGQGTLVTVSS

SEQ ID NO: 122 - protein, 5 aa, Mus musculus
  MISC_FEATURE (1)..(5): CDRH1 of PD-1 mAb 9
SYLVS

SEQ ID NO: 123 - protein, 17 aa, Mus musculus
  MISC_FEATURE (1)..(17): CDRH2 of PD-1 mAb 9
TISGGGGNTYYSDSVKG

SEQ ID NO: 124 - protein, 10 aa, Mus musculus
  MISC_FEATURE (1)..(10): CDRH3 of PD-1 mAb 9
YGFDGAWFAY

SEQ ID NO: 125 - protein, 107 aa, Mus musculus
  MISC_FEATURE (1)..(107): VL Domain of Murine Anti-Human PD-1 mAb 9
DIQMTQSPASLSASVGDIVTITCRASENIYSYLAWYQQKQEKSPQLLVYNAKTLAAGVPSRFSGSGSGTQFSLTINSLQPEDFGNYYCQHHYAVPWTFGGGTRLEIT

SEQ ID NO: 126 - protein, 11 aa, Mus musculus
  MISC_FEATURE (1)..(11): CDRL1 of PD-1 mAb 9
RASENIYSYLA

SEQ ID NO: 127 - protein, 7 aa, Mus musculus
  MISC_FEATURE (1)..(7): CDRL2 of PD-1 mAb 9
NAKTLAA

SEQ ID NO: 128 - protein, 9 aa, Mus musculus
  MISC_FEATURE (1)..(9): CDRL3 of PD-1 mAb 9
QHHYAVPWT

SEQ ID NO: 129 - protein, 119 aa, Artificial Sequence
  VH Domain of Humanized Anti-Human hPD-1 mAb 9 VH1
EVQLVESGGGLVRPGGSLKLSCAASGFTFSSYLVSWVRQAPGKGLEWVATISGGGGNTYYSDSVKGRFTISRDNAKNSLYLQMNSLRAEDTATYYCARYGFDGAWFAYWGQGTLVTVSS

SEQ ID NO: 130 - protein, 119 aa, Artificial Sequence
  VH Domain of Humanized Anti-Human hPD-1 mAb 9 VH2
EVQLVESGGGLARPGGSLKLSCAASGFTFSSYLVGWVRQAPGKGLEWTATISGGGGNTYYSDSVKGRFTISRDNAKNSLYLQMNSARAEDTATYYCARYGFDGAWFAYWGQGTLVTVSS

SEQ ID NO: 131 - protein, 5 aa, Artificial Sequence
  CDRH1 of hPD-1 mAb 9 VH2
SYLVG

SEQ ID NO: 132 - protein, 107 aa, Artificial Sequence
  VL Domain of Humanized Anti-Human hPD-1 mAb 9 VL1
DIQMTQSPSSLSASVGDRVTITCRASENIYSYLAWYQQKPGKAPKLLIYNAKTLAAGVPSRFSGSGSGTDFTLTISSLQPEDFATYYCQHHYAVPWTFGQGTKLEIK

SEQ ID NO: 133 - protein, 107 aa, Artificial Sequence
  VL Domain of Humanized Anti-Human hPD-1 mAb 9 VL2
DIQMTQSPSSLSASVGDRVTITCRASENIYNYLAWYQQKPGKAPKLLIYDAKTLAAGVPSRFSGSGSGTDFTLTISSLQPEDFATYYCQHHYAVPWTFGQGTKLEIK

SEQ ID NO: 134 - protein, 11 aa, Artificial Sequence
  CDRL1 of hPD-1 mAb 9 VL2
RASENIYNYLA

SEQ ID NO: 135 - protein, 7 aa, Artificial Sequence
  CDRL2 of hPD-1 mAb 9 VL2
DAKTLAA

SEQ ID NO: 136 - protein, 116 aa, Mus musculus
  MISC_FEATURE (1)..(116): VH Domain of Murine Anti-Human PD-1 mAb 10
EVILVESGGGLVKPGGSLKLSCAASGFTFSNYLMSWVRQTPEKRLEWVASISGGGSNIYYPDSVKGRFTISRDNAKNTLYLQMNSLRSEDTALYYCARQELAFDYWGQGTTLTVSS

SEQ ID NO: 137 - protein, 5 aa, Mus musculus
  MISC_FEATURE (1)..(5): CDRH1 of PD-1 mAb 10
NYLMS

SEQ ID NO: 138 - protein, 17 aa, Mus musculus
  MISC_FEATURE (1)..(17): CDRH2 of PD-1 mAb 10
SISGGGSNIYYPDSVKG

SEQ ID NO: 139 - protein, 7 aa, Mus musculus
  MISC_FEATURE (1)..(7): CDRH3 of PD-1 mAb 10
QELAFDY

SEQ ID NO: 140 - protein, 107 aa, Mus musculus
  MISC_FEATURE (1)..(107): VL Domain of Murine Anti-Human PD-1 mAb 10
DIQMTQTTSSLSASLGDRVTISCRTSQDISNFLNWYQQKPDGTIKLLIYYTSRLHSGVPSRFSGSGSGTDYSLTISNLEQEDIATYFCQQGSTLPWTFGGGTKLEII

SEQ ID NO: 141 - protein, 11 aa, Mus musculus
  MISC_FEATURE (1)..(11): CDRL1 of PD-1 mAb 10
RTSQDISNFLN

SEQ ID NO: 142 - protein, 7 aa, Mus musculus
  MISC_FEATURE (1)..(7): CDRL2 of PD-1 mAb 10
YTSRLHS

SEQ ID NO: 143 - protein, 9 aa, Mus musculus
  MISC_FEATURE (1)..(9): CDRL3 of PD-1 mAb 10
QQGSTLPWT

SEQ ID NO: 144 - protein, 117 aa, Mus musculus
  MISC_FEATURE (1)..(117): VH Domain of Murine Anti-Human PD-1 mAb 11
EVQLQQSGTVLARPGASVKMSCKTSGYTFTGYWMHWVKQRPGQGLKWMGAIYPGNSDTHYNQKFKGKAKLTAVTSASTAYMELSSLTNEDSAIYYCTTGTYSYFDVWGTGTTVTVSS

SEQ ID NO: 145 - protein, 5 aa, Mus musculus
  MISC_FEATURE (1)..(5): CDRH1 of PD-1 mAb 11
GYWMH

SEQ ID NO: 146 - protein, 17 aa, Mus musculus
  MISC_FEATURE (1)..(17): CDRH2 of PD-1 mAb 11
AIYPGNSDTHYNQKFKG

SEQ ID NO: 147 - protein, 8 aa, Mus musculus
  MISC_FEATURE (1)..(8): CDRH3 of PD-1 mAb 11
GTYSYFDV

SEQ ID NO: 148 - protein, 106 aa, Mus musculus
  MISC_FEATURE (1)..(106): VL Domain of Murine Anti-Human PD-1 mAb 11
DILLTQSPAILSVSPGERVSFSCRASQSIGTSIHWYQHRTNGSPRLLIKYASESISGIPSRFSGSGSGTDFTLSINSVESEDIADYYCQQSNSWLTFGAGTKLELK

SEQ ID NO: 149 - protein, 11 aa, Mus musculus
  MISC_FEATURE (1)..(11): CDRL1 of PD-1 mAb 11
RASQSIGTSIH

SEQ ID NO: 150 - protein, 7 aa, Mus musculus
  MISC_FEATURE (1)..(7): CDRL2 of PD-1 mAb 11
YASESIS

SEQ ID NO: 151 - protein, 8 aa, Mus musculus
  MISC_FEATURE (1)..(8): CDRL3 of PD-1 mAb 11
QQSNSWLT

SEQ ID NO: 152 - protein, 125 aa, Mus musculus
  MISC_FEATURE (1)..(125): VH Domain of Murine Anti-Human PD-1 mAb 12
QGHLQQSGAELVRPGASVTLSCKASGFTFTDYEMHWVKQTPVHGLEWIGTIDPETGGTAYNQKFKGKAILTVDKSSTTTYMELRSLTSEDSAVFYCSRERITTVVEGAYWYFDVWGTGTTVTVSS

SEQ ID NO: 153 - protein, 5 aa, Mus musculus
  MISC_FEATURE (1)..(5): CDRH1 of PD-1 mAb 12
DYEMH

SEQ ID NO: 154 - protein, 17 aa, Mus musculus
  MISC_FEATURE (1)..(17): CDRH2 of PD-1 mAb 12
TIDPETGGTAYNQKFKG

SEQ ID NO: 155 - protein, 16 aa, Mus musculus
  MISC_FEATURE (1)..(16): CDRH3 of PD-1 mAb 12
ERITTVVEGAYWYFDV

SEQ ID NO: 156 - protein, 112 aa, Mus musculus
  MISC_FEATURE (1)..(112): VL Domain of Murine Anti-Human PD-1 mAb 12
DVLMTQTPLSLPVSLGDQASISCRSSQNIVHSNGNTYLEWYLQKPGQSPKLLICKVSTRFSGVPDRFSGSGSGTDFTLKISRVEAEDLGVYYCFQGSHVPYTFGGGTKLEIK

SEQ ID NO: 157 - protein, 16 aa, Mus musculus
  MISC_FEATURE (1)..(16): CDRL1 of PD-1 mAb 12
RSSQNIVHSNGNTYLE

SEQ ID NO: 158 - protein, 7 aa, Mus musculus
  MISC_FEATURE (1)..(7): CDRL2 of PD-1 mAb 12
KVSTRFS

SEQ ID NO: 159 - protein, 9 aa, Mus musculus
  MISC_FEATURE (1)..(9): CDRL3 of PD-1 mAb 12
FQGSHVPYT

SEQ ID NO: 160 - protein, 121 aa, Mus musculus
  MISC_FEATURE (1)..(121): VH Domain of Murine Anti-Human PD-1 mAb 13
EVMLVESGGGLVKPGGSLKLSCAASGFTFSSHTMSWVRQTPEKRLEWVATISGGGSNIYYPDSVKGRFTISRDNAKNTLYLQMSSLRSEDTALYYCARQAYYGNYWYFDVWGTGTTVTVSS

SEQ ID NO: 161 - protein, 5 aa, Mus musculus
  MISC_FEATURE (1)..(5): CDRH1 of PD-1 mAb 13
SHTMS

SEQ ID NO: 162 - protein, 17 aa, Mus musculus
  MISC_FEATURE (1)..(17): CDRH2 of PD-1 mAb 13
TISGGGSNIYYPDSVKG

SEQ ID NO: 163 - protein, 12 aa, Mus musculus
  MISC_FEATURE (1)..(12): CDRH3 of PD-1 mAb 13
QAYYGNYWYFDV

SEQ ID NO: 164 - protein, 107 aa, Mus musculus
  MISC_FEATURE (1)..(107): VL Domain of Murine Anti-Human PD-1 mAb 13
DIQMTQSPATQSASLGESVTITCLASQTIGTWLAWYQQKPGKSPQLLIYAATSLADGVPSRFSGSGSGTKFSFKISSLQAEDFVSYYCQQLDSIPWTFGGGTKLEIK

SEQ ID NO: 165 - protein, 11 aa, Mus musculus
  MISC_FEATURE (1)..(11): CDRL1 of PD-1 mAb 13
LASQTIGTWLA

SEQ ID NO: 166 - protein, 7 aa, Mus musculus
  MISC_FEATURE (1)..(7): CDRL2 of PD-1 mAb 13
AATSLAD

SEQ ID NO: 167 - protein, 9 aa, Mus musculus
  MISC_FEATURE (1)..(9): CDRL3 of PD-1 mAb 13
QQLDSIPWT

SEQ ID NO: 168 - protein, 117 aa, Mus musculus
  MISC_FEATURE (1)..(117): VH Domain of Murine Anti-Human PD-1 mAb 14
QVQLQQPGAELVKPGASVKMSCKASGYNFISYWITWVKQRPGQGLQWIGNIYPGTDGTTYNEKFKSKATLTVDTSSSTAYMHLSRLTSEDSAVYYCATGLHWYFDVWGTGTTVTVSS

SEQ ID NO: 169 - protein, 5 aa, Mus musculus
  MISC_FEATURE (1)..(5): CDRH1 of PD-1 mAb 14
SYWIT

SEQ ID NO: 170 - protein, 17 aa, Mus musculus
  MISC_FEATURE (1)..(17): CDRH2 of PD-1 mAb 14
NIYPGTDGTTYNEKFKS

SEQ ID NO: 171 - protein, 8 aa, Mus musculus
  MISC_FEATURE (1)..(8): CDRH3 of PD-1 mAb 14
GLHWYFDV

SEQ ID NO: 172 - protein, 107 aa, Mus musculus
  MISC_FEATURE (1)..(107): VL Domain of Murine Anti-Human PD-1 mAb 14
DIVMTQSQKFMSTSVGDRVSVTCKASQSVGTNVAWYQQKPGQSPKALIYSASSRFSGVPDRFTGSGSGTDFTLTISNVQSEDLAEYFCQQYNSYPYTFGGGTKLEIK

SEQ ID NO: 173 - protein, 11 aa, Mus musculus
  MISC_FEATURE (1)..(11): CDRL1 of PD-1 mAb 14
KASQSVGTNVA

SEQ ID NO: 174 - protein, 7 aa, Mus musculus
  MISC_FEATURE (1)..(7): CDRL2 of PD-1 mAb 14
SASSRFS

SEQ ID NO: 175 - protein, 9 aa, Mus musculus
  MISC_FEATURE (1)..(9): CDRL3 of PD-1 mAb 14
QQYNSYPYT

SEQ ID NO: 176 - protein, 117 aa, Mus musculus
  MISC_FEATURE (1)..(117): VH Domain of Murine Anti-Human PD-1 mAb 15
EVMLVESGGGLVKPGGSLKLSCAASGFIFSSYLISWVRQTPEKRLEWVAAISGGGADTYYADSVKGRFTISRDNAKNTLYLQMSSLRSEDTALYYCTRRGTYAMDYWGQGTSVTVSS

SEQ ID NO: 177 - protein, 5 aa, Mus musculus
  MISC_FEATURE (1)..(5): CDRH1 of PD-1 mAb 15
SYLIS

SEQ ID NO: 178 - protein, 17 aa, Mus musculus
  MISC_FEATURE (1)..(17): CDRH2 of PD-1 mAb 15
AISGGGADTYYADSVKG

SEQ ID NO: 179 - protein, 8 aa, Mus musculus
  MISC_FEATURE (1)..(8): CDRH3 of PD-1 mAb 15
RGTYAMDY

SEQ ID NO: 180 - protein, 107 aa, Mus musculus
  MISC_FEATURE (1)..(107): VL Domain of Murine Anti-Human PD-1 mAb 15
DIQMTQSPASQSASLGESVTITCLASQTIGTWLAWYQQKPGKSPQLLIYAATSLADGVPSRFSGSGSGTKFSFKISSLQAEDFVNYYCQQLYSIPWTFGGGTKLEIK

SEQ ID NO: 181 - protein, 11 aa, Mus musculus
  MISC_FEATURE (1)..(11): CDRL1 of PD-1 mAb 15
LASQTIGTWLA

SEQ ID NO: 182 - protein, 7 aa, Mus musculus
  MISC_FEATURE (1)..(7): CDRL2 of PD-1 mAb 15
AATSLAD

SEQ ID NO: 183 - protein, 9 aa, Mus musculus
  MISC_FEATURE (1)..(9): CDRL3 of PD-1 mAb 15
QQLYSIPWT

SEQ ID NO: 184 - protein, 117 aa, Artificial Sequence
  VH Domain of Humanized Anti-Human hPD-1 mAb 15 VH1
EVQLVESGGGLVRPGGSLRLSCAASGFTFSSYLISWVRQAPGKGLEWVAAISGGGADTYYADSVKGRFTISRDNAKNSLYLQMNSLRAEDTATYYCARRGTYAMDYWGQGTLVTVSS

SEQ ID NO: 185 - protein, 107 aa, Artificial Sequence
  VL Domain of Humanized Anti-Human hPD-1 mAb 15 VL1
DIQMTQSPSSLSASVGDRVTITCLASQTIGTWLAWYQQKPGKAPKLLIYAATSLADGVPSRFSGSGSGTDFTFTISSLQPEDFATYYCQQLYSIPWTFGQGTKLEIK

SEQ ID NO: 186 - protein, 445 aa, Artificial Sequence
  Heavy Chain of Humanized Anti-Human PD-1 Antibody hPD-1 mAb7 (1.2) IgG4 (P)
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 187 - protein, 218 aa, Artificial Sequence
  Light Chain of Humanized Anti-Human PD-1 Antibody hPD-1 mAb7 (1.2) IgG4 (P)
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 176 aa, Homo sapiens
  MISC_FEATURE (1)..(176): Human B7-H1 (PD-L1) Polypeptide (NCBI Sequence NP_001254635.1), Including  Predicted 18 Amino Acid Signal Sequence
  MISC_FEATURE (1)..(18): Predicted Signal Sequence
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 189 - protein, 273 aa, Homo sapiens
  MISC_FEATURE (1)..(273): Human B7-DC (PD-L2) Polypeptide (NCBI Sequence NP_079515.2); Including  Predicted 18 Amino Acid Signal Sequence
  MISC_FEATURE (1)..(18): Predicted Signal Sequence
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 190 - protein, 118 aa, Mus musculus
  MISC_FEATURE (1)..(118): VH Domain of Anti-Human CD2 Antibody CD2 mAb Lo-CD2a
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 191 - protein, 112 aa, Mus musculus
  MISC_FEATURE (1)..(112): VL Domain of Anti-Human CD2 Antibody CD2 mAb Lo-CD2a
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 192 - protein, 125 aa, Mus musculus
  MISC_FEATURE (1)..(125): VH Domain of Anti-Human CD3 Antibody CD3 mAb 1
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 193 - protein, 110 aa, Mus musculus
  MISC_FEATURE (1)..(110): VL Domain of Anti-Human CD3 Antibody CD3 mAb 1
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 194 - protein, 125 aa, Mus musculus
  MISC_FEATURE (1)..(125): VH Domain of Anti-Human CD3 Antibody CD3 mAb 1 (D65G)
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 195 - protein, 125 aa, Mus musculus
  MISC_FEATURE (1)..(125): VH Domain of Anti-Human CD3 Antibody CD3 mAb 1 Low
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSS

SEQ ID NO: 196 - protein, 125 aa, Mus musculus
  MISC_FEATURE (1)..(126): VH Domain of Anti-Human CD3 Antibody CD3 mAb 1 Fast
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSS

SEQ ID NO: 197 - protein, 119 aa, Mus musculus [sequence omitted]

SEQ ID NO: 198 - protein, 107 aa, Mus musculus
  MISC_FEATURE (1)..(107): VL Domain of Anti-Human CD3 Antibody OKT3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 199 - protein, 120 aa, Mus musculus
  MISC_FEATURE (1)..(120): VH Domain of Anti-Human CD8 Antibody OKT8
QVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSS

SEQ ID NO: 200 - protein, 112 aa, Mus musculus
  MISC_FEATURE (1)..(112): VL Domain of Anti-Human CD8 Antibody OKT8
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKR

SEQ ID NO: 201 - protein, 121 aa, Mus musculus
  MISC_FEATURE (1)..(121): VH Domain of Anti-Human CD8 Antibody TRX2
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 202 - protein, 106 aa, Mus musculus
  MISC_FEATURE (1)..(106): VL Domain of Anti-Human CD8 Antibody TRX2
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 203 - protein, 118 aa, Mus musculus
  MISC_FEATURE (1)..(118): VH Domain of Anti-Human CD16 Antibody 3G8
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 204 - protein, 111 aa, Mus musculus
  MISC_FEATURE (1)..(111): VL Domain of Anti-Human CD16 Antibody 3G8
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 205 - protein, 117 aa, Mus musculus
  MISC_FEATURE (1)..(117): VH Domain of Anti-Human CD16 Antibody A9
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSS

SEQ ID NO: 206 - protein, 111 aa, Mus musculus
  MISC_FEATURE (1)..(111): VL Domain of Anti-Human CD16 Antibody A9
DIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

SEQ ID NO: 207 - protein, 120 aa, Mus musculus
  MISC_FEATURE (1)..(129): VH Domain of Anti-Human TCR Antibody BMA 031
QVQLVQSGAEVKKPGASVKVSCKASGYKFTSYVMHWVRQAPGQGLEWIGYINPYNDVTKYNEKFKGRVTITADKSTSTAYLQMNSLRSEDTAVHYCARGSYYDYDGFVYWGQGTLVTVSS

SEQ ID NO: 208 - protein, 106 aa, Mus musculus
  MISC_FEATURE (1)..(106): VL Domain of Anti-Human TCR Antibody BMA 031
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 209 - protein, 118 aa, Mus musculus
  MISC_FEATURE (1)..(118): VH Domain of Anti-Human NKG2D Antibody KYK-1.0
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 210 - protein, 108 aa, Mus musculus
  MISC_FEATURE (1)..(108): VL Domain of Anti-Human NKG2D Antibody KYK-1.0
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 211 - protein, 121 aa, Mus musculus
  MISC_FEATURE (1)..(121): VH Domain of Anti-Human NKG2D Antibody KYK-2.0
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 212 - protein, 110 aa, Mus musculus
  MISC_FEATURE (1)..(110): VL Domain of Anti-Human NKG2D Antibody KYK-2.0
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 213 - protein, 120 aa, Mus musculus
  MISC_FEATURE (1)..(120): VH Domain of Anti-Human B7-H3 Antibody B7-H3 mAb 1
QVQLQQSGAELARPGASVKLSCKASGYTFTSYWMQWVKQRPGQGLEWIGTIYPGDGDTRYTQKFKGKATLTADKSSSTAYMQLSSLASEDSAVYYCARRGIPRLWYFDVWGAGTTVTVSS

SEQ ID NO: 214 - protein, 107 aa, Mus musculus
  MISC_FEATURE (1)..(107): VL Domain of Anti-Human B7-H3 Antibody B7-H3 mAb 1
DIQMTQTTSSLSASLGDRVTISCRASQDISNYLNWYQQKPDGTVKLLIYYTSRLHSGVPSRFSGSGSGTDYSLTIDNLEQEDIATYFCQQGNTLPPTFGGGTKLEIK

SEQ ID NO: 215 - protein, 120 aa, Mus musculus
  MISC_FEATURE (1)..(120): VH Domain of Humanized Anti-Human B7-H3 Antibody hB7-H3 mAb 1 VH1
QVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSS

SEQ ID NO: 216 - protein, 120 aa, Mus musculus
  MISC_FEATURE (1)..(120): VH Domain of Humanized Anti-Human B7-H3 Antibody hB7-H3 mAb 1 VH2
QVQLVQSGAEVKKPGASVKVSCKASGYTFTSYWMQWVRQAPGQGLEWMGTIYPGGGDTRYTQKFQGRVTITADKSTSTAYMELSSLRSEDTAVYYCARRGIPRLWYFDVWGQGTTVTVSS

SEQ ID NO: 217 - protein, 107 aa, Mus musculus
  MISC_FEATURE (1)..(107): VL Domain of Humanized Anti-Human B7-H3 Antibody hB7-H3 mAb 1 VL1
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIK

SEQ ID NO: 218 - protein, 107 aa, Mus musculus
  MISC_FEATURE (1)..(107): VL Domain of Humanized Anti-Human B7-H3 Antibody hB7-H3 mAb 1 VL2
DIQMTQSPSSLSASVGDRVTITCRASQSISSYLNWYQQKPGKAPKLLIYYTSRLQSGVPSRFSGSGSGTDFTLTISSLQPEDIATYYCQQGNTLPPTFGGGTKLEIK

SEQ ID NO: 219 - protein, 122 aa, Mus musculus
  MISC_FEATURE (1)..(122): VH Domain of Anti-Human B7-H3 Antibody B7-H3 mAb 2
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 220 - protein, 107 aa, Mus musculus
  MISC_FEATURE (1)..(107): VL Domain of Anti-Human B7-H3 Antibody B7-H3 mAb 2
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 221 - protein, 122 aa, Artificial Sequence
  VH Domain of Humanized Anti-Human B7-H3 Antibody hB7-H3 mAb 2 VH1
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCARGRENIYYGSRLDYWGQGTTVTVSS

SEQ ID NO: 222 - protein, 122 aa, Artificial Sequence
  VH Domain of Humanized Anti-Human B7-H3 Antibody hB7-H3 mAb 2 VH2
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 223 - protein, 122 aa, Artificial Sequence
  VH Domain of Humanized Anti-Human B7-H3 Antibody hB7-H3 mAb 2 VH3
EVQLVESGGGLVQPGGSLRLSCAASGFTFSSFGMHWVRQAPGKGLEWVAYISSDSSAIYYADTVKGRFTISRDNAKNSLYLQMNSLRDEDTAMYYCGRGRENIYYGSRLDYWGQGTTVTVSS

SEQ ID NO: 224 - protein, 122 aa, Artificial Sequence
  VH Domain of Humanized Anti-Human B7-H3 Antibody hB7-H3 mAb 2 VH4
EVQLVESGGGLVQPGGSLRLSCAASGFTFSSFGMHWVRQAPGKGLEWVAYISSDSSAIYYADTVKGRFTISRDNAKNSLYLQMNSLRSEDTAVYYCARGRENIYYGSRLDYWGQGTTVTVSS

SEQ ID NO: 225 - protein, 107 aa, Artificial Sequence
  VL Domain of Humanized Anti-Human B7-H3 Antibody hB7-H3 mAb 2 VL1
DIQLTQSPSFLSASVGDRVTITCKASQNVDTNVAWYQQKPGKAPKLLIYSASYRYSGVPSRFSGSGSGTDFTLTISSLQPEDFATYYCQQYNNYPFTFGQGTKLEIK

SEQ ID NO: 226 - protein, 107 aa, Artificial Sequence
  VL Domain of Humanized Anti-Human B7-H3 Antibody hB7-H3 mAb 2 VL2
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 227 - protein, 107 aa, Artificial Sequence
  VL Domain of Humanized Anti-Human B7-H3 Antibody hB7-H3 mAb 2 VL3
DIQLTQSPSFLSASVGDRVSVTCKASQNVDTNVAWYQQKPGKAPKLLIYSASYRYSGVPSRFSGSGSGTDFTLTISSLQPEDFATYYCQQYNNYPFTFGQGTKLEIK

SEQ ID NO: 228 - protein, 107 aa, Artificial Sequence
  VL Domain of Humanized Anti-Human B7-H3 Antibody hB7-H3 mAb 2 VL4
DIQLTQSPSFLSASVGDRVTITCKASQNVDTNVAWYQQKPGQAPKLLIYSASYRYSGVPSRFSGSGSGTDFTLTISSLQPEDFATYYCQQYNNYPFTFGQGTKLEIK

SEQ ID NO: 229 - protein, 107 aa, Artificial Sequence
  VL Domain of Humanized Anti-Human B7-H3 Antibody hB7-H3 mAb 2 VL5
DIQLTQSPSFLSASVGDRVTITCKASQNVDTNVAWYQQKPGQAPKALIYSASYRYSGVPSRFSGSGSGTDFTLTISSLQPEDFATYYCQQYNNYPFTFGQGTKLEIK

SEQ ID NO: 230 - protein, 107 aa, Artificial Sequence
  VL Domain of Humanized Anti-Human B7-H3 Antibody hB7-H3 mAb 2 VL6
DIQLTQSPSFLSASVGDRVTITCKASQNVDTNVAWYQQKPGKAPKLLIYSASYRYSGVPSRFSGSGSGTDFTLTISSLQPEDFAEYYCQQYNNYPFTFGQGTKLEIK

SEQ ID NO: 231 - protein, 117 aa, Mus musculus
  MISC_FEATURE (1)..(117): VH Domain of Anti-Human B7-H3 Antibody B7-H3 mAb 3
EVQQVESGGDLVKPGGSLKLSCAASGFTFSSYGMSWVRQTPDKRLEWVATINSGGSNTYYPDSLKGRFTISRDNAKNTLYLQMRSLKSEDTAMYYCARHDGGAMDYWGQGTSVTVSS

SEQ ID NO: 232 - protein, 108 aa, Mus musculus
  MISC_FEATURE (1)..(108): VL Domain of Anti-Human B7-H3 Antibody B7-H3 mAb 3
DIQMTQSPASLSVSVGETVTITCRASESIYSYLAWYQQKQGKSPQLLVYNTKTLPEGVPSRFSGSGSGTQFSLKINSLQPEDFGRYYCQHHYGTPPWTFGGGTNLEIK

SEQ ID NO: 233 - protein, 121 aa, Artificial Sequence
  VH Domain of Humanized Anti-Human CEACAM5 / ANTI-CEACAM6 Antibody 16C3
QVQLQQSGPEVVRPGVSVKISCKGSGYTFTDYAMHWVKQSHAKSLEWIGLISTYSGDTKYNQNFKGKATMTVDKSASTAYMELSSLRSEDTAVYYCARGDYSGSRYWFAYWGQGTLVTVSS

SEQ ID NO: 234 - protein, 107 aa, Artificial Sequence
  VL Domain of Humanized Anti-Human CEACAM5 / ANTI-CEACAM6 Antibody 16C3
DIQMTQSPSSLSASVGDRVTITCGASENIYGALNWYQRKPGKSPKLLIWGASNLADGMPSRFSGSGSGRQYTLTISSLQPEDVATYYCQNVLSSPYTFGGGTKLEIK

SEQ ID NO: 235 - protein, 121 aa, Artificial Sequence
  VH Domain of Humanized Anti-Human CEACAM5 / ANTI-CEACAM6 Antibody hMN15
QVQLVESGGGVVQPGRSLRLSCSSSGFALTDYYMSWVRQAPGKGLEWLGFIANKANGHTTDYSPSVKGRFTISRDNSKNTLFLQMDSLRPEDTGVYFCARDMGIRWNFDVWGQGTPVTVSS

SEQ ID NO: 236 - protein, 107 aa, Artificial Sequence
  VL Domain of Humanized Anti-Human CEACAM5 / ANTI-CEACAM6 Antibody hMN15
DIQLTQSPSSLSASVGDRVTMTCSASSRVSYIHWYQQKPGKAPKRWIYGTSTLASGVPARFSGSGSGTDFTFTISSLQPEDIATYYCQQWSYNPPTFGQGTKVEIKR

SEQ ID NO: 237 - protein, 119 aa, Artificial Sequence
  VH Domain of Chimeric Anti-Human EGFR Antibody “Cetuximab”
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 238 - protein, 108 aa, Artificial Sequence
  VL Domain of Chimeric Anti-Human EGFR Antibody “Cetuximab”
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 239 - protein, 119 aa, Artificial Sequence [sequence omitted]

SEQ ID NO: 240 - protein, 108 aa, Artificial Sequence
  VL Domain of Humanized Anti-Human EGFR Antibody “Panitumumab”
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 241 - protein, 118 aa, Mus musculus
  MISC_FEATURE (1)..(118): VH Domain of Anti-Human EphA2 Antibody EphA2 mAb 1
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARKHGNYYTMDYWGQGTSVTVSS

SEQ ID NO: 242 - protein, 106 aa, Mus musculus
  MISC_FEATURE (1)..(106): VL Domain of Anti-Human EphA2 Antibody EphA2 mAb 1
DIQMTQTTSSLSASLGDRITISCRASQDISNYLNWYQQKPDGTVKLLIYYTSRLHSGVPSRFSGSGSGTDYSLTISNLEQEDIATYFCQQGYTLYTFGGGTKLEIK

SEQ ID NO: 243 - protein, 118 aa, Mus musculus
  MISC_FEATURE (1)..(118): VH Domain of Anti-Human EphA2 Antibody EphA2 mAb 2
QIQLVQSGPELKKPGETVKISCKASGFTFTNYGMNWVKQAPGKGLKWMGWINTYIGEPTYADDFKGRFVFSLETSASTAYLQINNLKNEDMATYFCARELGPYYFDYWGQGTTLTVSS

SEQ ID NO: 244 - protein, 111 aa, Mus musculus
  MISC_FEATURE (1)..(111): VL Domain of Anti-Human EphA2 Antibody EphA2 mAb 2
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPTFGSGTKLEIK

SEQ ID NO: 245 - protein, 118 aa, Mus musculus
  MISC_FEATURE (1)..(118): VH Domain of Anti-Human EphA2 Antibody EphA2 mAb 3
EVQLVESGGGSVKPGGSLKLSCAASGFTFTDHYMYWVRQTPEKRLEWVATISDGGSFTSYPDSVKGRFTISRDIAKNNLYLQMSSLKSEDTAMYYCTRDESDRPFPYWGQGTLVTVSS

SEQ ID NO: 246 - protein, 107 aa, Mus musculus
  MISC_FEATURE (1)..(107): VL Domain of Anti-Human EphA2 Antibody EphA2 mAb 3
DIVLTQSHRSMSTSVGDRVNITCKASQDVTTAVAWYQQKPGQSPKLLIFWASTRHAGVPDRFTGSGSGTDFTLTISSVQAGDLALYYCQQHYSTPYTFGGGTKLEIK

SEQ ID NO: 247 - protein, 119 aa, Mus musculus
  MISC_FEATURE (1)..(119): VH Domain of Anti-Human gpA33 Antibody gpA33 mAb 1
QVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSS

SEQ ID NO: 248 - protein, 106 aa, Mus musculus
  MISC_FEATURE (1)..(106): VL Domain of Anti-Human gpA33 Antibody gpA33 mAb 1
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIK

SEQ ID NO: 249 - protein, 120 aa, Artificial Sequence
  VH Domain of Affinity-Optimized Anti-Human Her2/Neu Antibody Margituximab
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 250 - protein, 107 aa, Artificial Sequence
  VL Domain of Affinity Optimized Anti-Human Her2/Neu Antibody Margituximab
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 251 - protein, 120 aa, Artificial Sequence [sequence omitted]

SEQ ID NO: 252 - protein, 107 aa, Artificial Sequence [sequence omitted]

SEQ ID NO: 253 - protein, 119 aa, Artificial Sequence [sequence omitted]

SEQ ID NO: 254 - protein, 107 aa, Artificial Sequence [sequence omitted]

SEQ ID NO: 255 - protein, 123 aa, Artificial Sequence [sequence omitted]

SEQ ID NO: 256 - protein, 108 aa, Artificial Sequence
  VL Domain of Humanized Anti-Human VEGF Antibody Bevacizumab
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 257 - protein, 118 aa, Mus musculus
  MISC_FEATURE (1)..(118): VH Domain of Anti-Human 5T4 Antibody 5T4 mAb 1
QVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSS

SEQ ID NO: 258 - protein, 107 aa, Mus musculus
  MISC_FEATURE (1)..(107): VL Domain of Anti-Human 5T4 Antibody 5T4 mAb 1
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIK

SEQ ID NO: 259 - protein, 127 aa, Mus musculus
  MISC_FEATURE (1)..(127): VH Domain of Anti-Human 5T4 Antibody 5T4 mAb 2
QVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSS

SEQ ID NO: 260 - protein, 112 aa, Mus musculus
  MISC_FEATURE (1)..(112): VL Domain of Anti-Human 5T4 Antibody 5T4 mAb 2
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 261 - protein, 122 aa, Mus musculus
  MISC_FEATURE (1)..(122): VH Domain of Anti-Human IL13Ra2 Antibody hu08
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSS

SEQ ID NO: 262 - protein, 107 aa, Mus musculus
  MISC_FEATURE (1)..(107): VL Domain of Anti-Human IL13Ra2 Antibody hu08
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIK

SEQ ID NO: 263 - protein, 120 aa, Mus musculus
  MISC_FEATURE (1)..(120): VH Domain of Anti-Human CD123 Antibody CD123 mAb 1
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 264 - protein, 113 aa, Mus musculus
  MISC_FEATURE (1)..(113): VL Domain of Anti-Human CD123 Antibody CD123 mAb 1
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 265 - protein, 120 aa, Mus musculus
  MISC_FEATURE (1)..(120): VH Domain of Anti-Human CD19 Antibody CD19 mAb 1
QVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSS

SEQ ID NO: 266 - protein, 106 aa, Mus musculus
  MISC_FEATURE (1)..(106): VL Domain of Anti-Human CD19 Antibody CD19 mAb 1
ENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIK

SEQ ID NO: 267 - protein, 126 aa, Mus musculus
  MISC_FEATURE (1)..(126): VH Domain of Anti-HIV env Antibody 7B2
QVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSS

SEQ ID NO: 268 - protein, 113 aa, Mus musculus
  MISC_FEATURE (1)..(113): VL Domain of Anti-HIV env Antibody 7B2
DIVMTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVEIK

SEQ ID NO: 269 - protein, 118 aa, Mus musculus
  MISC_FEATURE (1)..(118): VH Domain of Anti-HIV env Antibody h3G8
QVTLRESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSS

SEQ ID NO: 270 - protein, 111 aa, Mus musculus
  MISC_FEATURE (1)..(111): VL Domain of Anti-HIV env Antibody h3G8
DIVMTQSPDSLAVSLGERATINCKASQSVDFDGDSFMNWYQQKPGQPPKLLIYTTSNLESGVPDRFSGSGSGTDFTLTISSLQAEDVAVYYCQQSNEDPYTFGQGTKLEIK

SEQ ID NO: 271 - protein, 504 aa, Artificial Sequence
  First Polypeptide Chain of DART-A
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 274 aa, Artificial Sequence
  Second Polypeptide Chain od DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGCGGGKVAALKEKVAALKEKVAALKEKVAALKE

SEQ ID NO: 273 - protein, 227 aa, Artificial Sequence
  Third Polypeptide Chain of DART-A
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 274 - protein, 496 aa, Artificial Sequence
  First and Third Polypeptide Chains of DART-1
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 275 - protein, 271 aa, Artificial Sequence
  Second and Fourth Polypeptide Chains of DART-1
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE